(19) United States
(12) Patent Application Publication
(10) Pub. No.: US 2009/0186073 A1
(43) Pub. Date: Jul. 23, 2009

(54) Design of Delivery Vehicle Based On Rolling Model

(75) Inventors: Noboru Yamazaki, Ibaraki (JP); Shuuji Kojima, Ibaraki (JP)

Correspondence Address: SUGHRUE MION, PLLC, 2100 PENNSYLVANIA AVENUE, N.W., SUITE 800, WASHINGTON, DC, 20037, US

(73) Assignees: National Institute of Advanced Industrial Science and Technology, Tokyo (JP); Yamazaki- DDS Co., Ltd., Ibaraki (JP)

(21) Appl. No.: 11/990,300

(22) PCT Filed: Aug. 10, 2006
(86) PCT No.: PCT/JP2006/315885

§ 371 Date: Feb. 11, 2008

(30) Foreign Application Priority Data
Aug. 11, 2005 (JP) 2005-233829
Jul. 21, 2006 (JP) 2006-200093

(51) Int. Cl. A61K 9/127; A61P 43/00

(52) U.S. Cl. 424/450

(57) ABSTRACT

Objects of the present invention are to provide a method for designing an optimum delivery preparation with the use of a convenient experiment and/or assay and to search for the thus produced delivery vehicle. The present invention provides a method for producing a delivery vehicle for achieving the delivery of a desired substance to a desired site, which comprises the steps of:
A) measuring in vitro affinity of candidate delivery vehicles for a cell surface molecule such as a lectin associated with the site; and
B) selecting a delivery vehicle having in vitro affinity corresponding to delivery to the desired site.

### TECHNICAL FIELD

The present invention relates to the design of a remedy comprising an optimum probe that is selected via in vitro experiments from among probes that can be used as drug delivery systems for treatment (DDSs are used for recognizing target cells and/or tissues such as cancer and then locally delivering drugs or genes to affected parts) or as cell- and/or tissue-sensing probes for diagnosis, a sugar-chain-modified liposome produced with the use of such probe, and a liposome formulation in which a drug, a gene, or the like is encapsulated, which are applicable to medical and/or pharmaceutical fields in addition to the fields of pharmaceutical products and cosmetics.

### BACKGROUND ART

The National Nanotechnology Initiative (NNI) of the U.S.A. has set up “a drug or gene delivery system (DDS: drug delivery system) for taking a shot at cancer cells or target tissues,” as an example of a specific goal for realization. The Council for Science and Technology Policy in Japan has set up “ultra small system material for medical use and nanobiology for using and controlling biological mechanisms” as a prioritized area of the Promotion Strategy for Nanotechnology and Materials Area. One of the research and development objectives of the Council over the next 5 years is “Establishment of Basic Seeds for Technology Including Biofunctional Materials, Pinpoint Treatment, and the like for Extending Healthy Life Spans.” Meanwhile, incidences and mortality of cancer are increasing yearly as the aging society emerges. Development of a targeting DDS that is a novel material for treatment is expected. Importance of targeting DDS nanomaterials without side effects has attracted attention in regards to other diseases. The size of the market is expected to exceed 10 trillion yen in the near future. Moreover, these materials are also expected to be used for diagnosis in addition to treatment.

A therapeutic effect of a pharmaceutical product is achieved when the drug reaches a specific target site and then acts on the site. On the other hand, a side effect resulting from a pharmaceutical product means that a drug acts on undesired sites. Therefore, development of drug delivery systems is desired for effective and safe use of drugs. In particular, a targeting DDS is a concept whereby a drug is delivered to “in vivo sites that need treatment,” “in a necessary amount,” “for a required time length.” Liposomes, which are microparticulate carriers, have attracted attention as a typical material for use in such purposes. Passive targeting methods that involve varying lipid type, composition ratio, particle diameter, and surface charge of liposomes have been attempted to impart targeting functions to such particles. However, these methods are still insufficient and need further improvement.

Meanwhile, an active targeting method has also been attempted to enable highly-functional targeting. This method is also referred to as the “missile drug” and is an ideal targeting method. This method has not yet been completed either at home or abroad, and the future development thereof is greatly expected. This method comprises binding a ligand onto a liposome membrane surface, so as to cause a receptor existing on the cell membrane surfaces of a target tissue to specifically recognize the ligand. Active targeting is made possible by the use of this method. Possible ligands for such receptors existing on cell membrane surfaces to be targeted by the active targeting method are antigens, antibodies, peptides, glycolipids, glycoproteins, and the like. Of these, the fact that sugar chains of glycolipids or glycoproteins play important roles as information molecules in various cell-to-cell communications such as development or morphological formation of living body tissue, cell proliferation or differentiation, biophylaxis, the fertilization mechanism, canceration, and the metastasis mechanism thereof is being clarified.

Furthermore, research has advanced concerning various lectins (sugar-chain-recognizing proteins) such as selectin, siglec, and galectin as receptors existing on the cell membrane surfaces of each target tissue. Sugar chains having various molecular structures are attracting attention as new DDS ligands (see Yamazaki, N., Kojima, S., Bovin, N. V., Andre, S., Gabius, S, and Gabius, H.-J. (2000) Adv. Drug Delivery Rev. 43, 225-244; and Yamazaki, N., Jigami, Y., Gabius, H.-J., Kojima, S. (2001) Trends in Glycoscience and Glycotechnology 13, 319-329. http://www.gak.co.jp/TIGG/71 PDF/yamazaki.pdf).

Many studies have been conducted on liposomes (ligands bind onto the outer membrane surfaces of liposomes) as DDS materials for use in selective delivery of drugs or genes to a desired site, such as a cancer. However, most of these liposomes bind to target cells ex vivo, but are not targeted in vivo to expected target cells or tissues (see Forssen, E. and Willis, M. (1998) Adv. Drug Delivery Rev. 29, 249-271; and Edited by Toshio Takahashi-Mitsuru Hashida (1999), Today's DDS-drug delivery system, pp. 159-167, Iyaku (Medicine and Drug) Journal Co., Ltd., Osaka, Japan)). Also, in research and development concerning DDS materials using the molecular recognition functions of sugar chains, some studies concerning liposomes in which glycolipids having sugar chains have been introduced are known. The functional evaluation of these liposomes has been made ex vivo (in vitro) alone. Studies concerning liposomes into which glycoproteins having sugar chains are introduced have remained almost entirely unadvanced (see DeFrees, S. A., Phillips, L., Guo, L. and Zalipsky, S. (1996) J. Am. Chem. Soc. 118, 6101-6104; Spevak, W., Foxall, C., Charych, D. H., Dasqupta, F. and Nagy, J. O. (1996) J. Med. Chem. 39, 1018-1020; Stahn, R., Schafer, H., Kernchen, F. and Schreiber, J. (1998) Glycobiology 8, 311-319; and Yamazaki, N., Jigami, Y., Gabius, H.-J., Kojima, S. (2001) Trends in Glycoscience and Glycotechnology 13, 319-329. http://www.gak.co.jp/TIGG/71PDF/yamazaki.pdf). Therefore, systematic studies including methods for preparing liposomes to which a wide variety of sugar chains of glycolipids or glycoproteins are bound and in vivo kinetics (in vivo) analysis are important issues that have not been developed and are expected to be advanced in the future. As a study concerning a further new type of DDS material, development of a DDS material that can be used via oral administration, by which administration can be performed most conveniently at low cost, is also an important issue. For example, a peptidic pharmaceutical product is characterized by being generally water soluble, having a high molecular weight, and having low mucosal permeability in the alimentary canal (small intestine). Hence, such a product is digested by an enzyme, or the like, so that the product is almost never intestinally absorbed, even when it is administered orally. A study concerning ligand-bound liposomes is thus attracting attention, regarding a DDS material for delivery of high-molecular-weight pharmaceutical products, genes, or the like into blood from the intestinal tract (see Lehr, C.-M. (2000) J. Controlled Release 65, 19-29).

JP Patent Publication (Kohyo) No. 5-507519 A (1993) discloses a pharmaceutical composition having a pharmaceutically acceptable carrier and a compound that contains an ingredient selectively binding to a selectin receptor. However, in this pharmaceutical composition, a sugar chain is used for the purpose of oral administration as a remedy itself for inhibiting inflammatory disease and other diseases mediated by cell adhesion. Thus, the pharmaceutical composition is different from a sugar-chain-modified liposome.

JP Patent Publication (Kohyo) No. 2004-517835 A discloses a pharmaceutical composition for parenteral administration, which comprises a liposome comprising a non-charged vesicle-forming lipid that contains a polyethylene glycol derivatization amphiphilic vesicle-forming lipid and a negatively charged vesicle-forming lipid. However, this pharmaceutical composition is used for parenteral administration and thus is different from the sugar-chain-modified liposome of the present invention, which is particularly suitable for oral administration. This patent publication has no descriptions concerning sugar chains.

The present inventors have developed a sugar-chain-modified liposome to which a sugar chain is bound via a linker protein (JP Patent Publication (Kokai) No. 2003-226638 A). Moreover, the present inventors have also discovered that the type and binding amount of a sugar chain may be involved in tropism for each target cell or target tissue (JP Patent Publication (Kokai) No. 2003-226647 A; International Publication No. 2005/011632 Pamphlet; International Publication No. 2005/011633 Pamphlet; and Noboru Yamazaki (2005), Development of Active Targeting DDS Nanoparticle, Bulletin of the Society of Nano Science and Technology, 3, 97-102). However, to date, no optimum sugar-chain-modified liposome that can be used in various applications has been developed. Moreover, no systematic studies have been conducted concerning sugar chains useful for administration via various routes. It remains unknown about specifically what kind of sugar chain should be used.

Therefore, there is a demand for a method for designing an optimum delivery preparation with the use of a convenient experiment and assay.

### DISCLOSURE OF THE INVENTION

### Objects to be Achieved by the Invention

The objects of the present invention are to provide a method for designing an optimum delivery preparation with the use of a convenient experiment and/or assay and to search for the thus produced delivery vehicle.

### Means to Achieve the Objects

As a result of intensive studies to achieve the above objects, the present inventors have discovered that a delivery vehicle (hereinafter, also referred to as rolling model) that is useful in vivo can be produced with a constant probability based on results obtained by processing results obtained with the use of an in vitro assay system via specific information processing. Hence, the present inventors have achieved the above objects.

Therefore, the present invention provides the following (1) to (111).

(1) A delivery vehicle for achieving delivery to a desired site, in which binding takes place based on strong binding or weak binding to a cell surface molecule associated with a desired site.
(2) A delivery vehicle for achieving delivery to a desired site, in which binding to a cell surface molecule associated with a desired site is based on a rolling model.
(3) A delivery vehicle for achieving delivery to a desired site, in which a strong binding inhibitory concentration (IC) involved in in vitro affinity is low for a cell surface molecule associated with a desired site.
(4) A delivery vehicle for achieving delivery to a desired site, in which a weak binding IC involved in in vitro affinity is high for a cell surface molecule associated with a desired site.
(5) A delivery vehicle for achieving delivery to a desired site, in which a strong binding IC involved in in vitro affinity for a cell surface molecule associated with the desired site is low and a weak binding IC involved in in vitro affinity for the same is high.
(6) A delivery vehicle for achieving delivery to a desired site, which contains at least one binding type from among binding to a cell surface molecule associated with the desired site based on a rolling model and binding based on other forms of strong binding or weak binding.
(7) The delivery vehicle according to any one of (1) to (6), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(8) The delivery vehicle according to any one of (1) to (6), in which the cell surface molecule is a lectin.
(9) The delivery vehicle according to any one of (1) to (6), in which the boundary between the strong binding IC and the weak binding IC is between 30 and 31 for “n” of ICn.
(10) The delivery vehicle according to any one of (1) to (6), in which when the strong binding IC is measured under a condition such that “n” of ICn is 30 or less and the weak binding IC is measured under a condition such that “n” of ICn is 31 or more.
(11) The delivery vehicle according to any one of (1) to (6), in which the boundary between the strong binding IC and the weak binding IC is between 30 and 31 for “n” of ICn.
(12) The delivery vehicle according to (3), in which an inhibitory concentration at a strong IC which is IC30 or less is 10⁻⁹M or less as to in vitro affinity for a lectin associated with a desired site.
(13) The delivery vehicle according to (4), in which an inhibitory concentration at a weak binding IC which is approximately IC31 or more is 10⁻⁹M or more as to in vitro affinity for a lectin associated with a desired site.
(14) The delivery vehicle according to (5), in which an inhibitory concentration at a strong binding IC which is approximately IC30 or less is 10⁻⁹M or less and an inhibitory concentration at a weak binding IC which is approximately IC31 or more is 10⁻⁹M or more as to in vitro affinity for a lectin associated with a desired site.
(15) The delivery vehicle according to (14), which satisfies at least one condition selected from the group consisting of a condition that the inhibitory concentration at IC10 is 10⁻⁹M or less, a condition that an inhibitory concentration at IC20 is 10⁻⁹M or less and a condition that an inhibitory concentration at IC30 is 10⁻⁹M or less for the strong binding IC as to in vitro affinity for a lectin associated with a desired site, and satisfies at least one condition selected from the group consisting of a condition that the inhibitory concentration at IC40 is 10⁻⁹M or more, a condition that the inhibitory concentration at IC50 is 10⁻⁹M or more and a condition that the inhibitory concentration at IC60 is 10⁻⁹M or more for the weak binding IC as to in vitro affinity for a lectin associated with a desired site.
(16) The delivery vehicle according to (15), in which at least either the strong binding IC or the weak binding IC satisfies at least two of the above conditions.
(17) The delivery vehicle according to any one of (1) to (16), in which the delivery vehicle is a liposome.
(18) The delivery vehicle according to any one of (1) to (16), in which the delivery vehicle is a sugar-chain-modified liposome.
(19) The delivery vehicle according to any one of (1) to (16), in which the IC is measured based on affinity for E-selectin.
(20) The delivery vehicle according to any one of (1) to (16), in which the desired site is selected from the group consisting of an inflammation site and a cancer site.
(21) The delivery vehicle according to (12), which contains a liposome selected from the group consisting of liposome No. 16, liposome No. 27, liposome No. 29, liposome No. 41, liposome No. 45, liposome No. 53, liposome No. 69, liposome No. 71, liposome No. 76, liposome No. 80, liposome No. 87, liposome No. 91, liposome No. 93, liposome No. 96, liposome No. 105, liposome No. 106, liposome No. 117, liposome No. 125, liposome No. 127, liposome No. 137, liposome No. 139, liposome No. 142, liposome No. 146, liposome No. 150, liposome No. 151, liposome No. 152, liposome No. 153, liposome No. 154, liposome No. 184, liposome No. 186, liposome No. 191, liposome No. 195, liposome No. 199, liposome No. 204, liposome No. 207, liposome No. 213, liposome No. 224, liposome No. 225, liposome No. 229, liposome No. 230, liposome No. 234, liposome No. 235, liposome No. 239, liposome No. 240, liposome No. 263, liposome No. 273, liposome No. 285, and liposome No. 295.
(22) The delivery vehicle according to (13), which contains a liposome selected from the group consisting of liposome No. 3, liposome No. 16, liposome No. 27, liposome No. 29, liposome No. 38, liposome No. 40, liposome No. 41, liposome No. 45, liposome No. 50, liposome No. 53, liposome No. 56, liposome No. 60, liposome No. 68, liposome No. 69, liposome No. 70, liposome No. 71, liposome No. 76, liposome No. 80, liposome No. 87, liposome No. 91, liposome No. 93, liposome No. 96, liposome No. 105, liposome No. 106, liposome No. 111, liposome No. 116, liposome No. 117, liposome No. 120, liposome No. 125, liposome No. 127, liposome No. 129, liposome No. 130, liposome No. 137, liposome No. 139, liposome No. 141, liposome No. 142, liposome No. 146, liposome No. 150, liposome No. 151, liposome No. 152, liposome No. 153, liposome No. 154, liposome No. 155, liposome No. 175, liposome No. 178, liposome No. 183, liposome No. 184, liposome No. 186, liposome No. 191, liposome No. 195, liposome No. 197, liposome No. 199, liposome No. 204, liposome No. 207, liposome No. 209, liposome No. 213, liposome No. 218, liposome No. 220, liposome No. 224, liposome No. 225, liposome No. 229, liposome No. 230, liposome No. 233, liposome No. 234, liposome No. 235, liposome No. 236, liposome No. 237, liposome No. 239, liposome No. 240, liposome No. 254, liposome No. 263, liposome No. 273, liposome No. 285, liposome No. 290, liposome No. 292, and liposome No. 295.
(23) The delivery vehicle according to (14), which contains a liposome selected from the group consisting of liposome No. 16, liposome No. 27, liposome No. 29, liposome No. 41, liposome No. 45, liposome No. 53, liposome No. 69, liposome No. 71, liposome No. 76, liposome No. 80, liposome No. 87, liposome No. 91, liposome No. 93, liposome No. 96, liposome No. 105, liposome No. 106, liposome No. 111, liposome No. 125, liposome No. 127, liposome No. 137, liposome No. 139, liposome No. 142, liposome No. 146, liposome No. 150, liposome No. 151, liposome No. 152, liposome No. 153, liposome No. 154, liposome No. 184, liposome No. 186, liposome No. 191, liposome No. 195, liposome No. 199, liposome No. 204, liposome No. 207, liposome No. 213, liposome No. 224, liposome No. 225, liposome No. 229, liposome No. 230, liposome No. 234, liposome No. 235, liposome No. 239, liposome No. 240, liposome No. 263, liposome No. 273, liposome No. 285, and liposome No. 295.
(24) The delivery vehicle according to (15), which contains a liposome selected from the group consisting of liposome No. 3, liposome No. 16, liposome No. 27, liposome No. 29, liposome No. 38, liposome No. 40, liposome No. 41, liposome No. 45, liposome No. 50, liposome No. 53, liposome No. 56, liposome No. 60, liposome No. 68, liposome No. 69, liposome No. 70, liposome No. 71, liposome No. 76, liposome No. 80, liposome No. 87, liposome No. 91, liposome No. 93, liposome No. 96, liposome No. 105, liposome No. 106, liposome No. 111, liposome No. 116, liposome No. 117, liposome No. 120, liposome No. 125, liposome No. 127, liposome No. 129, liposome No. 130, liposome No. 137, liposome No. 139, liposome No. 141, liposome No. 142, liposome No. 146, liposome No. 150, liposome No. 151, liposome No. 152, liposome No. 153, liposome No. 154, liposome No. 155, liposome No. 175, liposome No. 178, liposome No. 183, liposome No. 184, liposome No. 186, liposome No. 191, liposome No. 195, liposome No. 197, liposome No. 199, liposome No. 204, liposome No. 207, liposome No. 209, liposome No. 213, liposome No. 218, liposome No. 220, liposome No. 224, liposome No. 225, liposome No. 229, liposome No. 230, liposome No. 233, liposome No. 234, liposome No. 235, liposome No. 236, liposome No. 237, liposome No. 239, liposome No. 240, liposome No. 254, liposome No. 263, liposome No. 273, liposome No. 285, liposome No. 290, liposome No. 292, and liposome No. 295.
(25) The delivery vehicle according to (16), which contains a liposome selected from the group consisting of liposome No. 3, liposome No. 16, liposome No. 27, liposome No. 29, liposome No. 38, liposome No. 40, liposome No. 41, liposome No. 45, liposome No. 50, liposome No. 53, liposome No. 56, liposome No. 60, liposome No. 68, liposome No. 69, liposome No. 70, liposome No. 71, liposome No. 76, liposome No. 80, liposome No. 87, liposome No. 91, liposome No. 93, liposome No. 96, liposome No. 105, liposome No. 106, liposome No. 111, liposome No. 116, liposome No. 117, liposome No. 120, liposome No. 125, liposome No. 127, liposome No. 129, liposome No. 130, liposome No. 137, liposome No. 139, liposome No. 141, liposome No. 142, liposome No. 146, liposome No. 150, liposome No. 151, liposome No. 152, liposome No. 153, liposome No. 154, liposome No. 155, liposome No. 175, liposome No. 178, liposome No. 183, liposome No. 184, liposome No. 186, liposome No. 191, liposome No. 195, liposome No. 197, liposome No. 199, liposome No. 204, liposome No. 207, liposome No. 209, liposome No. 213, liposome No. 218, liposome No. 220, liposome No. 224, liposome No. 225, liposome No. 229, liposome No. 230, liposome No. 233, liposome No. 234, liposome No. 235, liposome No. 236, liposome No. 237, liposome No. 239, liposome No. 240, liposome No. 254, liposome No. 263, liposome No. 273, liposome No. 285, liposome No. 290, liposome No. 292, and liposome No. 295.
(26) A method for producing a delivery vehicle for achieving the delivery of a desired substance to a desired site, which comprises the steps of:
A) measuring in vitro affinity of candidate delivery vehicles for a cell surface molecule associated with the site; and
B) selecting a delivery vehicle having in vitro affinity corresponding to delivery to the desired site.
(27) The method according to (26), in which the delivery vehicle contains a liposome.
(28) The method according to (26), in which the delivery vehicle contains a sugar-chain-modified liposome.
(29) The method according to (26), in which the candidates contain sugar-chain-modified liposomes containing a plurality of sugar chain type.
(30) The method according to (26), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(31) The method according to (26), in which the cell surface molecule is a lectin.
(32) The method according to (26), in which the cell surface molecule contains a lectin selected from the group consisting of E-selectin, P-selectin, L-selectin, galectin 1, galectin 2, galectin 3, galectin 4, galectin 5, galectin 6, galectin 7, galectin 8, galectin 9, galectin 10, galectin 11, galectin 12, galectin 13, galectin 14, mannose-6-phosphate receptor, calnexin, calreticulin, ERGIC-53, VIP53, interleukins, interferons, and growth factors.
(33) The method according to (26), in which the cell surface molecule contains E-selectin.
(34) The method according to (26), in which the cell surface molecule contains E-selectin and the site is selected from the group consisting of oral administration, a site of the liver, a site of the small intestine, a site of the large intestine, a site of the lymph node, a site of the heart, a site of the pancreas, a site of the lungs, a site of the brain, a site of the bone marrow, a site in blood, a site of the kidney, a site of the spleen, a site of the thymus gland, a site of muscle, an inflammation site, and a cancer site.
(35) The method according to (26), in which the cell surface molecule contains E-selectin and the site is selected from the group consisting of a tumor site and an inflammation site.
(36) The method according to (26), in which the affinity is represented by n % inhibitory concentration (ICn), wherein “n” ranges from 1 to 99.
(37) The method according to (26), in which the measurement of affinity comprises measurement at a strong binding IC that is approximately IC30 or less and measurement at a weak binding IC that is approximately IC31 or more.
(38) The method according to (26), in which the measurement of affinity comprises measurement at a strong binding IC that is at least one point between IC30 and IC10 and measurement at a weak binding IC that is at least one point between IC40 and IC60.
(39) The method according to (26), in which the measurement of affinity comprises measurement at a strong binding IC that is approximately IC30 or less and a candidate having a low inhibitory concentration at the strong binding IC is selected.
(40) The method according to (26), in which the measurement of affinity comprises measurement at a strong binding IC that is approximately IC30 or less and a candidate having an inhibitory concentration of 10⁻⁹M or less at the strong binding IC is selected.
(41) The method according to (26), in which the measurement of affinity comprises measurement at a strong binding IC that is approximately IC30 or less and a candidate having a low inhibitory concentration at the strong binding IC is selected, wherein
the selection is performed when at least one condition selected from the group consisting of a condition that the inhibitory concentration at IC10 is 10⁻⁹M or less, a condition that an inhibitory concentration at IC20 is 10⁻⁹M or less and a condition that an inhibitory concentration at IC30 is 10⁻⁹M or less is satisfied.
(42) The method according to (26), in which the measurement of affinity comprises measurement at a weak binding IC that is approximately IC31 or more and a candidate having a high inhibitory concentration at the strong binding IC is selected.
(43) The method according to (26), in which the measurement of affinity comprises measurement at a weak binding IC that is approximately IC31 or more and a candidate having an inhibitory concentration of 10⁻⁹M or more at the weak binding IC is selected.
(44) The method according to (26), in which the measurement of affinity comprises measurement at a weak binding IC that is approximately IC31 or more and a candidate having a high inhibitory concentration at the weak binding IC is selected, wherein
the selection is performed when at least one condition selected from the group consisting of a condition that the inhibitory concentration at IC40 is 10⁻⁹M or more, a condition that the inhibitory concentration at IC50 is 10⁻⁹M or more and a condition that the inhibitory concentration at IC60 is 10⁻⁹M or more is satisfied.
(45) The method according to (26), in which the measurement of affinity comprises measurement at a strong binding IC that is approximately IC30 or less and a weak binding IC that is approximately IC31 or more and a candidate having a low inhibitory concentration at the strong binding IC and a high inhibitory concentration at the weak binding IC is selected.
(46) The method according to (26), in which the measurement of affinity comprises measurement at a strong binding IC that is approximately IC30 or less and a weak binding IC that is approximately IC31 or more and a candidate having an inhibitory concentration of 10⁻⁹M or less at the strong binding IC and an inhibitory concentration of 10⁻⁹M or more at the weak binding IC is selected.
(47) The method according to (26), in which the measurement of affinity comprises measurement at a strong binding IC that is approximately IC30 or less and a weak binding IC that is approximately IC31 or more and a candidate having a low inhibitory concentration at the strong binding IC and a high inhibitory concentration at the weak binding IC is selected,
wherein
the selection is performed when at least one condition selected from the group consisting of a condition that the inhibitory concentration at IC10 is 10⁻⁹M or less, a condition that an inhibitory concentration at IC20 is 10⁻⁹M or less and a condition that an inhibitory concentration at IC30 is 10⁻⁹M or less is satisfied; and in terms of the weak binding IC, at least one condition selected from the group consisting of a condition that the inhibitory concentration at IC40 is 10⁻⁹M or more, a condition that the inhibitory concentration at IC50 is 10⁻⁹M or more and a condition that the inhibitory concentration at IC60 is 10⁻⁹M or more is satisfied.
(48) The method according to (26), in which the measurement of affinity is performed by a method selected from the group consisting of a competitive inhibition assay, a noncompetitive inhibition assay, and a binding assay.
(49) A method for producing a delivery vehicle for achieving delivery to a desired site, which comprises the steps of:
A) measuring in vitro affinity of candidate delivery vehicles for a cell surface molecule associated with the site; and
B) selecting a delivery vehicle having in vitro affinity corresponding to delivery to the desired site and analyzing the composition of the selected delivery vehicle; and
C) generating the thus selected delivery vehicle based on the composition.
(50) The method according to (49), which further comprises the step of causing the selected delivery vehicle to contain a substance to be delivered.
(51) The method according to (49), in which the delivery vehicle contains a liposome.
(52) The method according to (49), in which the delivery vehicle contains a sugar-chain-modified liposome.
(53) The method according to (49), in which the candidate contains a sugar-chain-modified liposome that contains a plurality of sugar chain types.
(54) The method according to (49), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(55) The method according to (49), in which the cell surface molecule is a lectin.
(56) The method according to (49), in which the cell surface molecule contains a lectin selected from the group consisting of E-selectin, P-selectin, L-selectin, galectin 1, galectin 2, galectin 3, galectin 4, galectin 5, galectin 6, galectin 7, galectin 8, galectin 9, galectin 10, galectin 11, galectin 12, galectin 13, galectin 14, a mannose-6-phosphate receptor, calnexin, calreticulin, ERGIC-53, VIP53, interleukins, interferons, and growth factors.
(57) The method according to (49), in which the cell surface molecule contains E-selectin.
(58) The method according to (49), in which the cell surface molecule contains E-selectin and the site is selected from the group consisting of oral administration, a site of the liver, a site of the small intestine, a site of the large intestine, a site of the lymph node, a site of the liver, a site of the heart, a site of the pancreas, a site of the lungs, a site of the brain, a site of the bone marrow, a site in blood, a site of the kidney, a site of the spleen, a site of the thymus gland, a site of muscle, an inflammation site, and a cancer site.
(59) The method according to (49), in which the cell surface molecule contains E-selectin and the site is selected from the group consisting of a tumor site and an inflammation site.
(60) The method according to (49), in which the affinity is represented by ICn, wherein “n” ranges from 1 to 99.
(61) The method according to (49), in which the measurement of affinity comprises measurement at a strong binding IC that is approximately IC30 or less and measurement at a weak binding IC that is approximately IC31 or more.
(62) The method according to (49), in which the measurement of affinity comprises measurement at a strong binding IC that is at least one between IC30 and IC10 and measurement at a weak binding IC that is at least one between IC40 and IC60.
(63) The method according to (49), in which the measurement of affinity comprises measurement at a strong binding IC that is approximately IC30 or less and a candidate having a low inhibitory concentration at the strong binding IC is selected.
(64) The method according to (49), in which the measurement of affinity comprises measurement at a strong binding IC that is approximately IC30 or less and a candidate having an inhibitory concentration of 10⁻⁹M or less at the strong binding IC is selected.
(65) The method according to (49), in which the measurement of affinity comprises measurement at a strong binding IC that is approximately IC30 or less and a candidate having a low inhibitory concentration at the strong binding IC is selected, wherein
the selection is performed when at least one condition selected from the group consisting of a condition that the inhibitory concentration at IC10 is 10⁻⁹M or less, a condition that an inhibitory concentration at IC20 is 10⁻⁹M or less and a condition that an inhibitory concentration at IC30 is 10⁻⁹M or less is satisfied.
(66) The method according to (49), in which the measurement of affinity comprises measurement at a weak binding IC that is approximately IC31 or more and a candidate having a high inhibitory concentration at the strong binding IC is selected.
(67) The method according to (49), in which the measurement of affinity comprises measurement at a weak binding IC that is approximately IC31 or more and a candidate having an inhibitory concentration of 10⁻⁹M or more at the weak binding IC is selected.
(68) The method according to (49), in which the measurement of affinity comprises measurement at a weak binding IC that is approximately IC31 or more and a candidate having a high inhibitory concentration at the weak binding IC is selected, wherein
the selection is performed when at least one condition selected from the group consisting of a condition that the inhibitory concentration at IC60 is 10⁻⁹M or more, a condition that the inhibitory concentration at IC50 is 10⁻⁹M or more and a condition that the inhibitory concentration at IC40 is 10⁻⁹M or more is satisfied.
(69) The method according to (49), in which the measurement of affinity comprises measurement at a strong binding IC that is approximately IC30 or less and a weak binding IC that is approximately IC31 or more and a candidate having a low inhibitory concentration at the strong binding IC and a high inhibitory concentratin at the weak binding IC is selected.
(70) The method according to (49), in which the measurement of affinity comprises measurement at a strong binding IC that is approximately IC30 or less and a weak binding IC that is approximately IC31 or more and a candidate having an inhibitory concentration of 10⁻⁹M or less at the strong binding IC and an inhibitory concentration of 10⁻⁹M or more at the weak binding IC is selected.
(71) The method according to (49), in which the measurement of affinity comprises measurement at a strong binding IC that is approximately IC30 or less and a weak binding IC that is approximately IC31 or more and a candidate having a low inhibitory concentration at the strong binding IC and a high inhibitory concentration at the weak binding IC is selected,
wherein
the selection is performed when at least one condition selected from the group consisting of a condition that the inhibitory concentration at IC10 is 10⁻⁹M or less, a condition that an inhibitory concentration at IC20 is 10⁻⁹M or less and a condition that an inhibitory concentration at IC30 is 10⁻⁹M or less is satisfied; and in terms of the weak binding IC, at least one condition selected from the group consisting of a condition that the inhibitory concentration at IC40 is 10⁻⁹M or more, a condition that the inhibitory concentration at IC50 is 10⁻⁹M or more and a condition that the inhibitory concentration at IC60 is 10⁻⁹M or more is satisfied.
(72) The method according to (49), in which the measurement of affinity is performed by a method selected from the group consisting of a competitive inhibition assay, a noncompetitive inhibition assay, and a binding assay.
(73) The method according to (49), which further comprises a step of determining a method for preparing a delivery vehicle having the composition in addition to the analysis of the composition.
(74) The method according to (49), in which the delivery vehicle contains a sugar-chain-modified liposome and the analysis of the composition comprises analysis of the sugar chain type and density of the sugar-chain-modified liposome.
(75) The method according to (49), in which the delivery vehicle contains a sugar-chain-modified liposome and the analysis of the composition comprises determination of a method for producing the sugar-chain-modified liposome.
(76) The method according to (49), in which the delivery vehicle contains a sugar-chain-modified liposome and which comprises determination of a method for producing the sugar-chain-modified liposome instead of the analysis of the composition.
(77) The method according to (49), in which the delivery vehicle contains a sugar-chain-modified liposome and which further comprises, when the sugar-chain-modified liposome is generated, a step of causing a reaction of a sugar chain, the type and the extent of which is determined based on the above composition, under appropriate conditions for binding to the liposome.
(78) The method according to (57), in which a linker is used in the binding.
(79) The method according to (78), in which the linker is a protein.
(80) The method according to (78), in which the linker is an albumin.
(81) The method according to (77), which further comprises a step of hydrophilizing the liposome.
(82) The method according to (49), which further comprises a step of confirming the in vivo dynamic state of the selected delivery vehicle.
(83) A method for producing a delivery vehicle by which delivery to an undesired site does not occur, which comprises the steps of:
A) measuring in vitro affinity of candidate delivery vehicles for a cell surface molecule associated with the site; and
B) selecting a delivery vehicle having in vitro affinity corresponding to non-delivery to the undesired site.
(84) The method according to (83), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(85) The method according to (83), in which the cell surface molecule is a lectin.
(86) A method for producing a delivery vehicle by which delivery to an undesired site does not occur, which comprises the steps of:
A) measuring in vitro affinity of candidate delivery vehicles for a cell surface molecule associated with the site;
B) selecting a delivery vehicle having in vitro affinity corresponding to non-delivery to the undesired site and analyzing the composition of the selected delivery vehicle; and
C) generating the selected delivery vehicle based on the composition.
(87) The method according to (86), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(88) The method according to (86), in which the cell surface molecule is a lectin.
(89) A method for producing a delivery vehicle for achieving specific delivery, which comprises the steps of:
A) measuring in vitro affinity of candidate delivery vehicles for a cell surface molecule associated with the site to which specific delivery is performed;
B) measuring in vitro affinity of the candidate delivery vehicles for a cell surface molecule associated with a site to which specific delivery is not performed; and
C) selecting a delivery vehicle having in vitro affinity corresponding to delivery to a desired site and corresponding to non-delivery to an undesired site.
(90) The method according to (89), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(91) The method according to (89), in which the cell surface molecule is a lectin.
(92) A method for producing a delivery vehicle for achieving specific delivery, which comprises the steps of:
A) measuring in vitro affinity of candidate delivery vehicles for a cell surface molecule associated with the site to which specific delivery is performed;
B) measuring in vitro affinity of the candidate delivery vehicles for a cell surface molecule associated with a site to which specific delivery is not performed;
C) selecting a delivery vehicle having in vitro affinity corresponding to delivery to a desired site and corresponding to non-delivery to an undesired site and analyzing the composition of the selected delivery vehicle; and
D) generating the selected delivery vehicle based on the composition.
(93) The method according to (92), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(94) The method according to (92), in which the cell surface molecule is a lectin.
(95) A delivery vehicle, which is produced by the method according to any one of (26) to (94).
(96) A method for preventing or treating a subject who requires delivery of a drug to a desired site, which comprises the steps of:
A) measuring in vitro affinity of candidate delivery vehicles, which are intended for achieving delivery to a desired site, for a cell surface molecule associated with the desired site;
B) selecting a delivery vehicle having in vitro affinity corresponding to delivery to the desired site; and
C) administering a drug required for prevention or treatment to the subject with the use of the selected delivery vehicle.
(97) The method according to (96), in which the cell surface molecule is a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(98) The method according to (96), in which the cell surface molecule is a lectin.
(99) A method for preventing or treating a subject who requires delivery of a drug to a desired site, which comprises the steps of:
A) measuring in vitro affinity of candidate delivery vehicles, which are intended for achieving delivery to a desired site, for a cell surface molecule associated with the site;
B) selecting a delivery vehicle having in vitro affinity corresponding to delivery to the desired site and analyzing the composition of the selected delivery vehicle;
C) generating the selected delivery vehicle containing a drug required for prevention or treatment based on the composition; and
D) administering the selected delivery vehicle to the subject.
(100) The method according to (99), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(101) The method according to (99), in which the cell surface molecule is a lectin.
(102) An apparatus for producing a delivery vehicle for achieving delivery to a desired site, which is provided with:
A) a means for measuring in vitro affinity of candidate delivery vehicles for a cell surface molecule associated with the site; and
B) a means for selecting a delivery vehicle having in vitro affinity corresponding to delivery to the desired site.
(103) The apparatus according to (102), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(104) The apparatus according to (102), in which the cell surface molecule is a lectin.
(105) An apparatus for producing a delivery vehicle for achieving delivery to a desired site, which is provided with:
A) a means for measuring in vitro affinity of candidate delivery vehicles for a cell surface molecule associated with the site;
B) a means for selecting a delivery vehicle having in vitro affinity corresponding to delivery to the desired site.
C) a means for analyzing the composition of the selected delivery vehicle; and
D) a means for generating the selected delivery vehicle based on the composition.
(106) The apparatus according to (105), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(107) The apparatus according to (105), in which the cell surface molecule is a lectin.
(108) Use of in vitro affinity for a cell surface molecule associated with a desired site, which is intended for producing a delivery vehicle for achieving delivery to the desired site.
(109) The use according to (108), in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
(110) The use according to (108), in which the cell surface molecule is a lectin.
(111) A pharmaceutical composition, which contains a drug to be used for prevention or treatment, and the delivery vehicle according to any one of (1) to (25) or a delivery vehicle that is produced by the method according to any one of (26) to (94).

Hereafter, preferred embodiments of the present invention will be described. It should be recognized that persons skilled in the art can adequately implement the embodiments according to the explanation and attached drawings of the present invention and known conventional technology in the art and can easily understand the action and effect exerted by the present invention.

### EFFECT OF THE INVENTION

According to the present invention, a method for designing a useful delivery vehicle such as a sugar-chain-modified liposome, a production method based on a rolling model, and a method for using them are provided. The delivery vehicle of the present invention significantly increases the range of development of a DDS formulation with which a desired drug can be provided to a target delivery site. The present invention enables development and practical application of a delivery system that is required for realization of new treatment in each field of cancer therapy, gene therapy, regeneration medicine, and the like. Such various delivery vehicles that are useful for oral administration are provided according to the present invention for the first time.

### BRIEF DESCRIPTION OF THE DRAWINGS

FIG. 1 is a schematic diagram showing the production of a sugar-chain-modified liposome that can be used in the present invention.

FIG. 2 is a graph for calculation of ICn. IC50 is shown in this figure. The concentration of a subject drug is plotted on the X axis and the amount of a bound ligand is plotted on the Y axis.

FIG. 3 is a graph for calculation of IC20. LNT-4, LY-1, and A6 are abbreviated names of liposomes.

FIG. 4 is a graph for calculation of IC30. LNT-4, LY-1, and A6 are abbreviated names of liposomes.

FIG. 5 is a graph for calculation of IC40. LNT-4, LY-1, and A6 are abbreviated names of liposomes.

FIG. 6 is a graph for calculation of IC50. LNT-4, LY-1, and A6 are abbreviated names of liposomes.

FIG. 7 shows the anticancer effect exerted by doxorubicin-encapsulated liposome No. 155 that was injected via tail vein to cancer-bearing mice.

FIG. 8 shows fluorescence microscopic photographs showing the effect of accumulating doxorubicin from tumor blood vessels to tumor tissues in cancer-bearing mice to which doxorubicin-encapsulated liposome No. 155 was injected via tail vein. Images on the left and images on the right are green and red fluorescence microscopic photographs of the same tumor tissue.

FIG. 9 shows the anticancer effect exerted by doxorubicin-encapsulated liposome No. 237 that was administered via oral administration to cancer-bearing mice.

FIG. 10 shows fluorescence microscopic photographs showing the effect of accumulating doxorubicin-encapsulated liposome No. 237 from tumor blood vessels to tumor tissue, which was administered via oral administration to cancer-bearing mice. Images on the left and images on the right are green and red fluorescence microscopic photographs of the same tumor tissue.

FIG. 11 is a typical graph of a rolling model showing concentration-inhibition % curves (substitution curves). A solid line is an example of a curve more preferred in the rolling model than a dotted line. A dotted line is an unpreferable example (e.g., non-specific binding or antigen antibody reaction).

FIG. 12 is a schematic diagram of Example 1 of the action mechanism of a delivery vehicle based on a rolling model.

FIG. 13 is a schematic diagram of Example 2 of the action mechanism of a delivery vehicle based on a rolling model.

FIG. 14 is a schematic diagram of Example 3 of the action mechanism of a delivery vehicle based on a rolling model.

FIG. 15 is a schematic diagram of Example 4 of the action mechanism of a delivery vehicle based on a rolling model.

### BEST MODE OF CARRYING OUT THE INVENTION

Hereinafter, the present invention will be explained by describing the best mode thereof. Throughout the description, expressions in the singular form should be understood as including the concept of the plural form thereof, unless otherwise specified. Therefore, articles in the singular form (e.g., in the case of English, “a,” “an,” and “the”) should be understood as including the concept of the plural form thereof, unless otherwise specified. Furthermore, it should be understood that terms used in the description have the same meanings as those that generally apply in the above fields, unless specified. Therefore, unless otherwise defined, all the technical terms and engineering and scientific terms that are used in the description have meanings that are generally understood by persons skilled in the fields to which the present invention corresponds. When there is a conflict, the description (including definitions) is prioritized.

Embodiments that are provided hereafter are provided for better understanding of the present invention. It is understood that the scope of the present invention should not be limited to the following description. Therefore, it is clear that persons skilled in the art can adequately make modifications within the scope of the present invention by taking the content of the description into consideration.

Preferred embodiments of the present invention are explained by adequately explaining the definitions of terms that are particularly used in the description. A sugar-chain-modified liposome is the main object of explanation in the present invention, but it will be understood that the rolling model of the present invention is not limited thereto.

### (In Vitro Assay Using Cell Surface Molecule)

According to an aspect of the present invention, a method for producing a delivery vehicle that is used for the achievement of delivery of a desired substance to a desired site is provided. The production method comprises the steps of: A) measuring in vitro affinity of delivery vehicle candidates for a cell surface molecule such as a lectin associated with the site; and B) selecting a delivery vehicle having in vitro affinity corresponding to delivery to the desired site.

Alternatively, the production method of the present invention comprises the steps of: A) measuring in vitro affinity of delivery vehicle candidates for a cell surface molecule such as a lectin associated with the site; B) selecting a delivery vehicle having in vitro affinity corresponding to delivery to the desired site and analyzing the composition of the selected delivery vehicle; and C) generating the selected delivery vehicle based on the composition.

In the description, “delivery vehicle” refers to a vehicle that mediates the delivery of a desired substance. If a substance to be delivered is a drug, the delivery vehicle is referred to as a “drug delivery vehicle.” Examples of the delivery vehicle can include a lipid vehicle, a liposome, a lipid micelle, a lipoprotein micelle, a lipid-stabilizing emulsion, cyclodextrin, a polymer nanoparticle, a polymer fine particle, a block copolymer micelle, a polymer-lipid hybrid system, and a derivatized single-chain polymer.

DDS is also referred to as a drug delivery system and may be classified into absorption-control DDS, release-control DDS, and targeting DDS. An ideal DDS is a system which sends a drug “to sites where it is needed in vivo,” “in a required amount,” and “only for required time.” Targeting DDS (Targeting DDS in English and “Hyo-teki shiko-sei DDS” in Japanese) is classified into passive targeting DDS and active targeting DDS. Passive targeting DDS uses the physicochemical properties (e.g., particle diameter and hydrophilicity) of a carrier (transporter of the drug) to control behavior in vivo. Active targeting DDS adds special mechanisms to the passive type to actively control the tropism for the target tissue. For example, there is a method referred to as “missile drug” using carriers consisting of combinations of antibodies, sugar chains, etc., that are capable of specifically recognizing target molecules of certain cells that make up the target tissue.

In the description, “encapsulation” means stable association with a delivery vehicle. When a vehicle is administered in vivo, encapsulation of one or a plurality of drugs is not required as long as one or a plurality of drugs are stably associated with the vehicle. Therefore, “stably associated with . . . ” and “encapsulated in . . . ” or “encapsulated together with . . . ” or “encapsulated in or encapsulated together with . . . ” are intended to be synonymous with each other. These terms are used in the description interchangeably. Stable association can be generated via various means including a covalent bond with a delivery vehicle, preferably a bond that can be cleaved, a non-covalent bond, capturing of a drug within a delivery vehicle, and the like. Association must be sufficiently stable to such a degree that association with a delivery vehicle is maintained at a noncompetitive rate until the drug is delivered to a target site in a subject to which the drug is administered.

It is understood that in the description, any substance can be used as a delivery vehicle, as long as it is compatible with a living body (in the description, referred to as “biocompatible substance”) into which the substance is delivered. Preferably, such a biocompatible substance can preferably be a biodegradable substance (e.g., biodegradable polymer) and may be any substance as long as it does not have any harmful effect on a living body. Examples of such substance include a lipid (e.g., a liposome), polyester, cyclodextrin, polyamino acid, silicon (e.g., porous silicon or a biosilicon material (for example, substances disclosed in WO99/53898, the disclosure of which is incorporated herein in its entirety as a reference)), mesoporous, microporous, or polycrystalline silicon), an ethylene vinyl acetate copolymer, and polyvinylalcohol. Examples of a biodegradable polymer include, but are not limited to, polyester (e.g., polylactic acid-glycolic acid copolymer (PLGA)), hydrophobic polyamino acid (e.g., polyalanine and polyleucine), polyanhydride, polyglycerol sebacate (PGS), and Biopol. “Hydrophobic polyamino acid” means a polymer that is prepared from hydrophobic amino acid. Examples of a non-biodegradable polymer that can be used in the present invention include, but are not limited to, silicon, polytetrafluoroethylene, polyethylene, polypropylene, polyurethane, polyacrylate, polymethacrylate (e.g., polymethylmethacrylate), and ethylene vinyl acetate copolymer.

The delivery vehicle of the present invention may also contain a water-soluble substance. A water-soluble substance plays a role in control of the water infiltration into a drug dispersion system. Such water-soluble substances are not limited in view of water-soluble substance and physiologically acceptable water-soluble substance as long as they are solid substances (in the forms of prepared products) at body temperatures of animals or humans to which they may be administered. One water-soluble substance or a combination of 2 or more water-soluble substances can also be used. Specifically, one or a plurality of water-soluble substances can be selected from the group consisting of synthetic polymers (e.g., polyethylene glycol and polyethylenepolypropyleneglycol), sugars (e.g., sucrose, mannitol, glucose, and sodium chondroitin sulfate), polysaccharides (e.g., dextran), amino acids (e.g., glycine and alanine), inorganic salts (e.g., sodium chloride), organic salts (e.g., sodium citrate) and proteins (e.g., gelatin and collagen and a mixture thereof). Moreover, when a water-soluble substance is an amphiphilic substance that can be dissolved in both organic solvent and water, the solubility of the substance is altered so as to have an effect of controlling the release of a lipophilic remedy. Such an amphiphilic substance may contain one or a plurality of substances selected from the group consisting of polyethylene glycol or a derivative thereof, polyoxyethylene polyoxypropylene glycol or a derivative thereof, and sugar ester of fatty acid and sodium alkyl sulfate, and further specifically, polyethylene glycol, polyoxystearate 40, polyoxyethylene, polyoxypropylene glycol, sucrose ester of fatty acid, sodium lauryl sulfate, sodium oleate, and sodium desoxycholate (deoxy sodium cholate (DCA)). However, the examples are not limited thereto. Such a water-soluble substance may contain a water-soluble substance having a kind of activity in vivo, such as a low-molecular-weight drug, a peptide, a polypeptide, a protein, a glycoprotein, polysaccharides or water-soluble drug (that is, antigenic substance that is used as a vaccine.

Cyclodextrin is a water-soluble polysaccharide capable of forming hollows, in which a water-insoluble drug can be contained. A drug can be encapsulated within cyclodextrin using procedures known by persons skilled in the art. For example, see ed., by Atwood et al., “Inclusion Compounds” vol. 2 and vol. 3, Academic Press, NY (1984); Bender et al., “Cyclodextrin Chemistry,” Springer-Verlag, Berlin (1978); Szeitli et al., “Cyclodextrins and Their Inclusion Complexes,” Akademiai Kiado, Budapest, Hungary (1982) and International Publication No. 00/40962.

A nanoparticle or a fine particle is a concentration core, which is enclosed by a polymer shell (nanocapsule) or a nanosphere, in which a solid or a liquid is dispersed in the entire polymer substrate. A general method for preparing nanoparticles and fine particles is described in Soppimath et al., J. Control Release (2001) 70(12): 1-20 which is incorporated herein by reference in their entirety. Another example of the polymer delivery vehicle that can be used herein is a block copolymer micelle containing a drug that contains a hydrophobic core enclosed by a hydrophilic shell. The micelle is generally used as a vehicle for a hydrophobic drug and can be prepared as described in Allen et al., “Colloids and Surface B), Biointerfaces (1999) Nov. 16 (1-4): 3-27. A polymer-lipid hybrid system comprises a polymer nanoparticle that is enclosed by a lipid monolayer. Polymer particles function as a cargo space for incorporation of a hydrophobic drug and a lipid monolayer functions as a stable boundary between the hydrophobic core and the external aqueous environment. As polymers, polycaprolactone, poly(d,l-lactide), and the like can be used. A lipid monolayer is generally composed of a lipid mixture. A suitable method therefor is similar to that described in the above reference concerning polymer nanoparticles. Derivatized single-chain polymer is a polymer that is modified to be suitable for the formation of a polymer-drug conjugate via covalent binding with a physiologically active substance. Various polymers including polyamino acids, polysaccharides (e.g., dextrin or dextran), and synthetic polymers (e.g., N-(2-hydroxypropyl)methacrylamide(HPMA) copolymer) are proposed as polymers for the synthesis of polymer-drug conjugates. A preparation method suitable therefor is described in detail in Veronese and Morpurgo, I L Farmaco (1999) 54(8): 497-516, which is incorporated herein by reference in their entirety.

In the description, “cell surface molecule” refers to an arbitrary molecule that is present on the surface of a cell. Examples of such cell surface molecule include, but are not limited to, lectins, cell adhesion molecules, receptors, proteins, lipids, phospholipids, transmembrane domains, and extracellular matrices.

In the description, “lectin” refers to a substance capable of binding to a sugar chain of a cell membrane glycoconjugate (glycoprotein and glycolipid) and having effects such as cell aggregation, division induction, activation of functions, and cell damage. If a sugar chain is assumed to be an information molecule transmitted from cells, it can be said that a lectin is a receiving molecule. Cells or tissues having a certain degree of properties have a pattern of lectin corresponding thereto. Lectins realize infection, biophylaxis, immune, fertilization, targeting target cells, cell differentiation, intercellular adhesion, quality control of nascent glycoprotein, intracellular selective transport, and the like. Lectins have a variety of sugar chain-binding property and unique physicochemical characteristics such as rapid binding and dissociation, so that the lectins are strictly controlled. Lectins are also referred to as sugar chain-recognizing proteins. Plant lectins have long been studied so that approximately 300 types of plant lectin have already been known. Recently, animal lectins have also been actively studied, so that novel lectins have been discovered one after another. A wide variety of sugar chain-recognizing functions based on a lectin group (approximately 100 types or more) of the major lectin family that is present on animal cell membranes have been studied. In particular, the functions as a receptor (an information-receiving protein or a target molecule) that receives the structural information of a sugar chain ligand varying in structure are attracting attention. In the description, explanation is given based mainly on lectins, however, it is understood that similar explanation is possible for cell surface molecules other than lectins.

In the description, “ligand” refers to a substance that specifically binds to a protein in biochemical fields. For example, substrates that bind to enzymes, peptides, hormones, neurotransmitters, and the like that bind to various receptor proteins (referred to as receptors) existing on the cell membranes are referred to as ligands to their corresponding proteins. Hence, in the case of this study, to use each type of lectin protein (that functions as a type of receptor that is present on the specific cell membranes of a target tissue) as a target molecule, a sugar chain that is a ligand of the protein is introduced onto a liposome surface, so that a DDS nanoparticle having an active targeting function imparted thereto is prepared.

A lectin is present on the cell membrane surface and a sugar chain that recognizes the lectin is present at the same time. In this case cell-to-cell interaction takes place. Lectin-positive cells may interact with sugar chain ligand-positive cells. A soluble glycoprotein may act on lectin-positive cells and a soluble lectin may act on sugar chain ligand-positive cells.

The binding dissociation constant (K_{D}) between a sugar chain and a lectin ranges from approximately 10⁻³M to 10⁻⁶M. Binding between a sugar chain and a lectin is far weaker than that of an antigen antibody reaction (K_{D}: 10⁻⁷ to 10⁻¹²). However, it is important for intercellular adhesion and selection to determine whether or not binding should be performed based on weak binding. Therefore, in a preferred embodiment, a cell surface molecule having binding dissociation constant at such a level can have an advantage, but the example is not limited thereto.

Sugar chains and lectins may have a plurality of binding sites in one molecule or may be present as molecular assemblies. Although their weak binding force, they are complemented by polyvalency (10⁻⁸M to 10⁻¹⁰M). Because of their strict binding specificity, they can be used for strict delivery of a substance to a specific tissue and the like.

When the present invention is implemented, in vitro affinity of delivery vehicle candidates for a cell surface molecule such as a lectin associated with a desired site is measured upon screening. It has been discovered that a delivery vehicle having a specific tendency in terms of in vitro affinity is a delivery vehicle preferred in vivo. This is referred to as “rolling model” in the description.

“Rolling model” of the present invention is based on a finding that a delivery vehicle having a low inhibitory concentration of strong binding (in which n of ICn is relatively small) and having a high inhibitory concentration of weak binding (in which n of ICn is relatively large) exerts good delivery to target organs or tissues. The rolling model has been completed by a discovery that a substance showing a gentle concentration curve in the vicinity of a threshold near the boundary between strong binding and weak binding is preferable as a delivery vehicle. A theory has thus been discovered that such a substance does not firmly bind in a target organ or tissue but should have a property of rolling while appropriately binding. When such substances have been actually screened for and tests have been conducted in vivo, all the substances have achieved in vivo preferable delivery results. Therefore, the present invention provides revolutionary assay system and screening system by which whether a delivery substance can show a preferable delivery result in a target organ or tissue can be determined by performing simple assay in vitro.

Therefore, in the description, “strong binding” refers to binding in which n of ICn is relatively small and specifically refers to binding with approximate ICn (here, n is typically smaller than approximately 30). Strong binding may be appropriate for testing relatively strong binding force (for example, antigen antibody reaction may be included). Here, “strong binding inhibitory concentration (IC)” refers to an inhibitory concentration with low percentage of inhibition.

Therefore, in the description, “weak binding” refers to binding in which n of ICn is relatively large and specifically refers to binding with approximate ICn (here, n is typically larger than approximately 31). Weak binding may be appropriate for testing relatively weak binding force (for example, binding that is almost non-specific binding is included herein). Here, “weak binding inhibitory concentration (IC)” refers to an inhibitory concentration with high percentage of inhibition.

These strong binding and weak binding are also assumed to vary depending on cell surface molecules such as lectins. It is understood that persons skilled in the art can appropriately determine n of ICn depending on a system to be used.

Preferably through combination of index numbers of the strong binding and weak binding, the present inventors have succeeded in discovery of an appropriate delivery vehicle for rolling with the use of an in vitro system.

According to the present invention, the thus selected delivery vehicle does not firmly bind to tissues and makes it possible to efficiently release active ingredients such as a drug that is delivered together with the vehicle into a target. Hence, the delivery vehicle can achieve far more efficient delivery compared with an antigen antibody reaction that is accompanied by firm binding to a tissue. With the “rolling model” theory of the present invention, such a system that can ideally achieve such delivery can be conveniently discovered.

No theoretical constraints are desired herein, however, three technological elements of DDS can mainly exist: drug-releasing technology, drug-targeting technology, and drug-absorption-controlling technology. The drug-releasing technology includes “release control technology” of dispersing a drug in a polymer matrix or the like so as to release a given amount of drug over a long time and “effective release technology” of encapsulating a protein formulation or the like that is almost insoluble in water within a vehicle such as a liposome so as to cause the effective expression in vivo of the medicinal effect.

Drug-targeting technology includes “active targeting technology” of actively delivering a drug to affected parts with the use of a ligand or an antibody that recognizes cancerous tissues and “passive targeting technology” of binding a polyethylene glycol or the like to a drug so that the drug that is injected into a blood vessel is not easily metabolized in the liver and the like and the drug is circulated in vivo for a long time, thereby causing accumulation of the drug in affected parts such as cancer.

The drug-absorption-controlling technology includes “drug introduction technology” of causing absorption of a drug via mucous membrane or skin and “gene introduction technology” of introducing a gene into cells, so as to treat the disease, which is employed for gene therapy and the like.

Among them, it has been revealed that an optimum combination of the active-targeting technology and the effective release technology can be conveniently discovered with the use of the rolling model of the present invention. It can be said that this is a special effect that has been unable to be discovered by conventional methods.

A typical example of a cell surface molecule that can be used in the present invention is a lectin. Examples of such a lectin include, but are not limited to, selectin (e.g., L-selectin, E-selectin, and P-selectin), lectins involved in intracellular transport and selection of glycoproteins (e.g., a mannose-6-phosphate receptor, calnexin, calreticulin, ERGIC-53, and VIP53), cytokines (e.g., interleukins, interferons, and growth factors), and galectin.

Among lectins, “selectin” in the description refers to a transmembrane type glycoprotein that is of a group of cell adhesion molecules that recognize sugar chains and has the N-terminus extracellularly and the C-terminus intracellularly. These molecules of this type have extracellularly, from the terminus in order, lectin domain (L) that recognizes sugar chains in a Ca²⁺-dependent manner, EGF (epidermal growth factor)-like domain (E) that has three disulfide bonds, and complement-binding domain (C) that has homology with a complement-binding protein. These molecules may also be referred to as LECAM (lectin-type cell adhesion molecules) or as an LECAM family. There are at least three types of molecules: L-selectin (LECAM-1) that is expressed by leukocytes, E-selectin (ELAM-1 and LECAM-2) that is expressed by activated vascular endothelial cells, P-selectin (GMP-140 and LECAM-3) that is expressed by activated blood platelet and activated vascular endothelial cells.

L-selectin is expressed constitutively in most leukocytes. As ligands for L-selectin, GlyCAM-1 (glycosylatkon-dependent cell adhesion molecule), CD34, MAdCAM-1 (mucosal addressin cell adhesion molecule), and the like are known. L-selectin achieves intercellular adhesion via its binding to sialyl-6-sulfo Lex and is involved in homing phenomenon by which lymphocytes in bloodstream assemble in specific lymphoid tissues.

E-selectin is often expressed on inflamed vascular endothelial cells, by which granulocytes, monocytes, and the like assemble at the inflammation sites. When vascular endothelial cells are stimulated with interleukin1 (IL-1), tumor necrosis factor α (TNF-α), endotoxin, and the like, expression of E-selectin is induced within several hours. Therefore, it is understood according to the rolling theory of the present invention, in vitro affinity for E-selectin is an indicator for delivery to vascular endothelial cells, inflammation sites, tumors, and the like.

P-selectin is contained in α granules of blood platelets and Weibel-Palade bodies of endothelial cells. Degranulation takes place due to stimulation with thrombin and then P-selectin is expressed after its transfer to the cell surface. P-selectin is known to recognize the sialyl Le^{X} sugar chain of P-selectin glycoprotein ligand (PSGL-1) molecule and N-terminal sulfated tyrosine residue together.

A mannose-6-phosphate receptor recognizes a structure in which a phosphate group is added at position 6 of non-reduction terminal mannose residue of a high-mannose type sugar chain. Such mannose-6-phosphate receptor is present in the trans-Golgi network. This is a reason why enzyme groups localized in lysosomes have specific sugar chains as labels for selective transport.

The mannose-6-phosphate receptor includes two types: Ca²⁺-independent receptor showing anti-affinity and Ca²⁺-dependent receptor showing low affinity. The former receptor is a 275-kDa transmembrane glycoprotein and the latter receptor is a 46-kDa transmembrane glycoprotein.

Calnexin and calreticulin are types of molecular chaperone and are lectins specifically binding to Glc₁Man₅₋₉GlcNAc sugar chain.

ERGIC-53 (ER-Golgi intermediate compartment) and VIP36 (vesicular integral protein 36) are intracellular lectins containing sugar-binding sites and calcium-binding sites. ERGIC-53 and VIP36 are present in a region ranging from the endoplasmic reticulum to cis-Golgi and a region ranging from the endoplasmic reticulum to the cell membrane, respectively.

The relationships between lectins and organs can be explained as follows. Various types of lectins (sugar chain-recognizing proteins) have been studied as receptors existing on cell membrane surfaces of various types of tissue in vivo, such as C-type lectins (e.g., selectin, DC-SIGN, DC-SGNR, collectin, asialoglycoprotein receptor, and mannose-binding protein), 1-type lectins (e.g., siglec), P-type lectins (e.g., mannose-6-phosphate receptor), R-type lectins, L-type lectins, M-type lectins, and galectin. Sugar chains having various types of molecular structure capable of binding to these lectins are attracting attention as new DDS ligands.

The relationships between lectins and organs are as listed below, in which the expression of lectins in human tissues has been revealed.

(1) Hemocytes and bone marrow cells: an asialoglycoprotein receptor, CD11b, CD18, CD22, CD23, CD31, CD69, galectin-5, galectin-10, interleukin-2, a macrophage mannose receptor, N-CAM (CD56), NKR-P1, and sialoadhesin
(2) Plasma and serum: C-reactive protein, P35, a mannan binding lectin, and serum amyloid P
(3) Bone and cartilages: aggrecan
(4) Epithelial cells of various types of tissue: an asialoglycoprotein receptor

(liver), C-reactive protein (liver), galectin-2 (intestine), galectin-4, and galectin-6 (intestine), galectin-7, HIP and PAP (intestine and pancreas), P35 (liver), a serum amyloid P component (liver), surfactant protein A (lung), and surfactant protein D (lung)

(5) Muscle: sarcolectin
(6) Nerve tissue: brevican, cerebellar-soluble lectin, myelin associated glycoprotein, and N-CAM
(7) Placenta: a placenta Gp120 receptor
Not specifically to tissue: calreticulin, CD44, CD54, ERGIC-53, galectin-1, galectin-3, galectin-8, galectin-9, interleukin1, phosphomannosyl receptor I, phosphomannosyl receptor II, tetranectin, thrombospondin, tumor necrosis factor, and versican

Regarding relationships between lectins and disease tissues, expression of E-selectin, P-selectin, and the like in all the general inflammatory diseases (e.g., encephalitis, chorioretinitis, pneumonia, hepatitis, and arthritis) and diseases that continuously cause inflammation (e.g., malignant tumor, rheumatism, cerebral infarction, diabetes, and Alzheimer disease) is being elucidated. Moreover, expression of various types of lectin including E-selectin, selectin, galectin, siglec, and the like in cancer, brain diseases, cardiac diseases, arteriosclerosis, and the like has been reported. A lot about relationships between lectins and organs or diseases remains unknown and is expected to be elucidated in the future.

When animal lectins are classified in terms of primary structure, they can be classified into the following 14 types of family, for example:

(1) C-type; (2) S-type (galectin); (2) 1-type (siglec and others); (4) P-type (phosphomannosyl receptor); (5) pentraxin; (6) egg lectin; (7) calreticulin and calnexin; (8) ERGIC-53 and VIP-36; (9) discoidin; (10) fucolectin; (11) annexin lectin; (12) ficolin; (13) tachylectin 5A and tachylectin 5B; and (14) slug lectin. The C-type family is classified into the following subfamilies: (1) hyalectin; (2) asialoglycoprotein receptor; (3) collectin; (4) selectin; (5) NK group transmembrane receptor; (6) macrophage mannose receptor; and (7) single domain lectin. Furthermore, as lectins of an orphan lectin group that has sugar chain-binding activity, although the biological significance of which has not yet been elucidated, the following lectins are known: (1) amphotericin; (2) CD11b and CD18; (3) CEL-III, (4) complement factor H; (5) Entamoeba adhesion lectin; (6) frog sialic acid-binding lectin; (7) tachylectin-1 and tachylectin-P; (8) tachylectin-2; (9) tachylectin-3; (10) thrombospondin; (11) interleukin-1; (12) interleukin-2; (13) interleukin-3; (14) interleukin-4; (15) interleukin-5; (16) interleukin-6; (17) interleukin-7; (18) interleukin-8; (19) interleukin-12; and (20) tumor necrosis factor.

As the relationships between a great variety of animal lectins and organs or diseases have been elucidated as described above, the usefulness of the delivery vehicle (e.g., sugar-chain-modified liposome) of the present invention in treatment and diagnosis of diseases will be increased. Furthermore, the delivery vehicle can be applied to a broader range of fields for treatment or diagnosis of diseases. Furthermore, the delivery vehicle of the present invention is also useful as a reagent for research for the purpose of elucidation of the biological significance of a variety of animal lectins.

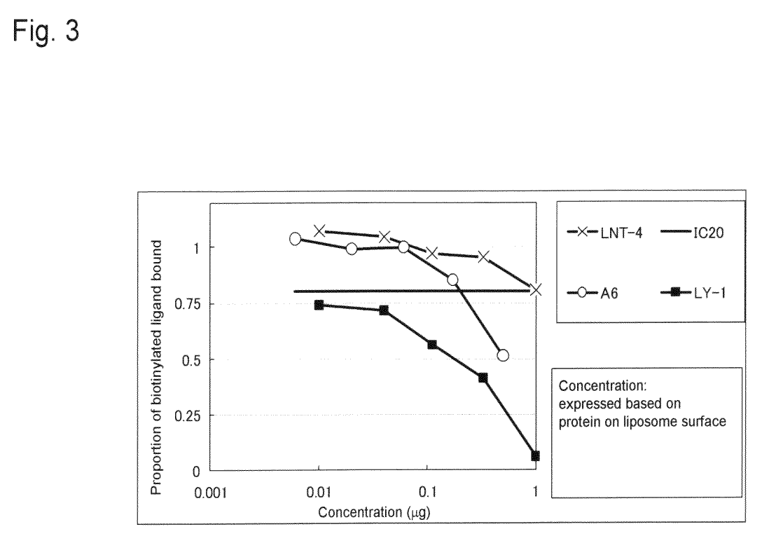

Research and development in the field of drug targeting system (DDS) is classified into passive-targeting DDS and active-targeting DDS in terms of targeting DDS. The passive-targeting DDS is a method for controlling in vivo behavior via alteration of physicochemical properties of a carrier (drug carrier), such as particle diameter or hydrophilicity. The active-targeting DDS is a method for actively enhancing the tropism for a target tissue through the use of a specific mechanism such as a molecular recognition function added to the mechanism of the former method. Research has been conducted concerning active-targeting to date. As a result, many studies have been conducted concerning DDS nanoparticles. For example, liposomes were prepared by binding various types of ligand (e.g., antibody, transferrin, folic acid, and monosaccharide) having molecular recognition functions to recognize various types of cell surface molecule. However, most of these nanoparticles bound to target cells in vitro (ex vivo), but were not targeted in vivo to target tissues or cells expected to be targeted (document 1 and document 2). For example, in vivo distribution of liposomes having an anti-HER2 antibody bound as a ligand (the “anti-HER2” antibody is an antibody against a protein referred to as “HER2” that is a cell surface molecule on breast cancer cell surfaces) thereto and that of liposomes having no such protein bound thereto were examined after their injection via mouse tail vein (document 3). As a result, accumulation of the former liposomes in cancer tissues was not improved because of the binding of anti-HER2. Moreover, in vivo distribution of liposomes having folic acid bound thereto and that of liposomes having no such acid bound thereto were examined using a folic acid receptor (a cell surface molecule of another cancer cell) as a target molecule, after their injection via mouse tail vein (document 4). As a result, the accumulation of the former liposomes in cancer tissues was not improved in spite of binding of folic acid thereto. The target type of these two types of ligand-bound liposome was not the type of cell surface molecule (1) in FIG. 3, but was the type of cell surface molecule (2).

(Document 1) Forssen E., Willis M., Adv. Drug Deliv. Rev., 29, 249-271 (1998).
(Document 2) Vyas S. P. et al., Crit. Rev. Ther. Drug Carrier Syst., 18, 1-76 (2001).

### (Document 3) Drummond D. C. et al., Pharmacological Rev. 51, 691-743 (1999).

(Document 4) Gabizon A. et al., Adv. Drug Deliv. Rev., 56, 1177-1192 (2004).

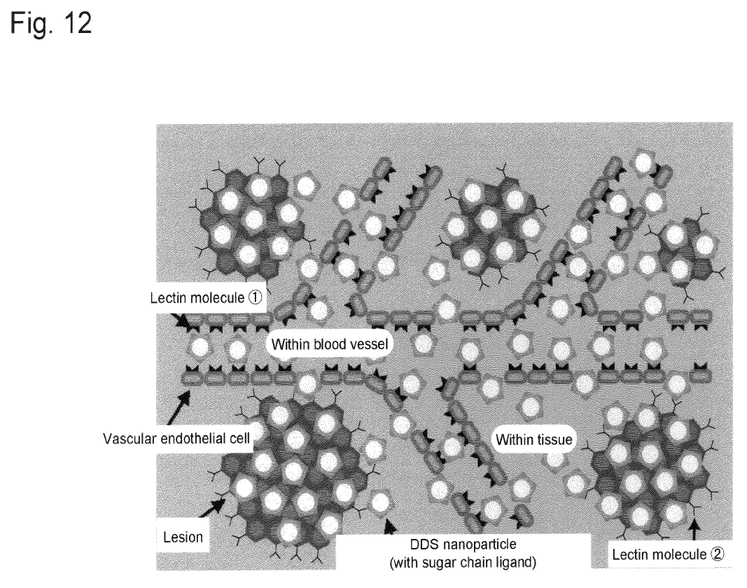

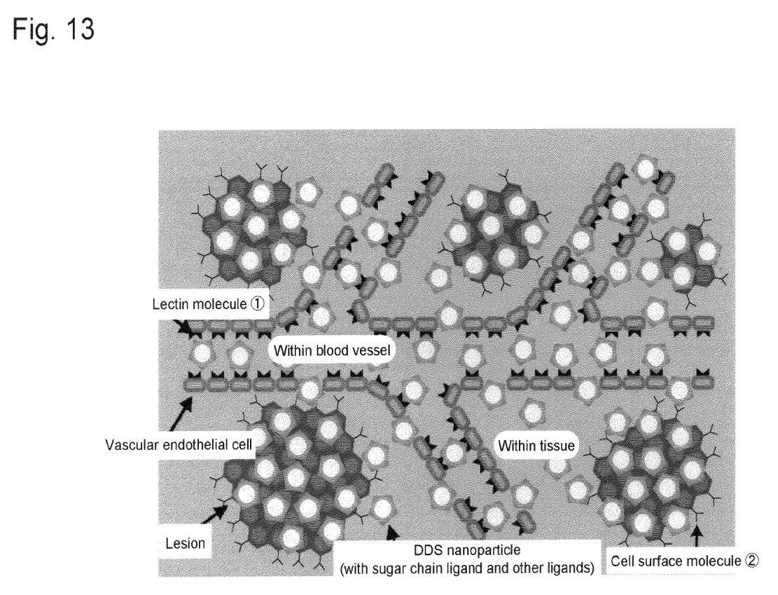

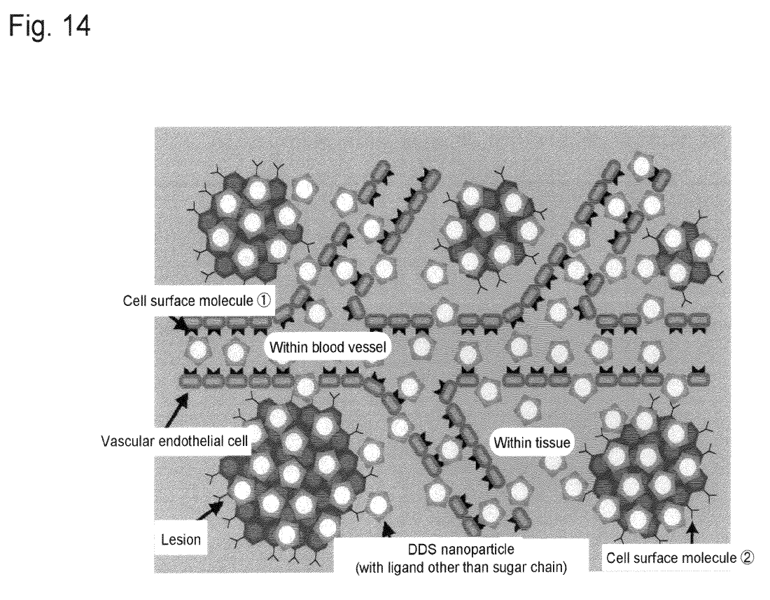

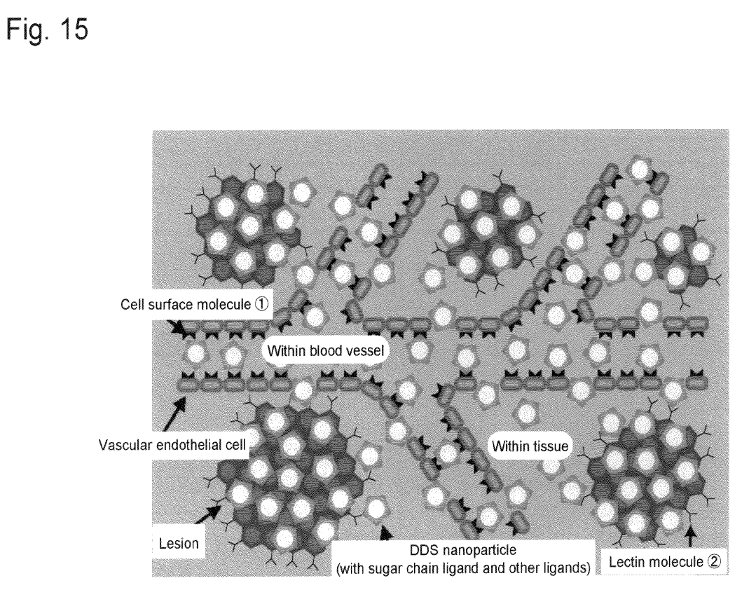

Examples of ligands that can be used in the present invention include, but are not limited to, various types of sugar chain ligand having molecular recognition functions to recognize various types of lectin molecule and various types of ligand having molecular recognition functions to recognize various types of cell surface molecule including many lectin molecules. A target molecule of the DDS delivery vehicle of the present invention is the type of lectin molecule (1) in FIG. 12, for example. Such a target molecule can be used in combination with the type of lectin molecule (2) if necessary. Furthermore, the type of lectin molecule (1) in FIG. 13 can also be used as such a target molecule and the lectin molecule (1) can also be used in combination with the type of cell surface molecule (2) if necessary. Moreover, the type of cell surface molecule (1) in FIG. 14 can also be used as such a target molecule and the molecule (1) can also be used in combination with the type of cell surface molecule (2) if necessary. Furthermore, the type of cell surface molecule (1) in FIG. 15 can also be used as such a target molecule and the molecule (1) can also be used in combination with the type of lectin molecule (2) if necessary.

Currently, lectin molecules have been elucidated as listed in Table 1A below. Lectin molecules will become increasingly elucidated. It is understood that persons skilled in the art having such knowledge will be able to implement various embodiments based on descriptions given for the present invention.

[Table 1A] Lectin molecules that have been elucidated to date
(Document 1) http://www.imperial.ac.uk/research/animallectins/
(Document 2) http://www.cermav.cnrs.fr/lectines/

TABLE 1A
Family/Subfamily/Lectin molecule

I-1. Animal lectins

Calnexin

| | |
|---|---|
| 1 | calnexin |
| 2 | calreticulin |

M-type lectins
L-type lectins(ERGIC, VIP)

| | |
|---|---|
| 1 | ERGIC-53 |

P-type lectins

| | |
|---|---|
| 1 | CD-mannose 6-phosphate receptor |
| 2 | CI-mannose 6-phosphate receptor |

C-type lectin

I - Proteoglycans

| | |
|---|---|
| 1 | Aggrecan |
| 2 | Versican |
| 3 | Neurocan |
| 4 | Brevican |

II - Type 2 receptors

| | |
|---|---|
| 1 | Hepatic Asialoglycoprotein Receptor 1 |
| 2 | Hepatic Asialoglycoprotein Receptor 2 |
| 3 | Macrophage Asialoglycoprotein Receptor |
| 4 | Macrophage Asialoglycoprotein Receptor 1 |
| 5 | Macrophage Asialoglycoprotein Receptor 2 |
| 6 | Scavenger receptor with CTLD, SRCL |
| 7 | Langerhans cell specific C-type lectin, Langerin |
| 8 | Kupffer Cell receptor |
| 9 | Macrophage inducible C-type lectin, Mincle |
| 10 | Blood Dendritic Cell Antigen 2, BDCA-2 |
| 11 | Macrophage C-type lectin, MCL |
| 12 | Denditic cell immunoreceptor, DCIR |
| 13 | Denditic cell immunoreceptor, DCIR1 |
| 14 | Denditic cell immunoreceptor, DCIR2 |
| 15 | Denditic cell immunoreceptor, DCIR3 |
| 16 | Denditic cell immunoreceptor, DCIR4 |
| 17 | Dendritic cell immunoactivating receptor DCAR1 |
| 18 | Dendritic cell immunoactivating receptor, DCAR2 |
| 19 | Dectin-2 |
| 20 | IgE Fc Receptor |
| 21 | DC-specific ICAM3-grabbing non-integrin, DC-SIGN |
| 22 | DC-SIGN-related, DC-SIGNR |
| 23 | Liver sinusoidal epithelial cell lectin, LSECtin |
| 24 | SIGNR1 |
| 25 | SIGNR2 |
| 26 | SIGNR3 |
| 27 | SIGNR4 |
| 28 | SIGNR5 |
| 29 | SIGNR6 - no transcript |
| 30 | SIGNR7 |
| 31 | SIGNR8 |

III - Collectins

| | |
|---|---|
| 1 | Mannose-binding protein C, MBP-C |
| 2 | Mannose-binding protein A, MBP-A |
| 3 | Pulmonary surfactant protein |
| 4 | Pulmonary surfactant protein, SP-D |
| 5 | Collectin-K1 |
| 6 | Collectin-L1 |

IV - Selectins

| | |
|---|---|
| 1 | L-selectin |
| 2 | E-Selectin |
| 3 | P-selectin |

V - NK receptors

| | |
|---|---|
| 1 | C-type lectin, CLEC2L |
| 2 | Myeloid DAP12-associating lectin, MDL-1 |
| 3 | CD72 |
| 4 | Mast cell function-associated antigen, MAFA |
| 5 | CD69 |
| 6 | Killer cell lectin receptor, KLRF1 |
| 7 | Activation inducible C-type lectin, AICL |
| 8 | Oxidised LDL receptor |
| 9 | CD94 |
| 10 | C-type lectin receptor, CLEC-1 |
| 11 | C-type lectin receptor, CLEC-2 |
| 12 | Similar to CLEC1B |
| 13 | CLEC1D |
| 14 | Dendritic cell-associated lectin, DCAL-1 |
| 15 | Dendritic cell-associated lectin, DCAL-2 |
| 16 | C-type lectin, CLEC9A |
| 17 | C-type lectin, CLEC2A |
| 18 | Dectin-1 |
| 19 | Osteoclast inhibitory lectin, Ocil |
| 20 | Ocil-related protein, OcilrP1 |
| 21 | C lectin related protein ClrC |
| 22 | Ocil-related protein, OcilrP2 |
| 23 | C lectin related protein ClrA |
| 24 | C lectin related protein ClrF |
| 25 | Similar to CLEC2G |
| 26 | CLEC15A, similar to ClrA |
| 27 | Killer cell lectin receptor, KLRH1 |
| 28 | Killer cell lectin receptor, KLRI1 |
| 29 | Killer cell lectin receptor, KLRI2 |
| 30 | Similar to KLRI1 |
| 31 | Natural killer cell receptor, NKR-P1A |
| 32 | Natural killer cell receptor, NKR-P1A |
| 33 | Natural killer cell receptor, NKR-P1B |
| 34 | Natural killer cell receptor, NKR-P1C |
| 35 | Natural killer cell receptor, NKR-P1D |
| 36 | Natural killer cell receptor, NKR-P1F |
| 37 | Natural killer group receptor, NKG2A |
| 38 | Natural killer group receptor, NKG2B |
| 39 | Natural killer group receptor, NKG2C |
| 40 | Natural killer group receptor, NKG2D |
| 41 | Natural killer group receptor, NKG2F |
| 42 | Natural killer group receptor, NKG2E |
| 43 | Natural killer group receptor, NKG2I |
| 44 | Lymphocyte antigen, LY49L |
| 45 | Lymphocyte antigen, Ly49a |
| 46 | Lymphocyte antigen, Ly49c |
| 47 | Lymphocyte antigen, Ly49h |
| 48 | Lymphocyte antigen, Ly49k |
| 49 | Lymphocyte antigen, Ly49n |
| 50 | Lymphocyte antigen, Ly49o |
| 51 | Lymphocyte antigen, Ly49u |
| 52 | Lymphocyte antigen, Ly49v |
| 53 | Lymphocyte antigen, Ly49w |
| 54 | Lymphocyte antigen, Ly49m |
| 55 | Lymphocyte antigen, Ly49l |
| 56 | Lymphocyte antigen, Ly49i |
| 57 | Lymphocyte antigen, Ly49b |
| 58 | Lymphocyte antigen, Ly49d |
| 59 | Lymphocyte antigen, Ly49p |
| 60 | Lymphocyte antigen, Ly49r |
| 61 | Lymphocyte antigen, Ly49e |
| 62 | Lymphocyte antigen, Ly49f |
| 63 | Lymphocyte antigen, Ly49s |
| 64 | Lymphocyte antigen, LY49g |
| 65 | Lymphocyte antigen, LY49t |
| 66 | Lymphocyte antigen, Ly49j |
| 67 | Lymphocyte antigen, Ly49q |

VI - MMR family

| | |
|---|---|
| 1 | Macrophage mannose receptor, MMR |
| 2 | Endo180 |
| 3 | Phospholipase A2 receptor |
| 4 | Dendritic and epithelial cell receptor, DEC-205 |

VII - Free CTLDs/Reg

| | |
|---|---|
| 1 | Lithostathine-alpha |
| 2 | Lithostathine-beta |
| 3 | Pancreatitis-associated protein, PAP/HIP |
| 4 | Pancreatitis-associated protein, PAPIB |
| 5 | Pancreatitis-associated protein, PAP1 |
| 6 | Pancreatitis-associated protein, PAP2 |
| 7 | Pancreatitis-associated protein, PAP3 |
| 8 | Reg 3-delta |
| 9 | Reg 4 |

VIII - Type 1 receptors

| | |
|---|---|
| 1 | Layilin |
| 2 | Chondrolectin |

IX - Tetranectin family

| | |
|---|---|
| 1 | Tetranectin (plasminogen-binding protein) |
| 2 | Cartilage-derived C-type lectin |
| 3 | Stem cell growth factor, SCGF |

X - Polycystin

| | |
|---|---|
| 1 | Polycystin-1 |

XI - Attractin family

| | |
|---|---|
| 1 | Attractin |
| 2 | Attractin-like protein, ALP |

XII - CTLD/acidic neck

| | |
|---|---|
| 1 | DEC-205-associated C-type lectin, DCL-1 |
| 2 | Eosinophil Major Basic Protein, EMBP |
| 3 | Eosinophil Major Basic Protein Homologue, EMBPH |

XIII - IDD

| | |
|---|---|
| 1 | Integral membrane protein, deleted in DiGeorge |
| | Syndrome, IDD |

XIV - Endosialin family

| | |
|---|---|
| 1 | Endosialin |
| 2 | CD93 |
| 3 | Thrombomodulin |
| 4 | C-type lectin, CLEC14A |

Others

| | |
|---|---|
| 1 | lung surfactant protein |
| 2 | rattlesnake venom lectin |
| 3 | sea cucumber CEL-I |
| 4 | tunicate |

Galectins

| | |
|---|---|
| 1 | galectin-1 |
| 2 | galectin-2 |
| 3 | galectin-3 |
| 4 | galectin-4 |
| 5 | galectin-5 |
| 6 | galectin-6 |
| 7 | galectin-7 |
| 8 | galectin-8 |
| 9 | galectin-9 |
| 10 | galectin-10 |
| 11 | galectin-11 |
| 12 | galectin-12 |
| 13 | galectin-13 |
| 14 | galectin-14 |
| 15 | galectin-15 |
| 16 | galectin-16 |
| 17 | chicken galectin |
| 18 | congerin-1 |
| 19 | congerin-2 |

I-type lectins (siglecs)

| | |
|---|---|
| 1 | Sialo adhesin or Siglec-1 |
| 2 | CD22 or Siglec-2 |
| 3 | MAG or Siglec-4 |
| 4 | CD33 or Siglec-3 |
| 5 | Siglec-5 |
| 6 | Siglec-6 |
| 7 | Siglec-7 |
| 8 | Siglec-8 |
| 9 | Siglec-9 |
| 10 | Siglec-10 |
| 11 | Siglec-11 |
| 12 | Siglec-12 |
| 13 | Siglec-12a |
| 14 | Siglec-12b |
| 15 | Siglec-13 |
| 16 | Siglec-E |
| 17 | Siglec-F |
| 18 | Siglec-G |
| 19 | Siglec-H |

R-type lectins

| | |
|---|---|
| 1 | Ricin 1 |
| 2 | Ricin 2 |
| 3 | Xylanase |
| 4 | Arabinosidase |
| 5 | Protease |
| 6 | Glucanase |
| 7 | HS GALNT |
| 8 | DM GALNT3 |
| 9 | DM GALNT6 |
| 10 | DM GALNT35 |
| 11 | DM GALNT5 |
| 12 | DM GALNT1 |
| 13 | DM GALNT7 |
| 14 | DM GALNT8 |
| 15 | DM GALNT2 |
| 16 | DM GALNT11 |
| 17 | DM GALNT13 |
| 18 | DM GALNT10 |
| 19 | DM GALNT4 |
| 20 | DM GALNT9 |
| 21 | DM GALNT12 |
| 22 | CE GALNT3 |
| 23 | CE GALNT4 |
| 24 | CE GALNT5a |
| 25 | CE GALNTb |
| 26 | CE GALNTc |
| 27 | CE GALNT6a |
| 28 | CE GALNT6b |
| 29 | CE GALNT7 |
| 30 | CE GALNT9 |
| 31 | CE GALNT10 |
| 32 | CE GALNT11 |
| 33 | MMR |
| 34 | Endo180 |
| 35 | PLA2R |
| 36 | DEC205 |

Other animal lectins

Chitin-binding protein

| | |
|---|---|
| 1 | tachycitin |

fucolectin

| | |
|---|---|
| 1 | eel lectin |

H-type lectin

| | |
|---|---|
| 1 | Helix pomatia agglutinin |

Pentraxin

| | |
|---|---|
| 1 | homo sapiens |
| 2 | limulus polyphemus |
| 3 | Mesocricetus auratus |
| 4 | serum amyloid P component |

R-type lectin (b-trefoil)

| | |
|---|---|
| 1 | Cys-rich domain man-receptor |
| 2 | sea cucumber CEL-III |

Spider Toxin

| | |
|---|---|
| 1 | selenocosmia huwena lectin-I |

Tachylectin

| | |
|---|---|
| 1 | tachylectin-2 |
| 2 | tachylectin-5A |

TIM-lectin

| | |
|---|---|
| 1 | Hcgp-39 |
| 2 | Ym1 |

Others

| | |
|---|---|
| 1 | Pentraxins |
| 2 | (Trout)egg lectins |
| 3 | Calreticulin and calnexin |
| 4 | Discoidins |
| 5 | Annexin lectins |
| 6 | Ficolins |
| 7 | achylectins 5A & 5B |
| 8 | Lima flavus agglutinin |

I-2. Bacterial lectins

1-Ca b-sandwich

| | |
|---|---|
| 1 | Pseudomonas PA-IL |

2-Ca b-sandwich

| | |
|---|---|
| 1 | Chromobacterium CV-IIL |
| 2 | Pseudomonas PA-IIL |
| 3 | Ralstonia RS-IIL |

ADP-ribosylating toxin (AB5 toxin)

| | |
|---|---|
| 1 | cholera toxin |
| 2 | heat-labile enterotoxin |
| 3 | pertussis toxin |
| 4 | verotoxin 1 (shiga-like) |

b-propeller

| | |
|---|---|
| 1 | Ralstonia lectin |

Bacterial neurotoxin

| | |
|---|---|
| 1 | Botulinum toxin |
| 2 | Clostridium hemagglutinin |
| 3 | Tetanus toxin |

cyanobacterial lectins

| | |
|---|---|
| 1 | cyanovirin-N |
| 2 | Mycroscystis lectin |

Pili adhesin

| | |
|---|---|
| 1 | FimH |
| 2 | GafD (F17-G) |
| 3 | PapG |

Staphylococcal toxin

| | |
|---|---|
| 1 | Staphylococcal enterotoxin B |

Toxin repetitive domain

| | |
|---|---|
| 1 | Clostridium repetitve domain |

I-3. Fungal lectins

6-blades b-propeller

| | |
|---|---|
| 1 | Aleuria aurantia lectin |

7-blades b-propeller

| | |
|---|---|
| 1 | integrin-like lectin |

actinoporin-like

| | |
|---|---|
| 1 | Agaricus lectin, Xerocomus lectin |

b-trefoil pore forming

| | |
|---|---|
| 1 | Laetiporus lectin |

galectin

| | |
|---|---|
| 1 | agrocybe galectin |
| 2 | coprinus galectin-2 |

Ig-like

| | |
|---|---|
| 1 | Fip-Fve |

I-4. Plant lectins

a-D-mannose-specific plant lectin (monocot lectin)

| | |
|---|---|
| 1 | amaryllis |
| 2 | Bluebell SCA-FET |
| 3 | Bluebell SCA-MAN |
| 4 | daffodil amaryllidaceae |
| 5 | garlic bulbs lectin |
| 6 | gastrodiadin |
| 7 | snowdrop lectin |

Agglutinin with hevein domain

| | |
|---|---|
| 1 | Amaranthus antimicrobial peptide |
| 2 | Hevein, Pokeweed lectin |
| 3 | Urtica dioica UDA |
| 4 | Wheat germ WGA-1 |
| 5 | Wheat germ WGA-2 |
| 6 | Wheat germ WGA-3 |

b-prism plant lectin

| | |
|---|---|
| 1 | artocarpin |
| 2 | Artocarpus hirsuta AHL |
| 3 | banana lectin |
| 4 | Calsepa |
| 5 | heltuba |
| 6 | jacalin |
| 7 | Maclura pornifera MPA |
| 8 | MornigaM |
| 9 | Parkia lectins |

b-trefoil lectin

| | |
|---|---|
| 1 | abrin-a |
| 2 | castor bean ricin B |
| 3 | amaranthin |
| 4 | ebulin |
| 5 | Misteltoe lectin |
| 6 | TKL-1 |

Legume lectin

| | |
|---|---|
| 1 | canavalia brasiliens |
| 2 | Canavalia maritima |
| 3 | concanavalin A |
| 4 | Cratylia mollis |
| 5 | Dioclea grandiflora DGL |
| 6 | Dioclea guianensis lectin |
| 7 | Dolichos biflorus DB58 |
| 8 | Dolichos biflorus DBL |
| 9 | Dolichos |
| 10 | lablab FRIL |
| 11 | Erythrina corallodendron EcorL |
| 12 | Erythrina cristagalli ECL |
| 13 | favin |
| 14 | Griffonia simplicifolia GS-I |
| 15 | Griffonia simplicifolia GS-IV |
| 16 | Lathyrus ochrus LOL-1 |
| 17 | Lathyrus ochrus LOL-2 |
| 18 | Lentil LCL, lima bean LBL |
| 19 | Maackia amurensis MAL |
| 20 | Pea PSL, Peanut PNA |
| 21 | Phaseolus vulgaris PHA-L |
| 22 | Pterocarpus angolensis |
| 23 | Robinia pseudoacacia bark lectin I |
| 24 | Soybean SBA |
| 25 | Ulex europaeus UEA-1 |
| 26 | Ulex europaeus UEA-2 |
| 27 | Vicia villosa VVL-B4 |
| 28 | winged bean agglutinin I |
| 29 | winged bean agglutinin II |

I-5. Virus lectins

Capsid spike protein

| | |
|---|---|
| 1 | rotavirus |

Coat protein

| | |
|---|---|
| 1 | foot-and-mouth disease virus |
| 2 | virus coat protein |

Fiber knob

| | |
|---|---|
| 1 | adenovirus |

Hemagglutinin

| | |
|---|---|
| 1 | influenza virus |

Tailspike protein

| | |
|---|---|
| 1 | phage P22 |

Cell surface molecules have been elucidated to date as listed in Table 1B below. Cell surface molecules will become increasingly elucidated. It is understood that persons skilled in the art having such knowledge will be able to implement various embodiments based on descriptions given for the present invention.

[Table 1B] Cell surface molecules that have been elucidated to date
(Document 3) http://www.hlda8.org/HLDAtoHCDM.htm

TABLE 1B

| | CD No. | Alternate term |
|---|---|---|
| 1 | CD1a | T6/leu-6, R4, HTA1 |
| 2 | CD1b | R1 |
| 3 | CD1c | M241, R7 |
| 4 | CD1d | R3 |
| 5 | CD1e | R2 |
| 6 | CD2 | T11, Tp50, sheep red blood cell (SRBC) receptor, LFA-2 |
| 7 | CD3d | CD3 complex, T3, Leu4 |
| 8 | CD3e | |
| 9 | CD3g | |
| 10 | CD4 | OKT4, Leu 3a, T4 |
| 11 | CD5 | Tp67, T1, Ly1, Leu-1 |
| 12 | CD6 | T12 |
| 13 | CD7 | Leu 9, 3A1, gp40, T cell leukemia antigen |
| 14 | CD8a | OKT8, LeuT, LyT2, T8 |
| 15 | CD8b | |
| 16 | CD9 | Drap-27, MRP-1, p24, leucocyte antigen MIC3 |
| 17 | CD10 | CALLA, membrane metallo-endopeptidase |
| 18 | CD11a | alphaL, LFA-1, gp180/95 |
| 19 | CD11b | alphaM, alpha-chain of C3bi receptor, gp155/95, Mac-1, Mo1 |
| 20 | CD11c | alphaX, a-chain of: complement receptor type 4 (CR4), gp150/95 |
| 21 | CD12a | TNFRI, TNFRp55 |
| 22 | CDw12 | P90-120 |
| 23 | CD13 | Aminopeptidase N, APN, gp150, EC 3.4.11.2 |
| 24 | CD14 | LPS receptor |
| 25 | CD15 | Lewis X, CD 15u: sulphated Lewis X. CD 15s: sialyl Lewis X |
| 26 | CD16a | Fc gamma R IIIa, |
| 27 | CD16b | Fc gamma R IIIb |
| 28 | CD17 | Lactosylceramide |
| 29 | CDw17 | LacCer, lactosylceramide |
| 30 | CD18 | β2-Integrin chain, macrophage antigen 1 (mac-1) |
| 31 | CD19 | Bgp95, B4 |
| 32 | CD20 | B1, membrane-spanning 4-domains, subfamily A, member 1 |
| 33 | CD21 | C3d receptor, CR2, gp140, EBV receptor |
| 34 | CD22 | Bgp135, BL-CAM, Siglec2 |
| 35 | CD23 | Low affinity IgE receptor, FceRII, gp50-45, Blast-2 |
| 36 | CD24 | heat stable antigen homologue (HSA), BA-1 |
| 37 | CD25 | Interleukin (IL)-2 receptor a-chain, Tac-antigen |
| 38 | CD26 | Dipeptidylpeptidase IV, gp120, Ta1 |
| 39 | CD27 | T14, S152 |
| 40 | CD28 | Tp44 |
| 41 | CD29 | Integrin β1 chain, platelet GPIIa, VLA (CD49) beta-chain |
| 42 | CD30 | Ki-1 antigen, Ber-H2 antigen |
| 43 | CD31 | PECAM-1, platelet GPIIa', endocam |
| 44 | CD32 | Fcgamma receptor type II (FcgRII), gp40 |
| 45 | CD33 | My9, gp67, p67 |
| 46 | CD34 | My10, gp105-120 |
| 47 | CD35 | C3b/C4b receptor, complement receptor type 1 (CR1) |
| 48 | CD36 | platelet GPIV, GPIIIb, OKM-5 antigen |
| 49 | CD37 | gp40-52 |
| 50 | CD38 | T10, gp45, ADP-ribosyl cyclase |
| 51 | CD39 | gp80, ectonucleoside triphosphate diphosphohydrolase 1 |
| 52 | CD40 | Bp50, TNF Receptor 5 |
| 53 | CD41 | platelet glycoprotein GPIIb |
| 54 | CD42a | platelet glycoprotein GPIX |
| 55 | CD42b | platelet glycoprotein GPIb-a |
| 56 | CD42c | platelet glycoprotein GPIb-β |
| 57 | CD42d | platelet glycoprotein GPV |
| 58 | CD43 | Leukosialin, gp95, sialophorin, leukocyte sialoglycoprotein |
| 59 | CD44R | CD44 variant, CD44v1-10 |
| 60 | CD44 | Pgp-1, gp80-95, Hermes antigen, ECMR-III and HUTCH-I. |
| 61 | CD45RA | Restricted T200, gp220, isoform of leukocyte common antigen |
| 62 | CD45RB | Restricted T200, isoform of leukocyte common antigen |
| 63 | CD45RC | Restricted T200, isoform of leukocyte common antigen |
| 64 | CD45RO | Restricted T200, gp180, |
| 65 | CD45 | LCA, B220, protein tyrosine phosphatase, receptor type, C |
| 66 | CD46 | Membrane cofactor potein (MCP) |
| 67 | CD47 | Integrin-associated protein (IAP), Ovarian carcinoma antigen OA3 |
| 68 | CD48 | BLAST-1, Hulym3, OX45, BCM1 |
| 69 | CD49a | Integrin a1 chain, very late antigen, VLA 1a |
| 70 | CD49b | Integrin a2 chain, VLA-2-alpha chain, platelet gpla |
| 71 | CD49c | Integrin a3 chain, VLA-3 alpha chain |
| 72 | CD49d | Integrin a4 chain, VLA-4-alpha chain |
| 73 | CD49e | Integrin a5 chain, VLA-5 alpha chain |
| 74 | CD49f | Integrin a6 chain, VLA-6 alpha chain, platelet gplc |
| 75 | CD50 | ICAM-3, intercellular adhesion molecule 3 |
| 76 | CD51 | Integrin alpha chain, vitronectin receptor a chain |
| 77 | CD52 | Campath-1, HE5 |
| 78 | CD53 | MRC OX-44 |
| 79 | CD54 | ICAM-1, intercellular adhesion molecule 1 |
| 80 | CD55 | DAF, Decay Accelerating Factor |
| 81 | CD56 | NKHI, Neural cell adhesion molecule (NCAM) |
| 82 | CD57 | HNK1 |
| 83 | CD58 | LFA-3, lymphocyte function associated antigen-3 |
| 84 | CD59 | MACIF, MIRL, P-18, protectin |
| 85 | CD60 | GD3 (CD60a), 9-0-acetyl GD3 (CD60b), 7-0-acetyl GD3 (CD60c) |
| 86 | CD61 | Glycoprotein IIIa, beta3 integrin |
| 87 | CD62E | E-selectin, LECAM-2, ELAM-1 |
| 88 | CD62L | L-selectin, LAM-1, Mel-14 |
| 89 | CD62P | P-selectin, granule membrane protein-140 (GMP-140) |
| 90 | CD63 | LIMP, gp55, LAMP-3 neuroglandular antigen, granulophysin |
| 91 | CD64 | FcgR1, FcgammaR1 |
| 92 | CD65s | Sialylated-CD65, VIM2 Antigen |
| 93 | CD65 | Ceramide dodecasaccharide 4c, VIM2 |
| 94 | CD66a | BGP, carcinoembryonic antigen-related cell adhesion molecule 1 |
| 95 | CD66b | CGM6, NCA-95 |
| 96 | CD66c | nonspecific crossreaction antigen, NCA-50/90 |
| 97 | CD66d | CGM1 |
| 98 | CD66e | CEA |
| 99 | CD66f | PSG, Sp-1, pregnancy specific (b1) glycoprotein |
| 100 | CD68 | gp110, macrosialin |
| 101 | CD69 | AIM, activation inducer molecule, MLR3, EA1, VEA |
| 102 | CD70 | CD27 ligand, KI-24 antigen |
| 103 | CD71 | Transferrin receptor |
| 104 | CD72 | Lyb-2, Ly-19.2, Ly32.2 |
| 105 | CD73 | Ecto-5′-nucleotidase |
| 106 | CD74 | MHC Class II associated invariant chain (li) |
| 107 | CD75s | Alpha-2, 6-sialylated lactosamines (formerly CDw75 and CDw76) |
| 108 | CD75 | Lactosamines |
| 109 | CDw76 | Since HLDA7, CDw76 has been renamed CD75s |
| 110 | CD77 | Pk blood group antigen, Burkitt's lymphoma associated antigen |
| 111 | CD79a | MB-1, Iga |
| 112 | CD79b | B29, Igβ |
| 113 | CD80 | B7-1, BB1 |
| 114 | CD81 | Target of an antiproliferative antibody (TAPA-1), M38 |
| 115 | CD82 | R2, 4F9, C33, IA4, kangai 1 |
| 116 | CD83 | HB15 |
| 117 | CD84 | p75, GR6 |
| 118 | CD85a | ILT5, LIR3, HL9 |
| 119 | CD85d | ILT4, LIR2, MIR10 |
| 120 | CD85j | LIR-1, ILT2 (immunoglobulin-like transcript 2), MIR7 |
| 121 | CD85k | ILT3, LIR5, HM18 |
| 122 | CD86 | B7-2, B70 |
| 123 | CD87 | Urokinase plasminogen activator-receptor (uPA-R) |
| 124 | CD88 | C5a-receptor |
| 125 | CD89 | Fca-receptor, IgA-receptor |
| 126 | CD90 | Thy-1 |
| 127 | CD91 | a2-macroglobulin receptor (ALPHA2M) |
| 128 | CD92 | P70, CHTL1 |
| 129 | CDw92 | p70 |
| 130 | CD93 | C1QR1 |
| 131 | CDw93 | GR11 |
| 132 | CD94 | kP43, killer cell lectin-like receptor subfamily D, member 1 |
| 133 | CD95 | APO-1, Fas, TNFRSF6 |
| 134 | CD96 | TACTILE (T cell activation increased late expression) |
| 135 | CD97 | BL-KDD/F12 |
| 136 | CD98 | 4F2, FRP-1 |
| 137 | CD99 | MIC2, E2 |
| 138 | CD100 | SEMA4D |
| 139 | CD101 | V7, P126 |
| 140 | CD102 | ICAM-2 |
| 141 | CD103 | Integrin alpha E subunit, HML-1 |
| 142 | CD104 | Integrin beta 4 subunit, TSP-1180 |
| 143 | CD105 | Endoglin |
| 144 | CD106 | VCAM-1 (vascular cell adhesion molecule-1), INCAM-110 |
| 145 | CD107a | Lysosomal associated membrane protein (LAMP)-1 |
| 146 | CD107b | Lysosomal associated membrane protein (LAMP)-2 |
| 147 | CD108 | GPI-gp80, John-Milton-Hagen (JMH) human blood group antigen |
| 148 | CD109 | Platelet activation factor, 8A3, E123 |
| 149 | CD110 | Thrombopoietin receptor, c-mpl |
| 150 | CD111 | PRR1, Nectin 1, Hve C1, poliovirus receptor related 1 protein |
| 151 | CD112 | PRR2, Nectin 2, Hve B, poliovirus receptor related 2 protein |
| 152 | CD113 | Nectin 3 |
| 153 | CDw113 | PVRL3, Nectin3 |
| 154 | CD114 | G-CSFR, HG-CSFR, CSFR3 |
| 155 | CD115 | M-CSFR, CSF-1, C-fms |
| 156 | CD116 | GMCSF R alpha subunit, |
| 157 | CD117 | SCFR, c-kit, stem cell factor receptor |
| 158 | CD118 | LIFR |
| 159 | CD119 | IFN gamma receptor alpha chain |
| 160 | CD119 | IFN γ receptor |
| 161 | CD120b | TNFRII, TNFRp75 |
| 162 | CD121a | Type I IL-1 receptor |
| 163 | CD121b | Type II IL-1 receptor |
| 164 | CD121b | Type II IL-1 receptor |
| 165 | CD122 | IL-2 receptor betachain, p75 |
| 166 | CD123 | Interleukin-3 receptor alpha chain (IL-3Ra) |
| 167 | CD124 | IL-4 R alpha chain |
| 168 | CD125 | IL-5R α chain |
| 169 | CDw125 | Interleukin-5 receptor α chain |
| 170 | CD126 | IL-6 receptor alpha chain |
| 171 | CD127 | IL-7 receptor alpha chain, p90 |
| 172 | CD129 | IL-9 receptor alpha chain |
| 173 | CD130 | gp130 |
| 174 | CD131 | Common β chain, low-affinity (granulocyte-macrophage) |
| 175 | CD131 | Common β chain |
| 176 | CD132 | Common gamma chain, interleukin 2 receptor, gamma |
| 177 | CD133 | AC133, PROML1, prominin 1 |
| 178 | CD134 | OX 40, TNFRSF4 |
| 179 | CD135 | FLT3, STK-1, flk-2 |
| 180 | CD136 | MSP-R, RON |
| 181 | CDw136 | Macrophage stimulating protein receptor, MSP-R, RON |
| 182 | CD137 | 4-1BB, TNFRSF9 |
| 183 | CDw137 | 4-1BB, Induced by lymphocyte activation (ILA) |
| 184 | CD138 | Syndecan-1, B-B4 |
| 185 | CD139 | |
| 186 | CD140a | a-platelet derived growth factor (PDGF) receptor |
| 187 | CD140b | b-platelet derived growth factor (PDGF) receptor |
| 188 | CD141 | Thrombomodulin (TM), fetomodulin |
| 189 | CD142 | Tissue factor, thromboplastin, coagulation factor III |
| 190 | CD143 | Angiotensin-converting enzyme (ACE), peptidyl dipeptidase A |
| 191 | CD144 | VE-cadherin, cadherin-5 |
| 192 | CDw145 | None |
| 193 | CD146 | Muc 18, MCAM, Mel-CAM, s-endo |
| 194 | CD147 | Basigin, M6, extracellular metalloproteinase inducer (EMMPRIN) |
| 195 | CD148 | DEP-1, HPTP-n, protein tyrosine phosphatase, receptor type, |
| 196 | CD150 | SLAM, signalling lymphocyte activation molecule, IPO-3 |
| 197 | CD151 | Platelet-endothelial tetra-span antigen (PETA)-3 |
| 198 | CD152 | Cytotoxic T lymphocyte antigen (CTLA)-4 |
| 199 | CD153 | CD30 Ligand |
| 200 | CD154 | CD40 Ligand, TRAP (TNF-related activation protein)-1, T-BAM |
| 201 | CD155 | Polio virus receptor (PVR) |
| 202 | CD156a | ADAM-8, a disintegrin and metalloproteinase domain 8 |
| 203 | CD156b | TACE, ADAM 17 snake venom like protease CSVP |
| 204 | CD156c | ADAM10 |
| 205 | CDw156C | ADAM10 |
| 206 | CD157 | BST-1 BP-3/IF7 Mo5 |
| 207 | CD158E1/2 | killer cell Ig-like receptor, three domains, long cytoplasmic tail, 1 |
| 208 | CD158i | killer cell Ig-like receptor, two domains, short cytoplasmic tail, 4 |
| 209 | CD158k | killer cell Ig-like receptor, two domains, long cytoplasmic tail, 2 |
| 210 | CD159a | killer cell lectin-like receptor subfamily C, member 1 |
| 211 | CD159c | NKG2C |
| 212 | CD160 | BY55, NK1, NK28 |
| 213 | CD161 | NKR-P1A, killer cell lectin-like receptor subfamily B, member 1 |
| 214 | CD162R | PEN5 |
| 215 | CD162 | P selectin glycoprotein ligand 1, PSGL-1 |
| 216 | CD163 | GHI/61, D11, RM3/1, M130 |
| 217 | CD164 | MUC-24, MGC 24, multi-glycosylated core protein 24 |
| 218 | CD165 | AD2, gp 37 |
| 219 | CD166 | ALCAM, KG-CAM, activated leukocyte cell adhesion molecule |
| 220 | CD167 | Discoidin receptor DDR1 (CD 167a) and DDR2 (CD 167b) |
| 221 | CD168 | RHAMM (receptor for hyaluronan involved in migration & motility) |
| 222 | CD169 | Sialoadhesin, Siglec-1 |
| 223 | CD170 | Siglec 5 (sialic acid binding Ig-like lectin 5) |
| 224 | CD171 | Neuronal adhesion molecule, LI |
| 225 | CD172a | SIRPa, signal inhibitory regulatory protein family member |
| 226 | CD172b | SIRPbeta |
| 227 | CD172g | SIRPgamma |
| 228 | CD173 | Blood Group H2 |
| 229 | CD174 | Lewis Y blood group, LeY, fucosyltransferase 3 |
| 230 | CD175s | Sialyl-Tn |
| 231 | CD175 | Tn Antigen (T-antigen novelle) |
| 232 | CD176 | Thomsen-Friedenreich antigen (TF) |
| 233 | CD177 | NB 1 |
| 234 | CD178 | FAS ligand, CD95 ligand |
| 235 | CD179a | V pre beta |
| 236 | CD179b | Lambda 5 |
| 237 | CD180 | RP105, Bgp95 |
| 238 | CD181 | CXCR1, (was CDw128A) |
| 239 | CD182 | CXCR2, (was CDw128B) |
| 240 | CD183 | CXCR3 chemokine receptor, G protein-coupled receptor 9 |
| 241 | CD184 | CXCR4 chemokine receptor, Fusin |
| 242 | CD185 | CXCR5 |
| 243 | CD186 | CXCR6 |
| 244 | CDw186 | CXCR6 |
| 245 | CD191 | CCR1 |
| 246 | CD192 | CCR2 |
| 247 | CD193 | CCR3 |
| 248 | CD194 | CCR4 |
| 249 | CD195 | CCR5 chemokine receptor |
| 250 | CD196 | CCR6 |
| 251 | CD197 | CCR7 |
| 252 | CDw198 | CCR8 |
| 253 | CDw199 | CCR9 |
| 254 | CD200 | MRC OX 2 |
| 255 | CD201 | Endothelial protein C receptor (EPCR) |
| 256 | CD202b | TIE2, TEK |
| 257 | CD203c | E-NPP3, PDNP3, PD-1beta |
| 258 | CD204 | MSR, SRA, Macrophage scavenger receptor |
| 259 | CD205 | DEC-205 |
| 260 | CD206 | Macrophage mannose receptor (MMR) |
| 261 | CD207 | Langerin |
| 262 | CD208 | DC-LAMP |
| 263 | CD209 | DC-SIGN |
| 264 | CDw210 | IL-10 receptor |
| 265 | CD212 | IL-12 receptor beta chain |
| 266 | CD213a1 | IL-13 receptor alpha 1 |
| 267 | CD213a2 | IL-13 R alpha 2 |
| 268 | CD217 | IL-17R |
| 269 | CDw217 | IL-17 receptor |
| 270 | CD218a | IL-18R α chain |
| 271 | CDw218a | IL18Ralpha |
| 272 | CD218b | IL-18R β chain |
| 273 | CDw218b | IL18Rbeta |
| 274 | CD220 | Insulin Receptor |
| 275 | CD221 | IGF I Receptor, type I IGF receptor |
| 276 | CD222 | Mannose-6-phosphate receptor, insulin like growth factor II R |
| 277 | CD223 | LAG-3 (Lymphocyte activation gene 3) |
| 278 | CD224 | Gamma-glutamyl transferase, GGT |
| 279 | CD225 | Leu-13, interferon-induced transmembrane protein 1 |
| 280 | CD226 | DNAM-1, DTA-1 |
| 281 | CD227 | MUC 1 |
| 282 | CD228 | Melanotransferrin, p97 |
| 283 | CD229 | Ly9 |
| 284 | CD230 | Prion protein, PrP(c), PrP(sc) abnormal form |
| 285 | CD231 | TALLA-1, TM4SF2 |
| 286 | CD232 | VESPR |
| 287 | CD233 | Band 3, AE1, anionexchanger 1, Diego blood group antigen |
| 288 | CD234 | DARC, Fy-glycoprotein, Duffy blood group antigen |
| 289 | CD235a | Glycophorin A |
| 290 | CD235b | Glycophorin B |
| 291 | CD236R | Glycophorin C |
| 292 | CD236 | Glycophorin C/D |
| 293 | CD238 | Kell blood group antigen |
| 294 | CD239 | B-CAM, lutheran glycoprotein |
| 295 | CD240CE | Rh blood group system, Rh30CE |
| 296 | CD240D | Rh blood group system, Rh30D |
| 297 | CD240DCE | Rh30D/CE crossreactive mabs |
| 298 | CD241 | RhAg, Rh50, Rh associated antigen |
| 299 | CD242 | LW blood group, Landsteiner-Wiener blood group antigens |
| 300 | CD243 | MDR-1, P-glycoprotein, pgp 170, multidrug resistance protein I |
| 301 | CD244 | 2B4 |
| 302 | CD245 | p220/240, DY12, DY35 |
| 303 | CD246 | Anaplastic lymphoma kinase (ALK) |
| 304 | CD247 | T cell receptor zeta chain, CD3 zeta |
| 305 | CD248 | TEM1, Endosialin |
| 306 | CD249 | Aminopeptidase A |
| 307 | CD252 | OX40L |
| 308 | CD253 | TRAIL |
| 309 | CD254 | TRANCE |
| 310 | CD256 | APRIL |
| 311 | CD257 | BLYS |
| 312 | CD258 | LIGHT |
| 313 | CD261 | TRAIL-R1 |
| 314 | CD262 | TRAIL-R2 |
| 315 | CD263 | TRAIL-R3 |
| 316 | CD264 | TRAIL-R4 |
| 317 | CD265 | TRANCE-R |
| 318 | CD266 | TWEAK-R |
| 319 | CD267 | TACI |
| 320 | CD268 | BAFFR |
| 321 | CD269 | BCMA |
| 322 | CD271 | NGFR (p75) |
| 323 | CD272 | BTLA |
| 324 | CD273 | B7DC, PDL2 |
| 325 | CD274 | B7H1, PDL1 |
| 326 | CD275 | B7H2, ICOSL |
| 327 | CD276 | B7H3 |
| 328 | CD277 | BT3.1 |
| 329 | CD278 | ICOS |
| 330 | CD279 | PD1 |
| 331 | CD280 | ENDO180 |
| 332 | CD281 | TLR1 |
| 333 | CD282 | TLR2 |
| 334 | CD283 | TLR3 |
| 335 | CD284 | TLR4 |
| 336 | CD286 | TLR6 |
| 337 | CD288 | TLR8 |
| 338 | CD289 | TLR9 |
| 339 | CD290 | TLR10 |
| 340 | CD292 | BMPR1A |
| 341 | CDw293 | BMPR1B |
| 342 | CD294 | CRTH2 |
| 343 | CD295 | LeptinR |
| 344 | CD296 | ART1 |
| 345 | CD297 | ART4 |
| 346 | CD298 | Na+/K+-ATPase b3 |
| 347 | CD299 | DCSIGN-related |
| 348 | CD300a | CMRF35H |
| 349 | CD300c | CMRF35A |
| 350 | CD300e | CMRF35L1 |
| 351 | CD301 | MGL, CLECSF14 |
| 352 | CD302 | DCL1 |
| 353 | CD303 | BDCA2 |
| 354 | CD304 | BDCA4, Neuropilin 1 |
| 355 | CD305 | LAIR1 |
| 356 | CD306 | LAIR2 |
| 357 | CD307 | IRTA2 |
| 358 | CD309 | VEGFR2, KDR |
| 359 | CD312 | EMR2 |
| 360 | CD314 | NKG2D |
| 361 | CD315 | CD9P1 |
| 362 | CD316 | EWI2 |
| 363 | CD317 | BST2 |
| 364 | CD318 | CDCP1 |
| 365 | CD319 | CRACC |
| 366 | CD320 | 8D6A |
| 367 | CD321 | JAM1 |
| 368 | CD322 | JAM2 |
| 369 | CD324 | E-Cadherin |
| 370 | CD325 | N-Cadherin |
| 371 | CDw325 | N-Cadherin |
| 372 | CD326 | Ep-CAM |
| 373 | CD327 | Siglec-6 |
| 374 | CDw327 | siglec6 |
| 375 | CD328 | Siglec-7 |
| 376 | CDw328 | siglec7 |
| 377 | CD329 | Siglec-9 |
| 378 | CDw329 | siglec9 |
| 379 | CD331 | FGFR1 |
| 380 | CD332 | FGFR2 |
| 381 | CD333 | FGFR3 |
| 382 | CD334 | FGFR4 |
| 383 | CD335 | NKp46 |
| 384 | CD336 | NKp44 |
| 385 | CD337 | NKp30 |
| 386 | CD338 | ABCG2, BCRP1 |
| 387 | CDw338 | ABCG2, BCRP |
| 388 | CD339 | Jagged-1 |
| 389 | CD340 | HER-2 |
| 390 | CD344 | Frizzled-4R |
| 391 | CD349 | Frizzled-9R |
| 392 | CD350 | Frizzled-10R |

Other cell surface molecules

| | |
|---|---|
| 1 | 10D1 |
| 2 | 175 Ag |
| 3 | 3H11 |
| 4 | A33 |
| 5 | A6H |
| 6 | AM-3K antigen |
| 7 | Apolipoprotein B48 receptor (apoB48R) |
| 8 | AY19 |
| 9 | BCL-10 |
| 10 | BENE |
| 11 | BLAME |
| 12 | BLTR-1 |
| 13 | CAR |
| 14 | Carboxy-peptidase M (MAX.1) |
| 15 | Chandra |
| 16 | CH-F42 |
| 17 | CLEC-1 |
| 18 | c-MET |
| 19 | Connexin 43 |
| 20 | CS1 |
| 21 | CTH |
| 22 | CX3CR1 |
| 23 | D8/17 |
| 24 | DCIR |
| 25 | DC-STAMP |
| 26 | Dectin-1 |
| 27 | EphB6 |
| 28 | EPR-1 |
| 29 | ESL-1 |
| 30 | F3Ag |
| 31 | Fca/mR |
| 32 | FDF03 |
| 33 | fMLP receptor |
| 34 | FRP-1, FRP-2 |
| 35 | Galectin family of S-type Lectins |
| 36 | GITR |
| 37 | Growth Hormone Receptor |
| 38 | gp200-MR6 |
| 39 | gp80 (phab V-3) |
| 40 | H47 antigen |
| 41 | Hal-1 |
| 42 | hBRAG |
| 43 | HTm4 |
| 44 | II11R alpha (E27) |
| 45 | IMN3.1 |
| 46 | JAM-3 |
| 47 | JL1 |
| 48 | LAR |
| 49 | Layilin |
| 50 | LLIR |
| 51 | Ly-49H |
| 52 | LYVE-1 |
| 53 | M160 |
| 54 | MAdCAM |
| 55 | MAFA-L |
| 56 | Man-6-P receptor (?CD222) |
| 57 | MARCO (Macrophage receptor with collagenous structure) |
| 58 | MC(1) receptors |
| 59 | MDC-L (ADAM-23) |
| 60 | MDL-1 |
| 61 | MER2 |
| 62 | MICA/B |
| 63 | MIC-1 |
| 64 | MIS |
| 65 | MRP1, 2, 3, 4, 14 and 18 |
| 66 | MRP8/14 |
| 67 | MTS23 |
| 68 | Nag-2 |
| 69 | Neurokinin-1 (Substance P Receptor) |
| 70 | Neuropilin-1 |
| 71 | Neuropillin-2 |
| 72 | Ninjurin1 |
| 73 | NK1 |
| 74 | NK-T |
| 75 | Notch receptors |
| 76 | NKp80 |
| 77 | NTB-A |
| 78 | ORL-1 |
| 79 | PAI-1 |
| 80 | PAF-R |
| 81 | p70 |
| 82 | PC-1 (CD203a-reserved) |
| 83 | Phosphatidylserine receptor |
| 84 | Porimin |
| 85 | Prolactin receptor |
| 86 | Prostaglandin E2 Receptors |
| 87 | PZR |
| 88 | RAGE |
| 89 | Sialoadhesin |
| 90 | Siglec-4a (?) (MAG) |
| 91 | Siglec 4b (?) (SMP) |
| 92 | Siglec-8 |
| 93 | Siglec-10 |
| 94 | Siglec-11 |
| 95 | Sphingosine 1-P (S1P) receptors |
| 96 | SRCL |
| 97 | SR-PSOX |
| 98 | ST2L |
| 99 | STRO1 |
| 100 | TABS |
| 101 | TAP1 and TAP2 |
| 102 | TCA1 |
| 103 | TCL1 |
| 104 | TH5.2 |
| 105 | TP |
| 106 | TIM-3 |
| 107 | TIRC7 |
| 108 | TLR-6 |
| 109 | Toso |
| 110 | TRAMP (DR3, LARD) |
| 111 | Transferrin Receptor 2 |
| 112 | TREM-1 and TREM-2 (Triggering receptors expressed on myeloid |
| | cells) |
| 113 | TWEAK |
| 114 | TWEAKR |
| 115 | VAP-1 (Vascular adhesion protein) |
| 116 | VEGFR-1 (FLT-1) |
| 117 | VEGFR-3 (FLT-4) |
| 118 | VPAC1 (VIP receptor) |
| 119 | Wue-1 antigen |
| 120 | XCR1 |

“Cytokine” that is used in the description is defined to be interpreted in the broadest sense in the art. “Cytokine” refers to a physiologically active substance that is produced from cells and acts on the same or different cells. Cytokines are generally proteins or polypeptides and have an effect of controlling immune response, an effect of regulating the endocrine system, an effect of regulating the nervous system, an anti-tumor effect, anti-virus effect, an effect of regulating cell proliferation, an effect of regulating cell differentiation, and the like. In the description, a cytokine may be in the form of protein or nucleic acid or in another form. At the time when a cytokine actually exerts an effect, the cytokine meant herein is generally in the form of protein. Some of cytokines can be defined as lectins.

“Growth factor” or “cell growth factor” to be used in the description refers to a substance that is used interchangeably in the description and promotes or controls cell proliferation. Growth factors are also referred to as Seicho inshi or Hatsuiku inshi. The effect of a growth factor can be an alternative for the effect of a serum macromolecular substance when the growth factor is added to vehicle upon cell culture or tissue culture. It has been revealed that many growth factors can function as factors for controlling cell differentiation in addition to cell proliferation. Such growth factor can also be defined as one of lectins.

Typical examples of cytokines include interleukins, chemokines, hematopoietic growth factors such as a colony-stimulating factor, tumor necrosis factors, and interferons. Typical examples of growth factors include factors having proliferation activity, such as a blood platelet-derived growth factor (PDGF), an epithelial growth factor (EGF), a fibroblast growth factor (FGF), a hepatic parenchymal cell growth factor (HGF), and a vascular endothelial growth factor (VEGF).

Physiologically active substances such as cytokines and growth factors generally undergo the phenomenon of functional redundancy (redundancy). Hence, even cytokines or growth factors that are known as other names and are known to have other functions, can be used in the present invention, as long as they have the activities of physiologically active substances that are used in the present invention. Moreover, cytokines or growth factors can be used in preferred embodiments of the treatment methods or remedies of the present invention, as long as they have activities preferred in the description.

“Galectin” is a generic term for lectins binding to β galactoside. Galectins are known to form a group of proteins having homology with the amino acid sequence of sugar chain-recognizing domains (CRDs). At least 14 types of galectin have been identified as having a molecular weight ranging from 14 kDa to 36 kDa and having 1 to 2 types of CRD. Galectins are soluble proteins having no membrane binding regions, but binding to various ligands in vivo. Galectins are involved in substitution of the hydroxyl group of β-galactose and in binding specificity to aglycone molecules. The presence of galectins has been confirmed in cytoplasms, nuclei, cell membranes, extracellular matrices, and the like. It is said that galectins are involved in cell-to-substrate interaction, cell proliferation control, control of RNA transport from the nucleus, cytoskeleton formation, apoptosis induction or suppression, neural induction, and the like. Galectins are classified into four types in terms of structure: galectins 1, 2, and 7 that are dimers of the same type; galectin 5 that is a monomer; galectins 4, 6, 8, and 9 that are single-chain polypeptides each having two binding regions linked via linker peptides; galectin 3 that is a protein having a single binding region and a short N-terminus. The expression and distribution of galectins in tissues differ depending on galectin types. Galectins are distributed tissue-specifically. In humans, galectin 1 is expressed in the skeletal muscle, neurons, kidney, placenta, and thymus gland, galectin 2 is expressed in tumors in the liver, galectin 3 is expressed by activated macrophages, eosinophils, neutrophils, mast cells, the small intestine, the epithelium of a respiratory organ, and sensory neurons, galectin 4 is expressed in the intestine or the epithelium of the oral cavity, galectin 5 is expressed by erythrocytes or reticulum cells, galectin 6 is expressed on the epithelium of the intestinal tract, galectin 7 is expressed by keratinocytes, galectin 8 is expressed in the lungs, liver, kidney, heart, and brain, and galectin 9 is expressed in the liver, small intestine, kidney, lymphoid tissue, lungs, cardiac muscle, and skeletal muscle.

Lectins that are specifically distributed will be described below.

A mannose-6-phosphate receptor is distributed in the trans-Golgi network of each cell and is known to recognize a high-mannose type sugar chain on a lysosome enzyme as a ligand.

Calnexin is distributed in the endoplasmic reticulum and is known to recognize a nascent glycoprotein that is an α glucosylation type N saccharine as a ligand.

Calreticulin is distributed in the endoplasmic reticulum and is known to recognize a nascent glycoprotein that is an α glucosylation type N saccharine as a ligand.

ERGIC-53 is distributed in a region ranging from the endoplasmic reticulum to cis-Golgi and is known to recognize a mannose-containing sugar chain as a ligand.

VIP36 is distributed in a region ranging from the endoplasmic reticulum to the cell membrane and is known to recognize a high-mannose type sugar chain as a ligand.

Galectins are distributed tissue-specifically as described above and are known to recognize a β-galactose-type sugar chain as a ligand.

Siglec1 (sialoadhesin) is distributed in macrophages and is known to recognize Siaα2-3Gal as a ligand.

Siglec2 (CD22) is distributed in lymphocytes (B cells) and is known to recognize Siaα-2-6Galβ1-4GlcNAc as a ligand.

Siglec3 (CD33) is distributed in myeloid cells and is known to recognize Siaα-2-3Gal as a ligand.

Siglec4a (MAG) is present in peripheral nerves and is known to recognize Siaα2-3Gal as a ligand.

Siglec5 (myelin protein) is present in monocytes and is known to recognize a sialic acid-containing sugar chain as a ligand.

N-CAM is distributed in peripheral nerves and is known to recognize a high-mannose type sugar chain as a ligand.

Po (mammalian peripheral myelin, an intercellular adhesion factor existing on mature Schwann cells) is distributed in peripheral nerves and is known to recognize an HNK-1 antigen as a ligand.

L-selectin is distributed in leukocytes and is known to recognize a sialyl 6-sulfo Le^{X} on vascular endothelial cells as a ligand.

E-selectin is present in vascular endothelial cells and is known to recognize a sialyl Le^{X} of lymphocytes as a ligand.

P-selectin is present in vascular endothelial cells and is known to recognize sialyl Le^{X} and tyrosine sulfate on lymphocyte PSGL-1 as ligands.

A mannose binding protein is present in lymphocytes (natural killer cells) and is known to recognize N-sugar chain as a ligand.

An asialo-glycoprotein receptor is distributed in the liver and recognizes triantenna and tetraantenna complex-type sugar chains of proteins such as serum as ligands.

A macrophage mannose receptor is distributed in macrophages and is known to recognize a mannose-containing sugar chain as a ligand.

Antithrombin (blood coagulation factor) is present in blood and is known to recognize heparin as a ligand.

FGF is distributed in blood and is known to recognize heparan sulfate as a ligand.

Interleukin2 (IL-2) is distributed in blood and is known to recognize a high-mannose type sugar chain on an IL-2 receptor α subunit as a ligand.

Interleukin1α (IL-1α) is distributed in blood and is known to recognize an asialo-biantenna sugar chain as a ligand.

Interleukin1β (IL-1β) is distributed in blood and is known to recognize GPI anchor sugar chain glycolipid GM4 as a ligand.

Interleukin3 (IL-3) is distributed in blood and is known to recognize heparan sulfate as a ligand.

Interleukin6 (IL-6) is distributed in blood and is known to recognize an HNK-1 antigen as a ligand.

Interleukin7 (IL-7) is distributed in blood and is known to recognize a sialyl Tn antigen as a ligand.

Tumor necrosis factor α (TNF-α) is distributed in blood and is known to recognize a mannose-containing sugar chain as a ligand.

“In vitro affinity” for a lectin in vitro in the description can be determined by measuring affinity (e.g., Example 7 in the description) for a lectin associated with a target site (e.g., E-selectin in the case of an inflammation site) in an in vitro experiment. The affinity can be measured by an inhibition experiment using lectin-immobilized microplates as described in Yamazaki, N. (1999) Drug Delivery System, 14, 498-505, for example. Specifically, a lectin (e.g., E-selectin; R&D Systems Co., U.S.A.; Various lectins can be used herein depending on target organs.) is immobilized on a 96-well microplate. Various types of sugar chain-bound liposome complex (protein amounts: 0.01 μg, 0.04 μg, 0.11 μg, 0.33 μg, and 1 μg) varying in concentration were added together with 0.1 μg of biotinylated and fucosylated fetuin as a comparative ligand to the lectin-immobilized plate, followed by 2 hours of incubation at 4° C. After 3 times of washing with PBS (pH 7.2), horseradish peroxidase (HRPO)-conjugated Streptavidin is added and then incubation is performed for 1 hour at 4° C. After 3 times of washing with PBS (pH 7.2), a peroxidase substrate is added and then the resultant is allowed to stand at room temperature. Absorbance at 405 nm can be measured using a microplate reader (Molecular Devices Corp., U.S.A.).

Binding constant can be expressed as ICn (“n” is an arbitrary number between 1 and 99, such as 10, 20, 30, 40, 50, and 60; unit: concentration (M)). The calculation method for ICn to be used herein is as described below. “IC” used herein indicates inhibitory concentration.

Binding index number (proportion) is measured at various concentrations. For example, the values of sample LY-1 were measured.

Table 1 shows the results. Here, in vitro lectin binding activity was determined by an inhibition experiment using lectin-immobilized microplates according to a standard method (Yamazaki, N. (1999) Drug Delivery System, 14, 498-505). Specifically, a lectin (e.g., E-selectin; R&D Systems Co., U.S.A.; Various lectins can be used herein depending on target organs.) was immobilized on a 96-well microplate. Various types of sugar chain-bound liposome complex (protein amounts: 0.01 μg, 0.04 μg, 0.11 μg, 0.33 μg, and 1 μg) varying in concentration were added together with 0.1 μg of biotinylated and fucosylated fetuin as a comparative ligand to the lectin-immobilized plate, followed by 2 hours of incubation at 4° C. After 3 times of washing with PBS (pH 7.2), horseradish peroxidase (HRPO)-conjugated Streptavidin was added and then incubation was performed for 1 hour at 4° C. After 3 times of washing with PBS (pH 7.2), a peroxidase substrate was added and then the resultant was allowed to stand at room temperature. Absorbance at 405 nm was measured using a microplate reader (Molecular Devices Corp., U.S.A.). Biotinylation of fucosylated fetuin was performed by performing treatment with a sulfo-NHS-biotin reagent (Pierce Co., U.S.A.) and then purification using Centricon-30 (Amicon Co., U.S.A.). HRPO-conjugated Streptavidin was prepared by oxidizing HRPO and binding of Streptavidin via reductive amination using NaBH₃CN. The measurement results were processed and calculated as described below.

TABLE 1
Values of sample LY-1 and IC50 at each concentration

| | Concentration | | | | |
|---|---|---|---|---|---|
| | 0.01 | 0.04 | 0.11 | 0.33 | 1 |
| Average value of sample LY-1 | 0.144 | 0.142 | 0.126 | 0.110 | 0.073 |
| Ratio of average value of sample | 0.739 | 0.715 | 0.562 | 0.414 | 0.060 |
| LY-1* | | | | | |
| Ratio of y coordinate of IC50 series | 0.500 | 0.500 | 0.500 | 0.500 | 0.500 |

*“Ratio of the average value of sample LY-1” or “Ratio of the y coordinate of IC50 series is the ratio of the same with respect to 1 (a difference between a “hot” value and a “cold” value of Control is determined to be “1.”)

TABLE 2
Control

| Hot | Cold |
|---|---|
| 0.171 | 0.067 |

Therefore, when the ratio of the average value of sample LY-1 is “W,” the average value of sample LY-1 is “X,” the value of “hot” is “Y,” the value of “cold” is “Z,” and the calculation formula can be expressed as

W=(X−Z)/(Y−Z)×100.

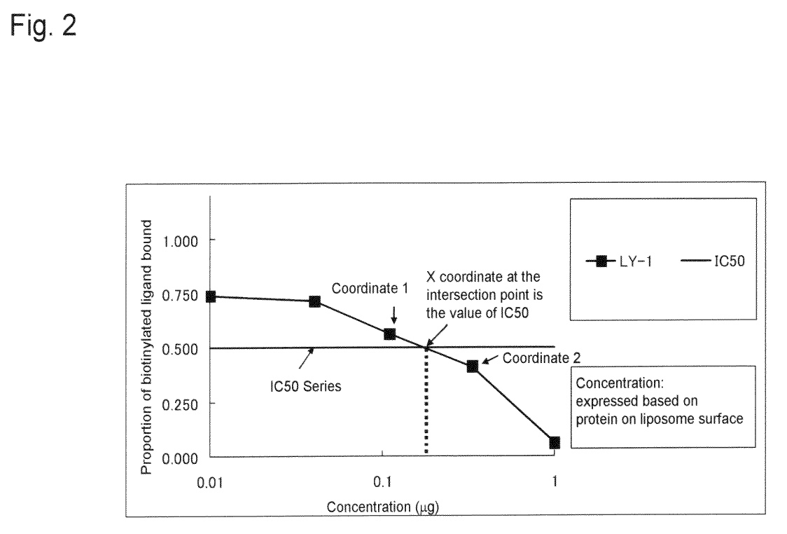

FIG. 2 shows Graph 1 in which the values of sample LY-1 obtained based on Table 1 and Table 2 and IC50 series are plotted.

Graph 1 was prepared based on Table 1. X axis is expressed using a logarithm scale. Each point on the line graph represents the ratio of the average measured value at each concentration (horizontal axis) of sample LY-1. Values of control differ depending on samples. To facilitate comparison, the longitudinal axis of the graph represents the ratios when a difference between the value of “hot” and the value of “cold” is determined to be “1.” X coordinate at the intersection point between the graph of Sample LY-1 and the graph of IC50 series represents the value of IC₅₀. The intersection point exists on the straight line including coordinate 1 (0.11, 0.562) and coordinate 2 (0.33, 0.414) and is represented by formula y=−0.673x+0.636. When y=0.5 (formula for IC50 series), X coordinate at the intersection point of two straight lines is 0.202. This value is divided by the molecular weight of the protein, 69000, and then the result is further divided by 300 (the number of protein per liposome). Thus 9.76E-09 is obtained.

These calculations can be automated using a computer program.

In the description, “in vivo affinity” refers to affinity for the destination to which a delivery vehicle is actually delivered in vivo. In vivo affinity can be determined by examining the biological dynamic state of the delivery vehicle that is transferred to each organ. As a specific example, in vivo affinity can be examined by administering a liposome via oral or intravenous administration and then evaluating its accumulation in each mouse organ. After intravenous injection or oral administration, all organs are each excised. Each organ is prepared as a tissue homogenate using 1% Triton X solution and HG30 homogenizer (Hitachi Koki Co., Ltd.). Liposomes contained in tissue homogenates are extracted using 100% methanol and chloroform. The amount of a liposome is measured as follows. The fluorescence intensity of FITC bound to the liposomes is measured using a fluorescent microplate reader Biolumin 960 (Molecular Dynamics), followed by measurement using excitation at 490 nm and emission at 520 nm. The results obtained by this method can also be represented by numerical figures, but can also be expressed comparatively such that evaluation can also be made using +++, ++, +, −, and the like.

In the present invention, in vitro affinity determined by in vitro assay using a cell surface molecule such as a specific lectin is represented by n % inhibitory concentration (ICn; herein, “n” ranges from 0 to 100). The present invention is partially based on the finding that the thus obtained numerical value correlates unexpectedly with in vivo affinity (delivery specificity). As a result, based on the rolling model, it has become possible to efficiently, conveniently, and precisely design a delivery vehicle. Such a simple design of a delivery vehicle has been impossible with the use of conventional technology and has been unknown. The present inventors have established the above theory and completed the present invention by examining and systematically studying several hundred delivery vehicle candidates.

Furthermore, as a result of systematic studies, the present inventors have discovered that ideal in vivo affinity can be predicted with high probability by: measuring in vitro affinity at least one strong binding IC in which n of ICn is smaller than a branch point that is a numerical figure between IC35 and IC30 (e.g., between approximately IC31 and approximately IC30) and measuring in vitro affinity at least one weak binding IC in which n of ICn is larger than the branch point; and comparing the results in a comprehensive manner. As a result, it has been demonstrated that a delivery vehicle (e.g., sugar-chain-modified liposome) showing a low inhibitory concentration at the strong binding IC and a high inhibitory concentration at the weak binding IC exerts high in vivo affinity. No theoretical constraints are desired herein. It is predicted that a delivery vehicle exerting such properties has preferred characteristics in view of “rolling.”

Realization of active targeting that involves actively delivering a desired substance has been conventionally attempted using molecules having high specificity to molecules existing in target cells. It has been thought that the higher the binding nature, the more sufficient, selective, and efficient delivery can be achieved. However, it has been increasingly revealed that active targeting based on such idea is unsuccessful. It has been revealed by the present invention that this may be because, although no theoretical constraints are desired, excessively high binding nature causes a delivery vehicle to remain bound on the target so that the molecule to be delivered cannot be efficiently delivered. When competitive inhibition takes place at a low concentration at both strong binding IC and weak binding IC, the binding in this case is thought to have the highest specificity. However, the resulting in vivo affinity is not so high in most cases. Therefore, strong binding is not always preferred. Rather, it is concluded that a delivery vehicle showing a low concentration at the strong binding IC, but a high concentration at the weak binding IC makes it possible to perform rolling at its target site and efficiently deliver a substance to be delivered.

Preferably, the measurement of in vitro affinity of the present invention comprises measurement at a strong binding IC that is at least one between IC30 and IC10 and measurement at a weak binding IC that is at least one between IC40 and IC60. In measurement at a strong binding IC that is at least one between IC30 and IC10, ICn within the range may be arbitrarily selected. Similarly, in measurement at a weak binding IC that is at least one between IC40 and IC60, ICn within the range may be arbitrarily selected.

In an embodiment, the measurement of in vitro affinity comprises measurement at a strong binding IC that is approximately IC30 or less. The selection comprises selecting a candidate showing a low inhibitory concentration at the strong binding IC. In this case, a delivery vehicle that enables good rolling; that is, has good in vivo affinity can be identified with at least a constant probability. Preferably, the measurement of affinity comprises measurement at a strong binding IC that is approximately IC31 or less. A candidate showing an inhibitory concentration of 10⁻⁹M or less typically at the strong binding IC is selected. Here, “binding of less than IC30” means that “n” of ICn has a numerical figure that is smaller than 30. In contrast, “binding of IC31 or more” means that “n” of ICn has a numerical figure that is 31 or more.

In a preferred embodiment, the above selection is made when the inhibitory concentration at IC30 is 10⁻⁹M or less, the inhibitory concentration at IC20 is 10⁻⁹M or less, and the inhibitory concentration at IC10 is 10⁻⁹M or less.

In another embodiment, the measurement of in vitro affinity of the present invention comprises measurement at a weak binding IC that is approximately IC31 or more. With this selection, a candidate showing a high inhibitory concentration at the strong binding IC is selected. Preferably, the measurement of in vitro affinity comprises measurement at a weak binding IC that is approximately IC31 or more. A candidate showing an inhibitory concentration of 10⁻⁹M or more at the weak binding IC is selected.

In a preferred embodiment, the selection is made when the inhibitory concentration at IC60 is 10⁻⁹M or more, the inhibitory concentration at IC50 is 10⁻⁹M or more, and the inhibitory concentration at IC40 is 10⁻⁹M or more.

In a preferred embodiment, the measurement of in vitro affinity of the present invention comprises measurement at a strong binding IC that is approximately IC30 or less and measurement at a weak binding IC that is approximately IC31 or more. The above selection is characterized by selecting a candidate showing a low inhibitory concentration at the strong binding IC and a high inhibitory concentration at the weak binding IC.

In a further preferred embodiment, the measurement of in vitro affinity of the present invention comprises measurement at a strong binding IC that is approximately IC30 or less and measurement at a weak binding IC that is approximately IC31 or more. The above selection is characterized by selecting a candidate showing an inhibitory concentration of 10⁻⁹M or less at the strong binding IC and inhibitory concentration of 10⁻⁹M or more at the weak binding IC.

In a further preferred embodiment, the measurement of in vitro affinity of the present invention comprises measurement at a strong binding IC that is approximately IC30 or less. The above selection is characterized by selecting a candidate showing a low inhibitory concentration at the strong binding IC. Here, the selection is further characterized by satisfying at least one condition selected from the group consisting of: a condition in which the inhibitory concentration at IC60 is 10⁻⁹M or more, a condition in which the inhibitory concentration at IC50 is 10⁻⁹M or more, a condition in which the inhibitory concentration at IC40 is 10⁻⁹M or more, a condition in which the inhibitory concentration at IC30 is 10⁻⁹M or less, a condition in which the inhibitory concentration at IC20 is 10⁻⁹M or less, and a condition in which the inhibitory concentration at IC20 is 10⁻⁹M or less.

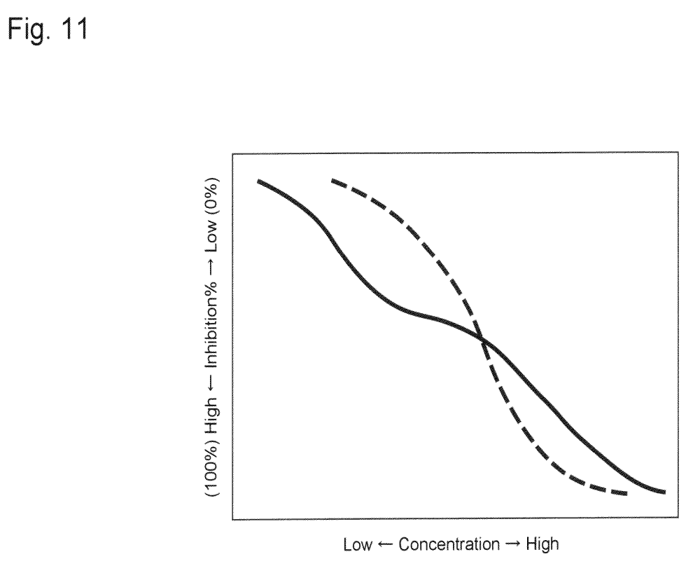

In a preferred embodiment, as shown in a graph showing concentration-inhibition % curves in FIG. 11, a possible advantageous form in the rolling model has a gentle curve as represented by the solid line. It was demonstrated by the present invention that possession of a curve represented by a dotted line (e.g., antigen antibody reaction) results in failed rolling and unsuccessful selective delivery.

A measurement method of in vitro affinity for determination of the above numerical figures is performed by competitive inhibition assay, noncompetitive inhibition assay, binding assay, or the like.

According to the present invention, once a preferred delivery vehicle is determined in vitro, the delivery vehicle can then be generated based on the composition. At this time, a substance desired to be delivered (e.g., a pharmaceutical composition) can be contained in the delivery vehicle.

Here, when the composition of a preferred delivery vehicle is unknown, the composition can be determined according to need. As a method for determination of such composition, an arbitrary method known in the art can be employed. When the composition is analyzed, a method for preparing a delivery vehicle having the composition can be determined. For determination of such a preparation method, WO2002/081723, JP Patent Publication (Kokai) No. 9-31095 A (1997), JP Patent Publication (Kokai) No. 11-42096 A (1999), JP Patent Publication (Kokai) No. 2004-180676 A, and Kenichi Hatanaka, Shinichiro Nishimura, Tatsuro Ohuchi, and Kazukiyo Kobayashi (1997) Science and Engineering of Sugar (To-shisu no kagaku to kogyo), Kodansha Ltd., Tokyo, Japan, and the like can be referred. According to the present invention, it has been revealed that when a delivery vehicle is a sugar-chain-modified liposome, not only the composition, but also the sugar chain type and density play important roles. Therefore, in a preferred embodiment, analysis of the composition can comprise analysis of the sugar chain types and densities of the sugar-chain-modified liposome. Once the type and density of a sugar chain are determined, persons skilled in the art can determine a method for producing a sugar-chain-modified liposome according to the techniques described for the present invention. An example of such a production method involves performing, upon generation of a sugar-chain-modified liposome, a reaction of sugar chains (the types and the amounts of which are determined based on the thus determined composition) under conditions adequate for binding to a liposome. Preferably, a linker can be used herein. As a linker, a protein such as albumin can be used, for example. A liposome can be hydrophilized, according to need.

In a preferred embodiment, the method of the present invention further comprises the step of confirming the in vivo dynamic state of the thus selected delivery vehicle.

In another aspect, a method for producing a delivery vehicle by which delivery to undesired sites is not performed is provided according to the present invention, which is analogous to the method for delivery to desired sites. Such production method comprises the steps of:

A) measuring in vitro affinity of candidate delivery vehicles for a cell surface molecule such as a lectin associated with an undesired site; and
B) selecting a delivery vehicle having in vitro affinity corresponding to non-delivery to the undesired site.

Alternatively, the production method can be a method that comprises the steps of:

A) measuring in vitro affinity of candidate delivery vehicles for a cell surface molecule such as a lectin associated with an undesired site;
B) selecting a delivery vehicle having in vitro affinity corresponding to non-delivery to the undesired site and analyzing the composition of the selected delivery vehicle; and
C) generating the selected delivery vehicle based on the composition.

In another aspect, the present invention provides a method for producing a delivery vehicle for achieving specific delivery. The production method comprises the steps of:

A) measuring in vitro affinity of candidate delivery vehicles for a cell surface molecule such as a lectin associated with a site to which specific delivery is performed;
B) measuring in vitro affinity of the candidate delivery vehicles for a cell surface molecule such as a lectin associated with a site to which specific delivery is not performed; and
C) selecting a delivery vehicle having in vitro affinity corresponding to delivery to the desired site and corresponding to non-delivery to the undesired site.

Alternatively, the method can be a method that comprises the steps of:

A) measuring in vitro affinity of candidate delivery vehicles for a cell surface molecule such as a lectin associated with a site to which specific delivery is performed;
B) measuring in vitro affinity of the candidate delivery vehicles for a cell surface molecule such as a lectin associated with a site to which specific delivery is not performed;
C) selecting a delivery vehicle having in vitro affinity corresponding to delivery to the desired site and corresponding to non-delivery to the undesired site and analyzing the composition of the selected delivery vehicle; and
D) generating the selected delivery vehicle based on the composition.

The thus produced delivery vehicle is also encompassed within the scope of the present invention.

### (Delivery Method)

In another aspect, the present invention provides a method for delivering a biological factor to a target site of a subject who needs the biological factor. This method comprises the step of: administering the sugar-chain-modified liposome of the present invention via oral administration, in which the sugar-chain-modified liposome contains an effective dose of the biological factor. As the sugar-chain-modified liposome, such a liposome in an arbitrary form as describe above (sugar-chain-modified liposome) can be used.

### (Specific Explanation of Delivery Vehicle)

In the description, “sugar chain” refers to a compound composed of one or more sugar units (monosaccharides and/or derivatives thereof) linked together. When two or more sugar units are linked, sugar units are each bound via dehydration and condensation due to glycosidic linkage. Examples of such sugar chains include, but are not limited to, a wide range of sugar chains, in addition to polysaccharides (glucose, galactose, mannose, fucose, xylose, N-acetylglucosamine, N-acetylgalactosamine, sialic acid, and their complexes and derivatives thereof) contained in vivo, sugar chains degraded or induced from complex biomolecules (e.g., degraded polysaccharide, glycoprotein, proteoglycan, glycosaminoglycan, and glycolipid). Therefore, in the description, “sugar chain” can be used interchangeably with “polysaccharide,” “sugar,” or “carbohydrate.” Furthermore, unless otherwise particularly noted, “sugar chain” in the description may refer to both a sugar chain and a sugar-chain-containing substance. A typical sugar chain is a substance composed of approximately 20 types of monosaccharide linked to form a chain (glucose, galactose, mannose, fucose, xylose, N-acetylglucosamine, N-acetylgalactosamine, and sialic acid, and complexes and derivatives thereof) that is bound to intracellular and extracellular proteins or lipids in vivo. The functions of such a sugar chain differ depending on the monosaccharide sequences. Furthermore, sugar chains are generally branched in a complicated manner. It is estimated that several hundred or more types of sugar chain varying in their structures are present in a human body. Furthermore, it is thought that there are several tens of thousands or more of types of useful sugar chain structure in a human body. It is thought that such sugar chain structures are involved in higher functions exerted by proteins or lipids in vivo, such as a function of recognizing molecules and a function of recognizing cells, which are exerted between cells. Most of such mechanism remains unelucidated. Sugar chains are attracting attention in the field of current life science as the 3^{rd} life chain following nucleic acids and proteins. In particular, the functions of sugar chains as ligands (information molecules) for cell recognition are expected and the application of such sugar chains to development of highly functional materials is studied.

In the description, “sugar” or “monosaccharide” refers to polyhydroxy aldehyde or polyhydroxy ketone containing at least one hydroxy group and at least one aldehyde group or ketone group and composes a basic unit of a sugar chain. In the description, sugar is also referred to as carbohydrate and both terms can be used interchangeably. In the description, when particularly mentioned, “sugar chain” refers to a chain or a sequence composed of one or more sugars linked. “Sugar” or “monosaccharide” refers to one unit composing a sugar chain.

“Sugar” or “monosaccharide” in which n=2, 3, 4, 5, 6, 7, 8, 9, and 10 are each referred to as diose, triose, tetrose, pentose, hexose, heptose, octose, nonose, and decose. In general, they correspond to aldehyde or ketone of chain polyhydric alcohol. The former is referred to as aldose and the latter is referred to as ketose. In the present invention, “sugar” or “monosaccharide” in any forms can be used.

Naming of sugars to be described in the present invention is performed according to the general nomenclature. Examples are as follows:

galactose is named

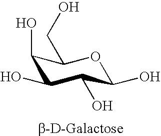

### Gal;

N-acetylgalactosamine is named

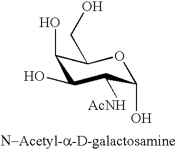

### GalNAc;

mannose is named

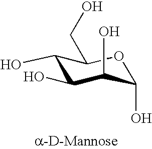

### Man;

glucose is named

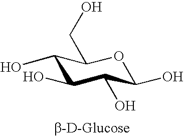

### Glc;

N-acetylglucosamine is named

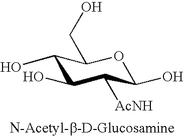

### GlcNAc;

fucose is named

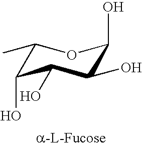

### Fuc;

N-acetylneuraminic acid is named

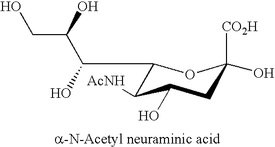

### Neu5Ac;

ceramide is named

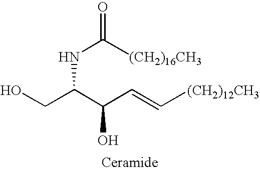

### Cer; L-serine

CH₂(OH)CH(COOH)NH₂ is named Ser. In addition, Cer is generally classified as a lipid. However, in the description, Cer is treated as a sugar unless otherwise particularly noted, since it meets the definition for a type of sugar composing a sugar chain. Furthermore, Ser is generally classified as an amino acid. However, in the description, Ser is treated as a sugar unless otherwise particularly noted, since it meets the definition for a type of sugar composing a sugar chain. Two circular anomers are represented by α and β or may also be represented by “a” or “b” because of reasons concerning representation. Therefore, in the description, “α” and “a” can be or “β” and “b” can be used interchangeably in terms of denotation of anomers.

In the description, galactose indicates an arbitrary isomer and typically indicates β-D-galactose. Galactose is used to indicate β-D-galactose unless otherwise particularly noted.

In the description, acetylgalactosamine indicates an arbitrary isomer and typically indicates N-acetyl α-D-galactosamine. Acetylgalactosamine is used to indicate N-acetyl α-D-galactosamine unless otherwise particularly noted.

In the description, mannose indicates an arbitrary isomer and typically indicates α-D-mannose. Mannose is used to indicate α-D-mannose unless otherwise particularly noted.

In the description, glucose indicates an arbitrary isomer and typically indicates β-D-glucose. Glucose is used to indicate β-D-glucose unless otherwise particularly noted.

In the description, acetylglucosamine indicates an arbitrary isomer and typically indicates N-acetyl-β-D-glucosamine. Acetylglucosamine is used to indicate N-acetyl β-D-glucosamine unless otherwise particularly noted.

In the description, fucose indicates an arbitrary isomer and typically indicates β-L-fucose. Fucose is used to indicate β-L-fucose unless otherwise particularly noted.

In the description, acetylneuraminic acid indicates an arbitrary isomer and typically indicates α-N-acetylneuraminic acid. Acetylneuraminic acid is used to indicate α-N-acetylneuraminic acid unless otherwise particularly noted.

In the description, serine indicates an arbitrary isomer and typically indicates L-serine. Serine is used to indicate L-serine unless otherwise particularly noted.

It should be noted in the description that denotational symbols, nominal designation, abbreviated expressions (e.g., Glc), and the like for sugars differ between a case when they are used to indicate monosaccharides are indicated and a case when they are used to indicate those in sugar chains. In a sugar chain, dehydration and condensation take place between a sugar unit and a sugar unit (to which the former sugar unit binds), so that the resultant is present as a result of removing hydrogen or hydroxy groups from the other sugar unit. Therefore, it is understood as follows. When the condensation codes of these sugars are used to represent monosaccharides, all the hydroxy groups are present. However, when such codes are used to represent those in a sugar chain, a condition is indicated wherein hydroxy groups of a sugar unit and hydroxy groups of the other sugar unit (to which the former sugar unit binds) are subjected to dehydration and condensation so that oxygen alone remains.

When a sugar is covalently bound to albumin, the reducing terminus of the sugar is aminated, so that the sugar can bind to another component such as albumin via the amine group. In this case, it should be noted that the term indicates the one in which the hydroxyl group of the reducing terminus is substituted with an amine group.

A monosaccharide generally forms a disaccharide or a polysaccharide via glycosidic linkage. The orientation of linkage with respect to the plane of the ring is denoted with “α” or “β.” Specific carbon atoms that form linkage between two carbons are also described.

In the description, a sugar chain is represented by:

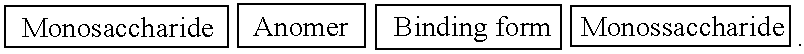

Therefore, for example, β glycosidic linkage between C-1 of galactose and C-4 of glucose is represented by Galβ1, 4Glc.

Sugar chain branches are represented using parentheses. Specifically, a sugar chain branch is denoted by locating a parenthese at a position immediately left of a sugar unit to be bound. For example, this is represented by:

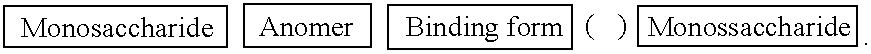

Such parenthesed part is represented by:

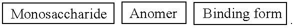

Therefore, for example, when β glycosidic linkage is formed between C-1 of galactose and C-4 of glucose and the C-3 of glucose and C-1 of fucose form α glycosidic linkage, this is represented by Galβ1, 4(Fucα1, 3)Glc. Monosaccharides are represented basically by numbering (potential) carbonyl atomic groups with numbers that should be as small as possible. Under the general standard of organic chemical nomenclature, a case in which an atomic group with superiority over a (potential) carbonyl atomic group is introduced into a molecule, generally the above numbering is used for representation.

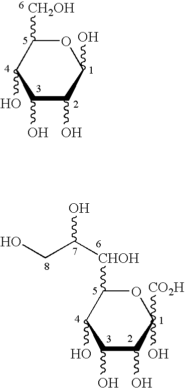

An example of a sugar chain that is used in the description is a sugar chain having at least one or at least two sugar units selected from the group consisting of Gal, GalNAc, Man, Glc, GlcNAc, Fuc, Neu5Ac, and Ser. The reason why a combination of two or more sugar units can be used is, which is not theoretically constrained, that each of the above sugar units has specificity to a cell surface molecule such as a lectin that is localized in a target delivery site in tissues or cells and may be able to exert its specificity even when they are mixed.

### (Liposome)

As a delivery vehicle that can be used in the rolling method of the present invention, any vehicle can also be used. A particularly preferred example is a delivery vehicle using a liposome.

In the description, “liposome” generally means a closed vesicle that is composed of a lipid layer and an internal aqueous layer, which assemble to form a membrane. In addition to a typically used phospholipid, cholesterol, a glycolipid, and the like can also be incorporated. A liposome is a closed vesicle containing water therewithin, so that it can also retain a water-soluble drug and the like within the vesicle. Therefore, with the use of such a liposome, a drug, a gene, or the like that is unable to pass through the cell membrane can be delivered into the cell. Furthermore, such liposome also has good biocompatibility, so that it is expected as a nanoparticular carrier material for DDS.

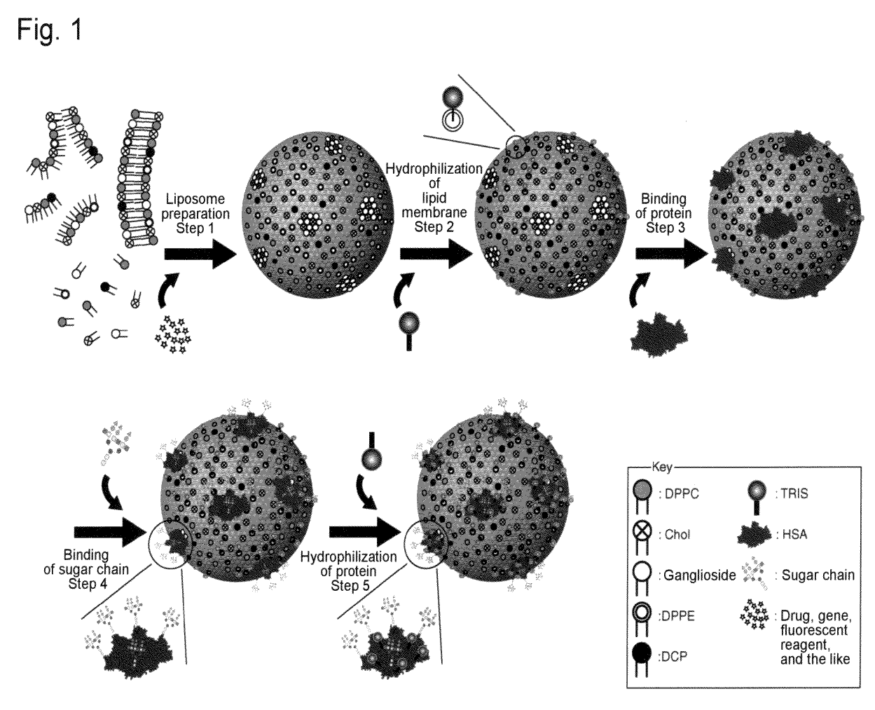

Liposomes can be produced by any techniques known in the art. An example of these techniques is a method that involves performing cholic acid dialysis. Production is performed via cholic acid dialysis that involves a) preparation of a mixed micelle of lipids and a surfactant and b) dialysis of the mixed micelle. Next, in a preferred embodiment of the sugar chain liposome of the present invention, it is preferable to use a protein as a linker. Coupling of a glycoprotein (in which a sugar chain is bound to the protein) to a liposome can be performed via the following two-stage reaction: a) periodate oxidation of the ganglioside portion on the liposome membrane; and b) coupling of a glycoprotein to the oxidized liposome via reductive amination reaction. FIG. 1 shows an example of the reaction flow. A glycoprotein containing a desired sugar chain can be bound to a liposome with the use of such a technique. Furthermore, a great variety of glycoprotein-liposome conjugates having desired sugar chains can be obtained. It is very important to examine particle diameter distribution to examine the purity and stability of liposomes. As methods to be employed for such examination, gel filtration chromatography (GPC), scanning electron microscopy (SEM), dynamic light scattering (DLS), and the like can be employed. A type of liposome in which the molar ratio of dipalmitoylphosphatidylcholine (DPPC), cholesterol, dicetylphosphate (DCP), and ganglioside is 35:45:5:15 can be produced. In addition, the liposome is stable even when it is stored at 4° C. for several months. The in vivo stability of a liposome can be examined using mice. A liposome is intravenously injected into a mouse. 3 hours later, blood is collected and then serum is prepared. Ultrafiltration is performed using a membrane with a pore size of 0.03 μm and then the liposome is purified and collected. Based on the result of SEM observance performed therefor, it can be confirmed that the liposome undergoes no morphological changes before and after 3 hours of treatment and collection in vivo.

Examples of lipids composing the sugar-chain-modified liposome of the present invention include phosphatidyl cholines, phosphatidyl ethanol amines, phosphatidic acids, long-chain alkyl phosphates, glycolipids (e.g., gangliosides), phosphatidyl glycerols, sphingomyelins, and cholesterols.

Examples of phosphatidyl cholines include dimyristoylphosphatidylcholine, dipalmitoylphosphatidylcholine, and distearoyl phosphatidylcholine.

Examples of phosphatidyl ethanol amines include dimyristoylphosphatidyl ethanol amine, dipalmitoyl phosphatidyl ethanol amine, and distearoyl phosphatidyl ethanol amine.

Examples of phosphatidic acids include dimyristoyl phosphatidic acid, dipalmitoyl phosphatidic acid, and distearoyl phosphatidic acid. Examples of long-chain alkyl phosphates include dicetylphosphate and the like.

Examples of glycolipids include galactosyl ceramide, glucosyl ceramide, lactosylceramide, phosphatide, globoside, and gangliosides. Examples of gangliosides include, ganglioside GM1 (Galβ1,3GalNAcβ1,4(NeuAα2,3)Galβ1,4Glcβ1,1′Cer), ganglioside GD1a, and ganglioside GT1b.

As phosphatidyl glycerols, dimyristoyl phosphatidyl glycerol, dipalmitoyl phosphatidyl glycerol, distearoylphosphatidylglycerol, and the like are preferred.

Of these, phosphatidic acids, long-chain alkyl phosphates, glycolipids, and cholesterols are desirably added as constituent lipids since they have effects of elevating the stability of the liposome. Examples of such a lipid composing the liposome of the present invention include: one or more types of lipid (mole percentage: 0% to 30%) selected from the group consisting of phosphatidyl cholines (mole percentage: 0% to 70%), phosphatidyl ethanol amines (mole percentage: 0% to 30%), phosphatidic acids, and long-chain alkyl phosphate; one or more types of lipid (mole percentage: 0% to 40%) selected from the group consisting of glycolipids, phosphatidyl glycerols, and sphingomyelins; and lipids containing cholesterols (mole percentage: 0% to 70%). Preferably, a glycolipid such as ganglioside is contained herein since it facilitates binding of a linker such as albumin.

In a preferred embodiment, ganglioside can be contained in the liposome of the present invention, a linker such as a peptide can be bound thereto, and then a sugar chain can be bound to the resultant.

With the use of a glycolipid for preparation of a liposome, the sugar-chain-modified liposome of the present invention containing a sugar chain contained in the glycolipid as a constituent can be prepared.

### (Sugar-Chain-Modified Liposome)

In an aspect, the present invention provides a sugar-chain-modified liposome. Liposomes capable of sufficiently targeting desired target cells or tissues in vivo have not been provided to date. The present invention has an effect of making it possible to perform targeting that has been impossible with the use of conventional DDS materials through provision of a sugar-chain-modified liposome having tropism for desired target cells or tissues in vivo. Further systematically, a sugar chain having at least one structure selected from the group consisting of Gal, GalNAc, Man, Glc, GlcNAc, Fuc, and Neu5Ac is bound to such sugar-chain-modified liposome.

In the description, “sugar-chain-modified liposome” refers to a substance containing a sugar chain and a liposome and preferably refers to a liposome that is modified by direct or indirect binding of a sugar chain thereto. Such a form in which a sugar chain is bound to a liposome is specifically represented by:

S-(M)-L

(S: sugar chain, M: linker (may be present or absent), L: liposome, -: bond such as a covalent bond or cross-linking agent (e.g., DTSSP)).

When ganglioside is contained in a liposome, the sugar-chain-modified liposome of the present invention is represented by

S-M-GS-L

(GS: sugar chain portion of ganglioside).

When this is more specifically described, in the description, “proximal end of sugar chain to the liposome” of a sugar-chain-modified liposome refers to a terminal portion of a sugar chain located most proximal to the liposome. When a sugar chain is branched, all the relevant terminal portions are referred to as proximal ends.

“Most proximal end of a sugar chain to the liposome” refers to a sugar (monosaccharide) located most proximal to the liposome. Therefore, in the description, it is understood that when “a sugar chain comprising a di- or more (multi-) saccharide is contained at the proximal end of the sugar chain to the liposome,” the proximal end of the sugar chain to the liposome contains, in addition to a saccharide (monosaccharide) at the most proximal end of the sugar chain to the liposome, another saccharide (monosaccharide that may be the same or different from the saccharide at the most proximal end) contained in the above sugar chain comprising a di or more (multi-) saccharide.

In the description, “distal end of a sugar chain to the liposome” of a sugar-chain-modified liposome refers to a terminal portion of the sugar chain, which is located most distal to the liposome. When a sugar chain is branched, all the relevant terminal portions are referred to as distal ends.

“Most distal end of a sugar chain to the liposome” refers to a saccharide (monosaccharide) that is located most distal to the liposome. Therefore, in the description, when “distal end of a sugar chain to the liposome contains a sugar chain comprising a di- or more (multi-) saccharide,” it is understood that the distal end of the sugar chain to the liposome contains, in addition to a saccharide (monosaccharide) located at the most distal end of the sugar chain to the liposome, another saccharide (monosaccharide that may be the same or different from the saccharide at the most distal end) contained in the above sugar chain comprising a di- or more (multi-) saccharide.

Examples adequate for the rolling model in the description are listed in the following Table using liposome numbers (Each liposome No. corresponds to the structure in the right column).

TABLE 3

| No. | Abbreviated name | Structure |
|---|---|---|
| 3 | EEGD1A-BH | Neu5Acα2,3Galβ1,3GalNAcβ1,4 |
| | | (Neu5Acα2,3)Galβ1,4Glcβ1,1Cer |
| 27 | LDF-5 | Fucα1,2Galβ1,4(Fucα1,3)Glc |
| 29 | 3SL-5 | Neu5Acα2,3Galβ1,4Glc |
| 37 | FEEGD1b | Galβ1,3GalNAcβ1,4(Neu5Acα2,8Neu5Acα2,3) |
| | | Galβ1,4Glcβ1,1Cer |
| 38 | G4GN-4 | Galβ1,4GlcNAc |
| 40 | BAT-1 | GalNAcα1,3(Fucα1,2)Gal |
| 41 | SOLX-4 | 3′-(O—SO3H)Galβ1,4(Fucα1,3)GlcNAc |
| 45 | BHD-1 | Fucα1,2Gal |
| 50 | Man8 | Manα1,2Manα1,6(Manα1,3)Manα1,6 |
| | | (Manα1,2Manα1,2Manα1,3)Manβ1,4GlcNAcβ1,4GlcNAc |
| 53 | L2F-1 | Fuca1,2Galb1,3GlcNAcb1,3Galb1,4Glc |
| 56 | Man9 | Manα1,2Manα1,6(Manα1,2Manα1,3)Manα1,6 |
| | | (Manα1,2Manα1,2Manα1,3)Manβ1,4GlcNAcβ1,4GlcNAc |
| 60 | 6SLN-1 | Neu5Acα2,6Galβ1,4GlcNAc |
| 67 | FEEaMDa | Galβ1,3GalNAcβ1,4Galβ1,4Glcβ |
| | | Neu5Acα2,3Galβ1,3GalNAcβ1,4(Neu5Acα2,3)Galβ1,4Glcβ |
| 68 | Man7 | Manα1,6(Manα1,3)Manα1,6(Manα1,2Manα1,2Manα1,3) |
| | | Manβ1,4GlcNAcβ1,4GlcNAc |
| 69 | L4F-1 | Galβ1,3(Fuca1,4)GlcNAcβ1,3Galβ1,4Glc |
| 70 | LNT-5 | Galβ1,3GlcNAcβ1,3Galβ1,4Glc |
| 71 | BBT-1 | Galα1,3(Fucα1,2)Gal |
| 76 | BHD-4 | Fucα1,2Gal |
| 80 | 6SLN-5 | Neu5Acα2,6Galβ1,4GlcNAc |
| 87 | TA-1 | Galβ1,3GalNAc |
| 91 | G6GN-4 | Galβ1,6GlcNAc |
| 93 | A36 | Manα1,6(Manα1,3)Man |
| 96 | TA-4 | Galb1,3GalNAc |
| 105 | Man6 | Manα1,6(Manα1,3)Manα1,6(Manα1,2Manα1,3)Manβ1,4 |
| | | GlcNAcβ1,4GlcNAc |
| 106 | LDF-4 | Fucα1,2Galβ1,4(Fucα1,3)Glc |
| 111 | LNB-5 | Galβ1,3GlcNAc |
| 116 | LDF-1 | Fucα1,2Galβ1,4(Fucα1,3)Glc |
| 117 | TA-5 | Galβ1,3GalNAc |
| 120 | A2 | Manα1,2Man |
| 125 | LX-1 | Galβ1,4(Fucα1,3)GlcNAc |
| 127 | G3G-4 | Galα1,3Gal |
| 129 | BBT-4 | Galα1,3(Fucα1,2)Gal |
| 130 | 2FL-4 | Fucα1,2Galβ1,4Glc |
| 137 | Man4B | Manα1,6(Manα1,3)Manα1,6Manβ1,4GlcNAcβ1,4GlcNAc |
| 139 | 3FL-5 | Galβ1,4(Fucα1,3)Glc |
| 141 | BBT-5 | Galα1,3(Fucα1,2)Gal |
| 142 | BHD-5 | Fucα1,2Gal |
| 146 | 3FL-4 | Galβ1,4(Fucα1,3)Glc |
| 150 | TNA-5 | GalNAcα1-O-L-serine |
| 151 | SLX-4 | Neu5Acα2,3Galβ1,4Fucα1,3)GlcNAc |
| 152 | LY-1 | Fucα1,2Galβ1,4(Fucα1,3)GlcNAc |
| 153 | G6GN-5 | Galβ1,6GlcNAc |
| 154 | Lac-5 | Galβ1,4Glc |
| 155 | A6 | Manα1,6Man |
| 175 | G3G-5 | Galα1,3Gal |
| 178 | LNT-4 | Galβ1,3GlcNAcβ1,3Galβ1,4Glc |
| 183 | G4GN-5 | Galβ1,4GlcNAc |
| 184 | STNA-1 | Neu5Acα2,6GalNAcα1-O-L-serine |
| 186 | Man3 | Manα1,6(Manα1,3)Manβ1,4GlcNAcβ1,4GlcNAc |
| 191 | LB-1 | Fucα1,2Galβ1,3(Fucα1,4)GlcNAc |
| 195 | TNA-4 | GalNAcα1-O-L-serine |
| 197 | A4 | Manα1,4Man |
| 199 | STNA-4 | Neu5Ac2,6GalNAcα1-O-L-serine |
| 204 | Lac-1 | Galβ1,4Glc |
| 207 | 6SL-5 | Neu5Acα2,6Galβ1,4Glc |
| 209 | LDFH-4 | Fuca1,2Galb1,3(Fuca1,4)GlcNAcb1,3Galb1,4Glc |
| 213 | LX-4 | Galβ1,4(Fucα1,3)GlcNAc |
| 218 | EEGM1-BH | Galβ1,3GalNAcβ1,4(Neu5Acα2,3)Galβ1,4Glcβ1,1Cer |
| 220 | LNB-4 | Galβ1,3GlcNAc |
| 224 | Man5 | Manα1,6(Manα1,3)Manα1,6(Manα1,3) |
| | | Manβ1,4GlcNAcβ1,4GlcNAc |
| 225 | 3FL-1 | Galβ1,4(Fucα1,3)Glc |
| 229 | G6GN-1 | Galβ1,6GlcNAc |
| 230 | G4GN-1 | Galβ1,4GlcNAc |
| 233 | 2FL-1 | Fucα1,2Galβ1,4Glc |
| 234 | TNA-1 | GalNAcα1-O-L-serine |
| 235 | Lac-4 | Galβ1,4Glc |
| 236 | 2FL-5 | Fucα1,2Galβ1,4Glc |
| 237 | A3 | Manα1,3Man |
| 239 | SLA-4 | Neu5Acα2,3Galβ1,3(Fucα1,4)GlcNAc |
| 240 | SOLA-5 | 3′-(O—SO3H)Galβ1,3(Fucα1,4)GlcNAc |
| 249 | 3SL-1 | Neu5Acα2,3Galβ1,4Glc |
| 254 | G3G-1 | Galα1,3Gal |
| 263 | 3SLN-1 | Neu5Acα2,3Galβ1,4GlcNAc |
| 273 | BAT-5 | GalNAcα1,3(Fucα1,2)Gal |
| 285 | SLX-5 | Neu5Acα2,3Galβ1,4(Fucα1,3)GlcNAc |
| 288 | LA-1 | Galβ1,3(Fucα1,4)GlcNAc |
| 290 | SLA-5 | Neu5Acα2,3Galβ1,3(Fucα1,4)GlcNAc |
| 292 | STNA-5 | Neu5Acα2,6GalNAcα1-O-L-serine |
| 295 | 6SLN-4 | Neu5Acα2,6Galβ1,4GlcNAc |
| 301 | TRIS | tris(hydroxymethyl)aminomethane |

When used in the description, “modification (binding) density” refers to an amount of a sugar chain that is used for preparation of a sugar-chain-modified liposome. The term is conveniently used to represent the density of a sugar chain (mg sugar chain/mg lipid) that binds per mg of lipids in a liposome in the description. Although no theoretical constraints are desired herein, regarding the binding density of the sugar-chain-modified liposome of the present invention, it is empirically known that the amount of a sugar chain that is used for preparation is almost proportional to the density of a sugar chain bound to the liposome. Therefore, in the description, unless otherwise particularly noted, binding density is determined depending on the amount used upon preparation. In vitro, for example, such density can be indirectly determined using E-selectin. In the present invention, when a delivery vehicle is a sugar-chain-modified liposome, tropism for a target delivery site can be controlled via selection of the type and binding density of a sugar chain to be bound to the liposome. Delivery vehicles and related organ tropism are listed as follows.

Definition of tropism evaluation (++, +) is as described below. Furthermore, evaluation (−) represents a negative result and NA represent “not measured.”

TABLE 4
Table 4 Definition for evaluation of tropism
(targeting property) (++ and +)

| | | Range of | |
|---|---|---|---|
| | Evaluation | standardized relative | |
| | of tropism | value (average | No. and |
| | (targeting | value of each | abbreviated |
| | property) | liposome/average | name |
| | (5 minutes after | value of | of negative |
| Organ | administration) | negative liposome) | liposome |
| Oral administration | ++ | 4~6 | 301 |
| (10 minutes after | + | 3~4 | TRIS |
| administration) | | | |
| Liver | ++ | 2~3 | 301 |
| | + | 1~2 | TRIS |
| Heart | ++ | 3~6 | 218 |
| | + | 2~3 | EEGM1-BH |
| Lung | ++ | 2~5 | 218 |
| | + | 1~2 | EEGM1-BH |
| Pancreas | ++ | 2~4 | 218 |
| | + | 1~2 | EEGM1-BH |
| Brain | ++ | 3~7 | 218 |
| | + | 2~3 | EEGM1-BH |
| Tumor | ++ | 2~4 | 218 |
| | + | 1~2 | EEGM1-BH |
| Inflammation site | ++ | 2~8 | 218 |
| | + | 1~2 | EEGM1-BH |
| Small intestine | ++ | 3~4 | 218 |
| | + | 2~3 | EEGM1-BH |
| Large intestine | ++ | 4~7 | 218 |
| | + | 3~4 | EEGM1-BH |
| Lymph node | ++ | 6~24 | 218 |
| | + | 4~6 | EEGM1-BH |
| Bone marrow | ++ | 10~14 | 301 |
| | + | 8~10 | TRIS |
| Blood (in blood) | ++ | 6~13 | 218 |
| | + | 4~6 | EEGM1-BH |
| Kidney | ++ | 2~3 | 218 |
| | + | 1~2 | EEGM1-BH |
| Spleen | ++ | 2~4 | 218 |
| | + | 1~2 | EEGM1-BH |
| Thymus gland | ++ | 4~9 | 218 |
| | + | 3~4 | EEGM1-BH |
| Muscle | ++ | 3~5 | 218 |
| | + | 2~3 | EEGM1-BH |

In addition, in the case of oral administration, the average value of each liposome that is absorbed via the intestinal tract at 10 minutes after administration is divided by the average value of the standard liposome and the result is shown in table (average value of each liposome/average value of the negative liposome).

This is represented as follows when the same Tris is used as a standard for all cases.

TABLE 4A
Definition for evaluation of tropism (targeting property)
(++ and +)

| | | Range of | |
|---|---|---|---|
| | Evaluation | standardized relative | |
| | of tropism | value (average | No. and |
| | (targeting | value of each | abbreviated |
| | property) | liposome/average | name |
| | (5 minutes after | value of | of negative |
| Organ | administration) | negative liposome) | liposome |
| Oral administration | ++ | 2.8~4.4 | 301 |
| (10 minutes after | + | 2.3~2.7 | TRIS |
| administration) | | | |
| Liver | ++ | 1.5~2.1 | 301 |
| | + | 1.2~1.5 | TRIS |
| Heart | ++ | 1.4~2.4 | 301 |
| | + | 1.1~1.4 | TRIS |
| Lung | ++ | 1.6~3.5 | 301 |
| | + | 1.3~1.6 | TRIS |
| Pancreas | ++ | 1.5~2.2 | 301 |
| | + | 1.2~1.5 | TRIS |
| Brain | ++ | 1.5~3.7 | 301 |
| | + | 1.1~1.4 | TRIS |
| Tumor | ++ | 1.5~2.5 | 301 |
| | + | 1.1~1.4 | TRIS |
| Inflammation site | ++ | 1.5~4.9 | 301 |
| | + | 1.2~1.5 | TRIS |
| Small intestine | ++ | 1.7~2.5 | 301 |
| | + | 1.4~1.7 | TRIS |
| Large intestine | ++ | 1.6~2.7 | 301 |
| | + | 1.2~1.5 | TRIS |
| Lymph node | ++ | 2.0~8.7 | 301 |
| | + | 1.6~2.0 | TRIS |
| Bone marrow | ++ | 1.3~1.9 | 301 |
| | + | 1.1~1.3 | TRIS |
| Blood (in blood) | ++ | 1~2.1 | 301 |
| | + | 0.6~0.9 | TRIS |
| Kidney | ++ | 1.7~2.7 | 301 |
| | + | 1.4~1.7 | TRIS |
| Spleen | ++ | 2.3~4.0 | 301 |
| | + | 1.6~2.2 | TRIS |
| Thymus gland | ++ | 2.1~4.3 | 301 |
| | + | 1.6~2.1 | TRIS |
| Muscle | ++ | 1.4~2.8 | 301 |
| | + | 1.1~1.4 | TRIS |

In the present invention, delivery vehicles that are provided based on the rolling model may be sugar-chain-modified liposomes with the sugar chain densities as listed in the following Table.

TABLE 5

| | | Modification |
|---|---|---|
| | | (binding) |
| | | density |
| | | (mg sugar |
| | Abbreviated | chain/ |
| Liposome No. | name | mg lipid) |
| 3 | EEGD1A-BH | 0.75 |
| 27 | LDF-5 | 0.5 |
| 29 | 3SL-5 | 0.5 |
| 37 | FEEGD1b | 0.75 |
| 38 | G4GN-4 | 0.25 |
| 40 | BAT-1 | 0.025 |
| 41 | SOLX-4 | 0.25 |
| 45 | BHD-1 | 0.025 |
| 50 | ManB | 0.0385 |
| 53 | L2F-1 | 0.025 |
| 56 | Man9 | 0.042 |
| 60 | 6SLN-1 | 0.025 |
| 67 | FEEaMDa | [symbol] 0.375 |
| 68 | Man7 | 0.035 |
| 69 | L4F-1 | 0.025 |
| 70 | LNT-5 | 0.5 |
| 71 | BBT-1 | 0.025 |
| 76 | BHD-4 | 0.25 |
| 80 | 6SLN-5 | 0.5 |
| 87 | TA-1 | 0.025 |
| 91 | G6GN-4 | 0.25 |
| 93 | A36 | 0.011 |
| 96 | TA-4 | 0.25 |
| 105 | Man6 | 0.031 |
| 106 | LDF-4 | 0.25 |
| 111 | LNB-5 | 0.5 |
| 116 | LDF-1 | 0.025 |
| 117 | TA-5 | 0.5 |
| 120 | A2 | 0.0075 |
| 125 | LX-1 | 0.025 |
| 127 | G3G-4 | 0.25 |
| 129 | BBT-4 | 0.25 |
| 130 | 2FL-4 | 0.25 |
| 137 | Man4B | 0.024 |
| 139 | 3FL-5 | 0.5 |
| 141 | BBT-5 | 0.5 |
| 142 | BHD-5 | 0.5 |
| 146 | 3FL-4 | 0.25 |
| 150 | TNA-5 | 0.5 |
| 151 | SLX-4 | 0.25 |
| 152 | LY-1 | 0.025 |
| 153 | G6GN-5 | 0.5 |
| 154 | Lac-5 | 0.5 |
| 155 | A6 | 0.0075 |
| 175 | G3G-5 | 0.5 |
| 178 | LNT-4 | 0.25 |
| 183 | G4GN-5 | 0.5 |
| 184 | STNA-1 | 0.025 |
| 186 | Man3 | 0.0205 |
| 191 | LB-1 | 0.025 |
| 195 | TNA-4 | 0.25 |
| 197 | A4 | 0.0075 |
| 199 | STNA-4 | 0.25 |
| 204 | Lac-1 | 0.025 |
| 207 | 6SL-5 | 0.5 |
| 209 | LDFH-4 | 0.25 |
| 213 | LX-4 | 0.25 |
| 218 | EEGM1-BH | 0.75 |
| 220 | LNB-4 | 0.25 |
| 224 | Man5 | 0.0275 |
| 225 | 3FL-1 | 0.025 |
| 229 | G6GN-1 | 0.025 |
| 230 | G4GN-1 | 0.025 |
| 233 | 2FL-1 | 0.025 |
| 234 | TNA-1 | 0.025 |
| 235 | Lac-4 | 0.25 |
| 236 | 2FL-5 | 0.5 |
| 237 | A3 | 0.0075 |
| 239 | SLA-4 | 0.25 |
| 240 | SOLA-5 | 0.5 |
| 249 | 3SL-1 | 0.025 |
| 254 | G3G-1 | 0.025 |
| 263 | 3SLN-1 | 0.025 |
| 273 | BAT-5 | 0.5 |
| 285 | SLX-5 | 0.5 |
| 288 | LA-1 | 0.025 |
| 290 | SLA-5 | 0.5 |
| 292 | STNA-5 | 0.5 |
| 295 | 6SLN-4 | 0.25 |
| 301 | TRIS | |

In the present invention, a sugar-chain-modified liposome that is a typical delivery vehicle can be produced by the following method. Specifically, the method comprises the steps of: (a) providing a liposome; (b) hydrophilizing the liposome; (c) generating a linker-bound liposome by binding a linker to the hydrophilized liposome according to need; and (d) generating a sugar-chain-modified liposome by binding a sugar chain listed in Table 3 above to the liposome.

Preferably, in the method: the step (b) of hydrophilizing a liposome is performed by directly or indirectly binding a low-molecular-weight hydrophilic compound onto a lipid membrane or linker of the liposome; the linker to be used in the step (c) is a human-derived protein (e.g., human serum albumin); and in the step (d), under conditions where a sugar chain is directly or indirectly bound to the liposome, a sugar-chain-modified liposome may be generated by binding the sugar chain.

Preferably, a liposome is bound to a linker or a linker is bound to a sugar chain using a bifunctional cross-linking agent (e.g., DTSSP) or the like.

A drug or a gene can be encapsulated in or bound to the delivery vehicle of the present invention. Examples of such a drug include, but are not limited to, alkylating anticancer agents, antimetabolites, plant-derived anticancer agents, anticancerous antibiotics, biological response modifiers (BRM) cytokines, drugs for tumors, such as platinum complex-based anticancer agents, immunotherapeutic agents, hormone-based anticancer agents, and a monoclonal antibody, drugs for the central nerve, drugs for the peripheral nerve system-sense organs, drugs for treating respiratory diseases, drugs for circulatory organs, drugs for digestive organs, drugs for hormone systems, drugs for urinary organs-reproductive organs, vitamins-revitalizers, metabolic pharmaceutical products, antibiotic chemotherapy drugs, drugs for examination, anti-inflammatory agents, drugs for eye disease, drugs for the central nervous system, drugs for the autoimmune system, drugs for the circulatory system, diabetes, drugs for lifestyle-related diseases such as hyperlipemia, adrenal cortex hormones, immunosuppresants, antimicrobial agents, antiviral agents, agents for suppressing vascularization, cytokines, chemokines, anti-cytokine antibodies, anti-chemokine antibodies, anti-cytokine-chemokine receptor antibodies, gene therapy-related nucleic acid formulations, such as siRNA, miRNA, smRNA, antisense oligodeoxynucleotide (ODN), and DNA, neuroprotective factors, antibody medicines, molecular target drugs, osteoporosis-bone metabolism improving drugs, neuropeptides, and physiologically active peptides-proteins.

When used in the description, “linker” refers to a molecule that mediates binding of a sugar chain to a liposome surface. In the sugar-chain-modified liposome of the present invention, a sugar chain may be bound to a liposome surface via a linker. A linker can be adequately selected by persons skilled in the art and is preferably biocompatible and more preferably pharmaceutically acceptable. A linker that is used in the description can be, for example, a protein derived from a living body, preferably a human-derived protein, more preferably a human-derived serum protein, and further more preferably human serum albumin or bovine serum albumin. In particular, when a human serum albumin is used, it has been confirmed by an experiment conducted using mice that such albumin is incorporated well into each tissue.

In the description, “cross-linking agent” refers to an agent by which a chemical bond is formed between molecules of chain polymers in a manner similar to that of the building of a bridge. Typically, such a cross-linking agent refers to an agent that acts between a polymer (e.g., a lipid, a protein, a peptide, or a sugar chain) and another molecule (e.g., a lipid, a protein, a peptide, or a sugar chain), so as to form a covalent bond that links within molecules or between molecules (between which no covalent bond is present previously). In the description, a covalent bond may be formed between a liposome and a sugar chain with the use of such a cross-linking agent. Alternatively, a liposome and a sugar chain may be linked via a linker, the linker and the sugar chain may be linked with the use of such a cross-linking agent, and the linker and the liposome may be linked with the use of such a cross-linking agent. Examples of such a cross-linking agent may be varied depending on targets to be cross-linked and include, but are not limited to, aldehydes (e.g., glutaraldehyde), carbodiimides, and imide esters. When an amino group-containing substance is subjected to cross-linking, an aldehyde-containing group, such as glutaraldehyde, can be used. Specifically, divalent reagents such as bissulfosuccinimidyl suberate, disuccinimidyl glutarate, dithiobissuccinimidyl propionate, disuccinimidyl suberate, 3,3′-dithiobissulfosuccinimidyl propionate, ethylene glycol bissuccinimidyl succinate, and ethylene glycol bissulfosuccinimidyl succinate can be used, for example.

Terms that are used in the description, “protein,” “polypeptide,” “oligopeptide,” and “peptide” are used in the same sense and refer to polymers having arbitrary lengths of amino acids. Such a polymer may be linear or branched or in the form of a ring. “Amino acid” may be a natural or non-natural or altered amino acid. Examples of the term may also include those assembled to form complexes with a plurality of polypeptide chains. Examples of the term also include natural or artificially altered amino acid polymers. Examples of such alteration include disulfide bond formation, glycosylation, lipidation, acetylation, phosphorylation or other arbitrary manipulation or alteration (e.g., formation of a conjugate with a labeling component). This definition can also be applied to a polypeptide (containing non-natural amino acid or the like, for example) containing 1, 2, or more analogs of amino acids, a peptide-like compound (e.g., peptoid), and other alterations known in the art.

In the description, it should be understood, when particularly noted: “protein” indicates an amino acid polymer having a relatively small molecular weight or an altered product thereof; and “peptide” indicates an amino acid polymer having a relatively large molecular weight or an altered product thereof. Examples of such a molecular weight include, but are not limited to, approximately 30 kDa, preferably approximately 20 kDa, and more preferably approximately 10 kDa.

When used in the description, “protein derived from a living body” refers to a protein derived from an organism. Such proteins may be derived from any organisms (e.g., arbitrary types of multicellular organism including animals such as vertebrates and invertebrates and plants such as monocotyledons and dicotyledons, for example). Preferably, proteins derived from vertebrates (e.g., Hyperotreta, Hyperoartia, Chondrichthyes, Osteichthyes, amphibians, reptiles, birds, and mammals) and more preferably, proteins derived from mammals (e.g., Monotreme, Marsupialia, Edentata, Dermoptera, Chiropteran, Carnivore, Insectivore, Proboscidean, Perissodactyla, Artiodactyla, Tubulidentata, Squamata, Sirenia, Cetacea, primates, rodents, and Lagomorpha) are used. Further preferably, proteins derived from primates (e.g., chimpanzee, Japanese monkey (Macaca fuscata), and human) are used. Most preferably, proteins derived from living bodies to which administration is performed are used.

When used in the description, “human-derived serum protein” refers to a protein that is contained in a liquid portion that remains when human blood naturally coagulates.

When used in the description, “human serum albumin” refers to an albumin contained in human serum, and “bovine serum albumin” refers to an albumin contained in bovine serum.

The sugar-chain-modified liposome of the present invention may be hydrophilized by binding a hydrophilic compound and preferably tris(hydroxyalkyl)aminoalkane to at least one of the liposome membrane and the linker.

When used in the description, “hydrophilization” means to bind a hydrophilic compound to a liposome surface. Examples of a compound to be used for hydrophilization include, a low-molecular-weight hydrophilic compound, preferably a low-molecular-weight hydrophilic compound having at least one OH group, and further preferably a low-molecular-weight hydrophilic compound having at least two OH groups. Another example of the same is a low-molecular-weight hydrophilic compound having at least one amino group; that is, a hydrophilic compound having at least one OH group and at least one amino group within the molecule. Such a hydrophilic compound has a low molecular weight, so that it hardly causes steric hindrance against sugar chains. Thus, such a hydrophilic compound will never hinder the progress of reaction for recognition of a sugar chain molecule, which is performed by a cell surface molecule such as a lectin on a target cell membrane. Furthermore, in the sugar-chain-modified liposome of the present invention, such a hydrophilic compound does not contain a sugar chain to which a cell surface molecule such as a lectin to be used for targeting a specific site can bind. Examples of such a hydrophilic compound include amino alcohols such as tris(hydroxyalkyl)aminoalkane containing tris(hydroxymethyl)aminomethane or the like. More specific examples of the same include tris(hydroxymethyl)aminoethane, tris(hydroxyethyl)aminoethane, tris(hydroxypropyl)aminoethane, tris(hydroxymethyl)aminomethane, tris(hydroxyethyl)aminomethane, tris(hydroxypropyl)aminomethane, tris(hydroxymethyl)aminopropane, tris(hydroxyethyl)aminopropane, and tris(hydroxypropyl)aminopropane. Moreover, a compound prepared by introduction of an amino group into a low-molecular-weight compound having an OH group can also be used as the hydrophilic compound of the present invention. An example of the compound is, but is not limited to, a compound prepared by introduction of an amino group into a sugar chain such as cellobiose to which a cell surface molecule (e.g., lectin) does not bind. For example, a liposome surface is hydrophilized by applying a divalent reagent for cross-linking and tris(hydroxymethyl)aminomethane onto lipid phosphatidyl ethanol amine of the liposome membrane. Such a hydrophilic compound is represented by the following general formula (1), (2), (3), or the like.

X—R¹(R²⁰H)n (formula (1))

H₂N—R³—(R¹⁰H)n (formula (2))

H₂N—R⁵(OH)n (formula (3))

Wherein R¹, R³, and R⁵ denote C₁ to C₄₀, preferably C₁ to C₂₀, further preferably C₁ to C₁₀ linear or branched hydrocarbon chains, R² and R⁴ are absent or denote C₁ to C₄₀, preferably C₁ to C₂₀, further preferably C₁ to C₁₀ linear or branched hydrocarbon chains. X denotes a reactive functional group directly binding to a liposome lipid or a divalent reagent for cross-linking. Examples of “X” include COOH, NH, NH₂, CHO, SH, NHS-ester, maleimide, imide ester, active halogen, EDC, pyridyl disulfide, azidophenyl, and hydrazide. N denotes a natural number. The surface of the liposome hydrophilized using such a hydrophilic compound is coated thinly with the hydrophilic compound. However, the coating thickness of the hydrophilic compound is thin, so that when a sugar chain is bound to the liposome, the reactivity of the sugar chain or the like is not suppressed.

Hydrophilization of a liposome is performed by a conventionally known method. For example, hydrophilization can be performed by employing a method that involves preparing a liposome using a phospholipid to which polyethylene glycol, polyvinylalcohol, maleic anhydride copolymer, and the like are covalently bound (JP Patent Publication (Kokai) No. 2000-302685 A discloses that a crude dispersion of a multilayered liposome was obtained by a method using, for example, CNDAC-containing liposome formulation dilauroylphosphatidylcholine, dimyristoylphosphatidylcholine, dipalmitoylphosphatidylcholine, distearoyl phosphatidylcholine; dipalmitoylphosphatidylglycerol, distearoylphosphatidylglycerol; sphingomyelin; cholesterol; N-monomethoxy polyethylene glycol succinyl-distearoyl phosphatidyl ethanol amine (hereinafter, referred to as PEG2000-DSPE) in which the molecular weight of the polyethylene glycol portion is approximately 2000; CNDAC hydrochloride, a glucose aqueous solution, and a trehalose aqueous solution according to the method of Bangham et al., (see J. Mol. Biol. 8, 660-668 (1964)), for example. Of these, it is particularly preferred that a liposome surface is hydrophilized using tris(hydroxymethyl)aminomethane. The technique of the present invention using tris(hydroxymethyl)aminomethane is preferred because of several reasons compared with conventional hydrophilization methods using polyethylene glycol and the like. For example, in the present invention, a sugar chain is bound onto a liposome and then the molecular recognition function is used for tropism. In such a case, tris(hydroxymethyl)aminomethane is particularly preferred since: tris(hydroxymethyl)aminomethane that is a low-molecular-weight substance hardly causes steric hindrance against sugar chains compared with a conventional method using a high-molecular-weight substance such as polyethylene glycol; and tris(hydroxymethyl)aminomethane does not hinder the progress of a sugar chain molecule recognition reaction that is performed by a cell surface molecule (sugar chain-recognizing protein) such as a lectin on a target cell membrane.

Furthermore, the liposome according to the present invention has good particle diameter distribution, component composition, and dispersion property even after hydrophilization and is also excellent in long-term storage stability and in vivo stability. Therefore, the liposome is preferred when it is formulated and used. To hydrophilize the surface of a liposome using tris(hydroxymethyl)aminomethane, for example, a divalent reagent (e.g., bissulfosuccinimidyl suberate, disuccinimidyl glutarate, dithiobissuccinimidyl propionate, disuccinimidyl suberate, 3,3′-dithiobissulfosuccinimidyl propionate, ethylene glycol bissuccinimidyl succinate, and ethylene glycol bissulfosuccinimidyl succinate) is added to a liposome solution obtained by a standard method with the use of lipids such as dimyristoylphosphatidyl ethanol amine, dipalmitoyl phosphatidyl ethanol amine, and distearoyl phosphatidyl ethanol amine, so as to perform a reaction. The divalent reagent is bound to lipids such as dipalmitoyl phosphatidyl ethanol amine on the liposome membrane and then tris(hydroxymethyl)aminomethane is caused to react with the other binding site of the divalent reagent, so that tris(hydroxymethyl)aminomethane is bound to the liposome surface.

As described above, the thus hydrophilized liposome is extremely stable in vivo. As described later, such a hydrophilic liposome has a long half-life in vivo without binding of a sugar chain having tropism, so that it can be appropriately used as a drug vehicle in a drug delivery system. The present invention also encompasses a liposome hydrophilized by treating the surface with such a low-molecular-weight compound.

In the design of the delivery vehicle based on the rolling model discovered in the description, in vitro evaluation standard can be determined by an experiment that is conducted for one type of lectin (e.g., E-selectin), for example. The results of the experiment can be listed as follows.

TABLE 6A

| Sample ID | IC10 | Sample ID | IC10 |
|---|---|---|---|
| 76 | <4.83E−10 | 224 | 2.97E−10 |
| 45 | <4.83E−10 | 105 | 4.81E−10 |
| 127 | <4.83E−10 | 71 | 5.99E−10 |
| 273 | <4.83E−10 | 137 | 6.02E−10 |
| 295 | <4.83E−10 | 186 | 8.41E−10 |
| 80 | <4.83E−10 | 93 | 9.73E−10 |
| 263 | <4.83E−10 | 142 | 9.87E−10 |
| 69 | <4.83E−10 | 40 | 1.17E−09 |
| 53 | <4.83E−10 | 116 | 1.21E−09 |
| 91 | <4.83E−10 | 60 | 1.25E−09 |
| 153 | <4.83E−10 | 38 | 1.39E−09 |
| 230 | <4.83E−10 | 233 | 1.43E−09 |
| 229 | <4.83E−10 | 141 | 1.66E−09 |
| 195 | <4.83E−10 | 50 | 1.80E−09 |
| 150 | <4.83E−10 | 290 | 1.84E−09 |
| 199 | <4.83E−10 | 254 | 2.03E−09 |
| 184 | <4.83E−10 | 68 | 2.11E−09 |
| 234 | <4.83E−10 | 292 | 2.26E−09 |
| 146 | <4.83E−10 | 111 | 2.62E−09 |
| 139 | <4.83E−10 | 288 | 2.63E−09 |
| 154 | <4.83E−10 | 129 | 2.86E−09 |
| 204 | <4.83E−10 | 70 | 3.34E−09 |
| 235 | <4.83E−10 | 207 | 3.76E−09 |
| 225 | <4.83E−10 | 209 | 3.78E−09 |
| 106 | <4.83E−10 | 220 | 3.83E−09 |
| 27 | <4.83E−10 | 237 | 4.04E−09 |
| 96 | <4.83E−10 | 130 | 4.12E−09 |
| 117 | <4.83E−10 | 3 | 4.93E−09 |
| 87 | <4.83E−10 | 197 | 5.01E−09 |
| 152 | <4.83E−10 | 155 | 6.47E−09 |
| 191 | <4.83E−10 | 178 | 6.84E−09 |
| 213 | <4.83E−10 | 120 | 7.02E−09 |
| 125 | <4.83E−10 | 218 | 7.23E−09 |
| 151 | <4.83E−10 | 236 | 7.44E−09 |
| 285 | <4.83E−10 | 301 | 8.56E−09 |
| 239 | <4.83E−10 | 56 | 1.61E−08 |
| 41 | <4.83E−10 | 183 | 1.99E−08 |
| 240 | <4.83E−10 | 175 | 2.01E−08 |
| 29 | 1.21E−11 | 302 | 3.97E−08 |

TABLE 6B

| Sample ID | IC20 | Sample ID | IC20 |
|---|---|---|---|
| 87 | <4.83E−10 | 50 | 2.78E−09 |
| 45 | <4.83E−10 | 71 | 2.78E−09 |
| 263 | <4.83E−10 | 230 | 2.85E−09 |
| 53 | <4.83E−10 | 154 | 3.08E−09 |
| 229 | <4.83E−10 | 234 | 3.41E−09 |
| 195 | <4.83E−10 | 233 | 3.41E−09 |
| 150 | <4.83E−10 | 116 | 3.78E−09 |
| 199 | <4.83E−10 | 129 | 3.83E−09 |
| 184 | <4.83E−10 | 111 | 3.94E−09 |
| 146 | <4.83E−10 | 288 | 4.00E−09 |
| 204 | <4.83E−10 | 254 | 4.05E−09 |
| 225 | <4.83E−10 | 68 | 4.28E−09 |
| 106 | <4.83E−10 | 224 | 4.43E−09 |
| 96 | <4.83E−10 | 207 | 4.64E−09 |
| 152 | <4.83E−10 | 292 | 4.66E−09 |
| 125 | <4.83E−10 | 70 | 4.86E−09 |
| 151 | <4.83E−10 | 60 | 5.30E−09 |
| 285 | <4.83E−10 | 197 | 5.85E−09 |
| 240 | <4.83E−10 | 220 | 6.57E−09 |
| 209 | 4.58E−10 | 237 | 7.43E−09 |
| 139 | 7.93E−10 | 91 | 7.48E−09 |
| 213 | 9.28E−10 | 290 | 7.78E−09 |
| 41 | 1.06E−09 | 130 | 7.91E−09 |
| 117 | 1.09E−09 | 120 | 8.70E−09 |
| 69 | 1.28E−09 | 178 | 1.01E−08 |
| 191 | 1.40E−09 | 155 | 1.01E−08 |
| 80 | 1.44E−09 | 137 | 1.04E−08 |
| 235 | 1.48E−09 | 93 | 1.06E−08 |
| 27 | 1.49E−09 | 186 | 1.12E−08 |
| 239 | 1.58E−09 | 3 | 1.13E−08 |
| 141 | 1.80E−09 | 236 | 1.18E−08 |
| 38 | 1.81E−09 | 29 | 1.23E−08 |
| 142 | 2.07E−09 | 127 | 1.27E−08 |
| 273 | 2.23E−09 | 301 | 2.02E−08 |
| 295 | 2.30E−09 | 218 | 2.03E−08 |
| 76 | 2.39E−09 | 56 | 2.35E−08 |
| 105 | 2.51E−09 | 302 | 2.37E−08 |
| 153 | 2.53E−09 | 175 | 2.49E−08 |
| 40 | 2.78E−09 | 183 | 2.55E−08 |

TABLE 6C

| Sample ID | IC30 | Sample ID | IC30 |
|---|---|---|---|
| 87 | <4.83E−10 | 93 | 9.45E−10 |
| 76 | <4.83E−10 | 142 | 9.87E−10 |
| 45 | <4.83E−10 | 240 | 1.06E−09 |
| 273 | <4.83E−10 | 40 | 1.17E−09 |
| 295 | <4.83E−10 | 116 | 1.21E−09 |
| 80 | <4.83E−10 | 60 | 1.25E−09 |
| 263 | <4.83E−10 | 151 | 1.37E−09 |
| 69 | <4.83E−10 | 38 | 1.39E−09 |
| 53 | <4.83E−10 | 233 | 1.43E−09 |
| 91 | <4.83E−10 | 141 | 1.66E−09 |
| 153 | <4.83E−10 | 50 | 1.80E−09 |
| 230 | <4.83E−10 | 254 | 2.03E−09 |
| 229 | <4.83E−10 | 68 | 2.11E−09 |
| 195 | <4.83E−10 | 292 | 2.26E−09 |
| 150 | <4.83E−10 | 111 | 2.62E−09 |
| 199 | <4.83E−10 | 285 | 2.70E−09 |
| 184 | <4.83E−10 | 129 | 2.86E−09 |
| 234 | <4.83E−10 | 70 | 3.34E−09 |
| 146 | <4.83E−10 | 209 | 3.78E−09 |
| 139 | <4.83E−10 | 220 | 3.83E−09 |
| 154 | <4.83E−10 | 237 | 4.04E−09 |
| 204 | <4.83E−10 | 130 | 4.12E−09 |
| 235 | <4.83E−10 | 213 | 4.94E−09 |
| 225 | <4.83E−10 | 197 | 5.01E−09 |
| 106 | <4.83E−10 | 288 | 5.54E−09 |
| 27 | <4.83E−10 | 155 | 6.47E−09 |
| 96 | <4.83E−10 | 178 | 6.84E−09 |
| 117 | <4.83E−10 | 120 | 7.02E−09 |
| 152 | <4.83E−10 | 239 | 7.28E−09 |
| 191 | <4.83E−10 | 236 | 7.44E−09 |
| 224 | 2.97E−10 | 41 | 1.03E−08 |
| 105 | 4.81E−10 | 290 | 1.16E−08 |
| 127 | 4.83E−10 | 56 | 1.61E−08 |
| 29 | 4.83E−10 | 183 | 1.99E−08 |
| 207 | 4.83E−10 | 175 | 2.01E−08 |
| 71 | 5.99E−10 | 3 | 2.21E−08 |
| 137 | 6.02E−10 | 301 | 3.18E−08 |
| 125 | 6.28E−10 | 302 | 4.05E−08 |
| 186 | 8.41E−10 | 218 | 6.08E−08 |

TABLE 6D

| Sample ID | IC40 | Sample ID | IC40 |
|---|---|---|---|
| 53 | 5.96E−10 | 224 | 1.49E−08 |
| 263 | 1.52E−09 | 80 | 1.52E−08 |
| 139 | 2.85E−09 | 237 | 1.53E−08 |
| 184 | 3.55E−09 | 290 | 1.54E−08 |
| 150 | 3.87E−09 | 130 | 1.70E−08 |
| 151 | 4.16E−09 | 41 | 1.71E−08 |
| 117 | 4.47E−09 | 60 | 1.84E−08 |
| 152 | 4.48E−09 | 29 | 1.90E−08 |
| 106 | 4.83E−09 | 207 | 1.99E−08 |
| 141 | 4.92E−09 | 178 | 2.01E−08 |
| 225 | 5.13E−09 | 155 | 2.01E−08 |
| 27 | 5.22E−09 | 38 | 2.06E−08 |
| 40 | 5.39E−09 | 91 | 2.09E−08 |
| 295 | 5.63E−09 | 186 | 2.09E−08 |
| 71 | 5.65E−09 | 69 | 2.13E−08 |
| 191 | 6.04E−09 | 153 | 2.16E−08 |
| 146 | 6.10E−09 | 233 | 2.17E−08 |
| 229 | 6.25E−09 | 235 | 2.18E−08 |
| 96 | 6.50E−09 | 125 | 2.30E−08 |
| 45 | 6.91E−09 | 236 | 2.34E−08 |
| 197 | 7.53E−09 | 127 | 2.39E−08 |
| 285 | 7.67E−09 | 50 | 2.52E−08 |
| 129 | 7.72E−09 | 154 | 2.59E−08 |
| 68 | 7.80E−09 | 240 | 2.61E−08 |
| 195 | 8.35E−09 | 199 | 2.66E−08 |
| 105 | 9.24E−09 | 175 | 3.45E−08 |
| 87 | 1.02E−08 | 183 | 3.67E−08 |
| 111 | 1.02E−08 | 204 | 3.82E−08 |
| 230 | 1.06E−08 | 234 | 3.86E−08 |
| 213 | 1.10E−08 | 70 | 3.98E−08 |
| 142 | 1.15E−08 | 3 | 4.17E−08 |
| 288 | 1.17E−08 | 301 | 4.34E−08 |
| 254 | 1.20E−08 | 218 | 1.43E−07 |
| 239 | 1.20E−08 | 120 | >4.83E−08 |
| 76 | 1.32E−08 | 56 | >4.83E−08 |
| 220 | 1.32E−08 | 137 | >4.83E−08 |
| 292 | 1.38E−08 | 93 | >4.83E−08 |
| 273 | 1.42E−08 | 209 | >4.83E−08 |
| 116 | 1.44E−08 | 302 | >4.83E−08 |

TABLE 6E

| Sample ID | IC50 | Sample ID | IC50 |
|---|---|---|---|
| 117 | 8.45E−09 | 80 | 2.43E−08 |
| 263 | 8.59E−09 | 29 | 2.51E−08 |
| 191 | 9.18E−09 | 150 | 2.63E−08 |
| 71 | 9.57E−09 | 146 | 2.70E−08 |
| 152 | 9.77E−09 | 199 | 2.70E−08 |
| 295 | 1.00E−08 | 130 | 2.73E−08 |
| 40 | 1.03E−08 | 233 | 2.75E−08 |
| 53 | 1.11E−08 | 106 | 2.76E−08 |
| 151 | 1.14E−08 | 125 | 2.84E−08 |
| 229 | 1.22E−08 | 91 | 2.89E−08 |
| 27 | 1.27E−08 | 60 | 3.03E−08 |
| 129 | 1.28E−08 | 236 | 3.05E−08 |
| 87 | 1.28E−08 | 153 | 3.09E−08 |
| 225 | 1.30E−08 | 127 | 3.16E−08 |
| 96 | 1.39E−08 | 235 | 3.33E−08 |
| 141 | 1.41E−08 | 207 | 3.48E−08 |
| 285 | 1.44E−08 | 240 | 3.52E−08 |
| 111 | 1.53E−08 | 154 | 3.66E−08 |
| 105 | 1.53E−08 | 175 | 3.93E−08 |
| 142 | 1.59E−08 | 183 | 4.23E−08 |
| 45 | 1.59E−08 | 178 | 4.24E−08 |
| 197 | 1.60E−08 | 38 | 4.50E−08 |
| 239 | 1.75E−08 | 234 | 4.72E−08 |
| 254 | 1.81E−08 | 3 | 6.88E−08 |
| 184 | 1.83E−08 | 155 | >2.42E−08 |
| 213 | 1.88E−08 | 120 | >2.42E−08 |
| 237 | 1.93E−08 | 68 | >2.42E−08 |
| 273 | 1.95E−08 | 50 | >2.42E−08 |
| 195 | 1.99E−08 | 56 | >2.42E−08 |
| 76 | 1.99E−08 | 137 | >2.42E−08 |
| 230 | 2.02E−08 | 186 | >2.42E−08 |
| 288 | 2.07E−08 | 93 | >2.42E−08 |
| 220 | 2.12E−08 | 69 | >4.83E−08 |
| 224 | 2.22E−08 | 70 | >4.83E−08 |
| 292 | 2.25E−08 | 209 | >4.83E−08 |
| 290 | 2.30E−08 | 204 | >4.83E−08 |
| 41 | 2.31E−08 | 218 | >2.42E−07 |
| 116 | 2.34E−08 | 301 | >4.83E−08 |
| 139 | 2.37E−08 | 302 | >4.83E−08 |

TABLE 6F

| Sample ID | IC60 | Sample ID | IC60 |
|---|---|---|---|
| 191 | 1.23E−08 | 127 | 3.54E−08 |
| 71 | 1.35E−08 | 288 | 3.61E−08 |
| 87 | 1.37E−08 | 150 | 3.66E−08 |
| 295 | 1.44E−08 | 91 | 3.69E−08 |
| 263 | 1.44E−08 | 199 | 3.70E−08 |
| 117 | 1.45E−08 | 130 | 3.75E−08 |
| 40 | 1.50E−08 | 106 | 3.75E−08 |
| 152 | 1.72E−08 | 236 | 3.76E−08 |
| 53 | 1.93E−08 | 111 | 3.82E−08 |
| 129 | 1.95E−08 | 153 | 4.02E−08 |
| 151 | 2.01E−08 | 146 | 4.11E−08 |
| 229 | 2.13E−08 | 60 | 4.23E−08 |
| 105 | 2.14E−08 | 175 | 4.40E−08 |
| 285 | 2.24E−08 | 240 | 4.43E−08 |
| 225 | 2.29E−08 | 235 | 4.48E−08 |
| 237 | 2.33E−08 | 154 | 4.73E−08 |
| 141 | 2.36E−08 | 183 | 4.79E−08 |
| 27 | 2.45E−08 | 3 | 6.96E−08 |
| 45 | 2.51E−08 | 155 | >2.42E−08 |
| 76 | 2.54E−08 | 197 | >2.42E−08 |
| 184 | 2.54E−08 | 120 | >2.42E−08 |
| 96 | 2.63E−08 | 68 | >2.42E−08 |
| 239 | 2.65E−08 | 50 | >2.42E−08 |
| 213 | 2.65E−08 | 56 | >2.42E−08 |
| 273 | 2.75E−08 | 224 | >2.42E−08 |
| 195 | 2.84E−08 | 137 | >2.42E−08 |
| 41 | 2.91E−08 | 186 | >2.42E−08 |
| 142 | 2.92E−08 | 93 | >2.42E−08 |
| 254 | 3.10E−08 | 207 | >4.83E−08 |
| 230 | 3.12E−08 | 69 | >4.83E−08 |
| 29 | 3.12E−08 | 178 | >4.83E−08 |
| 290 | 3.13E−08 | 70 | >4.83E−08 |
| 220 | 3.14E−08 | 209 | >4.83E−08 |
| 233 | 3.32E−08 | 38 | >4.83E−08 |
| 116 | 3.36E−08 | 234 | >4.83E−08 |
| 80 | 3.38E−08 | 204 | >4.83E−08 |
| 125 | 3.39E−08 | 301 | >4.830E−08 |
| 292 | 3.40E−08 | 302 | >4.83E−08 |
| 139 | 3.52E−08 | 218 | >2.42E−07 |

In particular, deeply involved inflammation sites and tumor sites were further examined in detail.

### Inflammation Sites

TABLE 6G

| | Tropism for | Abbreviated | | | | | | | | | | | |
|---|---|---|---|---|---|---|---|---|---|---|---|---|---|
| Liposome No. | organ | name | IC10 | IC20 | IC30 | IC31 | IC32 | IC33 | IC34 | IC35 | IC40 | IC50 | IC60 |
| 27 | ++ | LDF-5 | <4.83E−10 | 1.49E−09 | <4.83E−10 | 3.44E−09 | 3.64E−09 | 3.83E−09 | 4.03E−09 | 4.23E−09 | 5.22E−09 | 1.27E−08 | 2.45E−08 |
| 38 | ++ | G4GN-4 | 1.39E−09 | 1.81E−09 | 1.39E−09 | 2.85E−08 | 2.97E−08 | 3.08E−08 | 3.20E−08 | 3.32E−08 | 2.06E−08 | 4.50E−08 | >4.83E−08 |
| 53 | ++ | L2F-1 | <4.83E−10 | <4.83E−10 | <4.83E−10 | <4.83E−10 | <4.83E−10 | <4.83E−10 | <4.83E−10 | 5.96E−10 | 5.96E−10 | 1.11E−08 | 1.63E−08 |
| 60 | ++ | 6SLN-1 | 1.25E−09 | 5.30E−09 | 1.25E−09 | 1.18E−08 | 1.24E−08 | 1.30E−08 | 1.36E−08 | 1.42E−08 | 1.84E−08 | 3.03E−08 | 4.23E−08 |
| 69 | ++ | L4F-1 | <4.83E−10 | 1.28E−09 | <4.83E−10 | 1.11E−08 | 1.18E−08 | 1.25E−08 | 1.31E−08 | 1.38E−08 | 2.13E−08 | >4.83E−08 | >4.83E−08 |
| 70 | ++ | LNT-5 | 3.34E−09 | 4.86E−09 | 3.34E−09 | 2.72E−08 | 2.86E−08 | 3.00E−08 | 3.14E−08 | 3.28E−08 | 3.98E−08 | >4.83E−08 | >4.83E−08 |
| 87 | ++ | TA-1 | <4.83E−10 | <4.83E−10 | <4.83E−10 | 7.80E−09 | 8.06E−09 | 8.33E−09 | 8.59E−09 | 8.86E−09 | 1.02E−08 | 1.28E−08 | 1.37E−08 |
| 91 | ++ | G6GN-4 | <4.83E−10 | 7.48E−09 | <4.83E−10 | 1.42E−08 | 1.48E−08 | <4.83E−10 | 1.60E−08 | 1.68E−08 | 2.09E−08 | 2.89E−08 | 3.69E−08 |
| 93 | ++ | A36 | 9.73E−10 | 1.06E−08 | 9.45E−10 | >4.83E−08 | >4.83E−08 | >4.83E−08 | >4.83E−08 | >4.83E−08 | >4.83E−08 | >2.42E−08 | >2.42E−08 |
| 96 | ++ | TA-4 | <4.83E−10 | <4.83E−10 | <4.83E−10 | 3.89E−09 | 4.08E−09 | 4.28E−09 | 4.47E−09 | 4.66E−09 | 6.49E−09 | 1.39E−08 | 2.63E−08 |
| 105 | ++ | Man6 | 4.81E−10 | 2.51E−09 | 4.81E−10 | 5.90E−09 | 6.22E−09 | 6.53E−09 | 6.85E−09 | 7.16E−09 | 9.24E−09 | 1.53E−08 | 2.14E−08 |
| 106 | ++ | LDF-4 | <4.83E−10 | <4.83E−10 | <4.83E−10 | 2.16E−09 | 2.46E−09 | 2.75E−09 | 3.05E−09 | 3.35E−09 | 4.83E−09 | 2.76E−08 | 3.75E−08 |
| 116 | ++ | LDF-1 | 1.21E−09 | 3.78E−09 | 1.21E−09 | 9.06E−09 | 9.65E−09 | 1.02E−08 | 1.08E−08 | 1.14E−08 | 1.44E−08 | 2.34E−08 | 3.36E−08 |
| 125 | ++ | LX-1 | <4.83E−10 | <4.83E−10 | 6.28E−10 | 1.02E−09 | 1.86E−08 | 1.91E−08 | 1.97E−08 | 2.02E−08 | 2.30E−08 | 2.84E−08 | 3.39E−08 |
| 139 | ++ | 3FL-5 | <4.83E−10 | 7.93E−10 | <4.83E−10 | 1.49E−09 | 1.55E−09 | 1.61E−09 | 1.68E−09 | 1.74E−09 | 2.85E−09 | 2.37E−08 | 3.52E−08 |
| 146 | ++ | 3FL-4 | <4.83E−10 | <4.83E−10 | <4.83E−10 | 2.89E−09 | 3.29E−09 | 3.70E−09 | 4.11E−09 | 4.51E−09 | 6.10E−09 | 2.70E−08 | 4.11E−08 |
| 150 | ++ | TNA-5 | <4.83E−10 | <4.83E−10 | <4.83E−10 | <4.83E−10 | 2.90E−10 | 1.39E−10 | 8.09E−10 | 1.48E−09 | 3.86E−09 | 2.63E−08 | 3.66E−08 |
| 152 | ++ | LY-1 | <4.83E−10 | <4.83E−10 | <4.83E−10 | 2.49E−09 | 2.71E−09 | 2.93E−09 | 3.15E−09 | 3.37E−09 | 4.48E−09 | 9.77E−09 | 1.72E−08 |
| 153 | ++ | G6GN-5 | <4.83E−10 | 2.53E−09 | <4.83E−10 | 1.14E−08 | 1.29E−08 | 1.45E−08 | 1.60E−08 | 1.70E−08 | 2.16E−08 | 3.09E−08 | 4.02E−08 |
| 154 | ++ | Lac-5 | <4.83E−10 | 3.08E−09 | <4.83E−10 | 1.62E−08 | 1.73E−08 | 1.84E−08 | 1.94E−08 | 2.05E−08 | 2.59E−08 | 3.66E−08 | 4.73E−08 |
| 155 | ++ | A6 | 6.47E−09 | 1.01E−08 | 6.47E−09 | 1.58E−08 | 1.63E−08 | 1.68E−08 | 1.72E−08 | 1.77E−08 | 2.01E−08 | >2.42E−08 | >2.42E−08 |
| 184 | ++ | STNA-4 | <4.83E−10 | <4.83E−10 | <4.83E−10 | 1.26E−09 | 1.39E−09 | 1.52E−09 | 1.65E−09 | 1.77E−09 | 3.55E−09 | 1.83E−08 | 2.54E−08 |
| 191 | ++ | LB-1 | <4.83E−10 | 1.40E−09 | <4.83E−10 | 3.50E−09 | 3.77E−09 | 4.04E−09 | 4.31E−09 | 4.59E−09 | 6.04E−09 | 9.18E−09 | 1.23E−08 |
| 195 | ++ | TNA-4 | <4.83E−10 | <4.83E−10 | <4.83E−10 | 8.88E−10 | 1.11E−09 | 5.33E−09 | 1.56E−09 | 1.78E−09 | 8.35E−09 | 1.99E−08 | 2.84E−08 |
| 199 | ++ | STNA-4 | <4.83E−10 | <4.83E−10 | <4.83E−10 | 3.53E−09 | 3.79E−09 | 4.05E−09 | 4.31E−09 | 4.57E−09 | 2.66E−08 | 2.70E−08 | 3.70E−08 |
| 204 | ++ | Lac-1 | <4.83E−10 | <4.83E−10 | <4.83E−10 | 2.47E−08 | 2.62E−08 | 2.77E−08 | 2.92E−08 | 3.07E−08 | 3.82E−08 | >4.83E−08 | >4.83E−08 |
| 213 | ++ | LX-4 | <4.83E−10 | 9.28E−10 | 4.94E−09 | 5.08E−09 | 5.22E−09 | 5.35E−09 | 6.32E−09 | 7.10E−09 | 1.10E−08 | 1.88E−08 | 2.65E−08 |
| 220 | ++ | LNB-4 | 3.83E−09 | 6.57E−09 | 3.83E−09 | 1.06E−08 | 1.09E−08 | 1.11E−08 | 1.14E−08 | 7.27E−09 | 1.32E−08 | 2.12E−08 | 3.14E−08 |
| 224 | ++ | Man5 | 2.97E−10 | 4.43E−09 | 2.67E−10 | 8.44E−09 | 9.16E−09 | 9.88E−09 | 1.06E−08 | 1.13E−08 | 1.49E−08 | 2.22E−08 | >2.42E−08 |
| 229 | ++ | G6GN-1 | <4.83E−10 | <4.83E−10 | <4.83E−10 | 3.51E−09 | 3.76E−09 | <4.83E−10 | 4.24E−09 | 4.48E−09 | 6.25E−09 | 1.22E−08 | 2.13E−08 |
| 230 | ++ | G4GN-1 | <4.83E−10 | 2.85E−09 | <4.83E−10 | 4.77E−09 | 4.95E−09 | 5.12E−09 | 5.22E−09 | 6.11E−09 | 1.05E−08 | 2.02E−08 | 3.12E−08 |
| 233 | ++ | 2FL-1 | 1.43E−09 | 3.41E−09 | 1.43E−09 | 1.66E−08 | 1.71E−08 | 1.77E−08 | 1.83E−08 | 1.89E−08 | 2.17E−08 | 2.75E−08 | 3.32E−08 |
| 234 | ++ | TNA-1 | <4.83E−10 | 3.41E−09 | <4.83E−10 | 3.09E−08 | 3.17E−08 | 3.34E−08 | 3.34E−08 | 3.43E−08 | 3.86E−08 | 4.72E−08 | >4.83E−08 |
| 235 | ++ | Lac-1 | <4.83E−10 | 1.48E−09 | <4.83E−10 | 4.27E−09 | 4.46E−09 | 1.47E−08 | 1.54E−08 | 5.05E−09 | 2.18E−08 | 3.33E−08 | 4.48E−08 |
| 237 | ++ | A3 | 4.04E−09 | 7.43E−09 | 4.04E−09 | 1.17E−08 | 1.21E−08 | 1.25E−08 | 1.29E−08 | 1.33E−08 | 1.53E−08 | 1.93E−08 | 2.33E−08 |
| 263 | ++ | 3SLN-1 | <4.83E−10 | <4.83E−10 | <4.83E−10 | 7.20E−10 | 8.08E−10 | 8.96E−10 | 9.85E−10 | 1.07E−09 | 1.51E−09 | 8.59E−09 | 1.44E−08 |
| 290 | ++ | SLA-5 | 1.84E−09 | 7.78E−09 | 1.16E−08 | 1.19E−08 | 1.23E−08 | 1.27E−08 | 1.31E−08 | 1.35E−08 | 1.53E−08 | 2.30E−08 | 3.13E−08 |
| 292 | ++ | STNA-5 | 2.26E−09 | 4.66E−09 | 2.26E−09 | 9.39E−09 | 7.55E−09 | 1.04E−08 | 1.09E−08 | 1.14E−08 | 1.38E−08 | 2.25E−08 | 3.40E−08 |
| 40 | + | BAT-1 | 1.17E−09 | 2.78E−09 | 1.17E−09 | 4.22E−09 | 4.35E−09 | 4.48E−09 | 4.61E−09 | 4.74E−09 | 5.39E−09 | 1.03E−08 | 1.50E−08 |
| 41 | + | SOLX-4 | <4.83E−10 | 1.06E−09 | 1.03E−08 | 1.13E−08 | 1.17E−08 | 1.24E−08 | 1.31E−08 | 1.38E−08 | 1.71E−08 | 2.31E−08 | 2.91E−08 |
| 50 | + | Man8 | 1.80E−09 | 2.78E−09 | 1.80E−09 | 1.30E−08 | 1.44E−08 | 1.57E−08 | 1.71E−08 | 1.84E−08 | 2.52E−08 | >2.42E−08 | >2.42E−08 |
| 56 | + | Man9 | 1.61E−08 | 2.35E−08 | 1.61E−08 | >4.83E−08 | >4.83E−08 | >4.83E−08 | >4.83E−08 | >4.83E−08 | >4.83E−08 | >2.42E−08 | >2.42E−08 |
| 68 | + | Man7 | 2.11E−09 | 4.28E−09 | 2.11E−09 | >4.83E−08 | >4.83E−08 | >4.83E−08 | 1.68E−08 | 1.58E−08 | 7.80E−09 | >2.42E−08 | >2.42E−08 |
| 71 | + | BBT-1 | 5.99E−10 | 2.78E−09 | 5.99E−10 | 4.24E−09 | 4.37E−09 | 4.50E−09 | 4.63E−09 | 4.77E−09 | 5.65E−09 | 9.57E−09 | 1.35E−08 |
| 76 | + | BHD-4 | <4.83E−10 | 2.39E−09 | <4.83E−10 | 4.21E−09 | 5.21E−09 | 6.20E−09 | 7.19E−09 | 8.19E−09 | 1.32E−08 | 1.99E−08 | 2.54E−08 |
| 111 | + | LNB-5 | 2.62E−09 | 3.94E−09 | 2.62E−09 | 5.62E−09 | 6.13E−09 | 6.64E−09 | 7.15E−09 | 7.66E−09 | 1.02E−08 | 1.53E−08 | 3.82E−08 |
| 117 | + | TA-5 | <4.83E−10 | 1.09E−09 | <4.83E−10 | 2.88E−09 | 3.06E−09 | 3.24E−09 | 3.41E−09 | 3.59E−09 | 4.47E−09 | 8.45E−09 | 1.45E−08 |
| 120 | + | A2 | 7.02E−09 | 8.70E−09 | 7.02E−09 | 2.09E−08 | 2.20E−08 | 2.31E−08 | 2.42E−08 | >4.83E−08 | >4.83E−08 | >2.42E−08 | >2.42E−08 |
| 137 | + | Man4B | 6.02E−10 | 1.04E−08 | 6.02E−10 | 1.96E−08 | 2.04E−08 | 2.13E−08 | 2.21E−08 | 2.30E−08 | >4.83E−08 | >2.42E−08 | >2.42E−08 |
| 151 | + | SLX-4 | <4.83E−10 | <4.83E−10 | 1.37E−09 | 1.52E−09 | 1.67E−09 | 1.82E−09 | 1.98E−09 | 2.39E−09 | 4.16E−09 | 1.14E−08 | 2.01E−08 |
| 183 | + | G4GN-5 | 1.99E−08 | 2.55E−08 | 1.99E−08 | 3.16E−08 | 3.22E−08 | 3.28E−08 | 3.33E−08 | 3.39E−08 | 3.67E−08 | 4.23E−08 | 4.79E−08 |
| 186 | + | Man3 | 8.41E−10 | 1.12E−08 | 8.41E−10 | 1.66E−08 | 1.70E−08 | 1.75E−08 | 1.80E−08 | 1.85E−08 | 2.09E−08 | >2.42E−08 | >2.42E−08 |
| 197 | + | A4 | 5.01E−09 | 5.85E−09 | 5.01E−09 | 6.77E−09 | 6.88E−09 | 6.94E−09 | 7.03E−09 | 7.11E−09 | 7.53E−09 | 1.60E−08 | >2.42E−08 |
| 209 | + | LDFH-4 | 3.78E−09 | 4.58E−10 | 3.78E−09 | 4.23E−08 | 4.47E−08 | 4.70E−08 | 4.94E−08 | >4.83E−08 | >4.83E−08 | >4.83E−08 | >4.83E−08 |
| 240 | + | SOLA-5 | <4.83E−10 | <4.83E−10 | 1.06E−09 | 1.79E−08 | 1.88E−08 | 1.97E−08 | 2.07E−08 | 2.16E−08 | 2.61E−08 | 3.52E−08 | 4.43E−08 |
| 288 | + | LA-1 | 2.63E−09 | 4.00E−09 | 5.54E−09 | 6.16E−09 | 6.77E−09 | 7.39E−09 | 8.00E−09 | 8.62E−09 | 1.17E−08 | 2.07E−08 | 3.61E−08 |
| 295 | + | 6SLN-4 | <4.83E−10 | 2.30E−09 | <4.83E−10 | 4.18E−09 | 4.18E−09 | 4.33E−09 | 4.49E−09 | 4.65E−09 | 5.63E−09 | 1.00E−08 | 1.44E−08 |
| 16 | − | SOLX-5 | <4.83E−10 | 1.42E−08 | 2.37E−08 | 2.43E−08 | 2.49E−08 | 2.55E−08 | 2.61E−08 | 4.13E−09 | 2.97E−08 | 3.57E−08 | 4.16E−08 |
| 29 | − | 3SL-5 | 1.21E−11 | 1.23E−08 | 4.83E−10 | 1.50E−08 | 1.52E−08 | 1.55E−08 | 1.54E−08 | 1.60E−08 | 1.90E−08 | 2.51E−08 | 3.12E−08 |
| 127 | − | G3G-4 | <4.83E−10 | 1.27E−08 | 4.83E−10 | 1.54E−08 | 1.57E−08 | 1.59E−08 | 1.70E−08 | 1.82E−08 | 2.39E−08 | 3.16E−08 | 3.54E−08 |
| 129 | − | BBT-4 | 2.86E−09 | 3.83E−09 | 2.86E−09 | 4.90E−09 | 5.00E−09 | 5.09E−09 | 5.19E−09 | 5.29E−09 | 7.72E−09 | 1.28E−08 | 1.95E−08 |
| 130 | − | 2FL-4 | 4.12E−09 | 7.91E−09 | 4.12E−09 | 1.26E−08 | 1.30E−08 | 1.34E−08 | 1.38E−08 | 1.43E−08 | 1.70E−08 | 2.73E−08 | 3.75E−08 |
| 141 | − | BBT-5 | 1.66E−09 | 1.80E−09 | 1.66E−09 | 2.47E−09 | 2.74E−09 | 3.02E−09 | 3.29E−09 | 3.56E−09 | 4.92E−09 | 1.41E−08 | 2.36E−08 |
| 142 | − | BHD-5 | 9.87E−10 | 2.07E−09 | 9.87E−10 | 7.48E−09 | 7.92E−09 | 8.37E−09 | 8.81E−09 | 9.25E−09 | 1.15E−08 | 1.59E−08 | 2.92E−08 |
| 175 | − | G3G-5 | 2.01E−08 | 2.49E−08 | 2.01E−08 | 3.02E−08 | 3.06E−08 | 3.11E−08 | 3.16E−08 | 3.21E−08 | 3.45E−08 | 3.93E−08 | 4.40E−08 |
| 178 | − | LNT-4 | 6.84E−09 | 1.01E−08 | 6.84E−09 | 1.36E−08 | 1.40E−08 | 1.43E−08 | 1.46E−08 | 1.49E−08 | 2.01E−08 | 4.24E−08 | >4.83E−08 |
| 207 | − | 6SL-5 | 3.76E−09 | 4.64E−09 | 4.83E−10 | 8.94E−09 | 1.00E−08 | 1.11E−08 | 1.22E−08 | 1.34E−08 | 1.99E−08 | 3.48E−08 | >4.83E−08 |
| 236 | − | 2FL-5 | 7.44E−09 | 1.18E−08 | 7.44E−09 | 1.70E−08 | 1.77E−08 | 1.84E−08 | 1.92E−08 | 1.99E−08 | 2.34E−08 | 3.05E−08 | 3.76E−08 |
| 239 | − | SLA-4 | <4.83E−10 | 1.58E−09 | 7.28E−09 | 7.75E−09 | 8.23E−09 | 8.70E−09 | 9.17E−09 | 9.64E−09 | 1.20E−08 | 1.75E−08 | 2.65E−08 |
| 3 | − | EEGD1A-BH | 4.93E−09 | 1.13E−08 | 2.21E−08 | 2.32E−08 | 2.43E−08 | 2.53E−08 | 2.64E−08 | 2.81E−08 | 4.17E−08 | 6.88E−08 | 6.96E−08 |
| 218 | − | EEGMI-BH | 7.23E−09 | 2.03E−08 | 6.08E−08 | 6.49E−08 | 6.90E−08 | 7.31E−08 | 7.73E−08 | 8.45E−08 | 1.43E−07 | >2.42E−08 | >2.42E−07 |
| 254 | − | G3G-1 | 2.03E−09 | 4.05E−09 | 2.03E−09 | 7.61E−09 | 8.09E−09 | 8.57E−08 | 9.06E−09 | 9.54E−09 | 1.19E−08 | 1.81E−08 | 3.10E−08 |
| 301 | − | TRIS | 8.56E−09 | 2.02E−08 | 3.18E−08 | 3.30E−08 | 3.41E−08 | 3.53E−08 | 3.64E−08 | 3.78E−08 | 4.34E−08 | >4.83E−08 | >4.83E−08 |
| 302 | − | CEBI | 3.98E−08 | 2.37E−08 | 4.05E−08 | 4.22E−08 | 4.39E−08 | 4.58E−08 | 4.73E−08 | 4.90E−08 | >4.83E−08 | >4.83E−08 | >4.83E−08 |

### (Tumor Sites)

TABLE 6H

| | Tropism for | Abbreviated | | | | | | | | | | | |
|---|---|---|---|---|---|---|---|---|---|---|---|---|---|
| Liposome No. | organ | name | IC10 | IC20 | IC30 | IC31 | IC32 | IC33 | IC34 | IC35 | IC40 | IC50 | IC60 |
| 27 | ++ | LDF-5 | <4.83E−10 | 1.49E−09 | <4.83E−10 | 3.44E−09 | 3.64E−09 | 3.83E−09 | 4.03E−09 | 4.23E−09 | 5.22E−09 | 1.27E−08 | 2.45E−08 |
| 29 | ++ | 3SL-5 | 1.21E−11 | 1.23E−08 | 4.83E−10 | 1.50E−08 | 1.52E−08 | 1.55E−08 | 1.54E−08 | 1.60E−08 | 1.90E−08 | 2.51E−08 | 3.12E−08 |
| 38 | ++ | G4GN-4 | 1.39E−09 | 1.81E−09 | 1.39E−09 | 2.85E−08 | 2.97E−08 | 3.08E−08 | 3.20E−08 | 3.32E−08 | 2.08E−08 | 4.50E−08 | >4.83E−08 |
| 53 | ++ | L2F-1 | <4.83E−10 | <4.83E−10 | <4.83E−10 | <4.83E−10 | <4.83E−10 | <4.83E−10 | <4.83E−10 | 5.96E−10 | 5.96E−10 | 1.11E−08 | 1.93E−08 |
| 60 | ++ | 6SLN-1 | 1.25E−09 | 5.30E−09 | 1.25E−09 | 1.18E−08 | 1.24E−08 | 1.30E−08 | 1.36E−08 | 1.42E−08 | 1.84E−08 | 3.03E−08 | 4.23E−08 |
| 69 | ++ | L4F-1 | <4.83E−10 | 1.28E−09 | <4.83E−10 | 1.11E−08 | 1.18E−08 | 1.25E−08 | 1.31E−08 | 1.38E−08 | 2.13E−08 | >4.83E−08 | >4.83E−08 |
| 87 | ++ | TA-1 | <4.83E−10 | <4.83E−10 | <4.83E−10 | 7.80E−09 | 8.06E−09 | 8.33E−09 | 8.59E−09 | 8.86E−09 | 1.02E−08 | 1.28E−08 | 1.37E−08 |
| 93 | ++ | A36 | 9.73E−10 | 1.06E−08 | 9.45E−10 | >4.83E−08 | >4.83E−08 | >4.83E−08 | >4.83E−08 | >4.83E−08 | >4.83E−08 | >2.42E−08 | >2.42E−08 |
| 96 | ++ | TA-4 | <4.83E−10 | <4.83E−10 | <4.83E−10 | 3.89E−09 | 4.08E−09 | 4.28E−09 | 4.47E−09 | 4.66E−09 | 6.49E−09 | 1.39E−08 | 2.63E−08 |
| 116 | ++ | LDF-1 | 1.21E−09 | 3.78E−09 | 1.21E−09 | 9.06E−09 | 9.65E−09 | 1.02E−08 | 1.08E−08 | 1.14E−08 | 1.44E−08 | 2.34E−08 | 3.36E−08 |
| 125 | ++ | LX-1 | <4.83E−10 | <4.83E−10 | 6.28E−10 | 1.02E−09 | 1.86E−08 | 1.91E−08 | 1.97E−08 | 2.02E−08 | 2.30E−08 | 2.84E−08 | 3.39E−08 |
| 150 | ++ | TNA-5 | <4.83E−10 | <4.83E−10 | <4.83E−10 | <4.83E−10 | 2.90E−10 | 1.39E−10 | 8.09E−10 | 1.48E−09 | 3.86E−09 | 2.63E−08 | 3.66E−08 |
| 152 | ++ | LY-1 | <4.83E−10 | <4.83E−10 | <4.83E−10 | 2.49E−09 | 2.71E−09 | 2.93E−09 | 3.15E−09 | 3.37E−09 | 4.48E−09 | 9.77E−09 | 1.72E−08 |
| 154 | ++ | Lac-5 | <4.83E−10 | 3.08E−09 | <4.83E−10 | 1.62E−08 | 1.73E−08 | 1.84E−08 | 1.94E−08 | 2.05E−08 | 2.59E−08 | 3.66E−08 | 4.73E−08 |
| 155 | ++ | A6 | 6.47E−09 | 1.01E−08 | 6.47E−09 | 1.58E−08 | 1.63E−08 | 1.68E−08 | 1.72E−08 | 1.77E−08 | 2.01E−08 | >2.42E−08 | >2.42E−08 |
| 184 | ++ | STNA-1 | <4.83E−10 | <4.83E−10 | <4.83E−10 | 1.26E−09 | 1.39E−09 | 1.52E−09 | 1.65E−09 | 1.77E−09 | 3.55E−09 | 1.83E−08 | 2.54E−08 |
| 195 | ++ | TNA-4 | <4.83E−10 | <4.83E−10 | <4.83E−10 | 8.88E−10 | 1.11E−09 | 5.33E−09 | 1.56E−09 | 1.78E−09 | 8.35E−09 | 1.99E−08 | 2.84E−08 |
| 204 | ++ | Lac-1 | <4.83E−10 | <4.83E−10 | <4.83E−10 | 2.47E−08 | 2.62E−08 | 2.77E−08 | 2.92E−08 | 3.07E−08 | 3.82E−08 | >4.83E−08 | >4.83E−08 |
| 213 | ++ | LX-4 | <4.83E−10 | 9.28E−10 | 4.94E−09 | 5.08E−09 | 5.22E−09 | 5.35E−09 | 6.32E−09 | 7.10E−09 | 1.10E−08 | 1.88E−08 | 2.65E−08 |
| 224 | ++ | Man5 | 2.97E−10 | 4.43E−09 | 2.97E−10 | 8.44E−09 | 9.16E−09 | 9.88E−09 | 1.06E−08 | 1.13E−08 | 1.49E−08 | 2.22E−08 | >2.42E−08 |
| 229 | ++ | G6GN-1 | <4.83E−10 | <4.83E−10 | <4.83E−10 | 3.51E−09 | 3.76E−09 | <4.83E−10 | 4.24E−09 | 4.48E−09 | 6.25E−09 | 1.22E−08 | 2.13E−08 |
| 230 | ++ | G4GN-1 | <4.83E−10 | 2.65E−09 | <4.83E−10 | 4.77E−09 | 4.95E−09 | 5.12E−09 | 5.22E−09 | 6.11E−09 | 1.05E−08 | 2.02E−08 | 3.12E−08 |
| 233 | ++ | 2FL-1 | 1.43E−09 | 3.41E−09 | 1.43E−09 | 1.66E−08 | 1.71E−08 | 1.77E−08 | 1.83E−08 | 1.89E−08 | 2.17E−08 | 2.75E−08 | 3.32E−08 |
| 234 | ++ | TNA-1 | <4.83E−10 | 3.41E−09 | <4.83E−10 | 3.09E−08 | 3.17E−08 | 3.34E−08 | 3.34E−08 | 3.43E−08 | 3.86E−08 | 4.72E−08 | >4.83E−10 |
| 235 | ++ | Lac-4 | <4.83E−10 | 1.48E−09 | <4.83E−10 | 4.27E−09 | 4.46E−09 | 1.47E−08 | 1.54E−08 | 5.05E−09 | 2.18E−08 | 3.33E−08 | 4.48E−08 |
| 237 | ++ | A3 | 4.04E−09 | 7.43E−09 | 4.04E−09 | 1.17E−08 | 1.21E−08 | 1.25E−08 | 1.29E−08 | 1.33E−08 | 1.53E−08 | 1.93E−08 | 2.33E−08 |
| 263 | ++ | 3SLN-1 | <4.83E−10 | <4.83E−10 | <4.83E−10 | 7.20E−10 | 8.08E−10 | 8.96E−10 | 9.85E−10 | 1.07E−09 | 1.51E−09 | 8.59E−09 | 1.44E−08 |
| 285 | ++ | SLX-5 | <4.83E−10 | <4.83E−10 | 2.70E−09 | 3.10E−09 | 3.50E−09 | 3.90E−09 | 4.30E−09 | 4.71E−09 | 7.67E−09 | 1.44E−08 | 2.24E−08 |
| 288 | ++ | LA-1 | 2.63E−09 | 4.00E−09 | 5.54E−09 | 6.16E−09 | 6.77E−09 | 7.39E−09 | 8.00E−09 | 8.62E−09 | 1.17E−08 | 2.07E−08 | 3.61E−08 |
| 292 | ++ | STNA-5 | 2.26E−09 | 4.66E−09 | 2.26E−09 | 9.39E−09 | 7.55E−09 | 1.04E−08 | 1.09E−08 | 1.14E−08 | 1.38E−09 | 2.25E−08 | 3.40E−08 |
| 40 | + | BAT-1 | 1.17E−09 | 2.78E−09 | 1.17E−09 | 4.22E−09 | 4.35E−09 | 4.48E−09 | 4.61E−09 | 4.74E−09 | 5.39E−09 | 1.03E−08 | 1.50E−08 |
| 41 | + | SOLX-4 | <4.83E−10 | 1.06E−09 | 1.03E−08 | 1.13E−08 | 1.17E−08 | 1.24E−08 | 1.31E−08 | 1.38E−08 | 1.71E−08 | 2.31E−08 | 2.91E−08 |
| 45 | + | BHD-1 | <4.83E−10 | <4.83E−10 | <4.83E−10 | 1.91E−09 | 2.38E−09 | 2.85E−09 | 3.32E−09 | 3.79E−09 | 6.91E−09 | 1.59E−08 | 2.51E−08 |
| 68 | + | Man7 | 2.11E−09 | 4.28E−09 | 2.11E−09 | >4.83E−08 | >4.83E−08 | >4.83E−08 | 1.68E−08 | 1.58E−08 | 7.80E−09 | >2.42E−08 | >2.42E−08 |
| 71 | + | BBT-1 | 5.99E−10 | 2.78E−09 | 5.99E−10 | 4.24E−09 | 4.37E−09 | 4.50E−09 | 4.63E−09 | 4.77E−09 | 5.65E−09 | 9.57E−09 | 1.35E−08 |
| 91 | + | G6GN-4 | <4.83E−10 | 7.48E−09 | <4.83E−10 | 1.42E−08 | 1.48E−08 | <4.83E−10 | 1.60E−08 | 1.68E−08 | 2.09E−08 | 2.89E−08 | 3.69E−08 |
| 105 | + | Man6 | 4.81E−10 | 2.51E−09 | 4.81E−10 | 5.90E−09 | 6.22E−09 | 6.53E−09 | 6.85E−09 | 7.16E−09 | 9.24E−09 | 1.53E−08 | 2.14E−08 |
| 106 | + | LDF-4 | <4.83E−10 | <4.83E−10 | <4.83E−10 | 2.16E−09 | 2.46E−09 | 2.75E−09 | 3.05E−09 | 3.35E−09 | 4.83E−09 | 2.76E−08 | 3.75E−08 |
| 111 | + | LNB-5 | 2.62E−09 | 3.94E−09 | 2.62E−09 | 5.62E−09 | 6.13E−09 | 6.64E−09 | 7.15E−09 | 7.66E−09 | 1.02E−08 | 1.53E−08 | 3.82E−08 |
| 117 | + | TA-5 | <4.83E−10 | 1.09E−09 | <4.83E−10 | 2.88E−09 | 3.06E−09 | 3.24E−09 | 3.41E−09 | 3.59E−09 | 4.47E−09 | 8.45E−09 | 1.45E−08 |
| 120 | + | A2 | 7.02E−09 | 8.70E−09 | 7.02E−09 | 2.09E−08 | 2.20E−08 | 2.31E−08 | 2.42E−08 | >4.83E−08 | >4.83E−08 | >2.42E−08 | >2.42E−08 |
| 139 | + | 3FL-5 | <4.83E−10 | 7.93E−10 | <4.83E−10 | 1.49E−09 | 1.55E−09 | 1.61E−09 | 1.68E−09 | 1.74E−09 | 2.85E−09 | 2.37E−08 | 3.52E−08 |
| 151 | + | SLX-4 | <4.83E−10 | <4.83E−10 | 1.37E−09 | 1.52E−09 | 1.67E−09 | 1.82E−09 | 1.98E−09 | 2.39E−09 | 4.16E−09 | 1.14E−08 | 2.01E−08 |
| 153 | + | G6GN-5 | <4.83E−10 | 2.53E−09 | <4.83E−10 | 1.14E−08 | 1.29E−08 | 1.45E−08 | 1.60E−08 | 1.70E−08 | 2.16E−08 | 3.09E−08 | 4.02E−08 |
| 186 | + | Man3 | 8.41E−10 | 1.12E−08 | 8.41E−10 | 1.66E−08 | 1.70E−08 | 1.75E−08 | 1.60E−08 | 1.85E−08 | 2.09E−08 | >2.42E−08 | >2.42E−08 |
| 191 | + | LB-1 | <4.83E−10 | 1.40E−09 | <4.83E−10 | 3.50E−09 | 3.77E−09 | 4.04E−09 | 4.31E−09 | 4.59E−09 | 6.04E−09 | 9.18E−09 | 1.23E−08 |
| 197 | + | A4 | 5.01E−09 | 5.85E−09 | 5.01E−09 | 6.77E−09 | 6.86E−09 | 6.94E−09 | 7.03E−09 | 7.11E−09 | 7.53E−09 | 1.60E−08 | >2.42E−08 |
| 209 | + | LDFH-4 | 3.78E−09 | 4.58E−10 | 3.78E−09 | 4.23E−08 | 4.47E−08 | 4.70E−08 | 4.94E−08 | >4.83E−08 | >4.83E−08 | >4.83E−08 | >4.83E−08 |
| 220 | + | LNB-4 | 3.83E−09 | 6.57E−09 | 3.83E−09 | 1.06E−08 | 1.09E−08 | 1.11E−08 | 1.14E−08 | 7.27E−09 | 1.32E−08 | 2.12E−08 | 3.14E−08 |
| 225 | + | 3FL-1 | <4.83E−10 | <4.83E−10 | <4.83E−10 | 1.69E−09 | 1.80E−09 | 1.92E−09 | 2.33E−09 | 2.80E−09 | 5.13E−09 | 1.30E−08 | 2.29E−08 |
| 236 | + | 2FL-5 | 7.44E−09 | 1.18E−08 | 7.44E−09 | 1.70E−08 | 1.77E−08 | 1.84E−08 | 1.92E−08 | 1.99E−08 | 2.34E−08 | 3.05E−08 | 3.76E−08 |
| 240 | + | SOLA-5 | <4.83E−10 | <4.83E−10 | 1.06E−09 | 1.79E−08 | 1.88E−08 | 1.97E−08 | 2.07E−08 | 2.16E−08 | 2.61E−08 | 3.52E−08 | 4.43E−08 |
| 290 | + | SLA-5 | 1.84E−09 | 7.78E−09 | 1.16E−08 | 1.19E−08 | 1.23E−08 | 1.27E−08 | 1.31E−08 | 1.35E−08 | 1.53E−08 | 2.30E−08 | 3.13E−08 |
| 295 | + | 6SLN-4 | <4.83E−10 | 2.30E−09 | <4.83E−10 | 4.18E−09 | 4.18E−09 | 4.33E−09 | 4.49E−09 | 4.65E−09 | 5.63E−09 | 1.00E−08 | 1.44E−08 |
| 50 | − | Man8 | 1.80E−09 | 2.78E−09 | 1.80E−09 | 1.30E−08 | 1.44E−08 | 1.57E−08 | 1.71E−08 | 1.84E−08 | 2.52E−08 | >2.42E−08 | >2.42E−08 |
| 56 | − | Man9 | 1.61E−08 | 2.35E−08 | 1.61E−08 | >4.83E−08 | >4.83E−08 | >4.83E−08 | >4.83E−08 | >4.83E−08 | >4.83E−08 | >2.42E−08 | >2.42E−08 |
| 70 | − | LNT-5 | 3.34E−09 | 4.86E−09 | 3.34E−09 | 2.72E−08 | 2.86E−08 | 3.00E−08 | 3.14E−08 | 3.28E−08 | 3.98E−08 | >4.83E−08 | >4.83E−08 |
| 127 | − | G3G-4 | <4.83E−10 | 1.27E−08 | 4.83E−10 | 1.54E−08 | 1.57E−08 | 1.59E−08 | 1.70E−08 | 1.82E−08 | 2.39E−08 | 3.16E−08 | 3.54E−08 |
| 129 | − | BBT-4 | 2.86E−09 | 3.83E−09 | 2.86E−09 | 4.90E−09 | 5.00E−09 | 5.09E−09 | 5.19E−09 | 5.29E−09 | 7.72E−09 | 1.28E−08 | 1.95E−08 |
| 137 | − | Man4B | 6.02E−10 | 1.04E−08 | 6.02E−10 | 1.96E−08 | 2.04E−08 | 2.13E−08 | 2.21E−08 | 2.30E−08 | >4.83E−08 | >2.42E−08 | >2.42E−08 |
| 141 | − | BBT-5 | 1.66E−09 | 1.80E−09 | 1.66E−09 | 2.47E−09 | 2.74E−09 | 3.02E−09 | 3.29E−09 | 3.56E−09 | 4.92E−09 | 1.41E−08 | 2.36E−08 |
| 142 | − | BHD-5 | 9.87E−10 | 2.07E−09 | 9.87E−10 | 7.48E−09 | 7.92E−09 | 8.37E−09 | 8.81E−09 | 9.25E−09 | 1.15E−08 | 1.59E−08 | 2.92E−08 |
| 175 | − | G3G-5 | 2.01E−08 | 2.49E−08 | 2.01E−08 | 3.02E−08 | 3.06E−08 | 3.11E−08 | 3.16E−08 | 3.21E−08 | 3.45E−08 | 3.93E−08 | 4.40E−08 |
| 183 | − | G4GN-5 | 1.99E−08 | 2.55E−08 | 1.99E−08 | 3.16E−08 | 3.22E−08 | 3.28E−08 | 3.33E−08 | 3.39E−08 | 3.67E−08 | 4.23E−08 | 4.79E−08 |
| 207 | − | 6SL-5 | 3.76E−09 | 4.64E−09 | 4.83E−10 | 8.94E−09 | 1.00E−08 | 1.11E−08 | 1.22E−08 | 1.34E−08 | 1.99E−08 | 3.48E−08 | >4.83E−08 |
| 254 | − | G3G-1 | 2.03E−09 | 4.05E−09 | 2.03E−09 | 7.61E−09 | 8.09E−09 | 8.57E−09 | 9.06E−09 | 9.54E−09 | 1.19E−08 | 1.81E−08 | 3.10E−08 |
| 3 | − | EEGD1A-BH | 4.93E−09 | 1.13E−08 | 2.21E−08 | 2.32E−08 | 2.43E−08 | 2.53E−08 | 2.64E−08 | 2.81E−08 | 4.17E−08 | 6.88E−08 | 6.96E−08 |
| 12 | − | 3SL-4 | 1.18E−08 | 1.79E−09 | 1.18E−08 | 1.24E−08 | 1.26E−08 | 1.28E−08 | 1.29E−08 | 1.43E−08 | 1.48E−08 | 6.38E−10 | >4.83E−08 |
| 130 | − | 2FL-4 | 4.12E−09 | 7.91E−09 | 4.12E−09 | 1.26E−08 | 1.30E−08 | 1.34E−08 | 1.38E−08 | 1.43E−08 | 1.70E−08 | 2.73E−08 | 3.75E−08 |
| 178 | − | LNT-4 | 6.84E−09 | 1.01E−08 | 6.84E−09 | 1.36E−08 | 1.40E−08 | 1.43E−08 | 1.46E−08 | 1.49E−08 | 2.01E−08 | 4.24E−08 | >4.83E−08 |
| 218 | − | EEGM1-BH | 7.23E−09 | 2.03E−08 | 6.80E−08 | 6.49E−08 | 6.90E−08 | 7.31E−08 | 7.73E−08 | 8.45E−08 | 1.43E−07 | >2.42E−07 | >2.42E−07 |
| 301 | − | TRIS | 8.56E−09 | 2.02E−08 | 3.18E−08 | 3.30E−08 | 3.41E−08 | 3.53E−08 | 3.64E−08 | 3.76E−08 | 4.34E−08 | >4.83E−08 | >4.83E−08 |
| 302 | − | CEBI | 3.98E−08 | 2.37E−08 | 4.05E−08 | 4.22E−08 | 4.39E−08 | 4.58E−08 | 4.73E−08 | 4.90E−08 | >4.83E−08 | >4.83E−08 | >4.83E−08 |

When used in the description, “liposome Nos.” refer to numbers corresponding to sugar-chain-modified liposomes to which sugar chains listed in the following Table 7, Table 8, Table 9, Table 10, Table 11, and Table 12 are bound.

A sugar-chain-modified liposome to be used in the description can contain a sugar chain shown in Table 7 with an appropriate density for transfer from the intestinal tract into blood, for example.

When used in the description, “modification (binding) density” is an amount of a sugar chain that is used when a sugar-chain-modified liposome is prepared and is represented by a density (mg sugar chain/mg lipid) of a sugar chain that is bound per mg of the lipids of the liposome. Regarding the binding density of the sugar-chain-modified liposome of the present invention, it is empirically known that, although no theoretical constraints are desired herein, the amount of a sugar chain that is used for preparation is almost proportional to the density of the sugar chain bound to the liposome. Therefore, in the description, unless otherwise particularly noted, binding density is determined depending on an amount that is used upon preparation. In vitro, for example, such binding density can be indirectly determined using E-selectin. The tropism (targeting) of the sugar-chain-modified liposome of the present invention for a target delivery site can be controlled by selecting the type and binding density of a sugar chain to be bound to a liposome. Liposome Nos., sugar chain structures, modification (binding) densities, and tropism in the case of oral administration (or enteral administration) are listed in Table 7 below.

TABLE 7

| | | Modification | |
|---|---|---|---|
| | | (binding) | |
| | | density | |
| | | (mg sugar | Tropism |
| Liposome | | chain/ | for |
| No. | Sugar chain structure | mg lipid) | organ |
| 22 | Fuca1,2Galb1,3(Fuca1,4)GlcNAcb1,3,Galb1,4Glc | 0.75 | ++ |
| 27 | Fuca1,2Galb1,4(Fuca1,3)Glc | 0.5 | + |
| 29 | Neu5Aca2,3Galb1,4Glc | 0.5 | + |
| 40 | GalNAca1,3(Fuca1,2)Gal | 0.025 | ++ |
| 45 | Fuca1,2Gal | 0.025 | + |
| 50 | Mana1,2Mana1,6(Mana1,3)Mana1,6(Mana1,2Mana1,2Mana1,3)Manb1,4GlcNAcb1,4GlcNAc | 0.0385 | + |
| 53 | Fuca1,2Galb1,3GlcNAcb1,3Galb1,4Glc | 0.025 | + |
| 56 | Mana1,2Mana1,6(Mana1,2Mana1,3)Mana1,6(Mana1,2Mana1,2Mana1,3)Manb1,4GlcNAcb1, | 0.042 | ++ |
| | 4GlcNAc | | |
| 67 | Galb1,3GalNAcb1,4Galb1,4Glcb + | 0.375 each | ++ |
| | Neu5Aca2,3Galb1,3GalNAcb1,4(Neu5Aca2,3)Galb1,4Glcb | | |
| 68 | Mana1,6(Mana1,3)Mana1,6(Mana1,2Mana1,2Mana1,3)Manb1,4GlcNAcb1,4GlcNAc | 0.035 | + |
| 69 | Galb1,3(Fuca1,4)GlcNAcb1,3Galb1,4Glc | 0.025 | ++ |
| 70 | Galb1,3GlcNAcb1,3Galb1,4Glc | 0.5 | ++ |
| 71 | Gala1,3(Fuca1,2)Gal | 0.025 | + |
| 87 | Galb1,3GalNAc | 0.025 | + |
| 105 | Mana1,6(Mana1,3)Mana1,6(Mana1,2Mana1,3)Manb1,4GlcNAcb1,4GlcNAc | 0.031 | ++ |
| 117 | Galb1,3GalNAc | 0.5 | + |
| 120 | Mana1,2Man | 0.0075 | + |
| 125 | Galb1,4(Fuca1,3)GlcNAc | 0.025 | ++ |
| 139 | Galb1,4(Fuca1,3)Glc | 0.5 | ++ |
| 142 | Fuca1,2Gal | 0.5 | ++ |
| 150 | GalNAca1-O-L-serine | 0.5 | + |
| 152 | Fuca1,2Galb1,4(Fuca1,3)GlcNAc | 0.025 | ++ |
| 153 | Galb1,6GlcNAc | 0.5 | ++ |
| 154 | Galb1,4GlcNAc | 0.5 | + |
| 175 | Gala1,3Gal | 0.5 | ++ |
| 184 | Neu5Aca2,6GalNAca1-O-L-serine | 0.025 | ++ |
| 186 | Mana1,6(Mana1,3)Manb1,4GlcNAcb1,4GlcNAc | 0.0205 | ++ |
| 197 | Mana1,4Man | 0.0075 | ++ |
| 204 | Galb1,4Glc | 0.025 | ++ |
| 224 | Mana1,6(Mana1,3)Mana1,6(Mana1,3)Manb1,4GlcNAcb1,4GlcNAc | 0.0275 | ++ |
| 225 | Galb1,4(Fuca1,3)Glc | 0.025 | ++ |
| 230 | Galb1,4GlcNAc | 0.025 | ++ |
| 236 | Fuca1,2Galb1,4Glc | 0.5 | + |
| 237 | Mana1,3Man | 0.0075 | ++ |
| 240 | 3′-(O—SO3H)Galb1,3(Fuca1,4)GlcNAc | 0.5 | ++ |
| 273 | GalNAca1,3(Fuca1,2)Gal | 0.5 | ++ |
| 285 | Neu5Aca2,3Galb1,4(Fuca1,3)GlcNAc | 0.5 | + |
| 288 | Galβ1,3(Fucα1,4)GlcNAc | 0.025 | ++ |
| 290 | Neu5Aca2,3Galb1,3(Fuca1,4)GlcNAc | 0.5 | ++ |

“++” indicates that when a liposome (standard liposome) to which tris(hydroxymethyl)aminomethane is bound instead of a sugar chain is administered via oral administration, the average value of the liposome absorbed via the intestinal tract is 4 to 6 times greater than that of the standard liposome at 10 minutes after administration.
“+” indicates that when a liposome (standard liposome) to which tris(hydroxymethyl)aminomethane is bound instead of a sugar chain is administered via oral administration, the average value of the liposome absorbed via the intestinal tract is 3 to 4 times greater than that of the standard liposome at 10 minutes after administration.

Preferably, the sugar-chain-modified liposome of the present invention, which is appropriate for the oral administration, can be prepared using sugar chain types and modification (binding) densities listed in Table 7 above and combinations thereof. Once the tropism is found to be + or ++, the similar effect can be expected, which is not theoretically constrained, even when two or more types of sugar chain are combined. This is because a sugar chain that is recognized to be preferable by a lectin of target tissues or target cells is also recognized to be preferable even when two or more types of sugar chain are combined.

A sugar-chain-modified liposome appropriate for oral administration, which is used in the present invention, can be, preferably, liposome No. 27, 29, 40, 45, 50, 53, 56, 67, 68, 69, 70, 71, 87, 105, 117, 120, 125, 139, 142, 150, 152, 153, 154, 175, 184, 186, 197, 204, 224, 225, 230, 236, 237, 240, 273, 285, 288, or 290.

A sugar-chain-modified liposome to be used in the description can contain a sugar chain that is shown in Table 8, for example, at an adequate density for delivery to tumors.

When used in the description, delivery to a tumor refers to the delivery to a tumor that is selected from the group consisting of fibrosarcoma, myosarcoma, liposarcoma, chondrosarcoma, osteogenic sarcoma, chordoma, angiosarcoma, lymphatic sarcoma, periosteal tumor, mesothelioma, leiomyosarcoma, rhabdomyoblastoma, gastric cancer, esophageal cancer, rectal cancer, pancreatic cancer, ovarian cancer, prostate cancer, uterine cancer, cancer of the head or cervical parts, skin cancer, brain cancer, carcinoma planoepitheliale, sebaceous gland carcinoma, papillary carcinoma, cystadenocarcinoma, medullary cancer, bronchogenic cancer, renal cell carcinoma, hepatocarcinoma, cholangiocarcinoma, chorioepithelioma (choriocarcinoma), seminoma, embryonal carcinoma, Wilms' tumor, cervical cancer, testicular cancer, small cell lung carcinoma, non-small cell lung carcinoma, bladder carcinoma, epithelial cell carcinoma, glioma, spongiocytoma, medulloblastoma, craniopharingioma, ependymoma, pinealoma, hemangioblastoma, acoustic nerve tumor, oligodendroglioma, meningioma, melanoma, neuroblastoma, retinoblastoma, leukemia, lymphoma, and Kaposi's sarcoma.

When used in the description, “modification (binding) density” refers to an amount of a sugar chain that is used when a sugar-chain-modified liposome is prepared. It is represented by the density (mg sugar chain/mg lipid) of a bound sugar chain per mg of the lipids of the liposome. Although no theoretical constraints are desired herein, regarding the binding density of the sugar-chain-modified liposome of the present invention, it is empirically known that the amount of the sugar chain, which is used for preparation, is almost proportional to the density of the sugar chain bound to the liposome. Therefore, in the description, unless otherwise particularly noted, such binding density is determined depending on an amount that is used upon preparation. In vitro, for example, such binding density can be indirectly determined using E-selectin. The tropism for a target delivery site of the sugar-chain-modified liposome of the present invention can be controlled by selecting the type and binding density of a sugar chain to be bound to the liposome. Liposome numbers, sugar chain structures, modification (binding) densities, and related tropism for tumors are listed in Table 8 below.

TABLE 8

| | | Modification | |
|---|---|---|---|
| | | (binding) | |
| | | density | |
| | | (mg sugar | Tropism |
| Liposome | | chain/ | for |
| No. | Sugar chain structure | mg lipid) | organ |
| 22 | Fuca1,2Galb1,3(Fuca1,4)GlcNAcb1,3,Galb1,4Glc | 0.75 | + |
| 27 | Fuca1,2Galb1,4(Fuca1,3)Glc | 0.5 | ++ |
| 29 | Neu5Aca2,3Galb1,4Glc | 0.5 | ++ |
| 38 | Galb1,4GlcNAc | 0.25 | ++ |
| 40 | GalNAca1,3(Fuca1,2)Gal | 0.025 | + |
| 41 | 3′-(O—SO3H)Galb1,4(Fuca1,3)GlcNAc | 0.25 | + |
| 45 | Fuca1,2Gal | 0.025 | + |
| 53 | Fuca1,2Galb1,3GlcNAcb1,3Galb1,4Glc | 0.025 | + |
| 60 | Neu5Aca2,6Galb1,4GlcNAc | 0.025 | ++ |
| 68 | Mana1,6(Mana1,3)Mana1,6(Mana1,2Mana1,2Mana1,3)Manb1,4GlcNAcb1,4GlcNAc | 0.035 | + |
| 69 | Galb1,3(Fuca1,4)GlcNAcb1,3Galb1,4Glc | 0.025 | ++ |
| 71 | Gala1,3(Fuca1,2)Gal | 0.025 | + |
| 87 | Galb1,3GalNAc | 0.025 | ++ |
| 91 | Galb1,6GlcNAc | 0.25 | + |
| 93 | Mana1,6(Mana1,3)Man | 0.011 | ++ |
| 96 | Galb1,3GalNAc | 0.25 | ++ |
| 105 | Manα1,6(Manα1,3)Manα1,6(Manα1,2Manα1,3)Manβ1,4GlcNAcβ1,4GlcNAc | 0.031 | + |
| 106 | Fuca1,2Galb1,4(Fuca1,3)Glc | 0.25 | + |
| 111 | Galb1,3GlcNAc | 0.5 | + |
| 116 | Fuca1,2Galb1,4(Fuca1,3)Glc | 0.025 | ++ |
| 117 | Galb1,3GalNAc | 0.5 | + |
| 120 | Mana1,2Man | 0.0075 | + |
| 125 | Galb1,4(Fuca1,3)GlcNAc | 0.025 | ++ |
| 139 | Galb1,4(Fuca1,3)Glc | 0.5 | + |
| 150 | GalNAca1-O-L-serine | 0.5 | ++ |
| 151 | Neu5Aca2,3Galb1,4(Fuca1,3)GlcNAc | 0.25 | + |
| 152 | Fuca1,2Galb1,4(Fuca1,3)GlcNAc | 0.025 | ++ |
| 153 | Galb1,6GlcNAc | 0.5 | + |
| 154 | Galb1,4GlcNAc | 0.5 | ++ |
| 155 | Mana1,6Man | 0.0075 | ++ |
| 184 | Neu5Aca2,6GalNAca1-O-L-serine | 0.025 | ++ |
| 186 | Mana1,6(Mana1,3)Manb1,4GlcNAcb1,4GlcNAc | 0.0205 | + |
| 189 | Neu5Aca2,6Galb1,4Glc | 0.0205 | ++ |
| 191 | Fuc_{α}1,2Gal_{β}1,3(Fuc_{α}1,4)GlcNAc | 0.025 | + |
| 195 | GalNAca1-O-L-serine | 0.25 | ++ |
| 197 | Mana1,4Man | 0.0075 | + |
| 204 | Galb1,4Glc | 0.025 | ++ |
| 209 | Fuca1,2Galb1,3(Fuca1,4)GlcNAcb1,3,Galb1,4Glc | 0.25 | + |
| 213 | Galb1,4(Fuca1,3)GlcNAc | 0.25 | ++ |
| 218 | Galb1,3GalNAcb1,4(Neu5Aca2,3)Galb1,4Glcb1,1Cer | 0.75 | − |
| 220 | Galb1,3GlcNAc | 0.25 | + |
| 224 | Mana1,6(Mana1,3)Mana1,6(Mana1,3)Manb1,4GlcNAcb1,4GlcNAc | 0.0275 | ++ |
| 225 | Galb1,4(Fuca1,3)Glc | 0.025 | + |
| 229 | Galb1,6GlcNAc | 0.025 | ++ |
| 230 | Galb1,4GlcNAc | 0.025 | ++ |
| 233 | Fuca1,2Galb1,4Glc | 0.025 | ++ |
| 234 | GalNAca1-O-L-serine | 0.025 | ++ |
| 235 | Galb1,4Glc | 0.25 | ++ |
| 236 | Fuca1,2Galb1,4Glc | 0.5 | + |
| 237 | Manα1,3Man | 0.0075 | ++ |
| 240 | 3′-(O—SO3H)Galb1,3(Fuca1,4)GlcNAc | 0.5 | + |
| 263 | Neu5Ac_{α}2,3Gal_{β}1,4GlcNAc | 0.025 | ++ |
| 285 | Neu5Aca2,3Galb1,4(Fuca1,3)GlcNAc | 0.5 | ++ |
| 288 | Galb1,3(Fuca1,4)GlcNAc | 0.025 | ++ |
| 290 | Neu5Aca2,3Galb1,3(Fuca1,4)GlcNAc | 0.5 | + |
| 292 | Neu5Aca2,6GalNAca1-O-L-serine | 0.5 | ++ |
| 295 | Neu5Aca2,6Galb1,4GlcNAc | 0.25 | + |

“++” indicates that when a liposome (standard liposome) to which Galβ1,3GalNAcβ1,4(Neu5Acα2,3)Galβ1,4Glcβ1,1Cer is bound instead of a sugar chain is administered via intravenous injection, the average value of the liposome delivered to the tumor is 2 to 4 times greater than that of the standard liposome at 5 minutes after intravenous injection.
“+” indicates that when a liposome (standard liposome) to which Galβ1,3GalNAcβ1,4(Neu5Acα2,3)Galβ1,4Glcβ1,1Cer is bound instead of a sugar chain is administered via intravenous injection, the average value of the liposome delivered to the tumor is 1 to 2 times greater than that of the standard liposome at 5 minutes after intravenous injection.

When a liposome to which tris(hydroxymethyl)aminomethane is bound is used as a standard liposome, “++” indicates that the average value of the liposome delivered to the tumor is 1.5 to 2.5 times greater than that of the standard liposome at 5 minutes after intravenous injection.

When a liposome to which tris(hydroxymethyl)aminomethane is bound is used as a standard liposome, “+” indicates that the average value of the liposome delivered to the tumor is 1.1 to 1.4 times greater than that of the standard liposome at 5 minutes after intravenous injection.

The liposome to which tris(hydroxymethyl)aminomethane is bound has slight tumor tropism. Thus, Galβ1,3GalNAcβ1,4(Neu5Acα2,3)Galβ1,4Glcβ1,1Cer was used as a standard liposome.

Preferably, the sugar-chain-modified liposome of the present invention, which is appropriate for delivery to tumors, can be prepared using sugar chain types and modification (binding) densities listed in Table 8 above and combinations thereof. Once the tropism is found to be + or ++, the similar effect can be expected, although no theoretical constraints are desired, even when two or more types of sugar chain are combined. This is because a sugar chain that is recognized to be preferable by a lectin of target tissues or target cells is also recognized to be preferable even when two or more types of sugar chain are combined.

A sugar-chain-modified liposome appropriate for delivery to tumors, which is used in the present invention, can be, preferably, liposome No. 22, 27, 29, 38, 40, 41, 45, 53, 60, 68, 69, 71, 87, 91, 93, 96, 105, 106, 111, 116, 117, 120, 125, 139, 150, 151, 152, 153, 154, 155, 184, 186, 189, 191, 195, 197, 204, 209, 213, 218, 220, 224, 225, 229, 230, 233, 234, 235, 236, 237, 240, 263, 285, 288, 290, 292, or 295.

A sugar-chain-modified liposome to be used in the description can contain a sugar chain that is shown in Table 9, for example, at an adequate density for delivery to inflammation sites.

When used in the description, “delivery to an inflammation site” refers to the delivery to a region where a basic pathological process (in which dynamic complexes are formed by cytological and histological reactions that take place in blood vessels or tissues adjacent thereto affected by injuries due to or abnormal stimulation with physical, chemical, or biological action substances, for example) takes place. Whether or not a site is an inflammation site can be confirmed by detecting inflammatory substances (e.g., prostaglandins and leukotrienes).

When used in the description, “modification (binding) density” refers to an amount of a sugar chain that is used when a sugar-chain-modified liposome is prepared. It is represented by the density (mg sugar chain/mg lipid) of a sugar chain that is bound per mg of the lipid of the liposome. Although it is not desired to be theoretically constrained, regarding the binding density of the sugar-chain-modified liposome of the present invention, it is empirically known that the amount of a sugar chain, which is used for preparation, is almost proportional to the density of the sugar chain bound to the liposome. Therefore, in the description, unless otherwise particularly noted, such binding density is determined depending on an amount that is used upon preparation. In vitro, for example, such binding density can be indirectly determined using E-selectin. The tropism for a target delivery site of the sugar-chain-modified liposome of the present invention can be controlled by selecting the type and binding density of a sugar chain to be bound to the liposome. Liposome numbers, sugar chain structures, modification (binding) densities, and related tropism for tumors are listed in Table 9 below.

TABLE 9

| | | Modification | |
|---|---|---|---|
| | | (binding) | |
| | | density | Tropism |
| | | (mg sugar | for |
| Liposome | | chain/ | inflammation |
| No. | Sugar chain structure | mg lipid) | site |
| 22 | Fuca1,2Galb1,3(Fuca1,4)GlcNAcb1,3,Galb1,4Glc | 0.75 | ++ |
| 27 | Fuca1,2Galb1,4(Fuca1,3)Glc | 0.5 | ++ |
| 38 | Gal_{β1,4}GlcNAc | 0.25 | ++ |
| 40 | GalNAca1,3(Fuca1,2)Gal | 0.025 | + |
| 41 | 3′-(O—SO3H)Galb1,4(Fuca1,3)GlcNAc | 0.25 | + |
| 50 | Mana1,2Mana1,6(Mana1,3)Mana1,6(Mana1,2Mana1,2Mana1,3)Manb1,4GlcNAcb1,4GlcNAc | 0.0385 | + |
| 53 | Fuca1,2Galb1,3GlcNAcb1,3Galb1,4Glc | 0.025 | + |
| 56 | Mana1,2Mana1,8(Mana1,2Mana1,3)Mana1,6(Mana1,2Mana1,2Mana1,3)Manb1,4GlcN | 0.042 | + |
| | Acb1,4GlcNAc | | |
| 60 | Neu5Aca2,6Galb1,4GlcNAc | 0.025 | ++ |
| 68 | Mana1,6(Mana1,3)Mana1,6(Mana1,2Mana1,2Mana1,3)Manb1,4GlcNAcb1,4GlcNAc | 0.035 | + |
| 69 | Galb1,3(Fuca1,4)GlcNAcb1,3Galb1,4Glc | 0.025 | ++ |
| 70 | Galb1,3GlcNAcb1,3Galb1,4Glc | 0.5 | ++ |
| 71 | Gala1,3(Fuca1,2)Gal | 0.025 | + |
| 76 | Fuca1,2Gal | 0.25 | + |
| 87 | Galb1,3GalNAc | 0.025 | ++ |
| 91 | Galb1,6GlcNAc | 0.25 | ++ |
| 93 | Mana1,6(Mana1,3)Man | 0.011 | ++ |
| 96 | Galb1,3GalNAc | 0.25 | ++ |
| 105 | Mana1,6(Mana1,3)Mana1,6(Mana1,2Mana1,3)Manb1,4GlcNAcb1,4GlcNAc | 0.031 | ++ |
| 106 | Fuca1,2Galb1,4(Fuca1,3)Glc | 0.25 | ++ |
| 111 | Galb1,3GlcNAc | 0.5 | + |
| 116 | Fuca1,2Galb1,4(Fuca1,3)Glc | 0.025 | ++ |
| 117 | Galb1,3GalNAc | 0.5 | + |
| 120 | Mana1,2Man | 0.0075 | + |
| 125 | Galb1,4(Fuca1,3)GlcNAc | 0.025 | ++ |
| 137 | Mana1,6(Mana1,3)Mana1,6Manb1,4GlcNAcb1,4GlcNAc | 0.024 | + |
| 139 | Galb1,4(Fuca1,3)Glc | 0.5 | ++ |
| 146 | Galb1,4(Fuca1,3)Glc | 0.25 | ++ |
| 150 | GalNAca1-O-L-serine | 0.5 | ++ |
| 151 | Neu5Aca2,3Galb1,4(Fuca1,3)GlcNAc | 0.25 | + |
| 152 | Fuca1,2Galb1,4(Fuca1,3)GlcNAc | 0.025 | ++ |
| 153 | Galb1,6GlcNAc | 0.5 | ++ |
| 154 | Galb1,4GlcNAc | 0.5 | ++ |
| 155 | Mana1,6Man | 0.0075 | ++ |
| 183 | Galb1,4GlcNAc | 0.5 | + |
| 184 | Neu5Aca2,6GalNAca1-O-L-serine | 0.025 | ++ |
| 186 | Mana1,6(Mana1,3)Manb1,4GlcNAcb1,4GlcNAc | 0.0205 | + |
| 189 | Neu5Aca2,6Galb1,4Glc | 0.0205 | ++ |
| 191 | Fuc_{α}1,2Gal_{β}1,3(Fuc_{α}1,4)GlcNAc | 0.025 | + |
| 195 | GalNAca1-O-L-serine | 0.25 | ++ |
| 197 | Mana1,4Man | 0.0075 | + |
| 199 | Neu5Ac2,6GalNAc_{α}1-O-L-serine | 0.25 | ++ |
| 204 | Galb1,4Glc | 0.025 | ++ |
| 209 | Fuca1,2Galb1,3(Fuca1,4)GlcNAcb1,3,Galb1,4Glc | 0.25 | + |
| 213 | Galb1,4(Fuca1,3)GlcNAc | 0.25 | ++ |
| 218 | Galb1,3GalNAcb1,4(Neu5Aca2,3)Galb1,4Glcb1,1Cer | 0.75 | − |
| 220 | Galb1,3GlcNAc | 0.25 | ++ |
| 224 | Mana1,6(Mana1,3)Mana1,6(Mana1,3)Manb1,4GlcNAcb1,4GlcNAc | 0.0275 | ++ |
| 229 | Galb1,6GlcNAc | 0.025 | ++ |
| 230 | Galb1,4GlcNAc | 0.025 | ++ |
| 233 | Fuca1,2Galb1,4Glc | 0.025 | ++ |
| 234 | GalNAca1-O-L-serine | 0.025 | ++ |
| 235 | Galb1,4Glc | 0.25 | ++ |
| 237 | Mana1,3Man | 0.0075 | ++ |
| 240 | 3′-(O—SO3H)Galb1,3(Fuca1,4)GlcNAc | 0.5 | + |
| 263 | Neu5Ac_{α}2,3Gal_{β1,4}GlcNAc | 0.025 | ++ |
| 288 | Galb1,3(Fuca1,4)GlcNAc | 0.025 | + |
| 290 | Neu5Aca2,3Galb1,3(Fuca1,4)GlcNAc | 0.5 | ++ |
| 292 | Neu5Ac_{α}2,6GalNAc_{α}1-O-L-serine | 0.5 | ++ |
| 295 | Neu5Aca2,6Galb1,4GlcNAc | 0.25 | + |

“++” indicates that when a liposome (standard liposome) to which Galβ1,3GalNAcβ1,4(Neu5Acα2,3)Galβ1,4Glcβ1,1Cer is bound instead of a sugar chain is administered via intravenous injection, the average value of the liposome delivered to the inflammation site is 2 to 8 times greater than that of the standard liposome at 5 minutes after intravenous injection.
“+” indicates that when a liposome (standard liposome) to which Galβ1,3GalNAcβ1,4(Neu5Acα2,3)Galβ1,4Glcβ1,1Cer is bound instead of a sugar chain is administered via intravenous injection, the average value of the liposome delivered to the inflammation site is 1 to 2 times greater than that of the standard liposome at 5 minutes after intravenous injection.

When a liposome to which tris(hydroxymethyl)aminomethane is bound is used as a standard liposome, “++” indicates that the average value of the liposome delivered to the inflammation site is 1.5 to 4.9 times greater than that of the standard liposome at 5 minutes after intravenous injection.

When a liposome to which tris(hydroxymethyl)aminomethane is bound is used as a standard liposome, “+” indicates that the average value of the liposome delivered to the inflammation site is 1.2 to 1.5 times greater than that of the standard liposome at 5 minutes after intravenous injection.

The liposome to which tris(hydroxymethyl)aminomethane is bound has slight tumor tropism. Thus, Galβ1,3GalNAcβ1,4(Neu5Acα2,3)Galβ1,4Glcβ1,1Cer was used as a standard liposome.

Preferably, the sugar-chain-modified liposome of the present invention, which is appropriate for delivery to inflammation sites, can be prepared using sugar chain types and modification (binding) densities listed in Table 9 above and combinations thereof. Once the tropism is found to be + or ++, the similar effect can be expected, which is not theoretically constrained, even when two or more types of sugar chain are combined. This is because a sugar chain that is recognized to be preferable by a lectin of target tissues or target cells is also recognized to be preferable even when two or more types of sugar chain are combined.

A sugar-chain-modified liposome appropriate for delivery to inflammation sites, which is used in the present invention, can be, preferably, liposome No. 22, 27, 38, 40, 41, 50, 53, 56, 60, 68, 69, 70, 71, 76, 87, 91, 93, 96, 105, 106, 111, 116, 117, 120, 125, 137, 139, 146, 150, 151, 152, 153, 154, 155, 183, 184, 186, 189, 191, 195, 197, 199, 204, 209, 213, 218, 220, 224, 229, 230, 233, 234, 235, 237, 240, 263, 288, 290, 292, or 295.

A sugar-chain-modified liposome to be used in the description can contain a sugar chain that is shown in Table 10, for example, at an adequate density for delivery to the liver.

When used in the description, “delivery to the liver” refers to the delivery to an area ranging from the right hypochondrium below the diaphragm to the upper part of the epigastrium.

When used in the description, “modification (binding) density” refers to an amount of a sugar chain that is used when a sugar-chain-modified liposome is prepared and is represented by the density (mg sugar chain/mg lipid) of a sugar chain that is bound per mg of the lipid of the liposome. Although no theoretical constraints are desired herein, regarding the binding density of the sugar-chain-modified liposome of the present invention, it is empirically known that the amount of a sugar chain, which is used for preparation, is almost proportional to the density of the sugar chain bound to the liposome. Therefore, in the description, unless otherwise particularly noted, such binding density is determined depending on an amount that is used upon preparation. In vitro, for example, such binding density can be indirectly determined using E-selectin. The tropism for a target delivery site of the sugar-chain-modified liposome of the present invention can be controlled by selecting the type and binding density of a sugar chain to be bound to the liposome. Liposome Nos., sugar chain structures, modification (binding) densities, and their tropism for the liver are listed in Table 10 below.

TABLE 10

| | | Modification | |
|---|---|---|---|
| | | (binding) | |
| | | density | |
| | | (mg sugar | Tropism |
| Liposome | | chain/ | for |
| No. | Sugar chain structure | mg lipid) | organ |
| 3 | Neu5Aca2,3Galb1,3GalNAcb1,4(Neu5Aca2,3)Galb1,4Glcb1,1Cer | 0.75 | ++ |
| 16 | 3′-(O—SO3H)Galb1,4(Fuca1,3)GlcNAc | 0.75 | ++ |
| 22 | Fuca1,2Galb1,3(Fuca1,4)GlcNAcb1,3,Galb1,4Glc | 0.75 | + |
| 27 | Fuca1,2Galb1,4(Fuca1,3)Glc | 0.5 | + |
| 29 | Neu5Aca2,3Galb1,4Glc | 0.5 | + |
| 37 | Galb1,3GalNAcb1,4(Neu5Aca2,8Neu5Aca2,3)Galb1,4Glcb1,1Cer | 0.75 | ++ |
| 38 | Galb1,4GlcNAc | 0.25 | ++ |
| 41 | 3′-(O—SO3H)Galb1,4(Fuca1,3)GlcNAc | 0.25 | + |
| 53 | Fuca1,2Galb1,3GlcNAcb1,3Galb1,4Glc | 0.025 | + |
| 67 | Galb1,3GalNAcb1,4Galb1,4Glcb + | 0.375 each | + |
| | Neu5Aca2,3Galb1,3GalNAcb1,4(Neu5Aca2,3)Galb1,4Glcb | | |
| 70 | Galb1,3GlcNAcb1,3Galb1,4Glc | 0.5 | + |
| 91 | Gal_{β}1,6GlcNAc | 0.25 | + |
| 96 | Galb1,3GalNAc | 0.25 | + |
| 106 | Fuca1,2Galb1,4(Fuca1,3)Glc | 0.25 | + |
| 111 | Galb1,3GlcNAc | 0.5 | + |
| 117 | Galb1,3GalNAc | 0.5 | + |
| 130 | Fuca1,2Galb1,4Glc | 0.25 | + |
| 139 | Galb1,4(Fuca1,3)Glc | 0.5 | + |
| 146 | Galb1,4(Fuca1,3)Glc | 0.25 | ++ |
| 150 | GalNAca1-O-L-serine | 0.5 | + |
| 151 | Neu5Aca2,3Galb1,4(Fuca1,3)GlcNAc | 0.25 | ++ |
| 152 | Fuca1,2Galb1,4(Fuca1,3)GlcNAc | 0.025 | + |
| 154 | Galb1,4GlcNAc | 0.5 | ++ |
| 178 | Galb1,3GlcNAcb1,3Galb1,4Glc | 0.25 | + |
| 183 | Galb1,4GlcNAc | 0.5 | ++ |
| 184 | Neu5Aca2,6GalNAca1-O-L-serine | 0.025 | + |
| 195 | GalNAca1-O-L-serine | 0.25 | ++ |
| 199 | Neu5Ac2,6GalNAca1-O-L-serine | 0.25 | + |
| 209 | Fuca1,2Galb1,3(Fuca1,4)GlcNAcb1,3,Galb1,4Glc | 0.25 | ++ |
| 218 | Galb1,3GalNAcb1,4(Neu5Aca2,3)Galb1,4Glcb1,1Cer | 0.75 | ++ |
| 225 | Galb1,4(Fuca1,3)Glc | 0.025 | ++ |
| 229 | Galb1,6GlcNAc | 0.025 | + |
| 230 | Galb1,4GlcNAc | 0.025 | + |
| 234 | GalNAca1-O-L-serine | 0.025 | ++ |
| 236 | Fuca1,2Galb1,4Glc | 0.5 | + |
| 239 | Neu5Aca2,3Galb1,3(Fuca1,4)GlcNAc | 0.25 | ++ |
| 240 | 3′-(O—SO3H)Galb1,3(Fuca1,4)GlcNAc | 0.5 | ++ |
| 285 | Neu5Aca2,3Galb1,4(Fuca1,3)GlcNAc | 0.5 | ++ |
| 290 | Neu5Aca2,3Galb1,3(Fuca1,4)GlcNAc | 0.5 | ++ |
| 292 | Neu5Aca2,6GalNAca1-O-L-serine | 0.5 | + |

When a liposome to which tris(hydroxymethyl)aminomethane is bound is used as a standard liposome, “++” indicates that the average value of the liposome delivered to the liver is 1.5 to 2.1 times greater than that of the standard liposome at 5 minutes after intravenous injection.

When a liposome to which tris(hydroxymethyl)aminomethane is bound is used as a standard liposome, “+” indicates that the average value of the liposome delivered to the liver is 1.2 to 1.5 times greater than that of the standard liposome at 5 minutes after intravenous injection.

The liposome to which tris(hydroxymethyl)aminomethane is bound has slight liver tropism. Thus, Galβ1,3GalNAcβ1,4(Neu5Acα2,3)Galβ1,4Glcβ1,1Cer was used as a standard liposome.

Preferably, the sugar-chain-modified liposome of the present invention, which is appropriate for delivery into the liver, can be prepared using sugar chain types and modification (binding) densities listed in Table 10 above and combinations thereof. Once the tropism is found to be + or ++, the similar effect can be expected, which is not theoretically constrained, even when two or more types of sugar chain are combined. This is because a sugar chain that is recognized to be preferable by a lectin of target tissues or target cells is also recognized to be preferable even when two or more types of sugar chain are combined.

A sugar-chain-modified liposome appropriate for delivery into the liver, which is used in the present invention, can be, preferably, liposome No. 3, 16, 22, 27, 29, 37, 38, 41, 53, 67, 70, 91, 96, 106, 111, 117, 130, 139, 146, 150, 151, 152, 154, 178, 183, 184, 195, 199, 209, 218, 225, 229, 230, 234, 236, 239, 240, 285, 290, or 292.

A sugar-chain-modified liposome to be used in the description can contain a sugar chain that is shown in Table 11, for example, at an adequate density for delivery into the pancreas.

When used in the description, “delivery to the pancreas” refers to the delivery to regions including long lobate glands (having no coat) that extend from the flexure of the duodenum to the spleen, the flat head part within the flexure of the duodenum, long and thin trihedral regions across the abdominal area, and the tail part that is in contact with the spleen.

When used in the description, “modification (binding) density” refers to an amount of a sugar chain that is used when a sugar-chain-modified liposome is prepared and is represented by the density (mg sugar chain/mg lipid) of a sugar chain that is bound per mg of the lipids of the liposome. Although no theoretical constraints are desired herein, regarding the binding density of the sugar-chain-modified liposome of the present invention, it is empirically known that the amount of a sugar chain, which is used for preparation, is almost proportional to the density of the sugar chain bound to the liposome. Therefore, in the description, unless otherwise particularly noted, such binding density is determined depending on an amount that is used upon preparation. In vitro, for example, such binding density can be indirectly determined using E-selectin. The tropism for a target delivery site of the sugar-chain-modified liposome of the present invention can be controlled by selecting the type and binding density of a sugar chain to be bound to the liposome. Liposome Nos., sugar chain structures, modification (binding) densities, and their pancreatic tropism are listed in Table 11 below.

TABLE 11

| | | Modification | |
|---|---|---|---|
| | | (binding) | |
| | | density | |
| | | (mg sugar | Tropism |
| Liposome | | chain/ | for |
| No. | Sugar chain structure | mg lipid) | Pancreas |
| 22 | Fuca1,2Galb1,3(Fuca1,4)GlcNAcb1,3,Galb1,4Glc | 0.75 | ++ |
| 27 | Fuca1,2Galb1,4(Fuca1,3)Glc | 0.5 | ++ |
| 29 | Neu5Aca2,3Galb1,4Glc | 0.5 | ++ |
| 38 | Galb1,4GlcNAc | 0.25 | ++ |
| 40 | GalNAc1,3(Fuca1,2)Gal | 0.025 | ++ |
| 56 | Mana1,2Mana1,6(Mana1,2Mana1,3)Mana1,6(Mana1,2Mana1,2Mana1,3)Manb1,4GlcNAcb1, | 0.042 | + |
| | 4GlcNAc | | |
| 60 | Neu5Aca2,6Galb1,4GlcNAc | 0.025 | + |
| 68 | Mana1,6(Mana1,3)Mana1,6(Mana1,2Mana1,2Mana1,3)Manb1,4GlcNAcb1,4GlcNAc | 0.035 | ++ |
| 69 | Galβ1,3(Fucα1,4)GlcNAcβ1,3Galβ1,4Glc | 0.025 | + |
| 70 | Galb1,3GlcNAcb1,3Galb1,4Glc | 0.5 | + |
| 71 | Gala1,3(Fuca1,2)Gal | 0.025 | ++ |
| 76 | Fuca1,2Gal | 0.25 | + |
| 87 | Galb1,3GalNAc | 0.025 | + |
| 91 | Galb1,6GlcNAc | 0.25 | ++ |
| 93 | Mana1,6(Mana1,3)Man | 0.011 | ++ |
| 96 | Galb1,3GalNAc | 0.25 | ++ |
| 105 | Mana1,6(Mana1,3)Mana1,6(Mana1,2Mana1,3)Manb1,4GlcNAcb1,4GlcNAc | 0.031 | ++ |
| 106 | Fuca1,2Galb1,4(Fuca1,3)Glc | 0.25 | ++ |
| 111 | Galb1,3GlcNAc | 0.5 | + |
| 116 | Fuca1,2Galb1,4(Fuca1,3)Glc | 0.025 | ++ |
| 117 | Galb1,3GalNAc | 0.5 | + |
| 120 | Mana1,2Man | 0.0075 | + |
| 125 | Galb1,4(Fuca1,3)GlcNAc | 0.025 | ++ |
| 127 | Gala1,3Gal | 0.25 | + |
| 130 | Fuca1,2Galb1,4Glc | 0.25 | + |
| 137 | Mana1,6(Mana1,3)Mana1,6Manb1,4GlcNAcb1,4GlcNAc | 0.024 | ++ |
| 139 | Galb1,4(Fuca1,3)Glc | 0.5 | ++ |
| 142 | Fuca1,2Gal | 0.5 | + |
| 146 | Galb1,4(Fuca1,3)Glc | 0.25 | + |
| 150 | GalNAca1-O-L-serine | 0.5 | + |
| 151 | Neu5Aca2,3Galb1,4(Fuca1,3)GlcNAc | 0.25 | ++ |
| 152 | Fuca1,2Galb1,4(Fuca1,3)GlcNAc | 0.025 | + |
| 153 | Galb1,6GlcNAc | 0.5 | ++ |
| 154 | Galb1,4GlcNAc | 0.5 | ++ |
| 155 | Mana1,6Man | 0.0075 | + |
| 175 | Gala1,3Gal | 0.5 | + |
| 184 | Neu5Aca2,6GalNAca1-O-L-serine | 0.025 | ++ |
| 191 | Fuc_{α}1,2Gal_{β}1,3(Fuc_{α}1,4)GlcNAc | 0.025 | + |
| 195 | GalNAca1-O-L-serine | 0.25 | ++ |
| 197 | Mana1,4Man | 0.0075 | + |
| 199 | Neu5Ac2,6GalNAc1-O-L-serine | 0.25 | ++ |
| 204 | Galb1,4Glc | 0.025 | + |
| 207 | Neu5Aca2,6Galb1,4Glc | 0.5 | + |
| 209 | Fuca1,2Galb1,3(Fuca1,4)GlcNAcb1,3,Galb1,4Glc | 0.25 | ++ |
| 213 | Galb1,4(Fuca1,3)GlcNAc | 0.25 | ++ |
| 218 | Galb1,3GalNAcb1,4(Neu5Aca2,3)Galb1,4Glcb1,1Cer | 0.75 | − |
| 220 | Galb1,3GlcNAc | 0.25 | ++ |
| 224 | Manα1,6(Manα1,3)Manα1,6(Manα1,3)Manβ1,4GlcNAcβ1,4GlcNAc | 0.0275 | ++ |
| 225 | Galb1,4(Fuca1,3)Glc | 0.025 | + |
| 229 | Galb1,6GlcNAc | 0.025 | ++ |
| 230 | Galb1,4GlcNAc | 0.025 | ++ |
| 233 | Fuca1,2Galb1,4Glc | 0.025 | ++ |
| 234 | GalNAca1-O-L-serine | 0.025 | + |
| 235 | Galb1,4Glc | 0.25 | ++ |
| 237 | Mana1,3Man | 0.0075 | ++ |
| 249 | Neu5Aca2,3Galb1,4Glc | 0.025 | ++ |
| 273 | GalNAca1,3(Fuca1,2)Gal | 0.5 | + |
| 292 | Neu5Aca2,6GalNAca1-O-L-serine | 0.5 | ++ |
| 295 | Neu5Aca2,6Galb1,4GlcNAc | 0.25 | ++ |

When a liposome (standard liposome) to which Galβ1,3GalNAcβ1,4(Neu5Acα2,3)Galβ1,4Glcβ1,1Cer is bound instead of a sugar chain is administered via intravenous injection, “++” indicates that the average value of the liposome delivered into the pancreas is 2 to 4 times greater than that of the standard liposome at 5 minutes after intravenous injection.

When a liposome (standard liposome) to which Galβ1,3GalNAcβ1,4(Neu5Acα2,3)Galβ1,4Glcβ1,1Cer is bound instead of a sugar chain is administered via intravenous injection, “+” indicates that the average value of the liposome delivered into the pancreas is 1 to 2 times greater than that of the standard liposome at 5 minutes after intravenous injection.

When a liposome to which tris(hydroxymethyl)aminomethane is bound is used as a standard liposome, “++” indicates that the average value of the liposome delivered into the pancreas is 1.5 to 2.2 times greater than that of the standard liposome at 5 minutes after intravenous injection.

When a liposome to which tris(hydroxymethyl)aminomethane is bound is used as a standard liposome, “+” indicates that the average value of the liposome delivered into the pancreas is 1.2 to 1.5 times greater than that of the standard liposome at 5 minutes after intravenous injection.

The liposome to which tris(hydroxymethyl)aminomethane is bound has slight pancreatic tropism. Thus, Galβ1,3GalNAcβ1,4(Neu5Acα2,3)Galβ1,4Glcβ1,1Cer was used as a standard liposome.

Preferably, the sugar-chain-modified liposome of the present invention, which is appropriate for delivery into the pancreas, can be prepared using sugar chain types and modification (binding) densities listed in Table 11 above and combinations thereof. Once the tropism is found to be + or ++, the similar effect can be expected, which is not theoretically constrained, even when two or more types of sugar chain are combined. This is because a sugar chain that is recognized to be preferable by a lectin of target tissues or target cells is also recognized to be preferable even when two or more types of sugar chain are combined.

A sugar-chain-modified liposome appropriate for delivery into the pancreas, which is used in the present invention, can be, preferably, liposome No. 22, 27, 29, 38, 40, 56, 60, 68, 69, 70, 71, 76, 87, 91, 93, 96, 105, 106, 111, 116, 117, 120, 125, 127, 130, 137, 139, 142, 146, 150, 151, 152, 153, 154, 155, 175, 184, 191, 195, 197, 199, 204, 207, 209, 213, 218, 220, 224, 225, 229, 230, 233, 234, 235, 237, 249, 273, 292, or 295.

A sugar-chain-modified liposome to be used in the description can contain a sugar chain that is shown in Table 12, for example, at an adequate density for delivery into the brain.

When used in the description, “delivery to the brain” refers to the delivery to regions (e.g., cerebrum, cerebellum, and medulla oblongata) of the entire central nerve system within the cranium.

When used in the description, “modification (binding) density” refers to an amount of a sugar chain that is used when a sugar-chain-modified liposome is prepared and is represented by the density (mg sugar chain/mg lipid) of a sugar chain that is bound per mg of the lipid of the liposome. Although no theoretical constraints are desired herein, regarding the binding density of the sugar-chain-modified liposome of the present invention, it is empirically known that the amount of a sugar chain, which is used for preparation, is almost proportional to the density of the sugar chain bound to the liposome. Therefore, in the description, unless otherwise particularly noted, such binding density is determined depending on an amount that is used upon preparation. In vitro, for example, such binding density can be indirectly determined using E-selectin. The tropism for a target delivery site of the sugar-chain-modified liposome of the present invention can be controlled by selecting the type and binding density of a sugar chain to be bound to the liposome. Liposome Nos., sugar chain structures, modification (binding) densities, and their tropism for the brain are listed in Table 12 below.

TABLE 12

| | | Modification | |
|---|---|---|---|
| | | (binding) | |
| | | density | |
| | | (mg sugar | Tropism |
| Liposome | | chain/ | for |
| No. | Sugar chain structure | mg lipid) | organ |
| 22 | Fuca1,2Galb1,3(Fuca1,4)GlcNAcb1,3,Galb1,4Glc | 0.75 | + |
| 29 | Neu5Aca2,3Galb1,4Glc | 0.5 | ++ |
| 38 | Galb1,4GlcNAc | 0.25 | + |
| 40 | GalNAca1,3(Fuca1,2)Gal | 0.025 | ++ |
| 45 | Fuca1,2Gal | 0.025 | ++ |
| 50 | Mana1,2Mana1,6(Mana1,3)Mana1,6(Mana1,2Mana1,2Mana1,3)Manb1,4GlcNAcb1,4GlcNAc | 0.0385 | ++ |
| 53 | Fuca1,2Galb1,3GlcNAcb1,3Galb1,4Glc | 0.025 | + |
| 56 | Mana1,2Mana1,6(Mana1,2Mana1,3)Mana1,6(Mana1,2Mana1,2Mana1,3)Manb1,4GlcNAcb1, | 0.042 | + |
| | 4GlcNAc | | |
| 60 | Neu5Aca2,6Galb1,4GlcNAc | 0.025 | ++ |
| 67 | Galb1,3GalNAcb1,4Galb1,4Glcb + | 0.375 each | + |
| | Neu5Aca2,3Galb1,3GalNAcb1,4(Neu5Aca2,3)Galb1,4Glcb | | |
| 68 | Mana1,6(Mana1,3)Mana1,6(Mana1,2Mana1,2Mana1,3)Manb1,4GlcNAcb1,4GlcNAc | 0.035 | ++ |
| 69 | Galb1,3(Fuca1,4)GlcNAcb1,3Galb1,4Glc | 0.025 | + |
| 70 | Galb1,3GlcNAcb1,3Galb1,4Glc | 0.5 | ++ |
| 71 | Gala1,3(Fuca1,2)Gal | 0.025 | ++ |
| 76 | Fuca1,2Gal | 0.25 | + |
| 80 | Neu5Aca2,6Galb1,4GlcNAc | 0.5 | + |
| 87 | Galb1,3GalNAc | 0.025 | + |
| 93 | Mana1,6(Mana1,3)Man | 0.011 | ++ |
| 105 | Mana1,6(Mana1,3)Mana1,6(Mana1,2Mana1,3)Manb1,4GlcNAcb1,4GlcNAc | 0.031 | ++ |
| 106 | Fuca1,2Galb1,4(Fuca1,3)Glc | 0.25 | + |
| 116 | Fuca1,2Galb1,4(Fuca1,3)Glc | 0.025 | + |
| 120 | Mana1,2Man | 0.0075 | ++ |
| 127 | Galα1,3Gal | 0.25 | + |
| 129 | Gala1,3(Fuca1,2)Gal | 0.25 | + |
| 137 | Mana1,6(Mana1,3)Mana1,6Manb1,4GlcNAcb1,4GlcNAc | 0.024 | ++ |
| 141 | Galb1,3(Fuca1,2)Gal | 0.5 | + |
| 142 | Fuca1,2Gal | 0.5 | + |
| 146 | Galb1,4(Fuca1,3)Glc | 0.25 | + |
| 150 | GalNAca1-O-L-serine | 0.5 | + |
| 151 | Neu5Aca2,3Galb1,4(Fuca1,3)GlcNAc | 0.25 | ++ |
| 152 | Fuca1,2Galb1,4(Fuca1,3)GlcNAc | 0.025 | + |
| 154 | Galb1,4GlcNAc | 0.5 | + |
| 155 | Mana1,6Man | 0.0075 | ++ |
| 175 | Gala1,3Gal | 0.5 | + |
| 178 | Galb1,3GlcNAcb1,3Galb1,4Glc | 0.25 | + |
| 184 | Neu5Aca2,6GalNAca1-O-L-serine | 0.025 | + |
| 186 | Mana1,6(Mana1,3)Manb1,4GlcNAcb1,4GlcNAc | 0.0205 | ++ |
| 189 | Neu5Aca2,6Galb1,4Glc | 0.0205 | ++ |
| 197 | Mana1,4Man | 0.0075 | ++ |
| 207 | Neu5Aca2,6Galb1,4Glc | 0.5 | ++ |
| 209 | Fuca1,2Galb1,3(Fuca1,4)GlcNAcb1,3,Galb1,4Glc | 0.25 | ++ |
| 213 | Galb1,4(Fuca1,3)GlcNAc | 0.25 | + |
| 218 | Galb1,3GalNAcb1,4(Neu5Aca2,3)Galb1,4Glcb1,1Cer | 0.75 | − |
| 224 | Mana1,6(Mana1,3)Mana1,6(Mana1,3)Manb1,4GlcNAcb1,4GlcNAc | 0.0275 | ++ |
| 229 | Galb1,6GlcNAc | 0.025 | + |
| 230 | Galb1,4GlcNAc | 0.025 | + |
| 233 | Fuca1,2Galb1,4Glc | 0.025 | ++ |
| 235 | Galb1,4Glc | 0.25 | + |
| 237 | Mana1,3Man | 0.0075 | ++ |
| 249 | Neu5Aca2,3Galb1,4Glc | 0.025 | ++ |
| 273 | GalNAc_{α}1,3(Fuc_{α}1,2)Gal | 0.5 | ++ |
| 290 | Neu5Aca2,3Galb1,3(Fuca1,4)GlcNAc | 0.5 | + |
| 292 | Neu5Aca2,6GalNAca1-O-L-serine | 0.5 | + |
| 295 | Neu5Aca2,6Galb1,4GlcNAc | 0.25 | ++ |

When a liposome (standard liposome) to which Galβ1,3GalNAcβ1,4(Neu5Acα2,3)Galβ1,4Glcβ1,1Cer is bound instead of a sugar chain is administered via intravenous injection, “++” indicates that the average value of the liposome delivered into the brain is 3 to 7 times greater than that of the standard liposome at 5 minutes after intravenous injection.

When a liposome (standard liposome) to which Galβ1,3GalNAcβ1,4(Neu5Acα2,3)Galβ1,4Glcβ1,1Cer is bound instead of a sugar chain is administered via intravenous injection, “+” indicates that the average value of the liposome delivered into the brain is 2 to 3 times greater than that of the standard liposome at 5 minutes after intravenous injection.

When a liposome to which tris(hydroxymethyl)aminomethane is bound is used as a standard liposome, “++” indicates that the average value of the liposome delivered into the brain is 1.7 to 3.7 times greater than that of the standard liposome at 5 minutes after intravenous injection.

When a liposome to which tris(hydroxymethyl)aminomethane is bound is used as a standard liposome, “+” indicates that the average value of the liposome delivered into the brain is 1.1 to 1.4 times greater than that of the standard liposome at 5 minutes after intravenous injection.

The liposome to which tris(hydroxymethyl)aminomethane is bound has slight tropism for the brain. Thus, Galβ1,3GalNAcβ1,4(Neu5Acα2,3)Galβ1,4Glcβ1,1Cer was used as a standard liposome.

Preferably, the sugar-chain-modified liposome of the present invention, which is appropriate for delivery into the brain, can be prepared using sugar chain types and modification (binding) densities listed in Table 12 above and combinations thereof. Once the tropism is found to be + or ++, the similar effect can be expected, which is not theoretically constrained, even when two or more types of sugar chain are combined. This is because a sugar chain that is recognized to be preferable by a lectin of target tissues or cells is also recognized to be preferable even when two or more types of sugar chain are combined.

A sugar-chain-modified liposome appropriate for delivery into the brain, which is used in the present invention, can be, preferably, liposome No. 22, 29, 38, 40, 45, 50, 53, 56, 60, 67, 68, 69, 70, 71, 76, 80, 87, 93, 105, 106, 116, 120, 127, 129, 137, 141, 142, 146, 150, 151, 152, 154, 155, 175, 178, 184, 186, 189, 197, 207, 209, 213, 218, 224, 229, 230, 233, 235, 237, 249, 273, 290, 292, or 295.

Sugar-chain-modified liposomes preferred in the present invention as listed in the above Tables can be produced by the following method. Specifically, the method comprises the steps of: (a) providing a liposome; (b) hydrophilizing the liposome; (c) generating a linker-bound liposome by binding a linker to the hydrophilized liposome according to need; and (d) generating a sugar-chain-modified liposome by binding a sugar chain shown in Table 3 above to the liposome.

Preferably, in this method, the step (b) of hydrophilizing a liposome is performed by directly or indirectly binding a low-molecular-weight hydrophilic compound onto the lipid membrane of the liposome or a linker. A linker that is used in the step (c) is a protein derived from a human. Furthermore, in the step (d), a sugar chain is bound to the liposome so as to generate a sugar-chain-modified liposome under conditions where the sugar chain is directly or indirectly bound to the liposome.

A liposome and a linker are and a linker and a sugar chain are preferably bound to each other using a bifunctional cross-linking agent (e.g., DTSSP) or the like.

A drug or a gene can be encapsulated into or bound to the delivery vehicle of the present invention. Examples of such a drug include, but are not limited to, biological and pharmaceutical products or substances for biological therapy (e.g., an siRNA, an shRNA, an siRNA derivative, an shRNA derivative, an RNA, an RNA derivative, a DNA, a DNA derivative, a monoclonal antibody, a vaccine, an interferon, a hormone, prostaglandin, a transcription factor, a recombinant protein, an antibody drug, a nucleic acid drug, and a gene therapeutic drug), alkylating anticancer agents, antimetabolites, plant-derived anticancer agents, anticancerous antibiotics, biological response modifiers (BRM) and/or cytokines, drugs for tumors (e.g., a platinum complex anticancer agent, an immunotherapeutic agent, a hormone-based anticancer agent, and a monoclonal antibody), drugs for the central nerve, drugs for the peripheral nerve system-sense organs, drugs for treatment of respiratory diseases, drugs for circulatory organs, drugs for digestive organs, drugs for the hormone system, drugs for urinary organs-reproductive organs, vitamins-revitalizers, metabolic pharmaceutical products, antibiotics-chemotherapeutic drugs, drugs for examination, anti-inflammatory agents, drugs for eye diseases, drugs for the central nerve system, drugs for the autoimmune system, drugs for the circulatory system, drugs for lifestyle-related diseases (e.g., diabetes and hyperlipemia), adrenal cortex hormones, immunosuppresants, antimicrobial agents, antiviral agents, agents for suppressing vascularization, cytokines, chemokines, anti-cytokine antibodies, anti-chemokine antibodies, anti-cytokine-chemokine receptor antibodies, nucleic acid formulations relating to gene therapy (e.g., siRNA, shRNA, miRNA, smRNA, antisense RNA, ODN, or DNA), neuroprotective factors, and antibody drugs.

The delivery vehicle of the present invention can be used for administering a biological factor to a subject who needs the biological factor via oral administration. Furthermore, the delivery vehicle can also be used for treating mammals having the disorder of the respiratory system, circulatory system, digestive system, urinary organ system-reproductive organ system, central nerve system, peripheral nerve system, or the like.

The ability to control absorbance in the intestinal tract and the specificity for the delivery to various organs of the delivery vehicle of the present invention can also be enhanced via regulation of the properties (e.g., depending on sugar chain types) and binding density of the delivery vehicle. Through binding of both a sugar chain that enhances the ability to control absorbance in the intestinal tract and a sugar chain having tropism for a specific tissue or organ to the delivery vehicle, a delivery vehicle having both properties of tropism for a specific tissue or organ and ability to control absorbance in the intestinal tract can also be prepared.

The delivery vehicle of the present invention can be easily prepared by persons skilled in the art in view of pH, isotonicity, stability, and the like. The delivery vehicle of the present invention can be compounded with a pharmaceutically acceptable carrier and then administered via oral administration in the form of solid formulation such as tablets, capsules, fine granules, powders, or powdered drugs or liquid formulation such as syrups, suspension agents, or solutions. The delivery vehicle can be prescribed in a form appropriate for administration through the use of a pharmaceutically acceptable carrier known in the art. The use of such a carrier makes it possible to prescribe the delivery vehicle in the form of liquid, gel, syrup, slurry, suspended matter, or the like that is appropriate for intake by a patient.

The delivery vehicle of the present invention contains a composition in which an active ingredient such as a drug or a biological factor is contained in a vehicle in an effective amount (dose) for achievement of the intended purposes. The term “effective amount (dose) for treatment” is sufficiently recognized by persons skilled in the art and refers to an amount of a drug, which is effective for providing intended pharmacological results (e.g., prevention, treatment, and prevention of recurrence). Therefore, such effective dose for treatment is an amount sufficient for alleviating the symptoms of disease to be treated. One useful assay for confirmation of such an effective dose (e.g., effective dose for treatment) for a given application is to measure the degree of recovery of a target disease. An actual dose to be administered depends on an individual body to be treated and preferably is an amount optimized for achieving desired effects without significant side effects. Determination of such effective dose is sufficiently within the capacity of persons skilled in the art.

A therapeutically effective dose, a prophylactically effective dose, and the like and toxicity can be determined by performing standard pharmaceutical procedures (e.g., ED₅₀ that is a dose therapeutically effective for 50% of a population; and LD₅₀ that is a dose lethal to 50% of a population) for cell culture or experimental animals. The ratio of a therapeutically effective dose to a toxic dose is a therapeutic index that can be represented by the ratio of ED₅₀/LD₅₀. A drug delivery vehicle with a small therapeutic index is preferred herein. Data obtained via a cell culture assay and an animal experiment can be used for formulation of the range of an amount to be used for a human. The dose of such a compound is, preferably, within the range of a circulation concentration including ED₅₀ with almost no or completely no toxicity. Such a dose is varied within the range depending on the form of administration, which is employed herein, susceptibility of a patient, and the route of administration. For example, such a dose is adequately selected depending on the conditions of a patient, such as age, disease types, cell types to be used herein, and the like.

The drug delivery vehicle of the present invention can be produced in a manner (e.g., mixing or dissolving) similar to that known in the art.

In the description, “instruction” describes a method or the like for administering the sugar-chain-modified liposome, the drug delivery vehicle for oral administration of the present invention, or the like is described for persons who perform administration such as doctors and patients and for persons who make diagnosis (can be patients themselves). Such instruction contains words for instructing the procedures for administration of the sugar-chain-modified liposome or the drug delivery vehicle for oral administration of the present invention. Such instruction is prepared according to the format as specified by the supervisory authorities (e.g., Health, Labour and Welfare Ministry in Japan and Food and Drug Administration (FDA) in the U.S.). Specifically, such instruction is prepared according to the format as specified by the supervisory authorities in the country in which the present invention is implemented and the approval by the supervisory authorities is also clearly stated therein. Such instruction is namely appended paper (package insert) and is generally provided in the form of paper medium. However, the form of such instruction is not limited to such form. The instruction can also be provided in the form of electronic medium (e.g., homepage provided via internet (web site) or e-mail), for example.

In the description, “subject” refers to an organism to which treatment of the present invention is applied and also refers to “patient.” Such patient or subject can be preferably a human.

The medicine of the present invention can be prepared in the form of freeze-dried cake or aqueous solution and then stored by mixing, according to need, a physiologically acceptable carrier, an excipient, or a stabilizing agent (e.g., see ver. 14 or the latest version of Japanese Pharmacopoeia and Remington's Pharmaceutical Sciences, 18^{th} Edition, A. R. Gennaro, ed., Mack Publishing Company, 1990) with a sugar chain composition having a desired degree of purity.

The medicine of the present invention can be administered via oral or parenteral administration. Alternatively, the medicine of the present invention can be administered intravenously or subcutaneously. When systemically-administered, a medicine to be used in the present invention can be in the form of pharmaceutically acceptable aqueous solution containing no pyrogen substance. Such a pharmaceutically acceptable composition can be easily prepared by persons skilled in the art in view of pH, isotonicity, stability, and the like. The route of administration to be employed in the description can be oral administration, parenteral administration (e.g., intravenous administration, intramuscular administration, subcutaneous administration, intradermal administration, transmucosal administration, intrarectal administration, intravaginal administration, local administration to affected parts, and skin administration). A product prescribed for such administration can be provided in an arbitrary form for formulation. Examples of such form for formulation include liquids, injections, and sustained preparations.

Examples of materials appropriate for such prescription or pharmaceutically acceptable carriers include, but are not limited to, anti-oxidants, preservatives, coloring agents, flavoring agents, diluents, emulsifiers, suspending agents, solvents, fillers, extending agents, buffers, delivery vehicles, diluents, excipients, and/or pharmaceutical adjuvants. Typically, the medicine of the present invention is administered in the form of composition containing an isolated multipotent stem cell or an altered product thereof or a derivative thereof in addition to one or more physiologically acceptable carriers, excipients, or diluents. For example, an appropriate vehicle can be water for injection, a physiological solution, or an artificial cerebrospinal fluid, which can be supplemented with other general substances for compositions for parenteral delivery.

An acceptable carrier, excipient, or stabilizing agent, which is used in the description is nontoxic for a recipient and is preferably inactive at the dosage and concentration to be employed herein. Examples of such carrier, excipient, or stabilizing agent include: phosphate, citrate, or other organic acids; ascorbic acid or α-tocopherol; low-molecular-weight polypeptide; proteins (e.g., serum albumin, gelatin or immunoglobulin); hydrophilic polymers (e.g., polyvinyl pyrrolidone); amino acids (e.g., glycine, glutamine, asparagine, arginine, or lysine); monosaccharides, disaccharides, and other carbohydrates (including glucose, mannose, or dextrin); chelating agents (e.g., EDTA); sugar alcohols (e.g., mannitol or sorbitol); salt-forming counter ions (e.g., sodium); and/or nonionic surface active agents (e.g., Tween, pluronic, or polyethylene glycol (PEG)).

Examples of an appropriate carrier include a neutral buffered physiological saline solution and a physiological saline solution mixed with serum albumin. Preferably, the generated product is prescribed as freeze-dried agent using an appropriate excipient (e.g., sucrose). Another standard carrier, a diluent, and an excipient can be contained if desired. Another example of a composition contains a Tris buffer (pH7.0 to 8.5) or an acetic acid buffer (pH4.0 to 5.5). Such an example can further contain sorbitol or an appropriate alternate thereof.

When the present invention is used for cosmetics, such cosmetics can be prepared in compliance with the regulations defined by the authority concerned.

The delivery vehicle or the composition of the present invention can also be used as components of agricultural chemicals. When the delivery vehicle or the composition of the present invention is prescribed as a composition for an agricultural chemical, it can contain an agriculturally acceptable carrier, an excipient, a stabilizing agent, or the like, according to need.

When the delivery vehicle or the composition of the present invention is used as an agricultural chemical, it can be mixed with herbicides (e.g., pyrazolate), insecticides-miticides (e.g., diazinon), microbicides (e.g., probenazole), plant growth adjustment agents (e.g., paclobutrazol), nematicides (e.g., benomyl), synergists (e.g., piperonyl butoxide), attractants (e.g., eugenol), repellents (e.g., creosote), dyes (e.g., food blue No. 1), fertilizers (e.g., urea) and the like, according to need.

### (Healthcare-Food)

The present invention can also be applied for use in the fields of healthcare and foods. In such cases, points that should be remembered when it is used as an oral medicine as described above should be taken into consideration according to need. In particular, when the present invention is applied for use as a functional food and/or health food such as a food for specified health use, it is preferably treated according to the manner employed for medicines. Preferably, the delivery vehicle of the present invention can also be used as a low allergic food.

The amount of a composition that is used in the treatment method of the present invention can be easily determined by persons skilled in the art in view of purpose for use, target disease (e.g., type and severity), patient's age, body weight, sex, and medical history, form or type of cell, and the like. Frequency for performing the treatment method of the present invention for a subject (or patient) can also be easily determined by persons skilled in the art in view of purpose for use, target disease (e.g., type and severity), patient's age, body weight, sex, medical history, therapeutic process, and the like. Such frequency for administration ranges from daily to once per several months (e.g., once a week to once a month), for example. It is preferable to perform administration once a week to once a month while observing the progress.

In another aspect, the present invention provides a method for preventing or treating a subject who needs the delivery of a drug to desired sites. This method comprises the steps of:

A) measuring in vitro affinity of candidate delivery vehicles for achievement of delivery to a desired site for a cell surface molecule such as a lectin associated with the site;
B) selecting a delivery vehicle having in vitro affinity corresponding to delivery to the desired site; and
C) administering a drug required for prevention or treatment to the subject using the selected delivery vehicle.

Alternatively, this method comprises the steps of:

A) measuring in vitro affinity of candidate delivery vehicles for achievement of delivery to a desired site for a cell surface molecule such as a lectin associated with the site;
B) selecting a delivery vehicle having in vitro affinity corresponding to delivery to the desired site and analyzing the composition of the selected delivery vehicle;
C) generating the selected delivery vehicle based on the composition, which contains a drug required for prevention or treatment; and
D) administering the selected delivery vehicle to the subject.

Examples of such delivery vehicle include, but are not limited to:

a delivery vehicle for achievement of delivery to a desired site, in which an inhibitory concentration at approximately strong binding IC30 or less is 10⁻⁹M or less in terms of in vitro affinity for a cell surface molecule such as a lectin associated with a desired site;
a delivery vehicle for achievement of delivery to a desired site, in which an inhibitory concentration at approximately weak binding IC31 or more is 10⁻⁹M or more in terms of in vitro affinity for a cell surface molecule such as a lectin associated with a desired site;
a delivery vehicle for achievement of delivery to a desired site, in which an inhibitory concentration at approximately strong binding IC30 or less is 10⁻⁹M or less, and an inhibitory concentration at approximately weak binding IC31 or more is 10⁻⁹M or more in terms of in vitro affinity for a cell surface molecule such as a lectin associated with a desired site; and
a delivery vehicle for achievement of delivery to a desired site, which satisfies at least one condition selected from the group consisting of a condition in which the inhibitory concentration at IC40 is 10⁻⁹M or more, a condition in which the inhibitory concentration at IC50 is 10⁻⁹M or more, and a condition in which the inhibitory concentration at IC40 is 10⁻⁹M or more, and satisfies at least one condition selected from the group consisting of a condition in which the inhibitory concentration at IC30 is 10⁻⁹M or less, a condition in which the inhibitory concentration at IC20 is 10⁻⁹M or less, and a condition in which the inhibitory concentration at IC10 is 10⁻⁹M in terms of in vitro affinity for a cell surface molecule such as a lectin associated with a desired site. Here, IC can be measured using affinity for E-selectin, but the example is not limited thereto. It is understood that these delivery vehicles are also encompassed within the scope of the present invention. In this case, since E-selectin closely correlates with at least inflammation sites and cancer sites, the delivery vehicles can be used for delivery to the inflammation sites and cancer sites.

Specific examples of sugar-chain-modified liposomes that satisfy the above conditions can be listed as follows.

TABLE 13

| No. | Abbreviated name | Structure |
|---|---|---|
| 3 | EEGD1A-BH | Neu5Acα2,3Galβ1,3GalNAcβ1,4 |
| | | (Neu5Acα2,3)Galβ1,4Glcβ1,1Cer |
| 27 | LDF-5 | Fucα1,2Galβ1,4(Fucα1,3)Glc |
| 29 | 3SL-5 | Neu5Acα2,3Galβ1,4Glc |
| 37 | FEEGD1b | Galβ1,3GalNacβ1,4(Neu5Acα2,8Neu5Acα2,3) |
| | | Galβ1,4Glcβ1,1Cer |
| 38 | G4GN-4 | Galβ1,4GlcNAc |
| 40 | BAT-1 | GalNAcα1,3(Fucα1,2)Gal |
| 41 | SOLX-4 | 3′-(O—SO3H)Galβ1,4(Fucα1,3)GlcNAc |
| 45 | BHD-1 | Fucα1,2Gal |
| 50 | ManB | Manα1,2Manα1,6(Manα1,3)Manα1,6 |
| | | (Manα1,2Manα1,2Manα1,3)Manβ1,4GlcNAcβ1,4GlcNAc |
| 53 | L2F-1 | Fuca1,2Galb1,3GlcNAcb1,3Galb1,4Glc |
| 56 | Man9 | Manα1,2Manα1,6(Manα1,2Manα1,3)Manα1,6 |
| | | (Manα1,2Manα1,2Manα1,3)Manβ1,4GlcNAcβ1,4GlcNAc |
| 60 | 6SLN-1 | Neu5Acα2,6Galβ1,4GlcNAc |
| 67 | FEEaMDa | Galβ1,3GalNAcβ1,4Galβ1,4Glcβ- |
| | | Neu5Acα2,3Galβ1,3GalNAcβ1,4(Neu5Acα2,3)Galβ1,4Glcβ |
| 68 | Man7 | Manα1,6(Manα1,3)Manα1,6(Manα1,2Manα1,2Manα1,3) |
| | | Manβ1,4GlcNAcβ1,4GlcNAc |
| 69 | L4F-1 | Galβ1,3(Fucα1,4)GlcNAcβ1,3Galβ1,4Glc |
| 70 | LNT-5 | Galβ1,3GlcNAcβ1,3Galβ1,4Glc |
| 71 | BBT-1 | Galα1,3(Fucα1,2)Gal |
| 76 | BHD-4 | Fucα1,2Gal |
| 80 | 6SLN-5 | Neu5Acα2,6Galβ1,4GlcNAc |
| 87 | TA-1 | Galβ1,3GalNAc |
| 91 | G6GN-4 | Galβ1,6GlcNAc |
| 93 | A36 | Manα1,6(Manα1,3)Man |
| 96 | TA-4 | Galb1,3GalNAc |
| 105 | Man6 | Manα1,6(Manα1,3)Manα1,6(Manα1,2Manα1,3)Manβ1,4 |
| | | GlcNAcβ1,4GlcNAc |
| 106 | LDF-4 | Fucα1,2Galβ1,4(Fucα1,3)Glc |
| 111 | LNB-5 | Galβ1,3GlcNAc |
| 116 | LDF-1 | Fucα1,2Galβ1,4(Fucα1,3)Glc |
| 117 | TA-5 | Galβ1,3GalNAc |
| 120 | A2 | Manα1,2Man |
| 125 | LX-1 | Galβ1,4(Fucα1,3)GlcNAc |
| 127 | G3G-4 | Galα1,3Gal |
| 129 | BBT-4 | Galα1,3(Fucα1,2)Gal |
| 130 | 2FL-4 | Fucα1,2Galβ1,4Glc |
| 137 | Man4B | Manα1,6(Manα1,3)Manα1,6Manβ1,4GlcNAcβ1,4GlcNAc |
| 139 | 3FL-5 | Galβ1,4(Fucα1,3)Glc |
| 141 | BBT-5 | Galα1,3(Fucα1,2)Gal |
| 142 | BHD-5 | Fucα1,2Gal |
| 146 | 3FL-4 | Galβ1,4(Fucα1,3)Glc |
| 150 | TNA-5 | GalNAcα1-O-L-serine |
| 151 | SLX-4 | Neu5Acα2,3Galβ1,4(Fucα1,3)GlcNAc |
| 152 | LY-1 | Fucα1,2Galβ1,4(Fucα1,3)GlcNAc |
| 153 | G6GN-5 | Galβ1,6GlcNAc |
| 154 | Lac-5 | Galβ1,4Glc |
| 155 | A6 | Manα1,6Man |
| 175 | G3G-5 | Galα1,3Gal |
| 178 | LNT-4 | Galβ1,3GlcNAcβ1,3Galβ1,4Glc |
| 183 | G4GN-5 | Galβ1,4GlcNAc |
| 184 | STNA-1 | Neu5Acα2,6GalNAcα1-O-L-serine |
| 186 | Man3 | Manα1,6(Manα1,3)Manβ1,4GlcNAcβ1,4GlcNAc |
| 191 | LB-1 | Fucα1,2Galβ1,3(Fucα1,4)GlcNAc |
| 195 | TNA-4 | GalNAcα1-O-L-serine |
| 197 | A4 | Manα1,4Man |
| 199 | STNA-4 | Neu5Ac2,6GalNAcα1-O-L-serine |
| 204 | Lac-1 | Galβ1,4Glc |
| 207 | 6SL-5 | Neu5Acα2,6Galβ1,4Glc |
| 209 | LDFH-4 | Fuca1,2Galb1,3(Fuca1,4)GlcNAcb1,3Galb1,4Glc |
| 213 | LX-4 | Galβ1,4(Fucα1,3)GlcNAc |
| 218 | EEGM1-BH | Galβ1,3GalNAcβ1,4(Neu5Acα2,3)Galβ1,4Glcβ1,1Cer |
| 220 | LNB-4 | Galβ1,3GlcNAc |
| 224 | Man5 | Manα1,6(Manα1,3)Manα1,6(Manα1,3) |
| | | Manβ1,4GlcNAcβ1,4GlcNAc |
| 225 | 3FL-1 | Galβ1,4(Fucα1,3)Glc |
| 229 | G6GN-1 | Galβ1,6GlcNAc |
| 230 | G4GN-1 | Galβ1,4GlcNAc |
| 233 | 2FL-1 | Fucα1,2Galβ1,4Glc |
| 234 | TNA-1 | GalNAcα1-O-L-serine |
| 235 | Lac-4 | Galβ1,4Glc |
| 236 | 2FL-5 | Fucα1,2Galβ1,4Glc |
| 237 | A3 | Manα1,3Man |
| 239 | SLA-4 | Neu5Acα2,3Galβ1,3(Fucα1,4)GlcNAc |
| 240 | SOLA-5 | 3′-(O—SO3H)Galβ1,3(Fucα1,4)GlcNAc |
| 249 | 3SL-1 | Neu5Acα2,3Galβ1,4Glc |
| 254 | G3G-1 | Galα1,3Gal |
| 263 | 3SLN-1 | Neu5Acα2,3Galβ1,4GlcNAc |
| 273 | BAT-5 | GalNAcα1,3(Fucα1,2)Gal |
| 285 | SLX-5 | Neu5Acα2,3Galβ1,4(Fucα1,3)GlcNAc |
| 288 | LA-1 | Galβ1,3(Fucα1,4)GlcNAc |
| 290 | SLA-5 | Neu5Acα2,3Galβ1,3(Fucα1,4)GlcNAc |
| 292 | STNA-5 | Neu5Acα2,6GalNAcα1-O-L-serine |
| 295 | 6SLN-4 | Neu5Acα2,6Galβ1,4GlcNAc |
| 301 | TRIS | tris(hydroxymethyl)aminomethane |

Preferably, a delivery vehicle can be a liposome (e.g., sugar-chain-modified liposome).

Therefore, the present invention also provides a pharmaceutical composition containing a drug that is used for prevention or treatment and the delivery vehicle of the present invention or a delivery vehicle that is produced by the method for producing a delivery vehicle of the present invention.

### (Alteration)

In the description, a delivery vehicle that is actually measured can be altered by substitution, if desired. In the case of a sugar chain, the specificity of the sugar chain can be altered by introducing a methyl group for substitution of a hydroxy group of the sugar chain, for example. The affinity of a product prepared by such alteration can be measured by in vitro screening according to the rolling model of the present invention.

In the description, unless otherwise particularly noted, “substitution” refers to substitution of 1, 2 or more (or several) hydrogen atoms in an organic compound or a substituent with other atoms or atomic groups. Substitution with a monovalent substituent can also be performed by the removal of one hydrogen atom. Furthermore, substitution with a divalent substituent can also be performed by the removal of two hydrogen atoms.

In the description, unless otherwise particularly noted, “substitution” refers to substitution of 1, 2, or more hydrogen atoms in an organic compound or a substituent with other atoms or atomic groups. Substitution with a monovalent substituent can also be performed by the removal of one hydrogen atom. Furthermore, substitution with a divalent substituent can also be performed by the removal of two hydrogen atoms.

Examples of a substituent to be used in the present invention include, but are not limited to, alkyl, cycloalkyl, alkenyl, cycloalkenyl, alkynyl, alkoxy, carbocyclic group, heterocyclic group, halogen, hydroxy, thiol, cyano, nitro, amino, carboxy, carbamoyl, acyl, acylamino, thio carboxy, amido, substituted carbonyl, substituted thio carbonyl, substituted sulfonyl, and substituted sulfinyl. Such substituent can be adequately used when amino acids are designed in the present invention.

Preferably, a plurality of such substituents are present, they can be each independently a hydrogen atom or alkyl. However, it is not required that all of these plural number of substituents are hydrogen atoms. More preferably, when a plurality of such substituents are independently present, they can be each independently selected from the group consisting of hydrogen and C1 to C6 alkyl. All of these substituents may have substituents other than hydrogen. Preferably, these substituents can have at least one hydrogen, more preferably, 2 to n (here “n” denotes the number of substituent) hydrogens. Preferably, the number of hydrogen may be greater than that of substituents other than hydrogen. This is because a small substituent or a substituent with polarity can cause damage to the effects of the present invention (particularly, interaction with an aldehyde group). Therefore, substituents other than hydrogen can be preferably C1 to C6 alkyl, C1 to C5 alkyl, C1 to C4 alkyl, C1 to C3 alkyl, C1 to C2 alkyl, methyl, and the like. Furthermore, such a small substituent can also enhance the effects of the present invention, so that possession of a small substituent is also preferred herein.

In the description, “C1, C2 . . . , and Cn” denote the number of carbon. Therefore, C1 is used to denote a substituent having one carbon.

In the description, “protection reaction” refers to a reaction by which a protecting group such as Boc is added to a functional group desired to be protected. When a functional group is protected with a protecting group, the reaction of functional groups with higher reactivity can be suppressed and only the functional groups with lower reactivity can be caused to react. Such a protection reaction can be performed by dehydration reaction, for example.

In the description, “deprotection reaction” refers to a reaction by which a protecting group such as Boc is deprotected. An example of such a deprotection reaction is a reduction reaction using Pd/C. Deprotection reaction can be performed by hydrolysis, for example.

In the description, examples of typical “protecting group” include fluorenylmethoxycarbonyl (Fmoc) group, acetyl group, benzyl group, benzoyl group, t-butoxy carbonyl group, t-butyl dimethyl group, silyl group, trimethyl silyl ethyl group, N-phthalimidyl group, trimethyl silyl ethyloxy carbonyl group, 2-nitro-4,5-dimethoxy benzyl group, 2-nitro-4,5-dimethoxy benzyloxycarbonyl group, and carbamate group. Such a protecting group can be used to protect a reactive functional group such as an amino group or a carboxyl group, for example. Various protecting groups can be separately used according to conditions or purposes for reaction. An acetyl group, a benzyl group, a silyl group, or a derivative thereof can be used as a protecting group for a hydroxy group. In addition to an acetyl group, a benzyloxycarbonyl group, a t-butoxy carbonyl group, or a derivative thereof can be used as a protecting group for an amino group. As a protecting group for an amino oxy group or an N-alkylamino oxy group, a trimethyl silyl ethyloxy carbonyl group, a 2-nitro-4,5-dimethoxy benzyloxycarbonyl group, or a derivative thereof is preferred.

In each method of the present invention, a product to be generated can be isolated by removing foreign substances (e.g., unreacted raw materials, by-products, and solvents) from the reaction solution by a method that is generally used in the art (e.g., extraction, distillation, washing, condensation, precipitation, filtration, and drying) and then performing a combination of post-treatment methods that are generally used in the art (e.g., adsorption, elution, distillation, precipitation, deposition, and chromatography).

In the present invention, it is understood that an addition reaction of a sugar chain proceeds in principle as long as contact takes place. Preferably, for example, it is understood that such reaction proceeds at 25° C. to 80° C. Examples of the upper limit of appropriate temperatures include, but are not limited to, 80° C., 70° C., 60° C., 50° C., 42° C., and 40° C. Such a temperature varies depending on the type of a protein. The upper limit of a protein that is easily heat-denatured can be 37° C., for example. The lower limit of appropriate temperatures can be 25° C., 30° C., 32° C., 37° C., or the like. The lower limit of appropriate temperatures can be adequately determined by persons skilled in the art in connection with reaction speed and in view of necessary time.

Reaction time (time required for reaction) can also be adequately determined by persons skilled in the art based on the information contained in the description. Such reaction time ranges from 6 hours to 5 days, for example, but the example is not limited thereto.

Examples of the lower limit of such reaction time include, but are not limited to, several hours (e.g., 1 hour, 2 hours, 3 hours, 4 hours, 5 hours, and 6 hours), 1 day, and several days (2 to 3 days). Persons skilled in the art can adequately determine the reaction time by taking reaction speed, efficiency, and the like into consideration based on the information contained in the description. Examples of the upper limit of the reaction time include, but are not limited to, several days (2 to 3 days), 5 days, 6 days, and 10 days. It is desired to determine the upper limit of the reaction time so that the thus produced glycoprotein is not degraded or denatured.

### (Method for Producing Sugar-Chain-Modified Liposome)

In another aspect, the present invention provides a method for producing a sugar-chain-modified liposome. This method comprises the steps of: (a) providing a liposome; (b) hydrophilizing the liposome; (c) binding a linker to the hydrophilized liposome according to need, so as to generate a linker-bound liposome; and (d) binding a sugar chain to the liposome so as to generate a sugar-chain-modified liposome. Preferably, in this method, the step (b) of hydrophilizing a liposome is performed by binding directly or indirectly a low-molecular-weight hydrophilic compound (e.g., tris(hydroxyalkyl)aminoalkane) onto the lipid membrane of the liposome or the linker, the linker that is used in the step (c) is a human-derived protein (e.g., human serum albumin), and a sugar-chain-modified liposome is generated in the step (d) of binding a sugar chain under conditions where the sugar chain is directly or indirectly bound to the liposome.

In another aspect, the present invention provides a method for producing a sugar-chain-modified liposome for delivery of a drug to a target delivery site. This method comprises the steps of: (a) providing sugar-chain-modified liposomes varying in sugar chain density for achievement of delivery to a target delivery site; (b) determining the sugar chain density of a sugar-chain-modified liposome for achievement of optimum delivery to the delivery site; and (c) incorporating the drug into the thus determined optimum sugar-chain-modified liposome so as to generate a drug-containing liposome.

### (Production of Liposome)

A liposome itself can be produced according to a known method. Examples of such a method include a thin film method, a reverse phase evaporation method, an ethanol injection method, and a dehydration-rehydration method.

Moreover, the particle diameter of a liposome can also be regulated using an ultrasonic irradiation method, an extrusion method, a French press method, a homogenization method, or the like. A method for producing the liposome itself of the present invention is more specifically described as follows. For example, first, a lipid(s) compounded with phosphatidyl cholines, cholesterol, phosphatidyl ethanol amines, phosphatidic acids, gangliosides, glycolipids, or phosphatidyl glycerols as an ingredient is mixed with surfactant sodium cholate to prepare a mixed micelle. In particular, compounding with phosphatidic acids or long-chain alkyl phosphates such as dicetylphosphate is essential for negatively charging the liposome. Compounding with phosphatidyl ethanol amines is essential as a hydrophilic reaction site. Compounding with gangliosides or glycolipids or phosphatidyl glycerols is essential as a binding site of a linker. At least one type of lipid selected from the group consisting of gangliosides, glycolipids, phosphatidyl glycerols, sphingomyelins, and cholesterols assembles in the liposome and then functions as a foothold (raft) for binding the linker. The liposome of the present invention is further stabilized by the formation of such raft to which a protein can bind. Specifically, an example of the liposome of the present invention is a liposome in which a raft (for binding with a linker) of at least one type of lipid selected from the group consisting of ganglioside, glycolipid, phosphatidyl glycerols, sphingomyelins, and cholesterols. The thus obtained mixed micelle is subjected to ultrafiltration, so that a liposome is prepared. A general liposome can be used in the present invention and the surface of such a liposome is desirably hydrophilized in advance. After a liposome is prepared as described above, the liposome surface is hyrophilized.

The present invention further encompasses a liposome itself, to which a hydrophilized sugar chain (hydrophilized using the above hydrophilic compound) is not bound. Such a hydrophilized liposome has advantages such that it has enhanced stability of its own or the recognition ability of a sugar chain is improved when the sugar chain is bound. The liposome of the present invention is a liposome that contains a constitutive lipid of the liposome that is at least one or more types of lipid (mole percentage: 0% to 30%) selected from the group consisting of phosphatidyl cholines (mole percentage: 0% to 70%), phosphatidyl ethanol amines (mole percentage: 0% to 30%), phosphatidic acids, long-chain alkyl phosphate, and dicetylphosphates, at least one or more types of lipid (mole percentage: 0% to 40%) selected from the group consisting of gangliosides, glycolipids, phosphatidyl glycerols, and sphingomyelins, and cholesterols (mole percentage: 0% to 70%), for example.

The present invention further encompasses a method for hydrophilizing a liposome by further binding the above hydrophilic compound to a liposome so as to hydrophilize the liposome. Moreover, the present invention encompasses a hydrophilized liposome having no sugar chain bound thereto. The targeting liposome or the intestinal tract absorbable liposome of the present invention can be produced by binding a sugar chain to a liposome having no sugar chain bound thereto.

### (Synthesis of Sugar Chain)

A sugar chain that can be used for the sugar-chain-modified liposome of the present invention can be synthesized by a general method for synthesizing a sugar chain. Examples of such method include (1) a method that involves chemical synthesis, (2) a fermentation method using gene recombinant cells or microorganisms, (3) a synthesis method using glycosidase, (4) and a synthesis method using glycosyltransferase. (see WO2002/081723, JP Patent Publication (Kokai) No. 9-31095 A (1997), JP Patent Publication (Kokai) No. 11-42096 A (1999), JP Patent Publication (Kokai) No. 2004-180676 A, and Kenichi Hatanaka, Shinichiro Nishimura, Tatsuro Ouchi, and Kazukiyo Kobayashi (1997) Glycoscience and Glycotechnology (To-shisu no kagaku to kogyo), Kodansha Scientific Ltd., Tokyo, for example). A sugar chain that is used in the sugar-chain-modified liposome of the present invention may be a sugar chain that is synthesized by the above method or a commercially available sugar chain.

### (Binding of Sugar Chain to Liposome)

In the present invention, any one of the above sugar chains may be directly bound or indirectly bound via a linker to a liposome prepared as described above. At this time, the number of types of sugar chain to be bound to a liposome is not limited to one type and a plurality of sugar chains may also be bound. Such a plurality of sugar chains may have activity of binding to different cell surface molecules such as lectins that commonly exist on the cell surfaces of the same tissues or organs. Alternatively, such a plurality of sugar chains may have activity of binding to different cell surface molecules such as lectins that exist on the cell surfaces of the different tissues or organs. Through the selection of the former (plurality of) sugar chains, targeting to specific target tissues or organs can be ensured. Through the selection of the latter (plurality of) sugar chains, a single type of liposome can be caused to target to a plurality of targets so that a multi-purpose targeting liposome can be obtained.

In addition, to bind a sugar chain to a liposome, a linker and/or a sugar chain is mixed when a liposome is produced and then the sugar chain can be caused to bind to the surface of the liposome while producing the liposome. It is desired that a liposome, a linker, and a sugar chain are separately prepared in advance and the linker and/or the sugar chain is bound to the produced liposome (production is completed). This is because the density of a sugar chain to be bound can be controlled by causing a linker and/or a sugar chain to bind to the liposome. Direct binding of a sugar chain to a liposome can be performed by a method described below.

A liposome is produced by mixing a sugar chain as a glycolipid. Alternatively, a sugar chain is bound to a phospholipid of the thus produced liposome while controlling the sugar chain density. When a sugar chain is bound using a linker, a living body-derived protein, and particularly a human-derived protein is preferably used as a linker. Examples of such a living body-derived protein include, but are not limited to proteins existing in blood, such as albumin and other physiologically active substances existing in a living body. Examples of such proteins include serum albumins of animals such as human serum albumin (HSA) and bovine serum albumin (BSA). Particularly, when human serum albumin is used, it has been confirmed in experiments using mice that the amount of HSA incorporated in each tissue is high. The liposome of the present invention is very stable so that it makes it possible to perform posttreatment such as binding of a protein, binding of a linker, or binding of a sugar chain after liposome formation. Therefore, when a large amount of a liposome is produced, different proteins are bound or a linker or a sugar chain is bound according to purposes, so that various liposomes can be produced for different purposes.

A sugar chain is bound indirectly via a linker or directly to the constituent lipids of the liposome of the present invention. The liposome of the present invention has a glyoconjugate ligand such as a glycolipid or a glycoprotein and is hydrophilized with the use of a low-molecular-weight compound.

Furthermore, when the targeting liposome of the present invention is used as a medicine as described later, the liposome is required to contain a compound with medicinal effects. Such a compound having medicinal effects may be encapsulated in a liposome or bound to the liposome surface. A protein having medicinal effects may be used as a linker. In this case, a protein plays both roles of a linker for binding a sugar chain to a liposome and a protein having medicinal effects. An example of such a protein having medicinal effects is a physiologically active protein.

Binding of a sugar chain to a liposome via a linker may be performed by a method described below.

First, a protein is bound to the surface of a liposome. The liposome is treated with an oxidant such as NaIO₄, Pb(O₂CCH₃)₄, and NaBiO₃, so as to oxidize liposome ganglioside existing on the liposome membrane surface. Next, the linker and the ganglioside on the liposome membrane surface are bound by a reductive amination reaction using a reagent such as NaBH₃CN and NaBH₄. The linker is also preferably hydrophilized. For this purpose, a compound having a hydroxy group is bound to the linker protein. For example, with the use of a divalent reagent such as bissulfosuccinimidyl suberate, disuccinimidyl glutarate, dithiobissuccinimidyl propionate, disuccinimidyl suberate, 3,3′-dithiobissulfosuccinimidyl propionate, ethylene glycol bissuccinimidyl succinate, and ethylene glycol bissulfosuccinimidyl succinate, the above compound to be used for hydrophilization, such as tris(hydroxymethyl)aminomethane may be bound to the linker on the liposome.

This process is more specifically described below. First, one end of a cross-linking divalent reagent is bound to all the amino groups of the linker. Next, sugar chain glycosylamine compounds are prepared by performing glycosylamination (reaction) of the reducing terminus of each type of sugar chain. The amino group of each sugar chain is bound to the other unreacted terminus (corresponding to a part of) of the above-bound cross-linking divalent reagent on the liposome. A covalent bond between the sugar chain and/or hydrophilic compound and the liposome or a covalent bond between the sugar chain and/or hydrophilic compound and the linker may be cleaved when the liposome is incorporated into cells. For example, when a linker and a sugar chain are covalently bound via a disulfide bond, the sugar chain is cleaved as a result of intracellular reduction. As a result of cleavage of the sugar chain, the liposome surface becomes hydrophobic and binds to biomembrane, so that membrane stability is disturbed and a drug contained in the liposome is released.

Next, hydrophilization treatment is performed using most of the thus obtained unreacted termini of the divalent reagent (which have remained unreacted; that is, no sugar chain is bound thereto) which have remained on the surfaces of proteins on the sugar chain-bound liposome membrane surfaces. Specifically, a binding reaction is performed between unreacted termini of the divalent reagent binding to the proteins on the liposome and the above compound to be used for hydrophilization, such as tris(hydroxymethyl)aminomethane. Thus, the entire liposome surface is hydrophilized. Hydrophilization of a liposome surface and a linker improves transferability to various types of tissue, retentivity in blood, and transferability to various types of tissue. This may be caused by that portions other than sugar chains in each tissue or the like are observed as if they are in vivo water contents as a result of hydrophilization of the liposome surface and the linker surface, so that tissues and the like other than the target are not recognized and only the sugar chain is recognized by a cell surface molecule such as a lectin (sugar chain-recognizing protein) of the target tissue.

Subsequently, the sugar chain is bound to the linker on the liposome. For this, the reducing termini of saccharides composing the sugar chain are glycosylaminated using an ammonium salt such as NH₄HCO₃ and NH₂COONH₄. Next, with the use of a divalent reagent such as bissulfosuccinimidyl suberate, disuccinimidyl glutarate, dithiobissuccinimidyl propionate, disuccinimidyl suberate, 3,3′-dithiobissulfosuccinimidyl propionate, ethylene glycol bissuccinimidyl succinate, and ethylene glycol bissulfosuccinimidyl succinate, the linker bound onto the liposome membrane surface is bound to the above glycosylaminated saccharide, so that a liposome complex as shown in FIG. 1 is obtained. In addition, these sugar chains are commercially available.

The particle diameter of the liposome or that of the liposome bound with a sugar chain or the like of the present invention ranges from 30 nm to 500 nm, and preferably 50 nm to 350 nm. Furthermore, desirably the liposome of the present invention is negatively charged. If the liposome is negatively charged, its interaction with negatively charged cells in vivo can be avoided. The zeta potential of the liposome surface of the present invention ranges from −50 mV to 10 mV, preferably −40 mV to 0 mV, and further preferably −30 mV to −10 mV at 37° C. in a physiological saline solution.

Examples of a drug to be contained in the sugar-chain-modified liposome of the present invention include biological and pharmaceutical products or substances for biological therapy (e.g., an siRNA, an siRNA derivative, an RNA, an RNA derivative, a DNA, a DNA derivative, a monoclonal antibody, a vaccine, an interferon, a hormone, prostaglandin, a transcription factor, a recombinant protein, an antibody drug, a nucleic acid drug, and a gene therapeutic drug), alkylating anticancer agents, antimetabolites, plant-derived anticancer agents, anticancerous antibiotics, BRM-cytokines, drugs for tumors (e.g., a platinum complex anticancer agent, an immunotherapeutic agent, a hormone-based anticancer agent, and a monoclonal antibody), drugs for the central nerve, drugs for the peripheral nerve system-sense organs, drugs for treatment of respiratory diseases, drugs for circulatory organs, drugs for digestive organs, drugs for the hormone system, drugs for urinary organs-reproductive organs, vitamins-revitalizers, metabolic pharmaceutical products, antibiotics-chemotherapeutic drugs, drugs for examination, anti-inflammatory agents, drugs for eye diseases, drugs for the central nerve system, drugs for the autoimmune system, drugs for the circulatory system, drugs for lifestyle-related diseases (e.g., diabetes and hyperlipemia), adrenal cortex hormones, immunosuppresants, antimicrobial agents, antiviral agents, agents for suppressing vascularization, cytokines, chemokines, anti-cytokine antibodies, anti-chemokine antibodies, anti-cytokine-chemokine receptor antibodies, nucleic acid formulations relating to gene therapy (e.g., siRNA, shRNA, miRNA, smRNA, antisense RNA, ODN, or DNA), neuroprotective factors, antibody drugs, molecular target drugs, drugs for improving osteoporosis-bone metabolism, neuropeptides, and physiologically active peptides-proteins. Examples of a drug for tumors include: alkylating agents such as nitrogen mustard hydrochloride-N-oxide, cyclofosfamide, ifosfamide, busulfan, nimustine hydrochloride, mitobronitol, melphalan, dacarbazine, ranimustine, estramustine sodium phosphate; antimetabolites such as mercapto purine, thioinosine(mercapto purineriboside), methotrexate, enocitabine, cytarabine, ancitabine hydrochloride(cyclocytidine hydrochloride), fluorouracil, 5-FU, tegafur, doxifluridine, and carmofur; plant-derived anticancer agents such as alkaloids (e.g., etoposide, vinblastine sulfate, vincristine sulfate, vindesine sulfate, paclitaxel, taxol, irinotecan hydrochloride, nogitecan hydrochloride); anticancerous antibiotics such as actinomycin D, mitomycin C, chromomycinA3, bleomycin hydrochloride, bleomycin sulfate, peplomycin sulfate, daunorubicin hydrochloride, doxorubicin hydrochloride, aclarubicin hydrochloride (aclacinomycin A), pirarubicine hydrochloride, epirubicin hydrochloride, neocarzinostatin; and other examples including mitoxantrone hydrochloride, carboplatin, cisplatin, L-asparaginase, aceglatone, procarbazine hydrochloride, tamoxifen citrate, ubenimex, lentinan, sizofuran, medroxyprogesterone acetate, fosfestrol, mepitiostane, and epitiostanol. In the present invention, the above examples of the drug also include derivatives thereof.

When the above drug is contained in the liposome of the present invention, the liposome can be used for treating diseases such as cancer and inflammation. Here, “cancer” includes diseases due to all neoplasms such as tumor and leukemia. When such drug is contained in the sugar-chain-modified liposome of the present invention and then the liposome is administered, the drug is accumulated in a cancer or an inflammation site at a higher level than that in a case in which the drug alone is administered. Specifically, the drug contained in the liposome of the present invention can be accumulated at a level 2 or more times, preferably 5 or more times, further preferably 10 or more times, and particularly preferably 50 or more times greater than that in the case in which the drug alone is administered.

Furthermore, a compound having medicinal effects may be encapsulated in a liposome or bound to the surface of a liposome. For example, a protein can be bound to such a surface by the same method as the above method for binding a linker. Other compounds can also be bound by a known method using functional groups of the compounds. Moreover, encapsulation into a liposome can be performed by the following method. A known method may be used for encapsulating a drug and the like into a liposome. For example, a liposome is formed using a solution containing a drug and the like and a lipid(s) including phosphatidyl cholines, phosphatidyl ethanol amines, phosphatidic acids or long-chain alkyl phosphates, gangliosides, glycolipids or phosphatidyl glycerols and cholesterols. A drug and the like are then encapsulated into the liposome.

Therefore, a liposome formulation is obtained by encapsulating a drug or a gene that can be used for treatment or diagnosis into the liposome of the present invention. The liposome formulation has selectively-controlled transferability to cancer tissues, inflammatory tissues, and various types of tissue. The liposome formulation can enable enhanced effects of a therapeutic drug or a diagnostic agent via its accumulation in a concentrated manner in target cells and tissues, alleviated side effects due to reduced incorporation of the drug in other cells and tissues, or the like.

Moreover, when the drug delivery vehicle for intravenous injection and oral administration of the present invention is used for diagnosis, a labeling compound such as a fluorescent pigment or a radioactive compound is encapsulated in or bound to a liposome. The labeling compound-bound liposome binds to an affected part, the labeling compound is incorporated into the cells of the affected part, and then disease can be detected and/or diagnosed using the presence of the labeling compound as an indicator.

When the present invention is applied for use in diagnosis, for example, it can be applied for DNA probe diagnostic agents, X-ray contrast materials, radioactive reagents, radioactive contrast materials, radioactive diagnostic agents, fluorescent reagents, fluorescent contrast materials, fluorescent diagnostic agents, contrast materials for CT, contrast materials for PET, contrast materials for SPECT, contrast materials for MRI, diagnostic agents for AIDS, reagents for hematological tests, reagents for functional tests, reagents for microbial tests, molecular imaging, in vivo imaging, fluorescent imaging, luminescence imaging, cell sorters, PET and SPECT, and the like. Examples of a research reagent include reagents that are used in DNA recombination technology, an immunoassay, a hybridization method, and an enzyme assay.

For example, as a result of the present invention; that is, as demonstrated in Example 9, Example 10, Example 22A, and Example 22B, the sugar-chain-modified liposome highly effectively accumulates and delivers drugs, fluorescent substances, radioactively labeled substances, or the like in parts affected by diseases and various organs based on active targeting using the functions of the sugar chains as ligands. Therefore, the sugar-chain-modified liposome of the present invention makes it possible to visualize the accumulation in target tissues such as tumors. Thus, according to the present invention, in addition to the use of the liposome as a delivery vehicle for delivering a drug for treatment, a delivery vehicle to be used as a reagent for research or a diagnostic agent is also provided.

When the present invention is applied for use in diagnosis, for example, it can be applied for DNA probe diagnostic agents, X-ray contrast materials, radioactive diagnostic agents, fluorescent diagnostic agents, contrast materials for CT, contrast materials for PET, contrast materials for SPECT, contrast materials for MRI, diagnostic agents for AIDS, reagents for hematological tests, reagents for functional tests, reagents for microbial tests, molecular imaging, in vivo imaging, fluorescent imaging, luminescence imaging, cell sorters, PET and SPECT, and the like. Examples of a research reagent include reagents that are used in DNA recombination technology, an immunoassay, a hybridization method, and an enzyme assay.

### (Beauty and Makeup)

In another embodiment, an object of the present invention can be therapy, treatment, or improvement for beauty. Examples of such an object include not only beauty and makeup treatment performed for purely healthy subjects, but also beauty treatment performed for postsurgical or posttraumatic deformity or for congenital deformity. For example, the present invention can be used for tissue augmentation of breast (breast augmentation), tissue augmentation for the recesses of cheek or upper and lower eyelids, and tissue augmentation for facial hemiatrophy, tissue atrophy after facial paralysis, funnel chest, and the like. Furthermore, the present invention can also be used for nose job, reduction rhinoplasty, genioplasty (tissue augmentation), metopoplasty (tissue augmentation), and otoplasty (plasty for auricular cartilage) that is performed for malformed ears-malformation such asmicrotia, but the examples are not limited thereto.

When the present invention is used in the fields of beauty and makeup, such composition can further contain a pharmaceutically acceptable carrier and the like. Examples of such a pharmaceutically acceptable carrier that is contained in the medicine of the present invention include arbitrary substances known in the art.

Examples of such materials appropriate for prescription or pharmaceutically acceptable carriers include, but are not limited to, anti-oxidants, preservatives, coloring agents, flavoring agents, and diluents, emulsifiers, suspending agents, solvents, fillers, extending agents, buffers, delivery vehicles, diluents, excipients and/or pharmaceutical adjuvants.

An acceptable carrier, excipient, or stabilizing agent, which is used in the description is nontoxic for a recipient and is preferably inactive at the dosage and concentration to be employed herein. Examples of such carrier, excipient, or stabilizing agent include, but are not limited to: phosphate, citrate, or other organic acids; ascorbic acid and α-tocopherol; low-molecular-weight polypeptide; proteins (e.g., serum albumin, gelatin or immunoglobulin); hydrophilic polymers (e.g., polyvinyl pyrrolidone); amino acids (e.g., glycine, glutamine, asparagines, arginine, or lysine); monosaccharides, disaccharides, and other carbohydrates (including glucose, mannose, or dextrin); chelating agents (e.g., EDTA); sugar alcohols (e.g., mannitol or sorbitol); salt-forming counter ions (e.g., sodium); and/or nonionic surface active agents (e.g., Tween, pluronic, or polyethylene glycol (PEG)).

Examples of an appropriate carrier include a neutral buffered physiological saline solution and a physiological saline solution mixed with serum albumin. Preferably, the generated product is prescribed as a freeze-dried agent using an appropriate excipient (e.g., sucrose). Another standard carrier, a diluent, and an excipient can be contained if desired. Another example of a composition contains a Tris buffer (pH7.0 to 8.5) or an acetic acid buffer (pH4.0 to 5.5). Such an example can further contain sorbitol or an appropriate alternate thereof.

(Healthcare and-Food)

The present invention can also be applied in the fields of healthcare and foods. In such cases, points that should be remembered when it is applied for oral medicines as described above should be taken into consideration according to need. In particular, when the present invention is used for functional foods and/or health foods such as a food for specified health use, it is preferably treated according to the manner employed for medicines. Preferably, the sugar-chain-modified liposome of the present invention into which a functional food, a nutritional supplement, or a health supplement is encapsulated or bound can be used as a food composition. Such a functional food, nutritional supplement, or health supplement that can be used in the present invention is not limited. Examples of such food include any foods as long as they are designed, processed, and then converted so that the food functions are effectively expressed after intake.

Examples of such a functional food, nutritional supplement, or health supplement that can be used in the present invention include ginkgo leaves, echinacea, saw palmetto, ST John's Wort, valerian, black cohosh, milk thistle, evening primrose, grape seed extracts, Vaccinium myrtillus, feverfew, Angelica root, soybean, French maritime pine bark, garlic, Asian ginseng, tea, ginger, Agaricus, mesimakobu, purple ipe, AHCC, yeast beta-glucan, Grifola frondosa, propolis, brewer's yeast, cereals, Japanese plum, chlorella, young leaves of barley, green juice, vitamins, collagen, glucosamine, mulberry leaves, Rooibos tea, amino acid, royal jelly, shiitake mushroom (mycelium) extracts, spirulina, Denshichi ginseng, cress, plant fermentation foods, DHA, EPA, ARA, Laminaria japonica (kombu), cabbage, aloe, megusurinoki (paperbark maple), hop, oyster extracts, pycnogenol, and sesame. They may be directly contained in liposomes or treated products such as extracts or the like obtained therefrom may also be contained. A food composition containing a liposome is orally ingested. A liposome to be used herein may be a liposome to which no sugar chain is bound or a liposome to which a sugar chain for enhancing intraintestinal absorption or a sugar chain targeting a specific tissue or organ is bound. When the liposome of the present invention is administered as a food composition, the liposome may be processed into a food such as a liquid beverage, a gelled food, or a solid food. The liposome may also be processed into tablets, granules, or the like. The food composition of the present invention can be used as a functional food, a nutritional supplement, or a health supplement according to the types of food in which the liposome is contained. For example, such foods may contain vitamins, minerals, amino acids, and carbohydrates.

For example a liposome containing DHA can be used as a functional food, a nutritional supplement, or a health supplement effective for mild senile dementia or memory improvement.

### (Production Apparatus)

In another aspect, the present invention provides an apparatus for producing a delivery vehicle for achieving delivery to a desired site. The apparatus is provided with:

A) a means for measuring in vitro affinity of candidate delivery vehicles for a cell surface molecule such as a lectin associated with the site; and
B) a means for selecting a delivery vehicle having in vitro affinity corresponding to delivery to the desired site.

Alternatively, the present invention provides an apparatus for producing a delivery vehicle for achieving delivery to a desired site. This apparatus is provided with:

A) a means for measuring in vitro affinity of candidate delivery vehicles for a cell surface molecule such as a lectin associated with the site;
B) a means for selecting a delivery vehicle having in vitro affinity corresponding to delivery to the desired site.
C) a means for analyzing the composition of the selected delivery vehicle; and
D) a means for generating the selected delivery vehicle based on the composition.

### (Use)

In another aspect, the present invention provides the use of in vitro affinity for a cell surface molecule such as a lectin associated with a desired site, for production of a delivery vehicle for achieving delivery to the desired site. Such in vitro affinity can be used for various purposes. For example, such in vitro affinity can be used not only for therapeutic drugs, but also for diagnostic agents (e.g., a contrast material for MRI), research reagents (e.g., a fluorescent probe), cosmetics, and functional foods. Furthermore, such in vitro affinity can also be used not only for products in the agricultural, medical, and pharmaceutical fields (including pharmaceutical products, cosmetic-agricultural chemical-foods), but also for reagents for assay. Such concept has been absent before disclosure of the present invention. Hence, the significance of such concept is of great significance.

References such as scientific publications, patents, and patent applications cited herein are incorporated herein by reference in their entirety to a degree such that each reference is specifically described.

As described above, the present invention is illustrated using the preferred embodiments of the present invention. However, the present invention should not be construed in a manner limited to these embodiments. It is understood that the scope of the present invention should be construed based only on the claims. It is understood that persons skilled in the art can implement the present invention within a scope equivalent to that based on specific descriptions of the preferred embodiments of the present invention and technical commonsense. It is understood that all patents, patent applications, and publications cited herein are incorporated herein by reference in their entirety to a degree such that the contents thereof are specifically described herein.

### EXAMPLES

The constitution of the present invention will be further described below specifically with reference to examples. However, the present invention is not limited by the following examples. Reagents used in the following examples were commercially available reagents, unless otherwise particularly noted.

### Example 1

### Preparation of Liposome

A liposome was prepared by the techniques of the previous report (Yamazaki, N., Kodama, M. and Gabius, H.-J. (1994) Methods Enzymol. 242, 56-65) using improved cholic acid dialysis. Specifically, dipalmitoylphosphatidylcholine, cholesterol, dicetylphosphate, ganglioside, and dipalmitoylphosphatidylethanol amine were mixed at a molar ratio of 35:40:5:15:5 so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, thereby obtaining a lipid membrane. The thus obtained lipid membrane was suspended in 3 ml of a TAPS buffer solution (pH 8.4) and then the resultant was ultrasonicated, so that a transparent micelle suspension was obtained. Furthermore, the micelle suspension was subjected to ultrafiltration using a PM10 membrane (Amicon Co., U.S.A.) and a phosphate buffer (pH 7.2, Phosphate Buffered Saline (PBS): Na₂HPO₄ (25.55 g)/KH₂PO₄ (2.72 g)/NaN₃ (0.8 g)/NaCl (35.4 g)). Thus, 10 ml of homogeneous liposome (average particle diameter: 100 nm) was prepared.

### Example 2

### Hydrophilization of Liposome Lipid Membrane Surface

10 ml of the liposome solution prepared in Example 1 was subjected to ultrafiltration using an XM300 membrane (Amicon Co., U.S.A.) and a CBS buffer solution (pH 8.5) and the pH of the solution was adjusted to pH 8.5. Next, 10 ml of a cross-linking reagent bis(sulfosuccinimidyl)suberate (BS3; Pierce Co., U.S.A.) was added, followed by 2 hours of agitation at 25° C. Subsequently, the solution was further agitated overnight at 7° C. so as to complete the chemical binding reaction between lipid dipalmitoylphosphatidylethanol amine on the liposome membrane and BS3. The liposome solution was then subjected to ultrafiltration using an XM300 membrane and a CBS buffer solution (pH 8.5). Next, 40 mg of tris(hydroxymethyl)aminomethane dissolved in 1 ml of a CBS buffer solution (pH 8.5) was added to 10 ml of the liposome solution, followed by 2 hours of agitation at 25° C. The solution was then agitated overnight at 7° C., so as to complete the chemical binding reaction between BS3 bound to the lipids on the liposome membrane and tris(hydroxymethyl)aminomethane. Thus, the hydroxy group of tris(hydroxymethyl)aminomethane was coordinated on the lipid dipalmitoylphosphatidylethanol amine of the liposome membrane, so that the liposome membrane surface was hydrated and hydrophilized.

### Example 3

### Binding of Human Serum Albumin (HSA) onto Liposome Membrane Surface

Human serum albumin (HSA) was bound onto a liposome membrane surface according to the technique of the previous report (Yamazaki, N., Kodama, M. and Gabius, H.-J. (1994) Methods Enzymol. 242, 56-65) using a coupling reaction method. Specifically, the reaction was performed as a two-step chemical reaction. First, ganglioside existing on the membrane surface of 10 ml of the liposome obtained in Example 2 was added to 43 mg of sodium metaperiodate dissolved in 1 ml of a TAPS buffer solution (pH8.4), followed by 2 hours of agitation at room temperature to perform periodate oxidation. The resultant was subjected to ultrafiltration using an XM300 membrane and a PBS buffer solution (pH8.0) so that 10 ml of the thus oxidized liposome was obtained. 20 mg of human serum albumin (HSA) was added to the liposome solution, followed by 2 hours of agitation at 25° C. Next, 100 μl of 2M NaBH₃CN was added to PBS (pH8.0) and then the solution was agitated overnight at 10° C. Thus, HSA was bound by the coupling reaction between ganglioside on the liposome and HSA. The resultant was then subjected to ultrafiltration using an XM300 membrane and a CBS buffer solution (pH8.5), so that 10 ml of an HSA-bound liposome solution was obtained.

### Example 4

### Preparation of Sugar Chain

Sugar chains listed in Table 4 below were used.

The mass of each sugar chain was measured and then pretreated for use in the following Example 5. When a combination of two or more sugar chains was used, these sugar chains were mixed with each other.

TABLE 14

| | Abbreviated | | | |
|---|---|---|---|---|
| No. | name | English name | Manufacture name | Product No. |
| 3 | EEGD1A-BH | Ganglioside GD1a | CALBIOCHEM | 345736 |
| 27 | LDF-5 | Lactodifucotetraose | Seikagaku Corporation | 420217 |
| 29 | 3SL-5 | 3′-Sialyllactose | Seikagaku Corporation | 420252 |
| 37 | FEEGD1b | Ganglioside GD1b | CALBIOCHEM | 345751 |
| 38 | G4GN-4 | N-Acetyllactosamine | CALBIOCHEM | 345250 |
| 40 | BAT-1 | Blood Group A Trisaccharide | CALBIOCHEM | 202758 |
| 41 | SOLX-4 | 3′-O-Sulfonato Lewis x Trisaccharide | CALBIOCHEM | 574218 |
| 45 | BHD-1 | Blood Group H Disaccharide | CALBIOCHEM | 202773 |
| 50 | ManB | Oligomannose-8 | Seikagaku Corporation | 420289 |
| 53 | L2F-1 | Lacto-N-Fucopentaose I | Seikagaku Corporation | 420221 |
| 56 | Man9 | Oligomannose-9 | Seikagaku Corporation | 420290 |
| 60 | 6SLN-1 | 6′-Sialyllactosamine | Seikagaku Corporation | 420251 |
| 67 | FEEaMDa | Asialo-Ganglioside GM1 + | CALBIOCHEM | 345747 |
| | | Ganglioside GD1a | CALBIOCHEM | 345736 |
| 68 | Man7 | Oligomannose-7 | Seikagaku Corporation | 420288 |
| 69 | L4F-1 | Lacto-N-Fucopentaose II | Seikagaku Corporation | 420386 |
| 70 | LNT-5 | Lacto-N-tetraose, | Seikagaku Corporation | 420234 |
| 71 | BBT-1 | Blood Group B Trisaccharide | CALBIOCHEM | 202758 |
| 76 | BHD-4 | Blood Group H Disaccharide | CALBIOCHEM | 202773 |
| 80 | 6SLN-5 | 6′-Sialyllactosamine | Seikagaku Corporation | 420251 |
| 87 | TA-1 | T Antigen | CALBIOCHEM | 575300 |
| 91 | G6GN-4 | Galactoseβ1,6-N-Acetylglucosamine | CALBIOCHEM | 345510 |
| 93 | A36 | α1-3,α1-6Mannotriose | Funakoshi (DEXITRA | M336DL1233-60 |
| | | | LABORATORIES, LTD.) | |
| 96 | TA-4 | T Antigen | CALBIOCHEM | 575300 |
| 105 | Man6 | Oligomannose-6 | Seikagaku Corporation | 420287 |
| 106 | LDF-4 | Lactodifucotetraose | Seikagaku Corporation | 420217 |
| 111 | LNB-5 | Lacto-N-blose I | CALBIOCHEM | 427313 |
| 116 | LDF-1 | Lactodifucotetraose | Seikagaku Corporation | 420217 |
| 117 | TA-5 | T Antigen | CALBIOCHEM | 575300 |
| 120 | A2 | α1-2Mannobiose | Funakoshi (DEXITRA | M202DL-1220-21 |
| | | | LABORATORIES, LTD.) | |
| 125 | LX-1 | Lewis x Trisaccharide | CALBIOCHEM | 434630 |
| 127 | G3G-4 | Galactoseα1,3-Galactose | CALBIOCHEM | 345520 |
| 129 | BBT-4 | Blood Group B Trisaccharide | CALBIOCHEM | 202758 |
| 130 | 2FL-4 | 2′-Fucosyllactose | CALBIOCHEM | 343328 |
| 137 | Man4B | Oligomannose-4(b) | Seikagaku Corporation | 420290 |
| 139 | 3FL-5 | 3-Fucosyllactose | CALBIOCHEM | 343329 |
| 141 | BBT-5 | Blood Group B Trisaccharide | CALBIOCHEM | 202758 |
| 142 | BHD-5 | Blood Group H Disaccharide | CALBIOCHEM | 202773 |
| 146 | 3FL-4 | 3-Fucosyllactose | CALBIOCHEM | 343329 |
| 150 | TNA-5 | N-Acetylgalactosamineα1-O-Serine | CALBIOCHEM | 110153 |
| 151 | SLX-4 | Sialyl Lewis x Tetrasaccharide | CALBIOCHEM | 565950 |
| 152 | LY-1 | Lewis y Tetrasaccharide | CALBIOCHEM | 434634 |
| 153 | G6GN-5 | Galactoseβ1,6-N-Acetylglucosamine | CALBIOCHEM | 345510 |
| 154 | Lac-5 | Lactose, Monnohydrate | CALBIOCHEM | 427502 |
| 155 | A6 | α1-6Mannobiose | Funakoshi (DEXITRA | DL-1220-60(M206) |
| | | | LABORATORIES, LTD.) | |
| 175 | G3G-5 | Galactoseα1,3-Galactose | CALBIOCHEM | 345520 |
| 178 | LNT-4 | Lacto-N-tetraose | Seikagaku Corporation | 420234 |
| 183 | G4GN-5 | N-Acetyllactosamine | CALBIOCHEM | 345250 |
| 184 | STNA-1 | STNEpitope | CALBIOCHEM | 750199 |
| 186 | Man3 | Oligomannose-3 | Seikagaku Corporation | 420283 |
| 191 | LB-1 | Lewis b Tetrasaccharide | CALBIOCHEM | 434628 |
| 195 | TNA-4 | N-Acetylgalactosamineα1-O-Serine | CALBIOCHEM | 110153 |
| 197 | A4 | α1-4Mannobiose | Funakoshi (DEXITRA | DL-1220-40(M204) |
| | | | LABORATORIES, LTD.) | |
| 199 | STNA-4 | STNEpitope | CALBIOCHEM | 750199 |
| 204 | Lac-1 | Lactose, Monnohydrate | CALBIOCHEM | 427502 |
| 207 | 6SL-5 | 6′-Sialyllactose | Seikagaku Corporation | 420253 |
| 209 | LDFH-4 | Lacto-N-Difucohexaose I | Seikagaku Corporation | 420212 |
| 213 | LX-4 | Lewis x Trisaccharide | CALBIOCHEM | 434630 |
| 218 | EEGM1-BH | Ganglioside GM1 | CALBIOCHEM | 345724 |
| 220 | LNB-4 | Lacto-N-biose I | CALBIOCHEM | 427313 |
| 224 | Man5 | Oligomannose-5 | Seikagaku Corporation | 420286 |
| 225 | 3FL-1 | 3-Fucosyllactose | CALBIOCHEM | 343329 |
| 229 | G6GN-1 | Galactoseβ1,6-N-Acetylglucosamine | CALBIOCHEM | 345510 |
| 230 | G4GN-1 | N-Acetyllactosamine | CALBIOCHEM | 345250 |
| 233 | 2FL-1 | 2′-Fucosyllactose | CALBIOCHEM | 343328 |
| 234 | TNA-1 | N-Acetylgalactosamineα1-O-Serine | CALBIOCHEM | 110153 |
| 235 | Lac-4 | Lactose, Monnohydrate | CALBIOCHEM | 427502 |
| 236 | 2FL-5 | 2′-Fucosyllactose | CALBIOCHEM | 343328 |
| 237 | A3 | α1-3Mannobiose | Funakoshi (DEXITRA | DL-1220-30(M203) |
| | | | LABORATORIES, LTD.) | |
| 239 | SLA-4 | 3-Sialyl Lewis a Tetrasaccharide | CALBIOCHEM | 565940 |
| 240 | SOLA-5 | 3′-O-Sulfonato Lewis a Trisaccharide | CALBIOCHEM | 574210 |
| 249 | 3SL-1 | 3′-Sialyllactose | Seikagaku Corporation | 420252 |
| 254 | G3G-1 | Galactoseα1,3-Galactose | CALBIOCHEM | 345520 |
| 263 | 3SLN-1 | 3′-Sialyllactosamine | Seikagaku Corporation | 420250 |
| 273 | BAT-5 | Blood Group A Trisaccharide | CALBIOCHEM | 202758 |
| 285 | SLX-5 | Sialyl Lewis x | CALBIOCHEM | 565950 |
| 288 | LA-1 | Lewis a Trisaccharide | CALBIOCHEM | 434626 |
| 290 | SLA-5 | 3-Sialyl Lewis a Tetrasaccharide | CALBIOCHEM | 565940 |
| 292 | STNA-5 | STNEpitope | CALBIOCHEM | 750199 |
| 295 | 6SLN-4 | 6′-Sialyllactosamine | Seikagaku Corporation | 420251 |
| 301 | TRIS | tris(hydroxymethyl)aminomethane | SIGMA | 30-5000-2-25G-J |

### Example 5

### Binding of Sugar Chain onto Liposome Membrane-Surface-Bound Human Serum Albumin (Hsa)

50 μg of each sugar chain prepared in Example 4 was added to 0.5 ml of an aqueous solution in which 0.25 g of NH₄HCO₃ had been dissolved, followed by 3 days of agitation at 37° C. The resultant was filtered with a 0.45-μm filter to complete the amination reaction of the reducing termini of the sugar chains. Thus, 50 μg of a glycosylamine compound of each sugar chain was obtained. Next, 1 mg of a cross-linking reagent 3,3′-dithiobis(sulfo succinimidyl propionate (DTSSP; Pierce Co., U.S.A.) was added to 1 ml of the liposome solution (a portion of the liposome solution) obtained in Example 3. The solution was agitated for 2 hours at 25° C. and then agitated overnight at 7° C. The resultant was subjected to ultrafiltration using an XM300 membrane and a CBS buffer solution (pH8.5), so that 1 ml of liposome was obtained on which DTSSP was bound to HSA on the liposome. Next, 50 μg of the above glycosylamine compound was added to the liposome solution. The solution was agitated for 2 hours at 25° C. and then agitated overnight at 7° C. The resultant was subjected to ultrafiltration using an XM300 membrane and a PBS buffer solution (pH7.2) so as to bind the glycosylated amine compound to DTSSP on the liposome membrane-surface-bound human serum albumin. As a result, as listed in Table 2, liposomes (2 ml each) (total amount of lipid: 2 mg, total amount of protein: 200 μg, and average particle diameter: 100 nm) were obtained, each of which is prepared by binding of a sugar chain and human serum albumin, and the liposome.

Table 10 below shows the results of binding of each sugar chain onto liposome membrane-surface-bound human serum albumin (HSA). Unless otherwise clearly specified, binding of these sugar chains onto liposome membrane-surface-bound human serum albumin was performed with a method and the conditions similar to those in Example 5.

TABLE 15

| | | Total | Total | Average |
|---|---|---|---|---|
| | | lipid | protein | particle |
| Liposome | Amount of sugar | amount | amount | diameter |
| No. | chain used (mg) | (mg) | (mg) | (μm) |
| 3 | 1.5 | 2 | 0.2 | 100 |
| 27 | 1 | 2 | 0.2 | 100 |
| 29 | 1 | 2 | 0.2 | 100 |
| 37 | 1.5 | 2 | 0.2 | 100 |
| 38 | 0.5 | 2 | 0.2 | 100 |
| 40 | 0.05 | 2 | 0.2 | 100 |
| 41 | 0.5 | 2 | 0.2 | 100 |
| 45 | 0.05 | 2 | 0.2 | 100 |
| 50 | 0.077 | 2 | 0.2 | 100 |
| 53 | 0.05 | 2 | 0.2 | 100 |
| 58 | 0.084 | 2 | 0.2 | 100 |
| 60 | 0.05 | 2 | 0.2 | 100 |
| 67 | aGM1 + GD1A0.75 each | 2 | 0.2 | 100 |
| 68 | 0.07 | 2 | 0.2 | 100 |
| 69 | 0.05 | 2 | 0.2 | 100 |
| 70 | 1 | 2 | 0.2 | 100 |
| 71 | 0.05 | 2 | 0.2 | 100 |
| 76 | 0.5 | 2 | 0.2 | 100 |
| 80 | 1 | 2 | 0.2 | 100 |
| 87 | 0.05 | 2 | 0.2 | 100 |
| 91 | 0.5 | 2 | 0.2 | 100 |
| 93 | 0.022 | 2 | 0.2 | 100 |
| 96 | 0.5 | 2 | 0.2 | 100 |
| 105 | 0.082 | 2 | 0.2 | 100 |
| 108 | 0.5 | 2 | 0.2 | 100 |
| 111 | 1 | 2 | 0.2 | 100 |
| 116 | 0.05 | 2 | 0.2 | 100 |
| 117 | 1 | 2 | 0.2 | 100 |
| 120 | 0.015 | 2 | 0.2 | 100 |
| 125 | 0.05 | 2 | 0.2 | 100 |
| 127 | 0.5 | 2 | 0.2 | 100 |
| 129 | 0.5 | 2 | 0.2 | 100 |
| 130 | 0.5 | 2 | 0.2 | 100 |
| 137 | 0.048 | 2 | 0.2 | 100 |
| 139 | 1 | 2 | 0.2 | 100 |
| 141 | 1 | 2 | 0.2 | 100 |
| 142 | 1 | 2 | 0.2 | 100 |
| 148 | 0.5 | 2 | 0.2 | 100 |
| 150 | 1 | 2 | 0.2 | 100 |
| 151 | 0.5 | 2 | 0.2 | 100 |
| 152 | 0.05 | 2 | 0.2 | 100 |
| 153 | 1 | 2 | 0.2 | 100 |
| 154 | 1 | 2 | 0.2 | 100 |
| 155 | 0.015 | 2 | 0.2 | 100 |
| 175 | 1 | 2 | 0.2 | 100 |
| 178 | 0.5 | 2 | 0.2 | 100 |
| 183 | 1 | 2 | 0.2 | 100 |
| 184 | 0.05 | 2 | 0.2 | 100 |
| 188 | 0.041 | 2 | 0.2 | 100 |
| 191 | 0.05 | 2 | 0.2 | 100 |
| 195 | 0.5 | 2 | 0.2 | 100 |
| 197 | 0.015 | 2 | 0.2 | 100 |
| 199 | 0.5 | 2 | 0.2 | 100 |
| 204 | 0.05 | 2 | 0.2 | 100 |
| 207 | 1 | 2 | 0.2 | 100 |
| 209 | 0.5 | 2 | 0.2 | 100 |
| 213 | 0.5 | 2 | 0.2 | 100 |
| 218 | 1.5 | 2 | 0.2 | 100 |
| 220 | 0.5 | 2 | 0.2 | 100 |
| 224 | 0.055 | 2 | 0.2 | 100 |
| 225 | 0.05 | 2 | 0.2 | 100 |
| 229 | 0.05 | 2 | 0.2 | 100 |
| 230 | 0.05 | 2 | 0.2 | 100 |
| 233 | 0.05 | 2 | 0.2 | 100 |
| 234 | 0.05 | 2 | 0.2 | 100 |
| 235 | 0.5 | 2 | 0.2 | 100 |
| 236 | 1 | 2 | 0.2 | 100 |
| 237 | 0.015 | 2 | 0.2 | 100 |
| 239 | 0.5 | 2 | 0.2 | 100 |
| 240 | 1 | 2 | 0.2 | 100 |
| 249 | 0.05 | 2 | 0.2 | 100 |
| 254 | 0.05 | 2 | 0.2 | 100 |
| 263 | 0.05 | 2 | 0.2 | 100 |
| 273 | 1 | 2 | 0.2 | 100 |
| 285 | 1 | 2 | 0.2 | 100 |
| 288 | 0.05 | 2 | 0.2 | 100 |
| 290 | 1 | 2 | 0.2 | 100 |
| 292 | 1 | 2 | 0.2 | 100 |
| 295 | 0.5 | 2 | 0.2 | 100 |

### Preparative Example 1

### Binding of Tris(Hydroxymethyl)Aminomethane onto Liposome Membrane-Surface-Bound Human Serum Albumin (HSA)

To prepare a liposome as a sample for comparison, 1 mg of a cross-linking reagent 3,3′-dithiobis(sulfosuccinimidyl propionate (DTSSP; Pierce Co., U.S.A.) was added to 1 ml of the liposome solution (a portion of the liposome solution) obtained in Example 3. The solution was agitated for 2 hours at 25° C. and then agitated overnight at 7° C. The resultant was subjected to ultrafiltration using an XM300 membrane and a CBS buffer solution (pH8.5), so that 1 ml of liposome in which DTSSP was bound to HSA on the liposome was obtained. Next, 13 mg of tris(hydroxymethyl)aminomethane (Wako Co., Japan) was added to the liposome solution. The solution was agitated for 2 hours at 25° C. and then agitated overnight at 7° C. The resultant was subjected to ultrafiltration using an XM300 membrane and a PBS buffer solution (pH7.2) so as to bind tris(hydroxymethyl)aminomethane to DTSSP on the liposome membrane-surface-bound human serum albumin. Because of the presence of 13 mg of tris(hydroxymethyl)aminomethane, which was already an extremely excessive amount in this step, hydrophilization of the liposome membrane-surface-bound human serum albumin (HSA) was also completed simultaneously. As a result, the final product, 2 ml of liposome (abbreviated name: TRIS) (total amount of lipid; 2 mg, total amount of protein: 200 μg, and average particle diameter: 100 nm) as a sample for comparison was obtained via binding of hydrophilized tris(hydroxymethyl)aminomethane, human serum albumin, and the liposome.

### Preparative Example 2

### Preparation of Glycolipid Liposome

Glycolipid liposomes (that begins with FEE or EE, such as liposome No. 3, liposome No. 37, liposome No. 67, and liposome No. 218) were prepared as follows.

Liposomes were prepared using cholic acid dialysis. Specifically, dipalmitoylphosphatidylcholine, cholesterol, dicetylphosphate, ganglioside (containing 100% GM1 as a glycolipid sugar chain), and dipalmitoylphosphatidylethanol amine were mixed at a molar ratio of 35:40:5:15:5 so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, so that a lipid membrane was obtained. The thus obtained lipid membrane was suspended in 3 ml of a TAPS buffer solution (pH8.4) and then the suspension was ultrasonicated. Thus, 3 ml of a transparent micelle suspension was obtained. A PBS buffer solution (pH7.2) was added to the micelle suspension to a total amount of 10 ml. The micelle suspension was subjected to ultrafiltration using a PM10 membrane (Amicon Co., U.S.A.) and a TAPS buffer solution (pH8.4). Thus, 10 ml of a homogeneous suspension of unhydrophilized liposome particles was prepared. 5 mg of Bolton-Hunter Reagent (BHR; Pierce Co., U.S.A.) was added to the liposome solution, so as to perform 2 hours of reaction at 25° C. and then to perform 4 hours of reaction at 7° C. Dipalmitoylphosphatidylethanol amine was thus modified with BH and then the resultant was subjected to ultrafiltration using a PBS buffer solution (PH7.2). As a result, 10 ml of liposome (abbreviated name: EEGM1-BH) (total amount of lipid: 45.6 mg and average particle diameter: 100 nm) was obtained as a sample for comparison.

### Example 6

### Hydrophilization of Liposome Membrane-Surface-Bound Human Serum Albumin (HSA)

Sugar chain-bound liposomes prepared by the means of Example 5 were separately subjected to hydrophilization of the HSA protein surfaces on the liposomes as described in the following procedures. 13 mg of tris(hydroxymethyl)aminomethane was added to 2 ml of each sugar chain-bound liposome. The solution was agitated for 2 hours at 25° C. and then agitated overnight at 7° C. The resultant was subjected to ultrafiltration using an XM300 membrane and a PBS buffer solution (pH7.2) and then unreacted substances were removed. As a result, the final product, 2 ml of each hydrophilized sugar chain-bound liposome complex (total amount of lipid; 2 mg, total amount of protein: 200 μg, and average particle diameter: 100 nm) was obtained.

### Example 7

### Determination of Inhibitory Effect of Various Sugar Chain-Bound Liposome Complexes on Lectin Binding Activity

The in vitro binding activity of each sugar chain-bound liposome complex (prepared by the means of Example 5 and Example 6); that is, the activity of binding to a cell surface molecule such as a lectin was determined in an inhibition experiment using a lectin-immobilized microplate according to a standard method (Yamazaki, N. (1999) Drug Delivery System, 14, 498-505). Specifically, a lectin (e.g., E-selectin; R&D Systems Co., U.S.A.; the lectin can be varied according to target organs) was immobilized on a 96-well microplate. 0.1 μg of biotinylated fucosylated fetuin (ligand for comparison) and each of various sugar chain-bound liposome complexes (amounts of protein: 0.01 μg, 0.04 μg, 0.11 μg, 0.33 μg, and 1 μg) varying in concentration were added to the lectin-immobilized plate, followed by 2 hours of incubation at 4° C. After 3 times of washing with PBS (pH7.2), horseradish peroxidase (HRPO)-conjugated streptavidin was added to the resultants. Incubation was further performed at 4° C. for 1 hour and then the resultants were washed 3 times with PBS (pH7.2). A peroxidase substrate was added, the resultants were allowed to stand at room temperature, and then absorbance was measured at 405 nm using a microplate reader (Molecular Devices Corp., U.S.A.). Fucosylated fetuin was biotinylated as follows. Treatment was performed with a sulfo-NHS-biotin reagent (Pierce Co., U.S.A.) and then purification was performed with Centricon-30 (Amicon Co., U.S.A.). HRPO-conjugated streptavidin was prepared by oxidation of HRPO and conjugation of streptavidin via reductive amination using NaBH₃CN. The determination results were subjected to the following treatment and calculation.

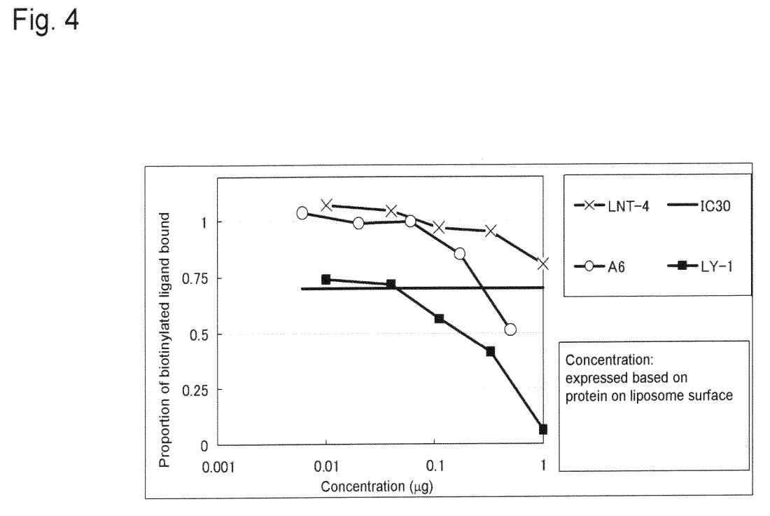

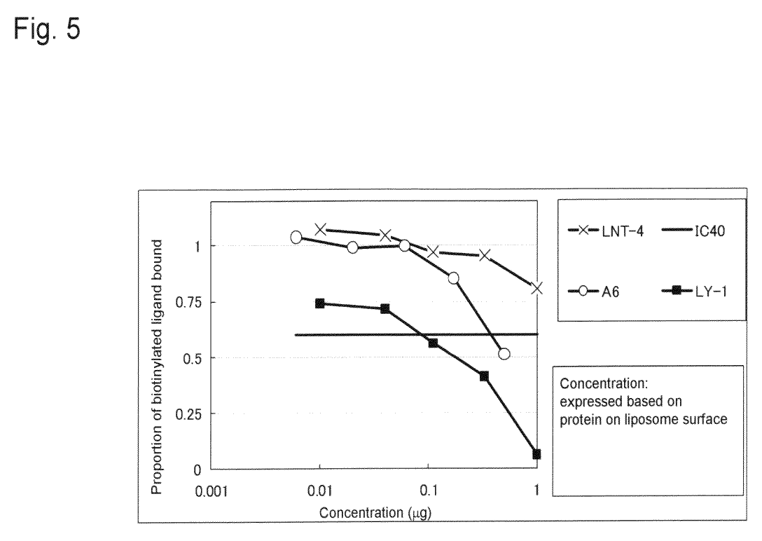

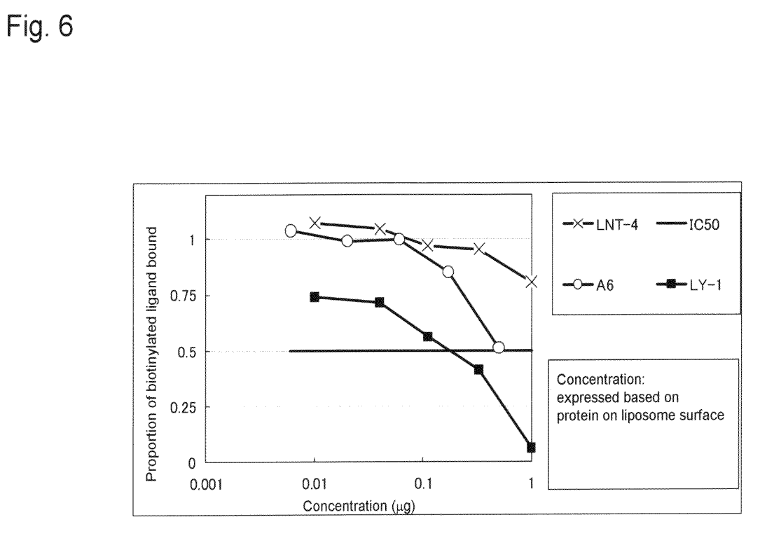

FIGS. 3 to 6 are schematic diagrams as to IC20 to IC50. FIG. 2 shows Graph 1 showing the results of an experiment conducted for Sample LY-1 and IC50.

The graph shown in FIG. 2 is a graph of sample LY-1 and IC50 series produced based on Table 16 and Table 17.

TABLE 16
Values of sample LY-1 and IC50 at each concentration

| | Concentration | | | | |
|---|---|---|---|---|---|
| | 0.01 | 0.04 | 0.11 | 0.33 | 1 |
| Average value of sample LY-1 | 0.144 | 0.142 | 0.126 | 0.110 | 0.073 |
| Ratio of average value of sample | 0.739 | 0.715 | 0.562 | 0.414 | 0.060 |
| LY-1* | | | | | |
| Ratio of y coordinate of IC50 series | 0.500 | 0.500 | 0.500 | 0.500 | 0.500 |

*“Ratio of average value of sample LY-1” or “Ratio of y coordinate of IC50 series” is the ratio of the same with respect to 1 (a difference between a “hot” value and a “cold” value of Control is determined to be “1.”)

TABLE 17
Control

| Hot | Cold |
|---|---|
| 0.171 | 0.067 |

Graph 1 was produced based on Table 1. X axis is expressed using a logarithmic scale. Each point on the line graph represents the ratio of the average value (of values measured at each concentration (horizontal axis)) of sample LY-1. The control value differs depending on samples. To facilitate comparison, the ratios (with respect to 1 (a difference between a “hot” value and a “cold” value of Control is determined to be “1.”)) are plotted on the longitudinal axis of the graph. The X coordinate of the intersection point of the Sample LY-1 graph and the IC50 series graph is the value of IC50. The intersection point is present on a line containing coordinate 1 (0.11, 0.562) and coordinate 2 (0.33, 0.414) and is represented by the formula: y=−0.673x+0.636. In the case of y=0.5 (the formula of IC50 series), X coordinate of the intersection point of two lines is 0.202. This value is divided by 69000 (the molecular weight of protein) and then the product is further divided by 300 (the number of protein per liposome). The result is 9.76E-09.

The thus obtained results (data) are as shown below.

TABLE 18A

| Sample ID | IC10 | Sample ID | IC10 |
|---|---|---|---|
| 76 | <4.83E−10 | 224 | 2.97E−10 |
| 45 | <4.83E−10 | 105 | 4.81E−10 |
| 127 | <4.83E−10 | 71 | 5.99E−10 |
| 273 | <4.83E−10 | 137 | 6.02E−10 |
| 295 | <4.83E−10 | 186 | 8.41E−10 |
| 80 | <4.83E−10 | 93 | 9.73E−10 |
| 263 | <4.83E−10 | 142 | 9.87E−10 |
| 69 | <4.83E−10 | 40 | 1.17E−09 |
| 53 | <4.83E−10 | 116 | 1.21E−09 |
| 91 | <4.83E−10 | 60 | 1.25E−09 |
| 153 | <4.83E−10 | 38 | 1.39E−09 |
| 230 | <4.83E−10 | 233 | 1.43E−09 |
| 229 | <4.83E−10 | 141 | 1.66E−09 |
| 195 | <4.83E−10 | 50 | 1.80E−09 |
| 150 | <4.83E−10 | 290 | 1.84E−09 |
| 199 | <4.83E−10 | 254 | 2.03E−09 |
| 184 | <4.83E−10 | 68 | 2.11E−09 |
| 234 | <4.83E−10 | 292 | 2.26E−09 |
| 146 | <4.83E−10 | 111 | 2.62E−09 |
| 139 | <4.83E−10 | 288 | 2.63E−09 |
| 154 | <4.83E−10 | 129 | 2.86E−09 |
| 204 | <4.83E−10 | 70 | 3.34E−09 |
| 235 | <4.83E−10 | 207 | 3.76E−09 |
| 225 | <4.83E−10 | 209 | 3.78E−09 |
| 106 | <4.83E−10 | 220 | 3.83E−09 |
| 27 | <4.83E−10 | 237 | 4.04E−09 |
| 96 | <4.83E−10 | 130 | 4.12E−09 |
| 117 | <4.83E−10 | 3 | 4.93E−09 |
| 87 | <4.83E−10 | 197 | 5.01E−09 |
| 152 | <4.83E−10 | 155 | 6.47E−09 |
| 191 | <4.83E−10 | 178 | 6.84E−09 |
| 213 | <4.83E−10 | 120 | 7.02E−09 |
| 125 | <4.83E−10 | 218 | 7.23E−09 |
| 151 | <4.83E−10 | 236 | 7.44E−09 |
| 285 | <4.83E−10 | 301 | 8.56E−09 |
| 239 | <4.83E−10 | 56 | 1.61E−08 |
| 41 | <4.83E−10 | 183 | 1.99E−08 |
| 240 | <4.83E−10 | 175 | 2.01E−08 |
| 29 | 1.21E−11 | 302 | 3.97E−08 |

TABLE 18B

| Sample ID | IC20 | Sample ID | IC20 |
|---|---|---|---|
| 87 | <4.83E−10 | 50 | 2.78E−09 |
| 45 | <4.83E−10 | 71 | 2.78E−09 |
| 263 | <4.83E−10 | 230 | 2.85E−09 |
| 53 | <4.83E−10 | 154 | 3.08E−09 |
| 229 | <4.83E−10 | 234 | 3.41E−09 |
| 195 | <4.83E−10 | 233 | 3.41E−09 |
| 150 | <4.83E−10 | 116 | 3.78E−09 |
| 199 | <4.83E−10 | 129 | 3.83E−09 |
| 184 | <4.83E−10 | 111 | 3.94E−09 |
| 146 | <4.83E−10 | 288 | 4.00E−09 |
| 204 | <4.83E−10 | 254 | 4.05E−09 |
| 225 | <4.83E−10 | 68 | 4.28E−09 |
| 106 | <4.83E−10 | 224 | 4.43E−09 |
| 96 | <4.83E−10 | 207 | 4.64E−09 |
| 152 | <4.83E−10 | 292 | 4.66E−09 |
| 125 | <4.83E−10 | 70 | 4.86E−09 |
| 151 | <4.83E−10 | 60 | 5.30E−09 |
| 285 | <4.83E−10 | 197 | 5.85E−09 |
| 240 | <4.83E−10 | 220 | 6.57E−09 |
| 209 | 4.58E−10 | 237 | 7.43E−09 |
| 139 | 7.93E−10 | 91 | 7.48E−09 |
| 213 | 9.28E−10 | 290 | 7.78E−09 |
| 41 | 1.06E−09 | 130 | 7.91E−09 |
| 117 | 1.09E−09 | 120 | 8.70E−09 |
| 69 | 1.28E−09 | 178 | 1.01E−08 |
| 191 | 1.40E−09 | 155 | 1.01E−08 |
| 80 | 1.44E−09 | 137 | 1.04E−08 |
| 235 | 1.48E−09 | 93 | 1.06E−08 |
| 27 | 1.49E−09 | 186 | 1.12E−08 |
| 239 | 1.58E−09 | 3 | 1.13E−08 |
| 141 | 1.80E−09 | 236 | 1.18E−08 |
| 38 | 1.81E−09 | 29 | 1.23E−08 |
| 142 | 2.07E−09 | 127 | 1.27E−08 |
| 273 | 2.23E−09 | 301 | 2.02E−08 |
| 295 | 2.30E−09 | 218 | 2.03E−08 |
| 76 | 2.39E−09 | 56 | 2.35E−08 |
| 105 | 2.51E−09 | 302 | 2.37E−08 |
| 153 | 2.53E−09 | 175 | 2.49E−08 |
| 40 | 2.78E−09 | 183 | 2.55E−08 |

TABLE 18C

| Sample ID | IC30 | Sample ID | IC30 |
|---|---|---|---|
| 87 | <4.83E−10 | 93 | 9.45E−10 |
| 76 | <4.83E−10 | 142 | 9.87E−10 |
| 45 | <4.83E−10 | 240 | 1.06E−09 |
| 273 | <4.83E−10 | 40 | 1.17E−09 |
| 295 | <4.83E−10 | 116 | 1.21E−09 |
| 80 | <4.83E−10 | 60 | 1.25E−09 |
| 263 | <4.83E−10 | 151 | 1.37E−09 |
| 69 | <4.83E−10 | 38 | 1.39E−09 |
| 53 | <4.83E−10 | 233 | 1.43E−09 |
| 91 | <4.83E−10 | 141 | 1.66E−09 |
| 153 | <4.83E−10 | 50 | 1.80E−09 |
| 230 | <4.83E−10 | 254 | 2.03E−09 |
| 229 | <4.83E−10 | 68 | 2.11E−09 |
| 195 | <4.83E−10 | 292 | 2.26E−09 |
| 150 | <4.83E−10 | 111 | 2.62E−09 |
| 199 | <4.83E−10 | 285 | 2.70E−09 |
| 184 | <4.83E−10 | 129 | 2.86E−09 |
| 234 | <4.83E−10 | 70 | 3.34E−09 |
| 146 | <4.83E−10 | 209 | 3.78E−09 |
| 139 | <4.83E−10 | 220 | 3.83E−09 |
| 154 | <4.83E−10 | 237 | 4.04E−09 |
| 204 | <4.83E−10 | 130 | 4.12E−09 |
| 235 | <4.83E−10 | 213 | 4.94E−09 |
| 225 | <4.83E−10 | 197 | 5.01E−09 |
| 106 | <4.83E−10 | 288 | 5.54E−09 |
| 27 | <4.83E−10 | 155 | 6.47E−09 |
| 96 | <4.83E−10 | 178 | 6.84E−09 |
| 117 | <4.83E−10 | 120 | 7.02E−09 |
| 152 | <4.83E−10 | 239 | 7.28E−09 |
| 191 | <4.83E−10 | 236 | 7.44E−09 |
| 224 | 2.97E−10 | 41 | 1.03E−08 |
| 105 | 4.81E−10 | 290 | 1.16E−08 |
| 127 | 4.83E−10 | 56 | 1.61E−08 |
| 29 | 4.83E−10 | 183 | 1.99E−08 |
| 207 | 4.83E−10 | 175 | 2.01E−08 |
| 71 | 5.99E−10 | 3 | 2.21E−08 |
| 137 | 6.02E−10 | 301 | 3.18E−08 |
| 125 | 6.28E−10 | 302 | 4.05E−08 |
| 186 | 8.41E−10 | 218 | 6.08E−08 |

TABLE 18D

| Sample ID | IC40 | Sample ID | IC40 |
|---|---|---|---|
| 53 | 5.96E−10 | 224 | 1.49E−08 |
| 263 | 1.52E−09 | 80 | 1.52E−08 |
| 139 | 2.85E−09 | 237 | 1.53E−08 |
| 184 | 3.55E−09 | 290 | 1.54E−08 |
| 150 | 3.87E−09 | 130 | 1.70E−08 |
| 151 | 4.16E−09 | 41 | 1.71E−08 |
| 117 | 4.47E−09 | 60 | 1.84E−08 |
| 152 | 4.48E−09 | 29 | 1.90E−08 |
| 106 | 4.83E−09 | 207 | 1.99E−08 |
| 141 | 4.92E−09 | 178 | 2.01E−08 |
| 225 | 5.13E−09 | 155 | 2.01E−08 |
| 27 | 5.22E−09 | 38 | 2.06E−08 |
| 40 | 5.39E−09 | 91 | 2.09E−08 |
| 295 | 5.63E−09 | 186 | 2.09E−08 |
| 71 | 5.65E−09 | 69 | 2.13E−08 |
| 191 | 6.04E−09 | 153 | 2.16E−08 |
| 146 | 6.10E−09 | 233 | 2.17E−08 |
| 229 | 6.25E−09 | 235 | 2.18E−08 |
| 96 | 6.50E−09 | 125 | 2.30E−08 |
| 45 | 6.91E−09 | 236 | 2.34E−08 |
| 197 | 7.53E−09 | 127 | 2.39E−08 |
| 285 | 7.67E−09 | 50 | 2.52E−08 |
| 129 | 7.72E−09 | 154 | 2.59E−08 |
| 68 | 7.80E−09 | 240 | 2.61E−08 |
| 195 | 8.35E−09 | 199 | 2.66E−08 |
| 105 | 9.24E−09 | 175 | 3.45E−08 |
| 87 | 1.02E−08 | 183 | 3.67E−08 |
| 111 | 1.02E−08 | 204 | 3.82E−08 |
| 230 | 1.06E−08 | 234 | 3.86E−08 |
| 213 | 1.10E−08 | 70 | 3.98E−08 |
| 142 | 1.15E−08 | 3 | 4.17E−08 |
| 288 | 1.17E−08 | 301 | 4.34E−08 |
| 254 | 1.20E−08 | 218 | 1.43E−07 |
| 239 | 1.20E−08 | 120 | >4.83E−08 |
| 76 | 1.32E−08 | 56 | >4.83E−08 |
| 220 | 1.32E−08 | 137 | >4.83E−08 |
| 292 | 1.38E−08 | 93 | >4.83E−08 |
| 273 | 1.42E−08 | 209 | >4.83E−08 |
| 116 | 1.44E−08 | 302 | >4.83E−08 |

TABLE 18E

| Sample ID | IC50 | Sample ID | IC50 |
|---|---|---|---|
| 117 | 8.45E−09 | 80 | 2.43E−08 |
| 263 | 8.59E−09 | 29 | 2.51E−08 |
| 191 | 9.18E−09 | 150 | 2.63E−08 |
| 71 | 9.57E−09 | 146 | 2.70E−08 |
| 152 | 9.77E−09 | 199 | 2.70E−08 |
| 295 | 1.00E−08 | 130 | 2.73E−08 |
| 40 | 1.03E−08 | 233 | 2.75E−08 |
| 53 | 1.11E−08 | 106 | 2.76E−08 |
| 151 | 1.14E−08 | 125 | 2.84E−08 |
| 229 | 1.22E−08 | 91 | 2.89E−08 |
| 27 | 1.27E−08 | 60 | 3.03E−08 |
| 129 | 1.28E−08 | 236 | 3.05E−08 |
| 87 | 1.28E−08 | 153 | 3.09E−08 |
| 225 | 1.30E−08 | 127 | 3.16E−08 |
| 96 | 1.39E−08 | 235 | 3.33E−08 |
| 141 | 1.41E−08 | 207 | 3.48E−08 |
| 285 | 1.44E−08 | 240 | 3.52E−08 |
| 111 | 1.53E−08 | 154 | 3.66E−08 |
| 105 | 1.53E−08 | 175 | 3.93E−08 |
| 142 | 1.59E−08 | 183 | 4.23E−08 |
| 45 | 1.59E−08 | 178 | 4.24E−08 |
| 197 | 1.60E−08 | 38 | 4.50E−08 |
| 239 | 1.75E−08 | 234 | 4.72E−08 |
| 254 | 1.81E−08 | 3 | 6.88E−08 |
| 184 | 1.83E−08 | 155 | >2.42E−08 |
| 213 | 1.88E−08 | 120 | >2.42E−08 |
| 237 | 1.93E−08 | 68 | >2.42E−08 |
| 273 | 1.95E−08 | 50 | >2.42E−08 |
| 195 | 1.99E−08 | 56 | >2.42E−08 |
| 76 | 1.99E−08 | 137 | >2.42E−08 |
| 230 | 2.02E−08 | 186 | >2.42E−08 |
| 288 | 2.07E−08 | 93 | >2.42E−08 |
| 220 | 2.12E−08 | 69 | >4.83E−08 |
| 224 | 2.22E−08 | 70 | >4.83E−08 |
| 292 | 2.25E−08 | 209 | >4.83E−08 |
| 290 | 2.30E−08 | 204 | >4.83E−08 |
| 41 | 2.31E−08 | 218 | >2.42E−07 |
| 116 | 2.34E−08 | 301 | >4.83E−08 |
| 139 | 2.37E−08 | 302 | >4.83E−08 |

TABLE 18F

| Sample ID | IC60 | Sample ID | IC60 |
|---|---|---|---|
| 191 | 1.23E−08 | 127 | 3.54E−08 |
| 71 | 1.35E−08 | 288 | 3.61E−08 |
| 87 | 1.37E−08 | 150 | 3.66E−08 |
| 295 | 1.44E−08 | 91 | 3.69E−08 |
| 263 | 1.44E−08 | 199 | 3.70E−08 |
| 117 | 1.45E−08 | 130 | 3.75E−08 |
| 40 | 1.50E−08 | 106 | 3.75E−08 |
| 152 | 1.72E−08 | 236 | 3.76E−08 |
| 53 | 1.93E−08 | 111 | 3.82E−08 |
| 129 | 1.95E−08 | 153 | 4.02E−08 |
| 151 | 2.01E−08 | 146 | 4.11E−08 |
| 229 | 2.13E−08 | 60 | 4.23E−08 |
| 105 | 2.14E−08 | 175 | 4.40E−08 |
| 285 | 2.24E−08 | 240 | 4.43E−08 |
| 225 | 2.29E−08 | 235 | 4.48E−08 |
| 237 | 2.33E−08 | 154 | 4.73E−08 |
| 141 | 2.36E−08 | 183 | 4.79E−08 |
| 27 | 2.45E−08 | 3 | 6.96E−08 |
| 45 | 2.51E−08 | 155 | >2.42E−08 |
| 76 | 2.54E−08 | 197 | >2.42E−08 |
| 184 | 2.54E−08 | 120 | >2.42E−08 |
| 96 | 2.63E−08 | 68 | >2.42E−08 |
| 239 | 2.65E−08 | 50 | >2.42E−08 |
| 213 | 2.65E−08 | 56 | >2.42E−08 |
| 273 | 2.75E−08 | 224 | >2.42E−08 |
| 195 | 2.84E−08 | 137 | >2.42E−08 |
| 41 | 2.91E−08 | 186 | >2.42E−08 |
| 142 | 2.92E−08 | 93 | >2.42E−08 |
| 254 | 3.10E−08 | 207 | >4.83E−08 |
| 230 | 3.12E−08 | 69 | >4.83E−08 |
| 29 | 3.12E−08 | 178 | >4.83E−08 |
| 290 | 3.13E−08 | 70 | >4.83E−08 |
| 220 | 3.14E−08 | 209 | >4.83E−08 |
| 233 | 3.32E−08 | 38 | >4.83E−08 |
| 116 | 3.36E−08 | 234 | >4.83E−08 |
| 80 | 3.38E−08 | 204 | >4.83E−08 |
| 125 | 3.39E−08 | 301 | >4.830E−08 |
| 292 | 3.40E−08 | 302 | >4.83E−08 |
| 139 | 3.52E−08 | 218 | >2.42E−07 |

In particular, detailed examination was performed concerning closely associated inflammation sites and tumor sites.

### (Inflammation Sites)

TABLE 18G

| | Tropism for | Abbreviated | | | | | | | | | | | |
|---|---|---|---|---|---|---|---|---|---|---|---|---|---|
| Liposome No. | organ | name | IC10 | IC20 | IC30 | IC31 | IC32 | IC33 | IC34 | IC35 | IC40 | IC50 | IC60 |
| 27 | ++ | LDF-5 | <4.83E−10 | 1.49E−09 | <4.83E−10 | 3.44E−09 | 3.64E−09 | 3.83E−09 | 4.03E−09 | 4.23E−09 | 5.22E−09 | 1.27E−08 | 2.45E−08 |
| 38 | ++ | G4GN-4 | 1.39E−09 | 1.81E−09 | 1.39E−09 | 2.85E−08 | 2.97E−08 | 3.08E−08 | 3.20E−08 | 3.32E−08 | 2.06E−08 | 4.50E−08 | >4.83E−08 |
| 53 | ++ | L2F-1 | <4.83E−10 | <4.83E−10 | <4.83E−10 | <4.83E−10 | <4.83E−10 | <4.83E−10 | <4.83E−10 | 5.96E−10 | 5.96E−10 | 1.11E−08 | 1.93E−08 |
| 60 | ++ | 6SLN-1 | 1.25E−09 | 5.30E−09 | 1.25E−09 | 1.18E−08 | 1.24E−08 | 1.30E−08 | 1.36E−08 | 1.42E−08 | 1.84E−08 | 3.03E−08 | 4.23E−08 |
| 69 | ++ | L4F-1 | <4.83E−10 | 1.28E−09 | <4.83E−10 | 1.11E−08 | 1.18E−08 | 1.25E−08 | 1.31E−08 | 1.38E−08 | 2.13E−08 | >4.83E−08 | >4.83E−08 |
| 70 | ++ | LNT-5 | 3.34E−09 | 4.86E−09 | 3.34E−09 | 2.72E−08 | 2.86E−08 | 3.00E−08 | 3.14E−08 | 3.28E−08 | 3.98E−08 | >4.83E−08 | >4.83E−08 |
| 87 | ++ | TA-1 | <4.83E−10 | <4.83E−10 | <4.83E−10 | 7.80E−09 | 8.06E−09 | 8.33E−09 | 8.59E−09 | 8.88E−09 | 1.02E−08 | 1.28E−08 | 1.37E−08 |
| 91 | ++ | G6GN-4 | <4.83E−10 | 7.48E−09 | <4.83E−10 | 1.42E−08 | 1.48E−08 | <4.83E−10 | 1.60E−08 | 1.68E−08 | 2.09E−08 | 2.89E−08 | 3.69E−08 |
| 93 | ++ | A36 | 9.73E−10 | 1.06E−08 | 9.45E−10 | >4.83E−08 | >4.83E−08 | >4.83E−08 | >4.83E−08 | >4.83E−08 | >4.83E−08 | >2.42E−08 | >2.42E−08 |
| 96 | ++ | TA-4 | <4.83E−10 | <4.83E−10 | <4.83E−10 | 3.89E−09 | 4.08E−09 | 4.28E−09 | 4.47E−09 | 4.66E−09 | 8.49E−09 | 1.39E−08 | 2.63E−08 |
| 105 | ++ | Man6 | 4.81E−10 | 2.51E−09 | 4.81E−10 | 5.90E−09 | 6.22E−09 | 6.53E−09 | 6.85E−09 | 7.16E−09 | 9.24E−09 | 1.53E−08 | 2.14E−08 |
| 106 | ++ | LDF-4 | <4.83E−10 | <4.83E−10 | <4.83E−10 | 2.16E−09 | 2.46E−09 | 2.75E−09 | 3.05E−09 | 3.35E−09 | 4.83E−09 | 2.76E−08 | 3.75E−08 |
| 116 | ++ | LDF-1 | 1.21E−09 | 3.78E−09 | 1.21E−09 | 9.06E−09 | 9.65E−09 | 1.02E−08 | 1.08E−08 | 1.14E−08 | 1.44E−08 | 2.34E−08 | 3.36E−08 |
| 125 | ++ | LX-1 | <4.83E−10 | <4.83E−10 | 6.28E−10 | 1.02E−09 | 1.86E−08 | 1.91E−08 | 1.97E−08 | 2.02E−08 | 2.30E−08 | 2.84E−08 | 3.39E−08 |
| 139 | ++ | 3FL-5 | <4.83E−10 | 7.93E−10 | <4.83E−10 | 1.49E−09 | 1.55E−09 | 1.61E−09 | 1.68E−09 | 1.74E−09 | 2.85E−09 | 2.37E−08 | 3.52E−08 |
| 146 | ++ | 3FL-4 | <4.83E−10 | <4.83E−10 | <4.83E−10 | 2.89E−09 | 3.29E−09 | 3.70E−09 | 4.11E−09 | 4.51E−09 | 6.10E−09 | 2.70E−08 | 4.11E−08 |
| 150 | ++ | TNA-5 | <4.83E−10 | <4.83E−10 | <4.83E−10 | <4.83E−10 | 2.90E−10 | 1.39E−10 | 8.09E−10 | 1.48E−09 | 3.86E−09 | 2.63E−08 | 3.66E−08 |
| 152 | ++ | LY-1 | <4.83E−10 | <4.83E−10 | <4.83E−10 | 2.49E−09 | 2.71E−09 | 2.93E−09 | 3.15E−09 | 3.37E−09 | 4.48E−09 | 9.77E−09 | 1.72E−08 |
| 153 | ++ | G6GN-5 | <4.83E−10 | 2.53E−09 | <4.83E−10 | 1.14E−08 | 1.29E−08 | 1.45E−08 | 1.60E−08 | 1.70E−08 | 2.16E−08 | 3.09E−08 | 4.02E−08 |
| 154 | ++ | Lac-5 | <4.83E−10 | 3.08E−09 | <4.83E−10 | 1.62E−08 | 1.73E−08 | 1.84E−08 | 1.94E−08 | 2.05E−08 | 2.59E−08 | 3.66E−08 | 4.73E−08 |
| 155 | ++ | A6 | 6.47E−09 | 1.01E−08 | 6.47E−09 | 1.58E−08 | 1.63E−08 | 1.68E−08 | 1.72E−08 | 1.77E−08 | 2.01E−08 | >2.42E−08 | >2.42E−08 |
| 184 | ++ | STNA-1 | <4.83E−10 | <4.83E−10 | <4.83E−10 | 1.26E−09 | 1.39E−09 | 1.52E−09 | 1.65E−09 | 1.77E−09 | 3.55E−09 | 1.83E−08 | 2.54E−08 |
| 191 | ++ | LB-1 | <4.83E−10 | 1.40E−09 | <4.83E−10 | 3.50E−09 | 3.77E−09 | 4.04E−09 | 4.31E−09 | 4.59E−09 | 6.04E−09 | 9.18E−09 | 1.23E−08 |
| 195 | ++ | TNA-4 | <4.83E−10 | <4.83E−10 | <4.83E−10 | 8.88E−10 | 1.11E−09 | 5.33E−09 | 1.58E−09 | 1.78E−09 | 8.35E−09 | 1.99E−08 | 2.84E−08 |
| 199 | ++ | STNA-4 | <4.83E−10 | <4.83E−10 | <4.83E−10 | 3.53E−09 | 3.79E−09 | 4.05E−09 | 4.31E−09 | 4.57E−09 | 2.66E−08 | 2.70E−08 | 3.70E−08 |
| 204 | ++ | Lac-1 | <4.83E−10 | <4.83E−10 | <4.83E−10 | 2.47E−08 | 2.62E−08 | 2.77E−08 | 2.92E−08 | 3.07E−08 | 3.82E−08 | >4.83E−08 | >4.83E−08 |
| 213 | ++ | LX-4 | <4.83E−10 | 9.28E−10 | 4.94E−09 | 5.08E−09 | 5.22E−09 | 5.35E−09 | 6.32E−09 | 7.10E−09 | 1.10E−08 | 1.88E−08 | 2.65E−08 |
| 220 | ++ | LNB-4 | 3.83E−09 | 6.57E−09 | 3.83E−09 | 1.06E−08 | 1.09E−08 | 1.11E−08 | 1.14E−08 | 7.27E−09 | 1.32E−08 | 2.12E−08 | 3.14E−08 |
| 224 | ++ | Man5 | 2.97E−10 | 4.43E−09 | 2.97E−10 | 8.44E−09 | 9.16E−09 | 9.88E−09 | 1.06E−08 | 1.13E−08 | 1.48E−08 | 2.22E−08 | >2.42E−08 |
| 229 | ++ | G6GN-1 | <4.83E−10 | <4.83E−10 | <4.83E−10 | 3.51E−09 | 3.76E−09 | <4.83E−10 | 4.24E−09 | 4.48E−09 | 6.25E−09 | 1.22E−08 | 2.13E−08 |
| 230 | ++ | G4GN-1 | <4.83E−10 | 2.85E−09 | <4.83E−10 | 4.77E−09 | 4.95E−09 | 5.12E−09 | 5.22E−09 | 6.11E−09 | 1.05E−08 | 2.02E−08 | 3.12E−08 |
| 233 | ++ | 2FL-1 | 1.43E−09 | 3.41E−09 | 1.43E−09 | 1.66E−08 | 1.71E−08 | 1.77E−08 | 1.83E−08 | 1.89E−08 | 2.17E−08 | 2.75E−08 | 3.32E−08 |
| 234 | ++ | TNA-1 | <4.83E−10 | 3.41E−09 | <4.83E−10 | 3.09E−08 | 3.17E−08 | 3.34E−08 | 3.34E−08 | 3.43E−08 | 3.86E−08 | 4.72E−08 | >4.83E−08 |
| 235 | ++ | Lac-4 | <4.83E−10 | 1.48E−09 | <4.83E−10 | 4.27E−09 | 4.46E−09 | 1.47E−08 | 1.54E−08 | 5.05E−09 | 2.18E−08 | 3.33E−08 | 4.48E−08 |
| 237 | ++ | A3 | 4.04E−09 | 7.43E−09 | 4.04E−09 | 1.17E−08 | 1.21E−08 | 1.25E−08 | 1.29E−08 | 1.33E−08 | 1.53E−08 | 1.93E−08 | 2.33E−08 |
| 263 | ++ | 3SLN-1 | <4.83E−10 | <4.83E−10 | <4.83E−10 | 7.20E−10 | 8.08E−10 | 8.96E−10 | 9.85E−10 | 1.07E−09 | 1.51E−09 | 8.59E−09 | 1.44E−08 |
| 290 | ++ | SLA-5 | 1.84E−09 | 7.78E−09 | 1.16E−08 | 1.19E−08 | 1.23E−08 | 1.27E−08 | 1.31E−08 | 1.35E−08 | 1.53E−08 | 2.30E−08 | 3.13E−08 |
| 292 | ++ | STNA-5 | 2.26E−09 | 4.66E−09 | 2.26E−09 | 9.39E−09 | 7.55E−09 | 1.04E−08 | 1.09E−08 | 1.14E−08 | 1.38E−08 | 2.25E−08 | 3.40E−08 |
| 292 | ++ | STNA-5 | 2.26E−09 | 4.66E−09 | 2.26E−09 | 9.39E−09 | 7.55E−09 | 1.04E−08 | 1.09E−08 | 1.14E−08 | 1.38E−08 | 2.25E−08 | 3.40E−08 |
| 40 | + | BAT-1 | 1.17E−09 | 2.78E−09 | 1.17E−09 | 4.22E−09 | 4.35E−09 | 4.48E−09 | 4.61E−09 | 4.74E−09 | 5.39E−09 | 1.03E−08 | 1.50E−08 |
| 41 | + | SOLX-4 | <4.83E−10 | 1.06E−09 | 1.03E−08 | 1.13E−08 | 1.17E−08 | 1.24E−08 | 1.31E−08 | 1.38E−08 | 1.71E−08 | 2.31E−08 | 2.91E−08 |
| 50 | + | Man8 | 1.80E−09 | 2.78E−09 | 1.80E−09 | 1.30E−08 | 1.44E−08 | 1.57E−08 | 1.71E−08 | 1.84E−08 | 2.52E−08 | >2.42E−08 | >2.42E−08 |
| 56 | + | Man9 | 1.61E−08 | 2.35E−08 | 1.61E−08 | >4.83E−08 | >4.83E−08 | >4.83E−08 | >4.83E−08 | >4.83E−08 | >4.83E−08 | >2.42E−08 | >2.42E−08 |
| 68 | + | Man7 | 2.11E−09 | 4.28E−09 | 2.11E−09 | >4.83E−08 | >4.83E−08 | >4.83E−08 | 1.68E−08 | 1.58E−08 | 7.80E−09 | >2.42E−08 | >2.42E−08 |
| 71 | + | BBT-1 | 5.99E−10 | 2.78E−09 | 5.99E−10 | 4.24E−09 | 4.37E−09 | 4.50E−09 | 4.63E−09 | 4.77E−09 | 5.65E−08 | 9.57E−09 | 1.35E−08 |
| 76 | + | BHD-4 | <4.83E−10 | 2.39E−09 | <4.83E−10 | 4.21E−09 | 5.21E−09 | 6.20E−09 | 7.19E−09 | 8.19E−09 | 1.32E−08 | 1.99E−08 | 2.54E−08 |
| 111 | + | LNB-5 | 2.62E−09 | 3.94E−09 | 2.62E−09 | 5.62E−09 | 6.13E−09 | 6.64E−09 | 7.15E−09 | 7.66E−09 | 1.02E−08 | 1.53E−08 | 3.82E−08 |
| 117 | + | TA-5 | <4.83E−10 | 1.09E−09 | <4.83E−10 | 2.88E−09 | 3.06E−09 | 3.24E−09 | 3.41E−09 | 3.59E−09 | 4.47E−09 | 8.45E−09 | 1.45E−08 |
| 120 | + | A2 | 7.02E−09 | 8.70E−09 | 7.02E−09 | 2.09E−08 | 2.20E−08 | 2.31E−08 | 2.42E−08 | >4.83E−08 | >4.83E−08 | >2.42E−08 | >2.42E−08 |
| 137 | + | Man4B | 6.02E−10 | 1.04E−08 | 6.02E−10 | 1.96E−08 | 2.04E−08 | 2.13E−08 | 2.21E−08 | 2.30E−08 | >4.83E−08 | >2.42E−08 | >2.42E−08 |
| 151 | + | SLX-4 | <4.83E−10 | <4.83E−10 | 1.37E−09 | 1.52E−09 | 1.67E−09 | 1.82E−09 | 1.98E−09 | 2.39E−09 | 4.16E−09 | 1.14E−08 | 2.01E−08 |
| 183 | + | G4GN-5 | 1.99E−08 | 2.55E−08 | 1.99E−08 | 3.16E−08 | 3.22E−08 | 3.28E−08 | 3.33E−08 | 3.39E−08 | 3.67E−08 | 4.23E−08 | 4.79E−08 |
| 186 | + | Man3 | 8.41E−10 | 1.12E−08 | 8.41E−10 | 1.66E−08 | 1.70E−08 | 1.75E−08 | 1.80E−08 | 1.85E−08 | 2.09E−08 | >2.42E−08 | >2.42E−08 |
| 197 | + | A4 | 5.01E−09 | 5.85E−09 | 5.01E−09 | 6.77E−09 | 6.86E−09 | 6.94E−09 | 7.03E−09 | 7.11E−09 | 7.53E−09 | 1.60E−08 | >2.42E−08 |
| 209 | + | LDFH-4 | 3.78E−09 | 4.58E−10 | 3.78E−09 | 4.23E−08 | 4.47E−08 | 4.70E−08 | 4.94E−08 | >4.83E−08 | >4.83E−08 | >4.83E−08 | >4.83E−08 |
| 240 | + | SOLA-5 | <4.83E−10 | <4.83E−10 | 1.06E−09 | 1.79E−08 | 1.88E−08 | 1.97E−08 | 2.07E−09 | 2.16E−08 | 2.61E−08 | 3.52E−08 | 4.43E−08 |
| 288 | + | LA-1 | 2.63E−09 | 4.00E−09 | 5.54E−09 | 6.16E−09 | 6.77E−09 | 7.39E−09 | 8.00E−09 | 8.62E−09 | 1.17E−08 | 2.07E−08 | 3.61E−08 |
| 295 | + | 6SLN-4 | <4.83E−10 | 2.30E−09 | <4.83E−10 | 4.18E−09 | 4.18E−10 | 4.33E−09 | 4.49E−09 | 4.65E−09 | 5.63E−09 | 1.00E−08 | 1.44E−08 |
| 16 | − | SOLX-5 | <4.83E−10 | 1.42E−08 | 2.37E−08 | 2.43E−08 | 2.49E−08 | 2.55E−08 | 2.61E−08 | 4.13E−09 | 2.97E−08 | 3.57E−08 | 4.16E−08 |
| 29 | − | 3SL-5 | 1.21E−11 | 1.23E−08 | 4.83E−10 | 1.50E−08 | 1.52E−08 | 1.55E−08 | 1.54E−08 | 1.60E−08 | 1.90E−08 | 2.51E−08 | 3.12E−08 |
| 127 | − | G3G-4 | <4.83E−10 | 1.27E−08 | 4.83E−10 | 1.54E−08 | 1.57E−08 | 1.59E−08 | 1.70E−08 | 1.82E−08 | 2.39E−08 | 3.16E−08 | 3.54E−08 |
| 129 | − | BBT-4 | 2.86E−09 | 3.83E−09 | 2.86E−09 | 4.90E−09 | 5.00E−09 | 5.09E−09 | 5.19E−09 | 5.29E−09 | 7.72E−09 | 1.28E−08 | 1.95E−08 |
| 130 | − | 2FL-4 | 4.12E−09 | 7.91E−09 | 4.12E−09 | 1.26E−08 | 1.30E−08 | 1.34E−08 | 1.38E−08 | 1.43E−08 | 1.70E−08 | 2.73E−08 | 3.75E−08 |
| 141 | − | BBT-5 | 1.66E−09 | 1.80E−09 | 1.66E−09 | 2.47E−09 | 2.74E−09 | 3.02E−09 | 3.29E−09 | 3.56E−09 | 4.92E−09 | 1.41E−08 | 2.38E−08 |
| 142 | − | BHD-5 | 9.87E−10 | 2.07E−09 | 9.87E−10 | 7.48E−09 | 7.92E−09 | 8.37E−09 | 8.81E−09 | 9.25E−09 | 1.15E−08 | 1.59E−08 | 2.92E−08 |
| 175 | − | G3G-5 | 2.01E−08 | 2.49E−08 | 2.01E−08 | 3.02E−08 | 3.06E−08 | 3.11E−08 | 3.16E−08 | 3.21E−08 | 3.45E−08 | 3.93E−08 | 4.40E−08 |
| 178 | − | LNT-4 | 6.84E−09 | 1.01E−08 | 6.84E−09 | 1.36E−08 | 1.40E−08 | 1.43E−08 | 1.46E−08 | 1.49E−08 | 2.01E−08 | 4.24E−08 | >4.83E−08 |
| 207 | − | 6SL-5 | 3.78E−09 | 4.64E−09 | 4.83E−10 | 8.94E−09 | 1.00E−08 | 1.11E−08 | 1.22E−08 | 1.34E−08 | 1.99E−08 | 3.48E−08 | >4.83E−08 |
| 236 | − | 2FL-5 | 7.44E−09 | 1.18E−08 | 7.44E−09 | 1.70E−08 | 1.77E−08 | 1.84E−08 | 1.92E−08 | 1.99E−08 | 2.34E−08 | 3.05E−08 | 3.76E−08 |
| 239 | − | SLA-4 | <4.83E−10 | 1.58E−09 | 7.28E−09 | 7.75E−09 | 8.23E−09 | 8.70E−09 | 9.17E−08 | 9.64E−09 | 1.20E−08 | 1.75E−08 | 2.65E−08 |
| 3 | − | EEGD1A-BH | 4.93E−09 | 1.13E−08 | 2.21E−08 | 2.32E−08 | 2.43E−08 | 2.53E−08 | 2.64E−08 | 2.81E−08 | 4.17E−08 | 6.88E−08 | 6.96E−08 |
| 218 | − | EEGM1-BH | 7.23E−09 | 2.03E−08 | 6.08E−08 | 6.49E−08 | 6.90E−08 | 7.31E−08 | 7.73E−08 | 8.45E−08 | 1.43E−07 | >2.42E−07 | >2.42E−07 |
| 254 | − | G3G-1 | 2.03E−09 | 4.05E−09 | 2.03E−09 | 7.61E−09 | 8.09E−09 | 8.57E−09 | 9.06E−09 | 9.54E−09 | 1.19E−08 | 1.81E−08 | 3.10E−08 |
| 301 | − | TRIS | 8.66E−09 | 2.02E−08 | 3.18E−08 | 3.30E−08 | 3.41E−08 | 3.53E−08 | 3.64E−08 | 3.76E−08 | 4.34E−08 | >4.83E−08 | >4.83E−08 |
| 302 | − | CEBI | 3.98E−08 | 2.37E−08 | 4.05E−08 | 4.22E−08 | 4.39E−08 | 4.56E−08 | 4.73E−08 | 4.90E−08 | >4.83E−08 | >4.83E−08 | >4.83E−08 |

### (Tumor Sites)

TABLE 18H

| | Tropism for | Abbreviated | | | | | | | | | | | |
|---|---|---|---|---|---|---|---|---|---|---|---|---|---|
| Liposome No. | organ | name | IC10 | IC20 | IC30 | IC31 | IC32 | IC33 | IC34 | IC35 | IC40 | IC50 | IC60 |
| 27 | ++ | LDF-5 | <4.83E−10 | 1.49E−09 | <4.83E−10 | 3.44E−09 | 3.64E−09 | 3.83E−08 | 4.03E−09 | 4.23E−09 | 5.22E−09 | 1.27E−08 | 2.45E−08 |
| 29 | ++ | 3SL-5 | 1.21E−11 | 1.23E−08 | 4.83E−10 | 1.50E−08 | 1.52E−08 | 1.55E−08 | 1.54E−08 | 1.60E−08 | 1.90E−08 | 2.51E−08 | 3.12E−08 |
| 38 | ++ | G4GN-4 | 1.39E−09 | 1.81E−09 | 1.39E−09 | 2.85E−08 | 2.97E−08 | 3.08E−08 | 3.20E−08 | 3.32E−08 | 2.06E−08 | 4.50E−08 | >4.83E−08 |
| 53 | ++ | L2F-1 | <4.83E−10 | <4.83E−10 | <4.83E−10 | <4.83E−10 | <4.83E−10 | <4.83E−10 | <4.83E−10 | 5.96E−10 | 5.96E−10 | 1.11E−08 | 1.93E−08 |
| 60 | ++ | 6SLN-1 | 1.25E−09 | 5.30E−09 | 1.25E−09 | 1.18E−08 | 1.24E−08 | 1.30E−08 | 1.36E−08 | 1.42E−08 | 1.84E−08 | 3.03E−08 | 4.23E−08 |
| 69 | ++ | L4F-1 | <4.83E−10 | 1.28E−09 | <4.83E−10 | 1.11E−08 | 1.18E−08 | 1.25E−08 | 1.31E−08 | 1.38E−08 | 2.13E−08 | >4.83E−08 | >4.83E−08 |
| 87 | ++ | TA-1 | <4.83E−10 | <4.83E−10 | <4.83E−10 | 7.80E−09 | 8.06E−09 | 8.33E−09 | 8.59E−09 | 8.86E−09 | 1.02E−08 | 1.28E−08 | 1.37E−08 |
| 93 | ++ | A36 | 9.73E−10 | 1.06E−08 | 9.45E−10 | >4.83E−08 | >4.83E−08 | >4.83E−08 | >4.83E−08 | >4.83E−08 | >4.83E−08 | >2.42E−08 | >2.42E−08 |
| 96 | ++ | TA-4 | <4.83E−10 | <4.83E−10 | <4.83E−10 | 3.89E−09 | 4.08E−09 | 4.28E−09 | 4.47E−09 | 4.66E−09 | 6.49E−09 | 1.39E−08 | 2.63E−08 |
| 116 | ++ | LDF-1 | 1.21E−09 | 3.78E−09 | 1.21E−09 | 9.06E−09 | 9.65E−09 | 1.02E−08 | 1.08E−08 | 1.14E−08 | 1.44E−08 | 2.34E−08 | 3.36E−08 |
| 125 | ++ | LX-1 | <4.83E−10 | <4.83E−10 | 6.28E−10 | 1.02E−09 | 1.86E−08 | 1.91E−08 | 1.97E−08 | 2.02E−08 | 2.30E−08 | 2.84E−08 | 3.39E−08 |
| 150 | ++ | TNA-5 | <4.83E−10 | <4.83E−10 | <4.83E−10 | <4.83E−10 | 2.90E−10 | 1.39E−10 | 8.09E−10 | 1.48E−09 | 3.86E−09 | 2.63E−08 | 3.66E−08 |
| 152 | ++ | LY-1 | <4.83E−10 | <4.83E−10 | <4.83E−10 | 2.49E−09 | 2.71E−09 | 2.93E−09 | 3.15E−09 | 3.37E−09 | 4.48E−09 | 9.77E−09 | 1.72E−08 |
| 154 | ++ | Lac-5 | <4.83E−10 | 3.08E−09 | <4.83E−10 | 1.62E−08 | 1.73E−08 | 1.84E−08 | 1.94E−08 | 2.05E−08 | 2.59E−08 | 3.66E−08 | 4.73E−08 |
| 155 | ++ | A6 | 6.47E−09 | 1.01E−08 | 6.47E−09 | 1.58E−08 | 1.63E−08 | 1.68E−08 | 1.72E−08 | 1.77E−08 | 2.01E−08 | >2.42E−08 | >2.42E−08 |
| 184 | ++ | STNA-1 | <4.83E−10 | <4.83E−10 | <4.83E−10 | 1.26E−09 | 1.39E−09 | 1.52E−09 | 1.65E−09 | 1.77E−09 | 3.55E−09 | 1.83E−08 | 2.54E−08 |
| 195 | ++ | TNA-4 | <4.83E−10 | <4.83E−10 | <4.83E−10 | 8.88E−10 | 1.11E−09 | 5.33E−09 | 1.56E−09 | 1.78E−09 | 8.35E−09 | 1.99E−08 | 2.84E−08 |
| 204 | ++ | Lac-1 | <4.83E−10 | <4.83E−10 | <4.83E−10 | 2.47E−08 | 2.62E−08 | 2.77E−08 | 2.92E−08 | 3.07E−08 | 3.82E−08 | >4.83E−08 | >4.83E−08 |
| 213 | ++ | LX-4 | <4.83E−10 | 9.28E−10 | 4.94E−09 | 5.08E−09 | 5.22E−09 | 5.35E−09 | 6.32E−09 | 7.10E−09 | 1.10E−08 | 1.88E−08 | 2.65E−08 |
| 224 | ++ | Man5 | 2.97E−10 | 4.43E−09 | 2.97E−10 | 8.44E−09 | 9.16E−09 | 9.88E−09 | 1.06E−08 | 1.13E−08 | 1.49E−08 | 2.22E−08 | >2.42E−08 |
| 229 | ++ | G6GN-1 | <4.83E−10 | <4.83E−10 | <4.83E−10 | 3.51E−09 | 3.76E−09 | <4.83E−10 | 4.24E−09 | 4.48E−09 | 6.25E−09 | 1.22E−08 | 2.13E−08 |
| 230 | ++ | G4GN-1 | <4.83E−10 | 2.85E−09 | <4.83E−10 | 4.77E−09 | 4.95E−09 | 5.12E−09 | 5.22E−09 | 6.11E−09 | 1.05E−08 | 2.02E−08 | 3.12E−08 |
| 233 | ++ | 2FL-1 | 1.43E−09 | 3.41E−09 | 1.43E−09 | 1.66E−08 | 1.71E−08 | 1.77E−08 | 1.83E−08 | 1.89E−08 | 2.17E−08 | 2.75E−08 | 3.32E−08 |
| 234 | ++ | TNA-1 | <4.83E−10 | 3.41E−09 | <4.83E−10 | 3.09E−08 | 3.17E−08 | 3.34E−08 | 3.34E−08 | 3.43E−08 | 3.86E−08 | 4.72E−08 | >4.83E−08 |
| 235 | ++ | Lac-4 | <4.83E−10 | 1.48E−09 | <4.83E−10 | 4.27E−09 | 4.46E−09 | 1.47E−08 | 1.54E−08 | 5.05E−09 | 2.18E−08 | 3.33E−08 | 4.48E−08 |
| 237 | ++ | A3 | 4.04E−09 | 7.43E−09 | 4.04E−09 | 1.17E−08 | 1.21E−08 | 1.25E−08 | 1.29E−08 | 1.33E−08 | 1.53E−08 | 1.93E−08 | 2.33E−08 |
| 263 | ++ | 3SLN-1 | <4.83E−10 | <4.83E−10 | <4.83E−10 | 7.20E−10 | 8.08E−10 | 8.96E−10 | 9.85E−10 | 1.07E−09 | 1.51E−09 | 8.59E−09 | 1.44E−08 |
| 285 | ++ | SLX-5 | <4.83E−10 | <4.83E−10 | 2.70E−09 | 3.10E−09 | 3.50E−09 | 3.90E−09 | 4.30E−09 | 4.71E−09 | 7.67E−09 | 1.44E−08 | 2.24E−08 |
| 288 | ++ | LA-1 | 2.63E−09 | 4.00E−09 | 5.54E−09 | 6.16E−09 | 6.77E−09 | 7.39E−09 | 8.00E−09 | 8.62E−09 | 1.17E−08 | 2.07E−08 | 3.61E−08 |
| 292 | ++ | STNA-5 | 2.26E−09 | 4.66E−09 | 2.26E−09 | 9.39E−09 | 7.55E−09 | 1.04E−08 | 1.09E−08 | 1.14E−08 | 1.38E−08 | 2.25E−08 | 3.40E−08 |
| 40 | + | BAT-1 | 1.17E−09 | 2.78E−09 | 1.17E−09 | 4.22E−09 | 4.35E−09 | 4.48E−09 | 4.61E−09 | 4.74E−09 | 5.39E−09 | 1.03E−08 | 1.50E−08 |
| 41 | + | SOLX-4 | <4.83E−10 | 1.06E−09 | 1.03E−08 | 1.13E−08 | 1.17E−08 | 1.24E−08 | 1.31E−08 | 1.38E−08 | 1.71E−08 | 2.31E−08 | 2.91E−08 |
| 45 | + | BHD-1 | <4.83E−10 | <4.83E−10 | <4.83E−10 | 1.91E−09 | 2.38E−09 | 2.85E−09 | 3.32E−09 | 3.79E−09 | 6.91E−09 | 1.59E−08 | 2.15E−08 |
| 68 | + | Man7 | 2.11E−09 | 4.28E−09 | 2.11E−09 | >4.83E−08 | >4.83E−08 | >4.83E−08 | 1.68E−08 | 1.58E−08 | 7.80E−09 | >2.42E−08 | >2.42E−08 |
| 71 | + | BBT-1 | 5.99E−10 | 2.78E−09 | 5.99E−10 | 4.24E−09 | 4.37E−09 | 4.50E−09 | 4.63E−09 | 4.77E−09 | 5.65E−09 | 9.57E−08 | 1.35E−08 |
| 91 | + | G6GN-4 | <4.83E−10 | 7.48E−09 | <4.83E−10 | 1.42E−08 | 1.48E−08 | <4.83E−10 | 1.60E−08 | 1.68E−08 | 2.09E−08 | 2.89E−08 | 3.69E−08 |
| 105 | + | Man6 | 4.81E−10 | 2.51E−09 | 4.81E−10 | 5.90E−09 | 6.22E−09 | 6.53E−09 | 6.85E−09 | 7.16E−09 | 9.24E−09 | 1.53E−08 | 2.14E−08 |
| 106 | + | LDF-4 | <4.83E−10 | <4.83E−10 | <4.83E−10 | 2.16E−09 | 2.46E−09 | 2.75E−09 | 3.05E−09 | 3.35E−09 | 4.83E−09 | 2.76E−08 | 3.75E−08 |
| 111 | + | LNB-5 | 2.62E−09 | 3.94E−09 | 2.62E−09 | 5.62E−09 | 6.13E−09 | 6.64E−09 | 7.15E−09 | 7.66E−09 | 1.02E−08 | 1.53E−08 | 3.82E−08 |
| 117 | + | TA-5 | <4.83E−10 | 1.09E−09 | <4.83E−10 | 2.88E−09 | 3.06E−09 | 3.24E−09 | 3.41E−09 | 3.59E−09 | 4.47E−09 | 8.45E−09 | 1.45E−08 |
| 120 | + | A2 | 7.02E−09 | 8.70E−09 | 7.02E−09 | 2.09E−08 | 2.20E−08 | 2.31E−08 | 2.42E−08 | >4.83E−08 | >4.83E−08 | >2.42E−08 | >2.42E−08 |
| 139 | + | 3FL-5 | <4.83E−10 | 7.93E−10 | <4.83E−10 | 1.49E−09 | 1.55E−09 | 1.61E−09 | 1.68E−09 | 1.74E−09 | 2.85E−09 | 2.37E−08 | 3.52E−08 |
| 151 | + | SLX-4 | <4.83E−10 | <4.83E−10 | 1.37E−09 | 1.52E−09 | 1.67E−09 | 1.82E−09 | 1.98E−09 | 2.39E−09 | 4.16E−09 | 1.14E−08 | 2.01E−08 |
| 153 | + | G6GN-5 | <4.83E−10 | 2.53E−09 | <4.83E−10 | 1.14E−08 | 1.29E−08 | 1.45E−08 | 1.60E−08 | 1.70E−08 | 2.16E−08 | 3.09E−08 | 4.02E−08 |
| 186 | + | Man3 | 8.41E−10 | 1.12E−08 | 8.41E−10 | 1.68E−08 | 1.70E−08 | 1.75E−08 | 1.80E−08 | 1.85E−08 | 2.09E−08 | >2.42E−08 | >2.42E−08 |
| 191 | + | LB-1 | <4.83E−10 | 1.40E−09 | <4.83E−10 | 3.50E−09 | 3.77E−09 | 4.04E−09 | 4.31E−09 | 4.59E−09 | 6.04E−09 | 9.18E−09 | 1.23E−08 |
| 197 | + | A4 | 5.01E−09 | 5.85E−09 | 5.01E−09 | 6.77E−09 | 6.86E−09 | 6.94E−09 | 7.03E−09 | 7.11E−09 | 7.53E−09 | 1.60E−08 | >2.42E−08 |
| 209 | + | LDFH-4 | 3.78E−09 | 4.58E−10 | 3.78E−09 | 4.23E−08 | 4.47E−08 | 4.70E−08 | 4.94E−08 | >4.83E−08 | >4.83E−08 | >4.83E−08 | >4.83E−08 |
| 220 | + | LNB-4 | 3.83E−09 | 6.57E−09 | 3.83E−09 | 1.06E−08 | 1.09E−08 | 1.11E−08 | 1.14E−08 | 7.27E−09 | 1.32E−08 | 2.12E−08 | 3.14E−08 |
| 225 | + | 3FL-1 | <4.83E−10 | <4.83E−10 | <4.83E−10 | 1.69E−09 | 1.80E−09 | 1.92E−09 | 2.33E−09 | 2.80E−09 | 5.13E−09 | 1.30E−08 | 2.29E−08 |
| 236 | + | 2FL-5 | 7.44E−09 | 1.18E−08 | 7.44E−09 | 1.70E−08 | 1.77E−08 | 1.84E−08 | 1.92E−08 | 1.99E−08 | 2.34E−08 | 3.05E−08 | 3.76E−08 |
| 240 | + | SOLA-5 | <4.83E−10 | <4.83E−10 | 1.06E−09 | 1.79E−08 | 1.88E−08 | 1.97E−08 | 2.07E−08 | 2.16E−08 | 2.61E−08 | 3.52E−08 | 4.43E−08 |
| 290 | + | SLA-5 | 1.84E−09 | 7.78E−09 | 1.16E−08 | 1.19E−08 | 1.23E−08 | 1.27E−08 | 1.31E−08 | 1.35E−08 | 1.53E−08 | 2.30E−08 | 3.13E−08 |
| 295 | + | 6SLN-4 | <4.83E−10 | 2.30E−09 | <4.83E−10 | 4.18E−09 | 4.18E−09 | 4.33E−09 | 4.49E−09 | 4.65E−09 | 5.63E−09 | 1.00E−08 | 1.44E−08 |
| 50 | − | Man8 | 1.80E−09 | 2.78E−09 | 1.80E−09 | 1.30E−08 | 1.44E−08 | 1.57E−08 | 1.71E−08 | 1.84E−08 | 2.52E−08 | >2.42E−08 | >2.42E−08 |
| 56 | − | Man9 | 1.61E−08 | 2.35E−08 | 1.61E−08 | >4.83E−08 | >4.83E−08 | >4.83E−08 | >4.83E−08 | >4.83E−08 | >4.83E−08 | >2.42E−08 | >2.42E−08 |
| 70 | − | LNT-5 | 3.34E−09 | 4.86E−09 | 3.34E−09 | 2.72E−08 | 2.86E−08 | 3.00E−08 | 3.14E−08 | 3.28E−08 | 3.88E−08 | >4.83E−08 | >4.83E−08 |
| 127 | − | G3G-4 | <4.83E−10 | 1.27E−08 | 4.83E−10 | 1.54E−08 | 1.57E−08 | 1.59E−08 | 1.70E−08 | 1.82E−08 | 2.39E−08 | 3.16E−08 | 3.54E−08 |
| 129 | − | BBT-4 | 2.86E−09 | 3.83E−09 | 2.86E−09 | 4.90E−09 | 5.00E−09 | 5.09E−09 | 5.19E−09 | 5.29E−09 | 7.72E−09 | 1.28E−08 | 1.95E−08 |
| 137 | − | Man4B | 6.02E−10 | 1.04E−08 | 6.02E−10 | 1.96E−08 | 2.04E−08 | 2.13E−08 | 2.21E−08 | 2.30E−08 | >4.83E−08 | >2.42E−08 | >2.42E−08 |
| 141 | − | BBT-5 | 1.66E−09 | 1.80E−09 | 1.66E−09 | 2.47E−09 | 2.74E−09 | 3.02E−09 | 3.29E−09 | 3.56E−09 | 4.92E−09 | 1.14E−08 | 2.36E−08 |
| 142 | − | BHD-5 | 9.87E−10 | 2.07E−09 | 9.87E−10 | 7.48E−09 | 7.92E−09 | 8.37E−09 | 8.81E−09 | 9.25E−09 | 1.15E−08 | 1.59E−08 | 2.92E−08 |
| 175 | − | G3G-5 | 2.01E−08 | 2.49E−08 | 2.01E−08 | 3.02E−08 | 3.06E−08 | 3.11E−08 | 3.16E−08 | 3.21E−08 | 3.45E−08 | 3.93E−08 | 4.40E−08 |
| 183 | − | G4GN-5 | 1.99E−08 | 2.55E−08 | 1.99E−08 | 3.16E−08 | 3.22E−08 | 3.28E−08 | 3.33E−08 | 3.39E−08 | 3.67E−08 | 4.23E−08 | 4.79E−08 |
| 207 | − | 6SL-5 | 3.76E−09 | 4.64E−09 | 4.83E−10 | 8.94E−09 | 1.00E−08 | 1.11E−08 | 1.22E−08 | 1.34E−08 | 1.99E−08 | 3.48E−08 | >4.83E−08 |
| 254 | − | G3G-1 | 2.03E−09 | 4.05E−08 | 2.03E−09 | 7.61E−09 | 8.09E−09 | 8.57E−09 | 9.06E−09 | 9.54E−09 | 1.19E−08 | 1.81E−08 | 3.10E−08 |
| 3 | − | EEGD1A-BH | 4.93E−09 | 1.13E−08 | 2.21E−08 | 2.32E−08 | 2.43E−08 | 2.53E−08 | 2.64E−08 | 2.81E−08 | 4.17E−08 | 6.88E−08 | 6.96E−08 |
| 12 | − | 3SL-4 | 1.18E−08 | 1.79E−09 | 1.18E−08 | 1.24E−08 | 1.26E−08 | 1.28E−08 | 1.29E−08 | 1.43E−08 | 1.48E−08 | 6.38E−10 | >4.83E−08 |
| 130 | − | 2FL-4 | 4.12E−09 | 7.91E−09 | 4.12E−09 | 1.26E−08 | 1.30E−08 | 1.34E−08 | 1.38E−08 | 1.43E−08 | 1.70E−08 | 2.73E−08 | 3.75E−08 |
| 178 | − | LNT-4 | 6.84E−09 | 1.01E−08 | 6.84E−09 | 1.36E−08 | 1.40E−08 | 1.43E−08 | 1.46E−08 | 1.49E−08 | 2.01E−08 | 4.24E−08 | >4.83E−08 |
| 218 | − | EEGM-BH | 7.23E−09 | 2.03E−08 | 6.08E−08 | 6.49E−08 | 6.90E−08 | 7.31E−08 | 7.73E−08 | 8.45E−08 | 1.43E−07 | >2.42E−07 | >2.42E−07 |
| 301 | − | TRIS | 8.56E−09 | 2.02E−08 | 3.18E−08 | 3.30E−08 | 3.41E−08 | 3.53E−08 | 3.64E−08 | 3.76E−08 | 4.34E−08 | >4.83E−08 | >4.83E−08 |
| 302 | − | CEBI | 3.98E−08 | 0.37E−08 | 4.05E−08 | 4.22E−08 | 4.39E−08 | 4.56E−08 | 4.73E−08 | 4.90E−08 | >4.83E−08 | >4.83E−08 | >4.83E−08 |

### Example 8

### ¹²⁵I Labeling of Various Sugar Chain-Bound Liposomes by Chloramine T Method

A chloramine T (Wako Pure Chemical Co., Japan) solution and a sodium disulfite solution were each prepared at 3 mg/ml and 5 mg/ml when they were used. The sugar chain-bound liposomes and tris(hydroxymethyl)aminomethane-bound liposomes prepared in Example 6 were separately added (50 μl each) to Eppen tubes. Subsequently, 15 μl of ¹²⁵I-NaI (NEN Life Science Product, Inc. U.S.A.) and 10 μl of a chloramine T solution were added to perform reaction. 10 μl of a chloramine T solution was added every 5 minutes. At 15 minutes after repeating twice this procedure, 100 μl of sodium disulfite was added as a reducing agent so as to stop the reaction. Next, the resultants were placed on a Sephadex G-50 (Pharmacia Biotech. Sweden) column for chromatography and then subjected to elution with PBS, so that labeled products were purified. Finally, an unlabeled-liposome complex was added and then specific activity (4×10⁶ Bq/mg protein) was adjusted. Thus, ¹²⁵-labeled liposome solutions were obtained.

### Example 9

### Measurement of the Amounts of Various Sugar Chain-Bound Liposome Complexes Transferred from the Intestinal Tract into Blood in Mice

0.2 ml of each ¹²⁵I-labeled sugar chain-bound and tris(hydroxymethyl)aminomethane-bound liposome complex prepared in Example 6 was forcedly administered intraintestinally using oral sonde for mice to male ddY mice (7-week-old) that had been fasted overnight (excluding water), so that the protein amount was 3 μg/mouse. 10 minutes later, 1 ml of blood was collected from inferior vena cava of each mouse under Nembutal anesthesia. ¹²⁵I radioactivity in blood sample was then measured using a gamma counter (Alola ARC300). Furthermore, to examine the in vivo stability of various liposome complexes, the serum of each blood was re-chromatographed using Sephadex G-50. In all cases, most of radioactivity was observed in high-molecular-weight void fractions. Various liposome complexes also had stability in vivo. In addition, each amount of radioactivity that had been transferred from the intestinal tract into blood is represented by the proportion of radioactivity per ml of blood (% dose/ml blood) with respect to the total radioactivity administered. The results are shown in Table 19 below.

TABLE 19

| | Oral |
|---|---|
| No. | administration |
| 3 | NA |
| 27 | + |
| 29 | + |
| 37 | − |
| 38 | NA |
| 40 | ++ |
| 41 | NA |
| 45 | + |
| 50 | + |
| 53 | + |
| 56 | ++ |
| 60 | − |
| 67 | ++ |
| 68 | + |
| 69 | ++ |
| 70 | ++ |
| 71 | + |
| 76 | NA |
| 80 | − |
| 87 | + |
| 91 | NA |
| 93 | − |
| 96 | NA |
| 105 | ++ |
| 106 | NA |
| 111 | − |
| 116 | − |
| 117 | + |
| 120 | + |
| 125 | ++ |
| 127 | NA |
| 129 | NA |
| 130 | NA |
| 137 | NA |
| 139 | ++ |
| 141 | − |
| 142 | ++ |
| 146 | NA |
| 150 | + |
| 151 | NA |
| 152 | ++ |
| 153 | ++ |
| 154 | + |
| 155 | − |
| 175 | ++ |
| 178 | NA |
| 183 | − |
| 184 | ++ |
| 186 | ++ |
| 191 | − |
| 195 | NA |
| 197 | ++ |
| 199 | NA |
| 204 | ++ |
| 207 | − |
| 209 | NA |
| 213 | NA |
| 218 | NA |
| 220 | NA |
| 224 | ++ |
| 225 | ++ |
| 229 | − |
| 230 | ++ |
| 233 | − |
| 234 | − |
| 235 | NA |
| 236 | + |
| 237 | ++ |
| 239 | NA |
| 240 | ++ |
| 249 | − |
| 254 | − |
| 263 | + |
| 273 | ++ |
| 285 | + |
| 288 | ++ |
| 290 | ++ |
| 292 | − |
| 295 | NA |
| 301 | − |

### Example 10

### Study of In Vivo Kinetics of Sugar Chain-Modified Liposome

Of the sugar chain-bound liposomes prepared in the above Examples, liposomes administered herein were: sugar chain-bound liposomes (hereinafter, sugar chain+liposome) and liposomes to which no sugar chain had been bound (hereinafter, sugar chain−liposome). The sugar chain+liposome and the sugar chain-liposome were separately administered to normal mice via intravenous injection or oral administration for the purpose of evaluating the accumulation of such liposome in each organ of the mice.

The experiment was conducted for normal mice and cancer-bearing mice. The procedures are as described below. The distribution amounts of various sugar chain-modified liposomes and liposomes not modified with sugar chains in each tissue were measured as follows. Mice used for this experiment were: normal mice; and male ddY mice (7-week-old) to which Ehrlich ascites tumor (EAT) cells (approximately 2×10⁷) had been transplanted subcutaneously into the thighs and then the cancerous tissue had grown to 0.3 g to 0.6 g (grown for 6 to 8 days). 0.2 ml each of various ¹²⁵I-labeled liposomes prepared in Example 8 was administered to these mice via oral administration or injection via tail vein so that the protein amount was 3 μg/mouse. At 10 or 5 minutes later, tissues (blood, liver, heart, lungs, pancreas, brain, cancerous tissue, inflammatory tissue around cancer, small intestine, large intestine, lymph node, bone marrow, kidney, spleen, thymus gland, and muscle) were excised. The radioactivity of each tissue was measured using a gamma counter (Aloka ARC 300). In addition, the amount of radioactivity distributed in each tissue was obtained by measuring the proportion of radioactivity per gram of each tissue (% dose/g of each tissue) with respect to the total radioactivity administered. After oral administration or intravenous administration of sugar chain-modified liposomes and liposomes (standard liposome) to which tris(hydroxymethyl)aminomethane had been bound instead of a sugar chain, the ratio (magnification) of an average measured value (obtained by averaging the measured values of the sugar chain-modified liposome delivered into blood or each tissue of four mice) to an average measured value (obtained by averaging the values of the standard liposome delivered into blood or each tissue of four mice) was calculated. Thus, transferability into blood and tropism (targeting) for each organ after oral administration were evaluated according to the definition in Table 20A. The results are shown in Table 20B below.

TABLE 20A
Definition for evaluation of tropism (targeting property) (++ and +)

| | | Range of | |
|---|---|---|---|
| | | standardized | |
| | Evaluation | relative value | |
| | of tropism | (average value | No. and |
| | (targeting | of each | abbreviated |
| | property) | liposome/average | name of |
| | (5 minutes after | value of | negative |
| Organ | administration) | negative liposome) | liposome |
| Oral administration | ++ | 2.8~4.4 | 301 |
| (10 minutes after | + | 2.3~2.7 | TRIS |
| administration) | | | |
| Liver | ++ | 1.5~2.1 | 301 |
| | + | 1.2~1.5 | TRIS |
| Heart | ++ | 1.4~2.4 | 301 |
| | + | 1.1~1.4 | TRIS |
| Lung | ++ | 1.6~3.5 | 301 |
| | + | 1.3~1.6 | TRIS |
| Pancreas | ++ | 1.5~2.2 | 301 |
| | + | 1.2~1.5 | TRIS |
| Brain | ++ | 1.5~3.7 | 301 |
| | + | 1.1~1.4 | TRIS |
| Tumor | ++ | 1.5~2.5 | 301 |
| | + | 1.1~1.4 | TRIS |
| Inflammation site | ++ | 1.5~4.9 | 301 |
| | + | 1.2~1.5 | TRIS |
| Small intestine | ++ | 1.7~2.5 | 301 |
| | + | 1.4~1.7 | TRIS |
| Large intestine | ++ | 1.6~2.7 | 301 |
| | + | 1.2~1.5 | TRIS |
| Lymph node | ++ | 2.0~8.7 | 301 |
| | + | 1.6~2.0 | TRIS |
| Bone marrow | ++ | 1.3~1.9 | 301 |
| | + | 1.1~1.3 | TRIS |
| Blood (in blood) | ++ | 1~2.1 | 301 |
| | + | 0.6~0.9 | TRIS |
| Kidney | ++ | 1.7~2.7 | 301 |
| | + | 1.4~1.7 | TRIS |
| Spleen | ++ | 2.3~4.0 | 301 |
| | + | 1.6~2.2 | TRIS |
| Thymus gland | ++ | 2.1~4.3 | 301 |
| | + | 1.6~2.1 | TRIS |
| Muscle | ++ | 1.4~2.8 | 301 |
| | + | 1.1~1.4 | TRIS |

TABLE 20B

| | Abbreviated | Oral | | | | | | | Inflammation | Small | Large |
|---|---|---|---|---|---|---|---|---|---|---|---|
| No. | name | administration | Liver | Heart | Lung | Pancreas | Brain | Tumor | site | intestine | intestine |
| 3 | EEGD1A-BH | NA | ++ | − | − | − | − | − | − | − | − |
| 27 | LDF-5 | + | + | − | + | ++ | − | ++ | ++ | ++ | ++ |
| 29 | 3SL-5 | + | + | ++ | ++ | ++ | ++ | ++ | − | ++ | + |
| 37 | FEEGD1b | − | ++ | − | − | − | − | − | − | − | ++ |
| 38 | G4GN-4 | NA | ++ | ++ | ++ | ++ | + | ++ | ++ | ++ | ++ |
| 40 | BAT-1 | ++ | − | ++ | + | ++ | ++ | + | + | ++ | + |
| 41 | SOLX-4 | NA | + | − | − | − | − | + | + | − | − |
| 45 | BHD-1 | + | − | + | − | − | ++ | + | − | − | ++ |
| 50 | Man8 | + | − | ++ | + | − | ++ | − | + | ++ | − |
| 53 | L2F-1 | + | + | + | ++ | ++ | + | ++ | ++ | ++ | ++ |
| 56 | Man9 | ++ | − | + | − | + | + | − | + | + | − |
| 60 | 6SLN-1 | − | − | ++ | ++ | + | ++ | ++ | ++ | ++ | ++ |
| 67 | FEEaMDa | ++ | + | ++ | + | − | + | − | − | − | − |
| 68 | Man7 | + | − | ++ | ++ | ++ | ++ | + | + | ++ | ++ |
| 69 | L4F-1 | ++ | − | − | ++ | + | + | ++ | ++ | ++ | + |
| 70 | LNT-5 | ++ | + | ++ | ++ | + | ++ | − | ++ | + | + |
| 71 | BBT-1 | + | − | + | + | ++ | ++ | + | + | + | + |
| 76 | BHD-4 | NA | − | + | − | + | + | − | + | − | − |
| 80 | 6SLN-5 | − | − | + | − | − | + | − | − | + | ++ |
| 87 | TA-1 | + | − | − | − | + | + | ++ | ++ | ++ | + |
| 91 | G6GN-4 | NA | + | + | ++ | ++ | − | + | ++ | ++ | ++ |
| 93 | A36 | − | − | ++ | ++ | ++ | ++ | ++ | ++ | ++ | ++ |
| 96 | TA-4 | NA | + | ++ | ++ | ++ | − | ++ | ++ | ++ | ++ |
| 105 | Man6 | ++ | − | ++ | + | ++ | ++ | + | ++ | ++ | ++ |
| 106 | LDF-4 | NA | + | + | + | ++ | + | + | ++ | ++ | ++ |
| 111 | LNB-5 | − | + | + | + | + | − | + | + | − | + |
| 116 | LDF-1 | − | − | NA | + | ++ | + | ++ | ++ | ++ | ++ |
| 117 | TA-5 | + | + | − | ++ | + | − | + | + | + | ++ |
| 120 | A2 | + | − | + | ++ | + | ++ | + | + | ++ | + |
| 125 | LX-1 | ++ | − | − | ++ | ++ | − | ++ | ++ | + | + |
| 127 | G3G-4 | NA | − | + | − | + | + | − | − | − | − |
| 128 | BBT-4 | NA | − | − | − | − | + | − | − | − | − |
| 130 | 2FL-4 | NA | + | − | − | + | − | − | − | + | + |
| 137 | Man4B | NA | − | ++ | ++ | ++ | ++ | − | + | ++ | + |
| 139 | 3FL-5 | ++ | + | + | + | ++ | − | + | ++ | + | ++ |
| 141 | BBT-5 | − | − | − | − | − | + | − | − | − | − |
| 142 | BHD-5 | ++ | − | + | − | + | + | − | − | − | − |
| 146 | 3FL-4 | NA | ++ | + | + | + | + | + | ++ | ++ | + |
| 150 | TNA-5 | + | + | + | ++ | + | + | ++ | ++ | + | − |
| 151 | SLX-4 | NA | ++ | ++ | ++ | ++ | ++ | + | + | ++ | ++ |
| 152 | LY-1 | ++ | + | + | ++ | + | + | ++ | ++ | ++ | + |
| 153 | G6GN-5 | ++ | − | + | + | ++ | − | + | ++ | + | ++ |
| 154 | Lac-5 | + | ++ | ++ | ++ | ++ | + | ++ | ++ | ++ | ++ |
| 155 | A6 | − | − | ++ | + | + | ++ | ++ | ++ | ++ | ++ |
| 175 | G3G-5 | ++ | − | ++ | + | + | + | − | − | + | − |
| 178 | LNT-4 | NA | + | + | ++ | − | + | − | − | − | − |
| 183 | G4GN-5 | − | ++ | + | ++ | − | − | − | + | − | − |
| 184 | STNA-1 | ++ | + | ++ | ++ | ++ | + | ++ | ++ | ++ | ++ |
| 186 | Man3 | ++ | − | ++ | + | − | ++ | + | + | ++ | + |
| 191 | LB-1 | − | − | − | + | + | − | + | ++ | + | + |
| 195 | TNA-4 | NA | ++ | ++ | ++ | ++ | − | ++ | ++ | ++ | ++ |
| 197 | A4 | ++ | − | ++ | + | + | ++ | + | + | + | + |
| 199 | STNA-4 | NA | + | ++ | ++ | ++ | − | ++ | ++ | ++ | ++ |
| 204 | Lac-1 | ++ | + | + | ++ | + | − | ++ | ++ | + | + |
| 207 | 6SL-5 | − | − | ++ | ++ | + | ++ | − | − | + | + |
| 209 | LDFH-4 | NA | ++ | ++ | ++ | ++ | ++ | + | + | ++ | ++ |
| 213 | LX-4 | NA | − | + | + | ++ | + | ++ | ++ | ++ | ++ |
| 218 | EEGM1-BH | NA | ++ | − | − | − | − | − | − | − | − |
| 220 | LNB-4 | NA | − | + | + | ++ | − | + | ++ | + | ++ |
| 224 | Man5 | ++ | − | + | + | ++ | ++ | ++ | ++ | ++ | ++ |
| 225 | 3FL-1 | ++ | ++ | − | − | + | − | + | − | + | + |
| 229 | G6GN-1 | − | + | ++ | ++ | ++ | + | ++ | ++ | ++ | ++ |
| 230 | G4GN-1 | ++ | + | + | − | ++ | + | ++ | ++ | ++ | ++ |
| 233 | 2FL-1 | − | − | − | ++ | ++ | ++ | ++ | ++ | ++ | + |
| 234 | TNA-1 | − | ++ | + | ++ | + | − | ++ | ++ | + | + |
| 235 | Lac-4 | NA | − | + | + | ++ | + | ++ | ++ | ++ | + |
| 236 | 2FL-5 | + | + | − | − | − | − | + | − | + | + |
| 237 | A3 | ++ | − | ++ | ++ | ++ | ++ | ++ | ++ | ++ | ++ |
| 239 | SLA-4 | NA | ++ | − | + | − | − | − | − | − | − |
| 240 | SOLA-5 | ++ | ++ | − | − | − | − | + | + | − | − |
| 249 | 3SL-1 | − | − | ++ | + | ++ | ++ | − | − | ++ | ++ |
| 254 | G3G-1 | − | − | + | − | − | − | − | − | − | − |
| 263 | 3SLN-1 | + | − | ++ | ++ | ++ | ++ | ++ | ++ | ++ | ++ |
| 273 | BAT-5 | ++ | − | ++ | − | + | ++ | − | − | − | − |
| 285 | SLX-5 | + | ++ | − | − | − | − | ++ | − | − | − |
| 288 | LA-1 | ++ | − | − | + | − | − | ++ | + | − | − |
| 290 | SLA-5 | ++ | ++ | − | − | − | + | + | ++ | − | − |
| 292 | STNA-5 | − | + | ++ | ++ | ++ | + | ++ | ++ | ++ | ++ |
| 295 | 6SLN-4 | NA | − | ++ | + | ++ | ++ | + | + | ++ | ++ |
| 301 | TRIS | − | − | − | − | − | − | − | − | − | − |

| | Abbreviated | | Bone | Blood | | | Thymus | |
|---|---|---|---|---|---|---|---|---|
| No. | name | Lymph node | marrow | (in blood) | Kidney | Spleen | gland | Muscle |
| 3 | EEGD1A-BH | − | ++ | − | − | ++ | − | − |
| 27 | LDF-5 | + | ++ | + | + | − | + | ++ |
| 29 | 3SL-5 | ++ | ++ | ++ | ++ | − | − | − |
| 37 | FEEGD1b | − | − | − | − | − | − | − |
| 38 | G4GN-4 | ++ | ++ | − | ++ | + | ++ | ++ |
| 40 | BAT-1 | ++ | + | ++ | + | − | ++ | + |
| 41 | SOLX-4 | − | − | − | − | − | − | − |
| 45 | BHD-1 | + | + | ++ | + | − | − | + |
| 50 | Man8 | − | ++ | ++ | + | − | − | + |
| 53 | L2F-1 | + | ++ | + | ++ | + | + | ++ |
| 56 | Man9 | − | − | ++ | + | − | − | + |
| 60 | 6SLN-5 | ++ | ++ | ++ | ++ | − | ++ | ++ |
| 67 | FEEaMDa | − | ++ | ++ | ++ | − | − | ++ |
| 68 | Man7 | + | + | ++ | ++ | − | − | + |
| 69 | L4F-1 | + | + | + | + | + | + | ++ |
| 70 | LNT-5 | + | ++ | + | ++ | − | − | ++ |
| 71 | BBT-1 | + | + | ++ | + | − | + | + |
| 76 | BHD-4 | − | − | ++ | + | − | + | + |
| 80 | 6SLN-5 | ++ | + | + | + | − | + | + |
| 87 | TA-1 | ++ | ++ | − | − | − | − | ++ |
| 91 | G6GN-4 | + | + | − | − | + | ++ | ++ |
| 93 | A36 | ++ | ++ | ++ | ++ | − | ++ | ++ |
| 96 | TA-4 | + | ++ | − | ++ | − | + | ++ |
| 105 | Man6 | − | ++ | ++ | ++ | − | ++ | ++ |
| 106 | LDF-4 | ++ | ++ | + | ++ | + | + | ++ |
| 111 | LNB-5 | − | − | − | − | − | − | + |
| 116 | LDF-1 | ++ | ++ | + | + | + | + | ++ |
| 117 | TA-5 | + | − | − | − | + | − | + |
| 120 | A2 | + | + | ++ | ++ | − | − | + |
| 125 | LX-1 | + | − | − | − | + | ++ | ++ |
| 127 | G3G-4 | − | − | ++ | + | + | − | − |
| 128 | BBT-4 | − | − | + | − | − | − | − |
| 130 | 2FL-4 | + | − | − | − | − | − | + |
| 137 | Man4B | + | ++ | ++ | ++ | − | + | ++ |
| 139 | 3FL-5 | + | − | − | + | + | + | ++ |
| 141 | BBT-5 | + | − | + | − | − | − | − |
| 142 | BHD-5 | ++ | − | ++ | + | − | − | − |
| 146 | 3FL-4 | ++ | ++ | + | ++ | + | ++ | + |
| 150 | TNA-5 | + | + | + | + | ++ | + | ++ |
| 151 | SLX-4 | ++ | + | ++ | ++ | ++ | ++ | + |
| 152 | LY-1 | − | ++ | − | − | ++ | ++ | ++ |
| 153 | G6GN-5 | + | + | − | + | + | + | ++ |
| 154 | Lac-5 | ++ | ++ | + | ++ | ++ | ++ | ++ |
| 155 | A6 | + | ++ | ++ | ++ | − | ++ | ++ |
| 175 | G3G-5 | + | + | ++ | ++ | − | − | − |
| 178 | LNT-4 | − | + | − | + | + | + | − |
| 183 | G4GN-5 | − | − | − | + | ++ | − | + |
| 184 | STNA-1 | ++ | + | + | + | ++ | ++ | ++ |
| 186 | Man3 | ++ | ++ | ++ | ++ | − | − | ++ |
| 191 | LB-1 | − | + | − | − | + | − | + |
| 195 | TNA-4 | + | + | + | ++ | ++ | ++ | ++ |
| 197 | A4 | − | ++ | ++ | ++ | − | − | + |
| 199 | STNA-4 | ++ | ++ | − | ++ | ++ | ++ | ++ |
| 204 | Lac-1 | + | + | + | + | + | − | + |
| 207 | 6SL-5 | ++ | + | ++ | + | − | + | − |
| 209 | LDFH-4 | ++ | ++ | + | ++ | + | − | ++ |
| 213 | LX-4 | + | + | − | − | + | ++ | ++ |
| 218 | EEGM1-BH | − | NA | − | − | − | − | − |
| 220 | LNB-4 | + | ++ | − | + | − | ++ | ++ |
| 224 | Man5 | − | ++ | ++ | ++ | − | − | ++ |
| 225 | 3FL-1 | + | − | + | − | ++ | − | + |
| 229 | G6GN-1 | + | ++ | − | + | ++ | + | ++ |
| 230 | G4GN-1 | + | ++ | − | + | ++ | + | ++ |
| 233 | 2FL-1 | + | ++ | + | + | − | ++ | ++ |
| 234 | TNA-1 | + | + | − | − | ++ | + | ++ |
| 235 | Lac-4 | + | + | + | + | − | ++ | ++ |
| 236 | 2FL-5 | + | − | − | − | − | − | + |
| 237 | A3 | ++ | ++ | ++ | ++ | − | + | ++ |
| 239 | SLA-4 | − | − | − | − | ++ | − | − |
| 240 | SOLA-5 | + | − | − | − | + | − | − |
| 249 | 3SL-1 | ++ | ++ | ++ | ++ | − | − | + |
| 254 | G3G-1 | − | − | + | − | − | − | − |
| 263 | 3SLN-1 | ++ | ++ | ++ | ++ | + | − | ++ |
| 273 | BAT-5 | − | − | ++ | + | − | ++ | + |
| 285 | SLX-5 | − | − | − | − | − | − | − |
| 288 | LA-1 | ++ | + | − | − | + | − | + |
| 290 | SLA-5 | − | + | − | − | + | ++ | − |
| 292 | STNA-5 | ++ | ++ | + | ++ | ++ | ++ | ++ |
| 295 | 6SLN-4 | ++ | ++ | ++ | + | − | ++ | ++ |
| 301 | TRIS | − | − | ++ | − | − | − | − |

Table 14 and Table 15B show the evaluation results showing the effects of accumulating radioactive sugar chain liposomes in each tissue when various radioactive sugar chain-modified liposomes were administered to mice via oral administration and intravenous injection. These results demonstrate that the sugar chain-modified liposomes can achieve highly efficient accumulation and delivery of drugs, fluorescent substances, radiolabeled substances, or the like via active targeting to parts affected by diseases or various organs with the use of the functions of a sugar chain as a ligand. Therefore, the sugar chain-modified liposomes of the present invention can visualize accumulation in a target tissue such as a tumor. Hence, according to the present invention, a delivery vehicle for use as therapeutic drugs and a delivery vehicle for use as a research reagent, a diagnostic agent, or the like are also provided.

### Example 11

### Preparation of Anticancer Agent Doxorubicin-Encapsulated Liposome

A liposome was prepared by the techniques of the previous report (Yamazaki, N., Kodama, M. and Gabius, H.-J. (1994) Methods Enzymol. 242, 56-65) using improved cholic acid dialysis. Specifically, dipalmitoylphosphatidylcholine, cholesterol, dicetylphosphate, ganglioside, and dipalmitoylphosphatidylethanol amine were mixed at a molar ratio of 35:40:5:15:5 so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, thereby obtaining a lipid membrane. The thus obtained lipid membrane was suspended in 10 ml of a TAPS buffered saline solution (pH8.4) and then the resultant was ultrasonicated, so that 10 ml of a transparent micelle suspension was obtained. An anticancer agent doxorubicin that had been completely dissolved in a TAPS buffer solution (pH8.4) to a concentration of 3 mg/1 ml was slowly added dropwise to the micelle suspension while agitating the suspension. After homogeneously mixing the suspension, the doxorubicin-containing micelle suspension was subjected to ultrafiltration using a PM10 membrane (Amicon Co., U.S.A.) and a TAPS buffered saline solution (pH8.4) so that 10 ml of a homogeneous anticancer agent doxorubicin-encapsulated liposome particle suspension was prepared.

The particle diameter and zeta potential of the anticancer agent doxorubicin-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV.

### Example 12

### Hydrophilization of the Anticancer Agent Doxorubicin-Encapsulated Liposome Lipid Membrane Surface

10 ml of the anticancer agent doxorubicin-encapsulated liposome solution prepared in Example 11 was subjected to ultrafiltration using an XM300 membrane (Amicon Co., U.S.A.) and a CBS buffer solution (pH8.5). The pH of the solution was adjusted to 8.5. Next, 10 ml of a cross-linking reagent bis(sulfo succinimidyl)suberate (BS3; Pierce Co., U.S.A.) was added. The solution was agitated for 2 hours of agitation at 25° C. and then agitated overnight at 7° C. Thus, chemical binding reaction between lipid dipalmitoylphosphatidylethanol amine on the liposome membrane and BS3 was completed. Subsequently, the liposome solution was subjected to ultrafiltration using an XM300 membrane and a CBS buffer solution (pH8.5). Next, 40 mg of tris(hydroxymethyl)aminomethane dissolved in 1 ml of a CBS buffer solution (pH8.5) was added to 10 ml of the liposome solution. The solution was agitated for 2 hours at 25° C. and then agitated overnight at 7° C. Thus, chemical binding reaction between BS3 bound to the lipids on the liposome membrane and tris(hydroxymethyl)aminomethane was completed. Therefore, the hydroxy group of tris(hydroxymethyl)aminomethane was coordinated on the lipid dipalmitoylphosphatidylethanol amine of the anticancer agent doxorubicin-encapsulated liposome membrane, so that the liposome membrane surface was hydrated and hydrophilized.

### Example 13

### Binding of Human Serum Albumin (HSA) onto Anticancer Agent Doxorubicin-Encapsulated Liposome Membrane Surface

Human serum albumin (HSA) was bound onto a liposome membrane surface according to the technique of the previous report (Yamazaki, N., Kodama, M. and Gabius, H.-J. (1994) Methods Enzymol. 242, 56-65) using a coupling reaction method. Specifically, the reaction was performed as a two-step chemical reaction. First, ganglioside existing on the membrane surface of 10 ml of the liposome obtained in Example 2 was added to 43 mg of sodium metaperiodate dissolved in 1 ml of a TAPS buffer solution (pH8.4), followed by 2 hours of agitation at room temperature to perform periodate oxidation. The resultant was subjected to ultrafiltration using an XM300 membrane and a PBS buffer solution (pH8.0) so that 10 ml of the thus oxidized liposome was obtained. 20 mg of human serum albumin (HSA) was added to the liposome solution, followed by 2 hours of agitation at 25° C. Next, 100 μl of 2M NaBH₃CN was added to PBS (pH8.0) and then the solution was agitated overnight at 10° C. Thus, HSA was bound by the coupling reaction between ganglioside on the liposome and HSA. The resultant was then subjected to ultrafiltration using an XM300 membrane and a CBS buffer solution (pH8.5), so that 10 ml of an HSA-bound anticancer agent doxorubicin-encapsulated liposome solution was obtained.

### Example 14

### Preparation of Sugar Chain

Sugar chains were prepared by the procedures similar to those in Example 4.

### Example 15

### Binding of Sugar Chain onto Anticancer Agent Doxorubicin-Encapsulated Liposome Membrane-Surface-Bound Human Serum Albumin (HSA) and Hydrophilization of Linker Protein (HSA)

50 μg of each sugar chain prepared in Example 14 was added to 0.5 ml of an aqueous solution in which 0.25 g of NH₄HCO₃ had been dissolved, followed by 3 days of agitation at 37° C. The resultant was filtered with a 0.45-μm filter to complete the amination reaction of the reducing termini of the sugar chains. Thus, 50 μg of a glycosylamine compound of each sugar chain was obtained. Next, 1 mg of a cross-linking reagent 3,3′-dithiobis(sulfo succinimidyl propionate (DTSSP; Pierce Co., U.S.A.) was added to 1 ml of the anticancer agent doxorubicin-encapsulated liposome solution (a portion of the liposome solution) obtained in Example 13. The solution was agitated for 2 hours at 25° C. and then agitated overnight at 7° C. The resultant was subjected to ultrafiltration using an XM300 membrane and a CBS buffer solution (pH8.5), so that 1 ml of liposome was obtained on which DTSSP had been bound to HSA on the liposome. Next, 50 μg of the glycosylamine compound of the above sugar chain was added to the liposome solution. The solution was agitated for 2 hours at 25° C. and then agitated overnight at 7° C. The resultant was subjected to ultrafiltration using an XM300 membrane and a PBS buffer solution (pH7.2) so as to bind each sugar chain to DTSSP on the liposome membrane-surface-bound human serum albumin. Next, 13 mg of tris(hydroxymethyl)aminomethane (Wako Co., Japan) was added to the liposome solution. The solution was agitated for 2 hours at 25° C. and then agitated overnight at 7° C. The resultant was subjected to ultrafiltration using an XM300 membrane and a PBS buffer solution (pH7.2), so as to bind the glycosylated amine compound to DTSSP on the liposome membrane-surface-bound human serum albumin. As a result, a liposome was obtained, in which tris(hydroxymethyl)aminomethane, human serum albumin, and the liposome were bound and the linker protein (HSA) was hydrophilized. As a result, 2 ml of an anticancer agent doxorubicin-encapsulated liposome (total amount of lipid: 2 mg and total amount of protein: 200 μg) was obtained, in which each sugar chain, human serum albumin, and the liposome were bound and the linker protein (HSA) was hydrophilized.

The particle diameter and zeta potential of the anticancer agent doxorubicin-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV.

### Comparative Example

### Hydrophilization of Linker Protein (HSA) Via Binding of Tris(Hydroxymethyl)Aminomethane to Anticancer Agent Doxorubicin-Encapsulated Liposome Membrane-Surface-Bound Human Serum Albumin (HSA)

To prepare an anticancer agent doxorubicin-encapsulated liposome as a sample for comparison, 1 mg of a cross-linking reagent 3,3′-dithiobis(sulfosuccinimidyl propionate (DTSSP; Pierce Co., U.S.A.) was added to 1 ml of the anticancer agent doxorubicin-encapsulated liposome solution (a portion of the liposome solution) obtained in Example 13. The solution was agitated for 2 hours at 25° C. and then agitated overnight at 7° C. The resultant was subjected to ultrafiltration using an XM300 membrane and a CBS buffer solution (pH8.5), so that 1 ml of liposome was obtained in which DTSSP was bound to HSA on the liposome and the linker protein (HSA) was hydrophilized. Next, 13 mg of tris(hydroxymethyl)aminomethane (Wako Co., Japan) was added to the liposome solution. The solution was agitated for 2 hours at 25° C. and then agitated overnight at 7° C. The resultant was subjected to ultrafiltration using an XM300 membrane and a PBS buffer solution (pH 7.2) so as to bind the glycosylation amine compound to DTSSP on the liposome membrane-surface-bound human serum albumin. As a result, 2 ml of an anticancer agent doxorubicin-encapsulated liposome (abbreviated name: DX-TRIS) (total amount of lipid; 2 mg and total amount of protein: 200 μg) as a sample for comparison was obtained, in which tris(hydroxymethyl)aminomethane, human serum albumin, and the liposome were bound and the linker protein (HSA) was hydrophilized.

The particle diameter and zeta potential of the anticancer agent doxorubicin-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV.

### Example 16

### Hydrophilization of Liposome Membrane-Surface-Bound Human Serum Albumin (HSA)

Sugar chain-bound liposomes prepared by the means of Example 15 were separately subjected to hydrophilization of the HSA protein surfaces on the liposomes as described in the following procedures. 13 mg of tris(hydroxymethyl)aminomethane was added to 2 ml of each sugar chain-bound liposome. The solution was agitated for 2 hours at 25° C. and then agitated overnight at 7° C. The resultant was subjected to ultrafiltration using an XM300 membrane and a PBS buffer solution (pH7.2) and then unreacted substances were removed. As a result, the final product, 2 ml of each hydrophilized sugar chain-bound liposome complex (total amount of lipid; 2 mg, total amount of protein: 200 μg, and average particle diameter: 100 nm) was obtained.

### Example 17

### Determination of Inhibitory Effect of Various Sugar Chain-Bound Liposome Complexes on Lectin Binding Activity)

The in vitro lectin binding activity of each sugar chain-bound liposome complex (prepared by the means of Example 16) was determined in an inhibition experiment using a lectin-immobilized microplate according to a standard method (Yamazaki, N. (1999) Drug Delivery System, 14, 498-505). Specifically, a lectin (e.g., E-selectin; R&D Systems Co., U.S.A.) was immobilized on a 96-well microplate. 0.1 μg of biotinylated fucosylated fetuin (ligand for comparison) and each of various sugar chain-bound liposome complexes (amounts of protein: 0.01 μg, 0.04 μg, 0.11 μg, 0.33 μg, and 1 μg) varying in concentration were added to the lectin-immobilized plate, followed by 2 hours of incubation at 4° C. After 3 times of washing with PBS (pH7.2), horse radish peroxidase (HRPO)-conjugated streptavidin was added to the resultants. Incubation was further performed at 4° C. for 1 hour and then the resultants were washed 3 times with PBS (pH7.2). Peroxidase substrate was added, the resultants were allowed to stand at room temperature, and then absorbance was measured at 405 nm using a microplate reader (Molecular Devices Corp., U.S.A.). Fucosylated fetuin was biotinylated as follows. Treatment was performed with a sulfo-NHS-biotin reagent (Pierce Co., U.S.A.) and then purification was performed with Centricon-30 (Amicon Co., U.S.A.). HRPO-conjugated streptavidin was prepared by oxidation of HRPO and conjugation of streptavidin via reductive amination using NaBH₃CN. The measurement results are as shown in Table 21 below.

TABLE 21A

| Sample ID | IC10 | Sample ID | IC10 |
|---|---|---|---|
| 76 | <4.83E−10 | 224 | 2.97E−10 |
| 45 | <4.83E−10 | 105 | 4.81E−10 |
| 127 | <4.83E−10 | 71 | 5.99E−10 |
| 273 | <4.83E−10 | 137 | 6.02E−10 |
| 295 | <4.83E−10 | 186 | 8.41E−10 |
| 80 | <4.83E−10 | 93 | 9.73E−10 |
| 263 | <4.83E−10 | 142 | 9.87E−10 |
| 69 | <4.83E−10 | 40 | 1.17E−09 |
| 53 | <4.83E−10 | 116 | 1.21E−09 |
| 91 | <4.83E−10 | 60 | 1.25E−09 |
| 153 | <4.83E−10 | 38 | 1.39E−09 |
| 230 | <4.83E−10 | 233 | 1.43E−09 |
| 229 | <4.83E−10 | 141 | 1.66E−09 |
| 195 | <4.83E−10 | 50 | 1.80E−09 |
| 150 | <4.83E−10 | 290 | 1.84E−09 |
| 199 | <4.83E−10 | 254 | 2.03E−09 |
| 184 | <4.83E−10 | 68 | 2.11E−09 |
| 234 | <4.83E−10 | 292 | 2.26E−09 |
| 146 | <4.83E−10 | 111 | 2.62E−09 |
| 139 | <4.83E−10 | 288 | 2.63E−09 |
| 154 | <4.83E−10 | 129 | 2.86E−09 |
| 204 | <4.83E−10 | 70 | 3.34E−09 |
| 235 | <4.83E−10 | 207 | 3.76E−09 |
| 225 | <4.83E−10 | 209 | 3.78E−09 |
| 106 | <4.83E−10 | 220 | 3.83E−09 |
| 27 | <4.83E−10 | 237 | 4.04E−09 |
| 96 | <4.83E−10 | 130 | 4.12E−09 |
| 117 | <4.83E−10 | 3 | 4.93E−09 |
| 87 | <4.83E−10 | 197 | 5.01E−09 |
| 152 | <4.83E−10 | 155 | 6.47E−09 |
| 191 | <4.83E−10 | 178 | 6.84E−09 |
| 213 | <4.83E−10 | 120 | 7.02E−09 |
| 125 | <4.83E−10 | 218 | 7.23E−09 |
| 151 | <4.83E−10 | 236 | 7.44E−09 |
| 285 | <4.83E−10 | 301 | 8.56E−09 |
| 239 | <4.83E−10 | 56 | 1.61E−08 |
| 41 | <4.83E−10 | 183 | 1.99E−08 |
| 240 | <4.83E−10 | 175 | 2.01E−08 |
| 29 | 1.21E−11 | 302 | 3.97E−08 |

TABLE 21B

| Sample ID | IC20 | Sample ID | IC20 |
|---|---|---|---|
| 87 | <4.83E−10 | 50 | 2.78E−09 |
| 45 | <4.83E−10 | 71 | 2.78E−09 |
| 263 | <4.83E−10 | 230 | 2.85E−09 |
| 53 | <4.83E−10 | 154 | 3.08E−09 |
| 229 | <4.83E−10 | 234 | 3.41E−09 |
| 195 | <4.83E−10 | 233 | 3.41E−09 |
| 150 | <4.83E−10 | 116 | 3.78E−09 |
| 199 | <4.83E−10 | 129 | 3.83E−09 |
| 184 | <4.83E−10 | 111 | 3.94E−09 |
| 146 | <4.83E−10 | 288 | 4.00E−09 |
| 204 | <4.83E−10 | 254 | 4.05E−09 |
| 225 | <4.83E−10 | 68 | 4.28E−09 |
| 106 | <4.83E−10 | 224 | 4.43E−09 |
| 96 | <4.83E−10 | 207 | 4.64E−09 |
| 152 | <4.83E−10 | 292 | 4.66E−09 |
| 125 | <4.83E−10 | 70 | 4.86E−09 |
| 151 | <4.83E−10 | 60 | 5.30E−09 |
| 285 | <4.83E−10 | 197 | 5.85E−09 |
| 240 | <4.83E−10 | 220 | 6.57E−09 |
| 209 | 4.58E−10 | 237 | 7.43E−09 |
| 139 | 7.93E−10 | 91 | 7.48E−09 |
| 213 | 9.28E−10 | 290 | 7.78E−09 |
| 41 | 1.06E−09 | 130 | 7.91E−09 |
| 117 | 1.09E−09 | 120 | 8.70E−09 |
| 69 | 1.28E−09 | 178 | 1.01E−08 |
| 191 | 1.40E−09 | 155 | 1.01E−08 |
| 80 | 1.44E−09 | 137 | 1.04E−08 |
| 235 | 1.48E−09 | 93 | 1.06E−08 |
| 27 | 1.49E−09 | 186 | 1.12E−08 |
| 239 | 1.58E−09 | 3 | 1.13E−08 |
| 141 | 1.80E−09 | 236 | 1.18E−08 |
| 38 | 1.81E−09 | 29 | 1.23E−08 |
| 142 | 2.07E−09 | 127 | 1.27E−08 |
| 273 | 2.23E−09 | 301 | 2.02E−08 |
| 295 | 2.30E−09 | 218 | 2.03E−08 |
| 76 | 2.39E−09 | 56 | 2.35E−08 |
| 105 | 2.51E−09 | 302 | 2.37E−08 |
| 153 | 2.53E−09 | 175 | 2.49E−08 |
| 40 | 2.78E−09 | 183 | 2.55E−08 |

TABLE 21C

| Sample ID | IC30 | Sample ID | IC30 |
|---|---|---|---|
| 87 | <4.83E−10 | 93 | 9.45E−10 |
| 76 | <4.83E−10 | 142 | 9.87E−10 |
| 45 | <4.83E−10 | 240 | 1.06E−09 |
| 273 | <4.83E−10 | 40 | 1.17E−09 |
| 295 | <4.83E−10 | 116 | 1.21E−09 |
| 80 | <4.83E−10 | 60 | 1.25E−09 |
| 263 | <4.83E−10 | 151 | 1.37E−09 |
| 69 | <4.83E−10 | 38 | 1.39E−09 |
| 53 | <4.83E−10 | 233 | 1.43E−09 |
| 91 | <4.83E−10 | 141 | 1.66E−09 |
| 153 | <4.83E−10 | 50 | 1.80E−09 |
| 230 | <4.83E−10 | 254 | 2.03E−09 |
| 229 | <4.83E−10 | 68 | 2.11E−09 |
| 195 | <4.83E−10 | 292 | 2.26E−09 |
| 150 | <4.83E−10 | 111 | 2.62E−09 |
| 199 | <4.83E−10 | 285 | 2.70E−09 |
| 184 | <4.83E−10 | 129 | 2.86E−09 |
| 234 | <4.83E−10 | 70 | 3.34E−09 |
| 146 | <4.83E−10 | 209 | 3.78E−09 |
| 139 | <4.83E−10 | 220 | 3.83E−09 |
| 154 | <4.83E−10 | 237 | 4.04E−09 |
| 204 | <4.83E−10 | 130 | 4.12E−09 |
| 235 | <4.83E−10 | 213 | 4.94E−09 |
| 225 | <4.83E−10 | 197 | 5.01E−09 |
| 106 | <4.83E−10 | 288 | 5.54E−09 |
| 27 | <4.83E−10 | 155 | 6.47E−09 |
| 96 | <4.83E−10 | 178 | 6.84E−09 |
| 117 | <4.83E−10 | 120 | 7.02E−09 |
| 152 | <4.83E−10 | 239 | 7.28E−09 |
| 191 | <4.83E−10 | 236 | 7.44E−09 |
| 224 | 2.97E−10 | 41 | 1.03E−08 |
| 105 | 4.81E−10 | 290 | 1.16E−08 |
| 127 | 4.83E−10 | 56 | 1.61E−08 |
| 29 | 4.83E−10 | 183 | 1.99E−08 |
| 207 | 4.83E−10 | 175 | 2.01E−08 |
| 71 | 5.99E−10 | 3 | 2.21E−08 |
| 137 | 6.02E−10 | 301 | 3.18E−08 |
| 125 | 6.28E−10 | 302 | 4.05E−08 |
| 186 | 8.41E−10 | 218 | 6.08E−08 |

TABLE 21D

| Sample ID | IC40 | Sample ID | IC40 |
|---|---|---|---|
| 53 | 5.96E−10 | 224 | 1.49E−08 |
| 263 | 1.52E−09 | 80 | 1.52E−08 |
| 139 | 2.85E−09 | 237 | 1.53E−08 |
| 184 | 3.55E−09 | 290 | 1.54E−08 |
| 150 | 3.87E−09 | 130 | 1.70E−08 |
| 151 | 4.16E−09 | 41 | 1.71E−08 |
| 117 | 4.47E−09 | 60 | 1.84E−08 |
| 152 | 4.48E−09 | 29 | 1.90E−08 |
| 106 | 4.83E−09 | 207 | 1.99E−08 |
| 141 | 4.92E−09 | 178 | 2.01E−08 |
| 225 | 5.13E−09 | 155 | 2.01E−08 |
| 27 | 5.22E−09 | 38 | 2.06E−08 |
| 40 | 5.39E−09 | 91 | 2.09E−08 |
| 295 | 5.63E−09 | 186 | 2.09E−08 |
| 71 | 5.65E−09 | 69 | 2.13E−08 |
| 191 | 6.04E−09 | 153 | 2.16E−08 |
| 146 | 6.10E−09 | 233 | 2.17E−08 |
| 229 | 6.25E−09 | 235 | 2.18E−08 |
| 96 | 6.50E−09 | 125 | 2.30E−08 |
| 45 | 6.91E−09 | 236 | 2.34E−08 |
| 197 | 7.53E−09 | 127 | 2.39E−08 |
| 285 | 7.67E−09 | 50 | 2.52E−08 |
| 129 | 7.72E−09 | 154 | 2.59E−08 |
| 68 | 7.80E−09 | 240 | 2.61E−08 |
| 195 | 8.35E−09 | 199 | 2.66E−08 |
| 105 | 9.24E−09 | 175 | 3.45E−08 |
| 87 | 1.02E−08 | 183 | 3.67E−08 |
| 111 | 1.02E−08 | 204 | 3.82E−08 |
| 230 | 1.06E−08 | 234 | 3.86E−08 |
| 213 | 1.10E−08 | 70 | 3.98E−08 |
| 142 | 1.15E−08 | 3 | 4.17E−08 |
| 288 | 1.17E−08 | 301 | 4.34E−08 |
| 254 | 1.20E−08 | 218 | 1.43E−07 |
| 239 | 1.20E−08 | 120 | >4.83E−08 |
| 76 | 1.32E−08 | 56 | >4.83E−08 |
| 220 | 1.32E−08 | 137 | >4.83E−08 |
| 292 | 1.38E−08 | 93 | >4.83E−08 |
| 273 | 1.42E−08 | 209 | >4.83E−08 |
| 116 | 1.44E−08 | 302 | >4.83E−08 |

TABLE 21E

| Sample ID | IC50 | Sample ID | IC50 |
|---|---|---|---|
| 117 | 8.45E−09 | 80 | 2.43E−08 |
| 263 | 8.59E−09 | 29 | 2.51E−08 |
| 191 | 9.18E−09 | 150 | 2.63E−08 |
| 71 | 9.57E−09 | 146 | 2.70E−08 |
| 152 | 9.77E−09 | 199 | 2.70E−08 |
| 295 | 1.00E−08 | 130 | 2.73E−08 |
| 40 | 1.03E−08 | 233 | 2.75E−08 |
| 53 | 1.11E−08 | 106 | 2.76E−08 |
| 151 | 1.14E−08 | 125 | 2.84E−08 |
| 229 | 1.22E−08 | 91 | 2.89E−08 |
| 27 | 1.27E−08 | 60 | 3.03E−08 |
| 129 | 1.28E−08 | 236 | 3.05E−08 |
| 87 | 1.28E−08 | 153 | 3.09E−08 |
| 225 | 1.30E−08 | 127 | 3.16E−08 |
| 96 | 1.39E−08 | 235 | 3.33E−08 |
| 141 | 1.41E−08 | 207 | 3.48E−08 |
| 285 | 1.44E−08 | 240 | 3.52E−08 |
| 111 | 1.53E−08 | 154 | 3.66E−08 |
| 105 | 1.53E−08 | 175 | 3.93E−08 |
| 142 | 1.59E−08 | 183 | 4.23E−08 |
| 45 | 1.59E−08 | 178 | 4.24E−08 |
| 197 | 1.60E−08 | 38 | 4.50E−08 |
| 239 | 1.75E−08 | 234 | 4.72E−08 |
| 254 | 1.81E−08 | 3 | 6.88E−08 |
| 184 | 1.83E−08 | 155 | >2.42E−08 |
| 213 | 1.88E−08 | 120 | >2.42E−08 |
| 237 | 1.93E−08 | 68 | >2.42E−08 |
| 273 | 1.95E−08 | 50 | >2.42E−08 |
| 195 | 1.99E−08 | 56 | >2.42E−08 |
| 76 | 1.99E−08 | 137 | >2.42E−08 |
| 230 | 2.02E−08 | 186 | >2.42E−08 |
| 288 | 2.07E−08 | 93 | >2.42E−08 |
| 220 | 2.12E−08 | 69 | >4.83E−08 |
| 224 | 2.22E−08 | 70 | >4.83E−08 |
| 292 | 2.25E−08 | 209 | >4.83E−08 |
| 290 | 2.30E−08 | 204 | >4.83E−08 |
| 41 | 2.31E−08 | 218 | >2.42E−07 |
| 116 | 2.34E−08 | 301 | >4.83E−08 |
| 139 | 2.37E−08 | 302 | >4.83E−08 |

TABLE 21F

| Sample ID | IC60 | Sample ID | IC60 |
|---|---|---|---|
| 191 | 1.23E−08 | 127 | 3.54E−08 |
| 71 | 1.35E−08 | 288 | 3.61E−08 |
| 87 | 1.37E−08 | 150 | 3.66E−08 |
| 295 | 1.44E−08 | 91 | 3.69E−08 |
| 263 | 1.44E−08 | 199 | 3.70E−08 |
| 117 | 1.45E−08 | 130 | 3.75E−08 |
| 40 | 1.50E−08 | 106 | 3.75E−08 |
| 152 | 1.72E−08 | 236 | 3.76E−08 |
| 53 | 1.93E−08 | 111 | 3.82E−08 |
| 129 | 1.95E−08 | 153 | 4.02E−08 |
| 151 | 2.01E−08 | 146 | 4.11E−08 |
| 229 | 2.13E−08 | 60 | 4.23E−08 |
| 105 | 2.14E−08 | 175 | 4.40E−08 |
| 285 | 2.24E−08 | 240 | 4.43E−08 |
| 225 | 2.29E−08 | 235 | 4.48E−08 |
| 237 | 2.33E−08 | 154 | 4.73E−08 |
| 141 | 2.36E−08 | 183 | 4.79E−08 |
| 27 | 2.45E−08 | 3 | 6.96E−08 |
| 45 | 2.51E−08 | 155 | >2.42E−08 |
| 76 | 2.54E−08 | 197 | >2.42E−08 |
| 184 | 2.54E−08 | 120 | >2.42E−08 |
| 96 | 2.63E−08 | 68 | >2.42E−08 |
| 239 | 2.65E−08 | 50 | >2.42E−08 |
| 213 | 2.65E−08 | 56 | >2.42E−08 |
| 273 | 2.75E−08 | 224 | >2.42E−08 |
| 195 | 2.84E−08 | 137 | >2.42E−08 |
| 41 | 2.91E−08 | 186 | >2.42E−08 |
| 142 | 2.92E−08 | 93 | >2.42E−08 |
| 254 | 3.10E−08 | 207 | >4.83E−08 |
| 230 | 3.12E−08 | 69 | >4.83E−08 |
| 29 | 3.12E−08 | 178 | >4.83E−08 |
| 290 | 3.13E−08 | 70 | >4.83E−08 |
| 220 | 3.14E−08 | 209 | >4.83E−08 |
| 233 | 3.32E−08 | 38 | >4.83E−08 |
| 116 | 3.36E−08 | 234 | >4.83E−08 |
| 80 | 3.38E−08 | 204 | >4.83E−08 |
| 125 | 3.39E−08 | 301 | >4.830E−08 |
| 292 | 3.40E−08 | 302 | >4.83E−08 |
| 139 | 3.52E−08 | 218 | >2.42E−07 |

In particular, detailed examination was performed concerning closely associated inflammation sites and tumor sites.

### (Inflammation Sites)

TABLE 21G

| | Tropism for | Abbreviated | | | | | | | | | | | |
|---|---|---|---|---|---|---|---|---|---|---|---|---|---|
| Liposome No. | organ | name | IC10 | IC20 | IC30 | IC31 | IC32 | IC33 | IC34 | IC35 | IC40 | IC50 | IC60 |
| 27 | ++ | LDF-5 | <4.83E−10 | 1.49E−09 | <4.83E−10 | 3.44E−09 | 3.64E−09 | 3.83E−09 | 4.03E−09 | 4.23E−09 | 5.22E−09 | 1.27E−08 | 2.45E−08 |
| 38 | ++ | G4GN-4 | 1.39E−09 | 1.81E−09 | 1.39E−09 | 2.85E−08 | 2.87E−08 | 3.08E−08 | 3.20E−08 | 3.32E−08 | 2.06E−08 | 4.50E−08 | >4.83E−08 |
| 53 | ++ | L2F-1 | <4.83E−10 | <4.83E−10 | <4.83E−10 | <4.83E−10 | <4.83E−10 | <4.83E−10 | <4.83E−10 | 5.96E−10 | 5.96E−10 | 1.11E−08 | 1.93E−08 |
| 60 | ++ | 6SLN-1 | 1.25E−09 | 5.30E−09 | 1.25E−09 | 1.18E−08 | 1.24E−08 | 1.30E−08 | 1.36E−08 | 1.42E−08 | 1.84E−08 | 3.03E−08 | 4.23E−08 |
| 69 | ++ | L4F-1 | <4.83E−10 | 1.28E−09 | <4.83E−10 | 1.11E−08 | 1.18E−08 | 1.25E−08 | 1.31E−08 | 1.38E−08 | 2.13E−08 | >4.83E−08 | >4.83E−08 |
| 70 | ++ | LNT-5 | 3.34E−09 | 4.86E−09 | 3.34E−09 | 2.72E−08 | 2.86E−08 | 3.00E−08 | 3.14E−08 | 3.28E−08 | 3.98E−08 | >4.83E−08 | >4.83E−08 |
| 87 | ++ | TA-1 | <4.83E−10 | <4.83E−10 | <4.83E−10 | 7.80E−09 | 8.06E−09 | 8.33E−09 | 8.59E−09 | 8.86E−09 | 1.02E−08 | 1.28E−08 | 1.37E−08 |
| 91 | ++ | G6GN-4 | <4.83E−10 | 7.48E−09 | <4.83E−10 | 1.42E−08 | 1.48E−08 | <4.83E−10 | 1.60E−08 | 1.68E−08 | 2.09E−08 | 2.89E−08 | 3.69E−08 |
| 93 | ++ | A36 | 9.73E−10 | 1.06E−08 | 9.45E−10 | >4.83E−08 | >4.83E−08 | >4.83E−08 | >4.83E−08 | >4.83E−08 | >4.83E−08 | >2.42E−08 | >2.42E−08 |
| 96 | ++ | TA-4 | <4.83E−10 | <4.83E−10 | <4.83E−10 | 3.89E−09 | 4.08E−09 | 4.28E−09 | 4.47E−09 | 4.66E−09 | 6.49E−09 | 1.39E−08 | 2.63E−08 |
| 105 | ++ | Man6 | 4.81E−10 | 2.51E−09 | 4.81E−10 | 5.90E−09 | 6.22E−09 | 6.53E−09 | 6.85E−09 | 7.16E−09 | 9.24E−09 | 1.53E−08 | 2.14E−08 |
| 106 | ++ | LDF-4 | <4.83E−10 | <4.83E−10 | <4.83E−10 | 2.16E−09 | 2.46E−09 | 2.75E−09 | 3.05E−09 | 3.35E−09 | 4.83E−09 | 2.76E−08 | 3.75E−08 |
| 116 | ++ | LDF-1 | 1.21E−09 | 3.78E−09 | 1.21E−09 | 9.06E−09 | 9.65E−09 | 1.02E−08 | 1.08E−08 | 1.14E−08 | 1.44E−08 | 2.34E−08 | 3.36E−08 |
| 125 | ++ | LX-1 | <4.83E−10 | <4.83E−10 | 6.28E−10 | 1.02E−09 | 1.86E−08 | 1.91E−08 | 1.97E−08 | 2.02E−08 | 2.30E−08 | 2.84E−08 | 3.39E−08 |
| 139 | ++ | 3FL-5 | <4.83E−10 | 7.93E−10 | <4.83E−10 | 1.49E−09 | 1.55E−09 | 1.61E−09 | 1.68E−09 | 1.74E−09 | 2.85E−09 | 2.37E−08 | 3.52E−08 |
| 146 | ++ | 3FL-4 | <4.83E−10 | <4.83E−10 | <4.83E−10 | 2.89E−09 | 3.29E−09 | 3.70E−09 | 4.11E−09 | 4.51E−09 | 6.10E−09 | 2.70E−08 | 4.11E−08 |
| 150 | ++ | TNA-5 | <4.83E−10 | <4.83E−10 | <4.83E−10 | <4.83E−10 | 2.90E−10 | 1.39E−10 | 8.09E−10 | 1.48E−09 | 3.86E−09 | 2.63E−08 | 3.66E−08 |
| 152 | ++ | LY-1 | <4.83E−10 | <4.83E−10 | <4.83E−10 | 2.49E−09 | 2.71E−09 | 2.93E−09 | 3.15E−09 | 3.37E−09 | 4.48E−09 | 9.77E−09 | 1.72E−08 |
| 153 | ++ | G6GN-5 | <4.83E−10 | 2.53E−09 | <4.83E−10 | 1.14E−08 | 1.29E−08 | 1.45E−08 | 1.60E−08 | 1.70E−08 | 2.16E−08 | 3.09E−08 | 4.02E−08 |
| 154 | ++ | Lac-5 | <4.83E−10 | 3.08E−09 | <4.83E−10 | 1.62E−08 | 1.73E−08 | 1.84E−08 | 1.94E−08 | 2.05E−08 | 2.59E−08 | 3.66E−08 | 4.73E−08 |
| 155 | ++ | A6 | 6.47E−09 | 1.01E−08 | 6.47E−09 | 1.58E−08 | 1.63E−08 | 1.68E−08 | 1.72E−08 | 1.77E−08 | 2.01E−08 | >2.42E−08 | >2.42E−08 |
| 184 | ++ | STNA-1 | <4.83E−10 | <4.83E−10 | <4.83E−10 | 1.26E−09 | 1.39E−09 | 1.52E−09 | 1.65E−09 | 1.77E−09 | 3.55E−09 | 1.83E−08 | 2.54E−08 |
| 191 | ++ | LB-1 | <4.83E−10 | 1.40E−09 | <4.83E−10 | 3.50E−09 | 3.77E−09 | 4.04E−09 | 4.31E−09 | 4.59E−09 | 6.04E−09 | 9.18E−09 | 1.23E−08 |
| 195 | ++ | TNA-4 | <4.83E−10 | <4.83E−10 | <4.83E−10 | 8.88E−10 | 1.11E−09 | 5.33E−09 | 1.56E−09 | 1.78E−09 | 8.35E−09 | 1.99E−08 | 2.84E−08 |
| 199 | ++ | STNA-4 | <4.83E−10 | <4.83E−10 | <4.83E−10 | 3.53E−09 | 3.79E−09 | 4.05E−09 | 4.31E−09 | 4.67E−09 | 2.66E−08 | 2.70E−08 | 3.70E−08 |
| 204 | ++ | Lac-1 | <4.83E−10 | <4.83E−10 | <4.83E−10 | 2.47E−08 | 2.62E−08 | 2.77E−08 | 2.92E−08 | 3.07E−08 | 3.82E−08 | >4.83E−08 | >4.83E−08 |
| 213 | ++ | LX-4 | <4.83E−10 | 9.28E−10 | 4.94E−09 | 5.08E−09 | 5.22E−09 | 5.35E−09 | 6.32E−09 | 7.10E−09 | 1.10E−08 | 1.68E−08 | 2.65E−08 |
| 220 | ++ | LNB-4 | 3.83E−09 | 6.57E−09 | 3.83E−09 | 1.06E−08 | 1.09E−08 | 1.11E−08 | 1.14E−08 | 7.27E−09 | 1.32E−08 | 2.12E−08 | 3.14E−08 |
| 224 | ++ | Man5 | 2.97E−10 | 4.43E−09 | 2.97E−10 | 8.44E−09 | 9.16E−09 | 9.88E−09 | 1.06E−08 | 1.13E−08 | 1.49E−08 | 2.22E−08 | >2.42E−08 |
| 229 | ++ | G6GN-1 | <4.83E−10 | <4.83E−10 | <4.83E−10 | 3.51E−09 | 3.76E−09 | <4.83E−10 | 4.24E−09 | 4.48E−09 | 6.25E−09 | 1.22E−08 | 2.13E−08 |
| 230 | ++ | G4GN-1 | <4.83E−10 | 2.85E−09 | <4.83E−10 | 4.77E−09 | 4.95E−09 | 5.12E−09 | 5.22E−09 | 6.11E−09 | 1.05E−08 | 2.02E−08 | 3.12E−08 |
| 233 | ++ | 2FL-1 | 1.43E−09 | 3.41E−09 | 1.43E−09 | 1.66E−08 | 1.71E−08 | 1.77E−08 | 1.83E−08 | 1.89E−08 | 2.17E−08 | 2.75E−08 | 3.32E−08 |
| 234 | ++ | TNA-1 | <4.83E−10 | 3.41E−09 | <4.83E−10 | 3.09E−08 | 3.17E−08 | 3.34E−08 | 3.34E−08 | 3.43E−08 | 3.86E−08 | 4.72E−08 | >4.83E−08 |
| 235 | ++ | Lac-4 | <4.83E−10 | 1.48E−09 | <4.83E−10 | 4.27E−09 | 4.46E−09 | 1.47E−08 | 1.54E−08 | 5.05E−09 | 2.18E−08 | 3.33E−08 | 4.48E−08 |
| 237 | ++ | A3 | 4.04E−09 | 7.43E−09 | 4.04E−09 | 1.17E−08 | 1.21E−08 | 1.25E−08 | 1.29E−08 | 1.33E−08 | 1.53E−08 | 1.93E−08 | 2.33E−08 |
| 263 | ++ | 3SLN-1 | <4.83E−10 | <4.83E−10 | <4.83E−10 | 7.20E−10 | 8.08E−10 | 8.96E−10 | 9.85E−10 | 1.07E−09 | 1.51E−09 | 8.59E−09 | 1.44E−08 |
| 290 | ++ | SLA-5 | 1.84E−09 | 7.78E−09 | 1.16E−08 | 1.19E−08 | 1.23E−08 | 1.27E−08 | 1.31E−08 | 1.35E−08 | 1.53E−08 | 2.30E−08 | 3.13E−08 |
| 292 | ++ | STNA-5 | 2.26E−09 | 4.66E−09 | 2.26E−09 | 9.39E−09 | 7.55E−09 | 1.04E−08 | 1.09E−08 | 1.14E−08 | 1.38E−08 | 2.25E−08 | 3.40E−08 |
| 292 | ++ | STNA-5 | 2.26E−09 | 4.66E−09 | 2.26E−09 | 9.39E−09 | 7.55E−09 | 1.04E−08 | 1.09E−08 | 1.14E−08 | 1.38E−08 | 2.25E−08 | 3.40E−08 |
| 40 | + | BAT-1 | 1.17E−09 | 2.78E−09 | 1.17E−09 | 4.22E−09 | 4.35E−09 | 4.48E−09 | 4.61E−09 | 4.74E−09 | 5.39E−09 | 1.03E−08 | 1.50E−08 |
| 41 | + | SOLX-4 | <4.83E−10 | 1.06E−09 | 1.03E−08 | 1.13E−08 | 1.17E−08 | 1.24E−08 | 1.31E−08 | 1.38E−08 | 1.71E−08 | 2.31E−08 | 2.91E−08 |
| 50 | + | Man8 | 1.80E−09 | 2.78E−09 | 1.80E−09 | 1.30E−08 | 1.44E−08 | 1.57E−08 | 1.71E−08 | 1.84E−08 | 2.52E−08 | >2.42E−08 | >2.42E−08 |
| 56 | + | Man9 | 1.61E−08 | 2.35E−08 | 1.61E−08 | >4.83E−08 | >4.83E−08 | >4.83E−08 | >4.83E−08 | >4.83E−08 | >4.83E−08 | >2.42E−08 | >2.42E−08 |
| 68 | + | Man7 | 2.11E−09 | 4.28E−09 | 2.11E−09 | >4.83E−08 | >4.83E−08 | >4.83E−08 | 1.68E−08 | 1.58E−08 | 7.80E−09 | >2.42E−08 | >2.42E−08 |
| 71 | + | BBT-1 | 5.99E−10 | 2.78E−09 | 5.99E−10 | 4.24E−09 | 4.37E−09 | 4.50E−09 | 4.63E−09 | 4.77E−09 | 5.65E−09 | 9.57E−09 | 1.35E−08 |
| 76 | + | BHD-4 | <4.83E−10 | 2.39E−09 | <4.83E−10 | 4.21E−09 | 5.21E−09 | 6.20E−09 | 7.19E−09 | 8.19E−09 | 1.32E−08 | 1.99E−08 | 2.54E−08 |
| 111 | + | LNB-5 | 2.62E−09 | 3.94E−09 | 2.62E−09 | 5.62E−09 | 6.13E−09 | 6.64E−09 | 7.15E−09 | 7.66E−09 | 1.02E−08 | 1.53E−08 | 3.82E−08 |
| 117 | + | TA-5 | <4.83E−10 | 1.09E−09 | <4.83E−10 | 2.88E−09 | 3.06E−09 | 3.24E−09 | 3.41E−09 | 3.59E−09 | 4.47E−09 | 8.45E−09 | 1.45E−08 |
| 120 | + | A2 | 7.02E−09 | 8.70E−09 | 7.02E−09 | 2.09E−08 | 2.20E−08 | 2.31E−08 | 2.42E−08 | >4.83E−08 | >4.83E−08 | >2.42E−08 | >2.42E−08 |
| 137 | + | Man4B | 6.02E−10 | 1.04E−08 | 6.02E−10 | 1.96E−08 | 2.04E−08 | 2.13E−08 | 2.21E−08 | 2.30E−08 | >4.83E−08 | >2.42E−08 | >2.42E−08 |
| 151 | + | SLX-4 | <4.83E−10 | <4.83E−10 | 1.37E−09 | 1.52E−09 | 1.67E−09 | 1.82E−09 | 1.98E−09 | 2.39E−09 | 4.16E−09 | 1.14E−08 | 2.01E−08 |
| 183 | + | G4GN-5 | 1.99E−08 | 2.55E−08 | 1.99E−08 | 3.16E−08 | 3.22E−08 | 3.28E−08 | 3.33E−08 | 3.39E−08 | 3.67E−08 | 4.23E−08 | 4.79E−08 |
| 186 | + | Man3 | 8.41E−10 | 1.12E−08 | 8.41E−10 | 1.66E−08 | 1.70E−08 | 1.75E−08 | 1.80E−08 | 1.85E−08 | 2.09E−08 | >2.42E−08 | >2.42E−08 |
| 197 | + | A4 | 5.01E−09 | 5.85E−09 | 5.01E−09 | 6.77E−09 | 6.86E−09 | 6.94E−09 | 7.03E−09 | 7.11E−09 | 7.53E−09 | 1.60E−08 | >2.42E−08 |
| 209 | + | LDFH-4 | 3.78E−09 | 4.58E−10 | 3.78E−09 | 4.23E−08 | 4.47E−08 | 4.70E−08 | 4.94E−08 | >4.83E−08 | >4.83E−08 | >4.83E−08 | >4.83E−08 |
| 240 | + | SOLA-5 | <4.83E−10 | <4.83E−10 | 1.06E−09 | 1.79E−08 | 1.88E−08 | 1.97E−08 | 2.07E−08 | 2.16E−08 | 2.61E−08 | 3.52E−08 | 4.43E−08 |
| 288 | + | LA-1 | 2.63E−09 | 4.00E−09 | 5.54E−09 | 6.16E−09 | 6.77E−09 | 7.39E−09 | 8.00E−09 | 8.62E−09 | 1.17E−08 | 2.07E−08 | 3.61E−08 |
| 295 | + | 6SLN-4 | <4.83E−10 | 2.30E−09 | <4.83E−10 | 4.18E−09 | 4.18E−09 | 4.33E−09 | 4.49E−09 | 4.65E−09 | 5.63E−09 | 1.00E−08 | 1.44E−08 |
| 16 | − | SOLX-5 | <4.83E−10 | 1.42E−08 | 2.37E−08 | 2.43E−08 | 2.49E−08 | 2.55E−08 | 2.61E−08 | 4.13E−09 | 2.97E−08 | 3.57E−08 | 4.16E−08 |
| 29 | − | 3SL-5 | 1.21E−11 | 1.23E−08 | 4.83E−10 | 1.50E−08 | 1.52E−08 | 1.55E−08 | 1.54E−08 | 1.60E−08 | 1.90E−08 | 2.51E−08 | 3.12E−08 |
| 127 | − | G3G-4 | <4.83E−10 | 1.27E−08 | 4.83E−10 | 1.54E−08 | 1.57E−08 | 1.59E−08 | 1.70E−08 | 1.82E−08 | 2.39E−08 | 3.16E−08 | 3.54E−08 |
| 129 | − | BBT-4 | 2.86E−09 | 3.83E−09 | 2.86E−09 | 4.90E−09 | 5.00E−08 | 5.09E−09 | 5.19E−09 | 5.29E−09 | 7.72E−09 | 1.28E−08 | 1.95E−08 |
| 130 | − | 2FL-4 | 4.12E−09 | 7.91E−09 | 4.12E−09 | 1.26E−08 | 1.30E−08 | 1.34E−08 | 1.38E−08 | 1.43E−08 | 1.70E−08 | 2.73E−08 | 3.75E−08 |
| 141 | − | BBT-5 | 1.66E−09 | 1.80E−09 | 1.66E−09 | 2.47E−09 | 2.74E−09 | 3.02E−09 | 3.29E−09 | 3.56E−09 | 4.92E−09 | 1.41E−08 | 2.36E−08 |
| 142 | − | BHD-5 | 9.87E−10 | 2.07E−09 | 9.87E−10 | 7.48E−09 | 7.92E−09 | 8.37E−09 | 8.81E−09 | 9.25E−09 | 1.15E−08 | 1.59E−08 | 2.92E−08 |
| 175 | − | G3G-5 | 2.01E−08 | 2.49E−08 | 2.01E−08 | 3.02E−08 | 3.06E−08 | 3.11E−08 | 3.16E−08 | 3.21E−08 | 3.45E−08 | 3.93E−08 | 4.40E−08 |
| 178 | − | LNT-4 | 6.84E−09 | 1.01E−08 | 6.84E−09 | 1.36E−08 | 1.40E−08 | 1.43E−08 | 1.46E−08 | 1.49E−08 | 2.01E−08 | 4.24E−08 | >4.83E−08 |
| 207 | − | 6SL-5 | 3.76E−09 | 4.64E−09 | 4.83E−10 | 8.94E−09 | 1.00E−08 | 1.11E−08 | 1.22E−08 | 1.34E−08 | 1.99E−08 | 3.48E−08 | >4.83E−08 |
| 236 | − | 2FL-5 | 7.44E−09 | 1.18E−08 | 7.44E−09 | 1.70E−08 | 1.77E−08 | 1.84E−08 | 1.92E−08 | 1.99E−08 | 2.34E−08 | 3.05E−08 | 3.76E−08 |
| 239 | − | SLA-4 | <4.83E−10 | 1.58E−09 | 7.28E−09 | 7.75E−09 | 8.23E−09 | 8.70E−09 | 9.17E−09 | 9.64E−09 | 1.20E−08 | 1.75E−08 | 2.65E−08 |
| 3 | − | EEGD1A-BH | 4.93E−09 | 1.13E−08 | 2.21E−08 | 2.32E−08 | 2.43E−08 | 2.53E−08 | 2.64E−08 | 2.81E−08 | 4.17E−08 | 6.88E−08 | 6.96E−08 |
| 218 | − | EEGMI-BH | 7.23E−09 | 2.03E−08 | 6.08E−08 | 6.49E−08 | 6.90E−08 | 7.31E−08 | 7.73E−08 | 8.45E−08 | 1.43E−07 | >2.42E−07 | >2.42E−07 |
| 254 | − | G3G-1 | 2.03E−09 | 4.05E−09 | 2.03E−09 | 7.61E−09 | 8.09E−09 | 8.57E−09 | 9.06E−09 | 9.54E−09 | 1.19E−08 | 1.61E−08 | 3.10E−08 |
| 301 | − | TRIS | 8.56E−09 | 2.02E−08 | 3.18E−08 | 3.30E−08 | 3.41E−08 | 3.53E−08 | 3.84E−08 | 3.76E−08 | 4.34E−08 | >4.83E−08 | >4.83E−08 |
| 302 | − | CEBI | 3.98E−08 | 2.37E−08 | 4.05E−08 | 4.22E−08 | 4.39E−08 | 4.56E−08 | 4.73E−08 | 4.90E−08 | >4.83E−08 | >4.83E−08 | >4.83E−08 |

### (Tumor Sites)

TABLE 21H

| | Tropism for | Abbreviated | | | | | | | | | | | |
|---|---|---|---|---|---|---|---|---|---|---|---|---|---|
| Liposome No. | organ | name | IC10 | IC20 | IC30 | IC31 | IC32 | IC33 | IC34 | IC35 | IC40 | IC50 | IC60 |
| 27 | ++ | LDF-5 | <4.83E−10 | 1.49E−09 | <4.83E−10 | 3.44E−09 | 3.64E−09 | 3.83E−09 | 4.03E−09 | 4.23E−09 | 5.22E−09 | 1.27E−08 | 2.45E−08 |
| 29 | ++ | 3SL-5 | 1.21E−11 | 1.23E−08 | 4.83E−10 | 1.50E−08 | 1.52E−08 | 1.55E−08 | 1.54E−08 | 1.60E−08 | 1.90E−08 | 2.51E−08 | 3.12E−08 |
| 38 | ++ | G4GN-4 | 1.39E−09 | 1.81E−09 | 1.39E−09 | 2.85E−08 | 2.97E−08 | 3.08E−08 | 3.20E−08 | 3.32E−08 | 2.06E−08 | 4.50E−08 | >4.83E−08 |
| 53 | ++ | L2F-1 | <4.83E−10 | <4.83E−10 | <4.83E−10 | <4.83E−10 | <4.83E−10 | <4.83E−10 | <4.83E−10 | 5.96E−10 | 5.96E−10 | 1.11E−08 | 1.93E−08 |
| 60 | ++ | 6SLN-1 | 1.25E−09 | 5.30E−09 | 1.25E−09 | 1.18E−08 | 1.24E−08 | 1.30E−08 | 1.36E−08 | 1.42E−08 | 1.84E−08 | 3.03E−08 | 4.23E−08 |
| 69 | ++ | L4F-1 | <4.83E−10 | 1.28E−09 | <4.83E−10 | 1.11E−08 | 1.18E−08 | 1.25E−08 | 1.31E−08 | 1.38E−08 | 2.13E−08 | >4.83E−08 | >4.83E−08 |
| 87 | ++ | TA-1 | <4.83E−10 | <4.83E−10 | <4.83E−10 | 7.08E−09 | 8.06E−09 | 8.33E−09 | 8.59E−09 | 8.86E−09 | 1.02E−08 | 1.28E−08 | 1.37E−08 |
| 93 | ++ | A36 | 9.73E−10 | 1.06E−08 | 9.45E−10 | >4.83E−08 | >4.83E−08 | >4.83E−08 | >4.83E−08 | >4.83E−08 | >4.83E−08 | >2.42E−08 | >2.42E−08 |
| 96 | ++ | TA-4 | <4.83E−10 | <4.83E−10 | <4.83E−10 | 3.89E−09 | 4.08E−09 | 4.28E−09 | 4.47E−09 | 4.66E−09 | 6.49E−09 | 1.39E−08 | 2.63E−08 |
| 116 | ++ | LDF-1 | 1.21E−09 | 3.78E−09 | 1.21E−09 | 9.06E−09 | 9.65E−09 | 1.02E−08 | 1.08E−08 | 1.14E−08 | 1.44E−08 | 2.34E−08 | 3.36E−08 |
| 125 | ++ | LX-1 | <4.83E−10 | <4.83E−10 | 6.28E−10 | 1.02E−09 | 1.86E−08 | 1.91E−08 | 1.97E−08 | 2.02E−08 | 2.30E−08 | 2.84E−08 | 3.39E−08 |
| 150 | ++ | TNA-5 | <4.83E−10 | <4.83E−10 | <4.83E−10 | <4.83E−10 | 2.90E−10 | 1.39E−10 | 8.09E−10 | 1.48E−09 | 3.86E−09 | 2.63E−08 | 3.66E−08 |
| 152 | ++ | LY-1 | <4.83E−10 | <4.83E−10 | <4.83E−10 | 2.49E−09 | 2.71E−09 | 2.93E−09 | 3.15E−09 | 3.37E−09 | 4.48E−09 | 9.77E−09 | 1.72E−08 |
| 154 | ++ | Lac-5 | <4.83E−10 | 3.08E−09 | <4.83E−10 | 1.62E−08 | 1.73E−08 | 1.84E−08 | 1.84E−08 | 2.05E−08 | 2.59E−08 | 3.66E−08 | 4.73E−08 |
| 155 | ++ | A6 | 6.47E−09 | 1.01E−08 | 6.47E−09 | 1.58E−08 | 1.63E−08 | 1.68E−08 | 1.72E−08 | 1.77E−08 | 2.01E−08 | >2.42E−08 | >2.42E−08 |
| 184 | ++ | STNA-1 | <4.83E−10 | <4.83E−10 | <4.83E−10 | 1.26E−09 | 1.39E−09 | 1.52E−09 | 1.65E−09 | 1.77E−09 | 3.55E−09 | 1.83E−08 | 2.54E−08 |
| 195 | ++ | TNA-4 | <4.83E−10 | <4.83E−10 | <4.83E−10 | 8.88E−10 | 1.11E−09 | 5.33E−09 | 1.56E−09 | 1.78E−09 | 8.35E−09 | 1.99E−08 | 2.84E−08 |
| 204 | ++ | Lac-1 | <4.83E−10 | <4.83E−10 | <4.83E−10 | 2.47E−08 | 2.62E−08 | 2.77E−08 | 2.92E−08 | 3.07E−08 | 3.82E−08 | >4.83E−08 | >4.83E−08 |
| 213 | ++ | LX-4 | <4.83E−10 | 9.28E−10 | 4.94E−09 | 5.08E−09 | 5.22E−09 | 5.35E−09 | 6.32E−09 | 7.10E−09 | 1.10E−08 | 1.88E−08 | 2.65E−08 |
| 224 | ++ | Man5 | 2.97E−10 | 4.43E−09 | 2.97E−10 | 8.44E−09 | 9.16E−09 | 9.88E−09 | 1.06E−08 | 1.13E−08 | 1.49E−08 | 2.22E−08 | >2.42E−08 |
| 229 | ++ | G6GN-1 | <4.83E−10 | <4.83E−10 | <4.83E−10 | 3.51E−09 | 3.76E−09 | <4.83E−10 | 4.24E−09 | 4.48E−09 | 6.25E−09 | 1.22E−08 | 2.13E−08 |
| 230 | ++ | G4GN-1 | <4.83E−10 | 2.85E−09 | <4.83E−10 | 4.77E−09 | 4.95E−09 | 5.12E−09 | 5.22E−09 | 6.11E−09 | 1.05E−08 | 2.02E−08 | 3.12E−08 |
| 233 | ++ | 2FL-1 | 1.43E−09 | 3.41E−09 | 1.43E−09 | 1.66E−08 | 1.71E−08 | 1.77E−08 | 1.83E−08 | 1.89E−08 | 2.17E−08 | 2.75E−08 | 3.32E−08 |
| 234 | ++ | TNA-1 | <4.83E−10 | 3.41E−09 | <4.83E−10 | 3.09E−08 | 3.17E−08 | 3.34E−08 | 3.34E−08 | 3.43E−08 | 3.86E−08 | 4.72E−08 | >4.83E−08 |
| 235 | ++ | Lac-4 | <4.83E−10 | 1.48E−09 | <4.83E−10 | 4.27E−09 | 4.46E−09 | 1.47E−08 | 1.54E−08 | 5.05E−09 | 2.18E−08 | 3.33E−08 | 4.48E−08 |
| 237 | ++ | A3 | 4.04E−09 | 7.43E−09 | 4.04E−09 | 1.17E−08 | 1.21E−08 | 1.25E−08 | 1.29E−08 | 1.33E−08 | 1.53E−08 | 1.93E−08 | 2.33E−08 |
| 263 | ++ | 3SLN-1 | <4.83E−10 | <4.83E−10 | <4.83E−10 | 7.20E−10 | 8.08E−10 | 8.96E−10 | 9.85E−10 | 1.07E−09 | 1.51E−09 | 8.59E−09 | 1.44E−08 |
| 285 | ++ | SLX-5 | <4.83E−10 | <4.83E−10 | 2.70E−09 | 3.10E−09 | 3.50E−09 | 3.90E−09 | 4.30E−09 | 4.71E−09 | 7.67E−09 | 1.44E−08 | 2.24E−08 |
| 288 | ++ | LA-1 | 2.63E−09 | 4.00E−09 | 5.54E−09 | 6.16E−09 | 6.77E−09 | 7.39E−09 | 8.00E−09 | 8.62E−09 | 1.17E−08 | 2.07E−08 | 3.61E−08 |
| 292 | ++ | STNA-5 | 2.26E−09 | 4.66E−09 | 2.26E−09 | 9.39E−08 | 7.55E−09 | 1.04E−08 | 1.09E−08 | 1.14E−08 | 1.38E−08 | 2.25E−08 | 3.40E−08 |
| 40 | + | BAT-1 | 1.17E−09 | 2.78E−09 | 1.17E−09 | 4.22E−09 | 4.35E−09 | 4.48E−09 | 4.61E−09 | 4.74E−09 | 5.39E−09 | 1.03E−08 | 1.50E−08 |
| 41 | + | SOLX-4 | <4.83E−10 | 1.06E−09 | 1.03E−08 | 1.13E−08 | 1.17E−08 | 1.24E−08 | 1.31E−08 | 1.38E−08 | 1.71E−08 | 2.31E−08 | 2.91E−08 |
| 45 | + | BHD-1 | <4.83E−10 | <4.83E−10 | <4.83E−10 | 1.91E−09 | 2.38E−09 | 2.85E−09 | 3.32E−09 | 3.79E−09 | 6.91E−09 | 1.59E−08 | 2.51E−08 |
| 68 | + | Man7 | 2.11E−09 | 4.28E−09 | 2.11E−09 | >4.83E−08 | >4.83E−08 | >4.83E−08 | 1.68E−08 | 1.58E−08 | 7.80E−09 | >2.42E−08 | >2.42E−08 |
| 71 | + | BBT-1 | 5.99E−10 | 2.78E−09 | 5.99E−10 | 4.24E−09 | 4.37E−09 | 4.50E−09 | 4.63E−09 | 4.77E−09 | 5.65E−09 | 9.57E−09 | 1.35E−08 |
| 91 | + | G6GN-4 | <4.83E−10 | 7.48E−09 | <4.83E−10 | 1.42E−08 | 1.48E−08 | <4.83E−10 | 1.60E−08 | 1.68E−08 | 2.09E−08 | 2.89E−08 | 3.69E−08 |
| 105 | + | Man6 | 4.81E−10 | 2.51E−09 | 4.81E−10 | 5.90E−09 | 6.22E−09 | 6.53E−09 | 6.85E−09 | 7.16E−09 | 9.24E−09 | 1.53E−08 | 2.14E−08 |
| 106 | + | LDF-4 | <4.83E−10 | <4.83E−10 | <4.83E−10 | 2.16E−09 | 2.46E−09 | 2.75E−09 | 3.05E−09 | 3.35E−09 | 4.83E−09 | 2.76E−08 | 3.75E−08 |
| 111 | + | LNB-5 | 2.62E−09 | 3.94E−09 | 2.62E−09 | 5.62E−09 | 6.13E−09 | 6.64E−09 | 7.15E−09 | 7.66E−09 | 1.02E−08 | 1.53E−08 | 3.82E−08 |
| 117 | + | TA-5 | <4.83E−10 | 1.09E−09 | <4.83E−10 | 2.88E−09 | 3.06E−09 | 3.24E−09 | 3.41E−09 | 3.59E−09 | 4.47E−09 | 8.45E−09 | 1.45E−08 |
| 120 | + | A2 | 7.02E−09 | 8.70E−09 | 7.02E−09 | 2.09E−08 | 2.20E−08 | 2.31E−08 | 2.42E−08 | >4.83E−08 | >4.83E−08 | >2.42E−08 | >2.42E−08 |
| 139 | + | 3FL-5 | <4.83E−10 | 7.93E−10 | <4.83E−10 | 1.49E−09 | 1.55E−09 | 1.61E−09 | 1.68E−09 | 1.74E−09 | 2.85E−09 | 2.37E−08 | 3.52E−08 |
| 151 | + | SLX-4 | <4.83E−10 | <4.83E−10 | 1.37E−09 | 1.52E−09 | 1.67E−09 | 1.82E−09 | 1.98E−09 | 2.39E−09 | 4.16E−09 | 1.14E−08 | 2.01E−08 |
| 153 | + | G6GN-5 | <4.83E−10 | 2.53E−09 | <4.83E−10 | 1.14E−08 | 1.29E−08 | 1.45E−08 | 1.60E−08 | 1.70E−08 | 2.16E−08 | 3.09E−08 | 4.02E−08 |
| 186 | + | Man3 | 8.41E−10 | 1.12E−08 | 8.41E−10 | 1.66E−08 | 1.70E−08 | 1.75E−08 | 1.80E−08 | 1.85E−08 | 2.09E−08 | >2.42E−08 | >2.42E−08 |
| 191 | + | LB-1 | <4.83E−10 | 1.40E−09 | <4.83E−10 | 3.50E−09 | 3.77E−09 | 4.04E−09 | 4.31E−09 | 4.59E−09 | 6.04E−09 | 9.18E−09 | 1.23E−08 |
| 197 | + | A4 | 5.01E−09 | 5.85E−09 | 5.01E−09 | 6.77E−09 | 6.86E−09 | 6.94E−09 | 7.03E−09 | 7.11E−09 | 7.53E−09 | 1.60E−08 | >2.42E−08 |
| 209 | + | LDFH-4 | 3.78E−09 | 4.58E−10 | 3.78E−09 | 4.23E−08 | 4.47E−08 | 4.70E−08 | 4.94E−08 | >4.83E−08 | >4.83E−08 | >4.83E−08 | >4.83E−08 |
| 220 | + | LNB-4 | 3.83E−09 | 6.57E−09 | 3.83E−09 | 1.06E−08 | 1.09E−08 | 1.11E−08 | 1.14E−08 | 7.27E−09 | 1.32E−08 | 2.12E−08 | 3.14E−08 |
| 225 | + | 3FL-1 | <4.83E−10 | <4.83E−10 | <4.83E−10 | 1.69E−09 | 1.80E−09 | 1.92E−09 | 2.33E−09 | 2.80E−09 | 5.13E−09 | 1.30E−08 | 2.29E−08 |
| 236 | + | 2FL-5 | 7.44E−09 | 1.18E−08 | 7.44E−09 | 1.70E−08 | 1.77E−08 | 1.84E−08 | 1.92E−08 | 1.99E−08 | 2.34E−08 | 3.05E−08 | 3.76E−08 |
| 240 | + | SOLA-5 | <4.83E−10 | <4.83E−10 | 1.06E−09 | 1.79E−08 | 1.88E−08 | 1.97E−08 | 2.07E−08 | 2.16E−08 | 2.61E−08 | 3.52E−08 | 4.43E−08 |
| 290 | + | SLA-5 | 1.84E−09 | 7.78E−09 | 1.16E−08 | 1.19E−08 | 1.23E−08 | 1.27E−08 | 1.31E−08 | 1.35E−08 | 1.53E−08 | 2.30E−08 | 3.13E−08 |
| 295 | + | 6SLN-4 | <4.83E−10 | 2.30E−09 | <4.83E−10 | 4.18E−09 | 4.18E−08 | 4.33E−09 | 4.49E−09 | 4.65E−09 | 5.63E−09 | 1.00E−08 | 1.44E−08 |
| 50 | − | Man8 | 1.80E−09 | 2.78E−09 | 1.80E−09 | 1.30E−08 | 1.44E−08 | 1.57E−08 | 1.71E−08 | 1.84E−08 | 2.52E−08 | >2.42E−08 | >2.42E−08 |
| 56 | − | Man9 | 1.61E−08 | 2.35E−08 | 1.61E−08 | >4.83E−08 | >4.83E−08 | >4.83E−08 | >4.83E−08 | >4.83E−08 | >4.83E−08 | >2.42E−08 | >2.42E−08 |
| 70 | − | LNT-5 | 3.34E−09 | 4.86E−09 | 3.34E−09 | 2.72E−08 | 2.86E−08 | 3.00E−08 | 3.14E−08 | 3.28E−08 | 3.98E−08 | >4.83E−08 | >4.83E−08 |
| 127 | − | G3G-4 | <4.83E−10 | 1.27E−08 | 4.83E−10 | 1.54E−08 | 1.57E−08 | 1.59E−08 | 1.70E−08 | 1.82E−08 | 2.39E−08 | 3.16E−08 | 3.54E−08 |
| 129 | − | BBT-4 | 2.86E−09 | 3.83E−09 | 2.86E−09 | 4.90E−09 | 5.00E−09 | 5.09E−09 | 5.19E−09 | 5.29E−09 | 7.72E−09 | 1.28E−08 | 1.95E−08 |
| 137 | − | Man4B | 6.02E−10 | 1.04E−08 | 6.02E−10 | 1.96E−08 | 2.04E−08 | 2.13E−08 | 2.21E−08 | 2.30E−08 | >4.83E−08 | >2.42E−08 | >2.42E−08 |
| 141 | − | BBT-5 | 1.66E−09 | 1.80E−09 | 1.66E−09 | 2.47E−09 | 2.74E−09 | 3.02E−09 | 3.29E−09 | 3.56E−09 | 4.92E−09 | 1.41E−08 | 2.36E−08 |
| 142 | − | BHD-5 | 9.87E−10 | 2.07E−09 | 9.87E−10 | 7.48E−09 | 7.92E−09 | 8.37E−09 | 8.81E−09 | 9.25E−09 | 1.15E−08 | 1.59E−08 | 2.92E−08 |
| 175 | − | G3G-5 | 2.01E−08 | 2.49E−08 | 2.01E−08 | 3.02E−08 | 3.06E−08 | 3.11E−08 | 3.16E−08 | 3.21E−08 | 3.45E−08 | 3.93E−08 | 4.40E−08 |
| 183 | − | G4GN-5 | 1.99E−08 | 2.55E−08 | 1.99E−08 | 3.16E−08 | 3.22E−08 | 3.28E−08 | 3.33E−08 | 3.39E−08 | 3.67E−08 | 4.23E−08 | 4.79E−08 |
| 207 | − | 6SL-5 | 3.76E−09 | 4.64E−08 | 4.83E−10 | 8.94E−09 | 1.00E−08 | 1.11E−08 | 1.22E−08 | 1.34E−08 | 1.99E−08 | 3.48E−08 | >4.83E−08 |
| 254 | − | G3G-1 | 2.03E−09 | 4.05E−09 | 2.03E−09 | 7.61E−09 | 8.09E−09 | 8.57E−09 | 9.06E−09 | 9.54E−09 | 1.19E−08 | 1.81E−08 | 3.10E−08 |
| 3 | − | EEGD1A-BH | 4.93E−09 | 1.13E−09 | 2.21E−08 | 2.32E−08 | 2.43E−08 | 2.53E−08 | 2.64E−08 | 2.81E−08 | 4.17E−08 | 6.88E−08 | 6.96E−08 |
| 12 | − | 3SL-4 | 1.18E−08 | 1.79E−08 | 1.18E−08 | 1.24E−08 | 1.26E−08 | 1.28E−08 | 1.29E−08 | 1.43E−08 | 1.48E−08 | 6.38E−10 | >4.83E−08 |
| 130 | − | 2FL-4 | 4.12E−09 | 7.91E−09 | 4.12E−09 | 1.26E−08 | 1.30E−08 | 1.34E−08 | 1.38E−08 | 1.43E−08 | 1.70E−08 | 2.73E−08 | 3.75E−08 |
| 178 | − | LNT-4 | 6.84E−09 | 1.01E−08 | 6.84E−09 | 1.36E−08 | 1.40E−08 | 1.43E−08 | 1.46E−08 | 1.49E−08 | 2.01E−08 | 4.24E−08 | >4.83E−08 |
| 218 | − | EEGM1-BH | 7.23E−09 | 2.03E−08 | 6.08E−08 | 6.49E−08 | 6.90E−08 | 7.31E−08 | 7.73E−08 | 8.45E−08 | 1.43E−07 | >2.42E−07 | >2.42E−07 |
| 301 | − | TRIS | 8.56E−09 | 2.02E−08 | 3.18E−08 | 3.30E−08 | 3.41E−08 | 3.53E−08 | 3.64E−08 | 3.76E−08 | 4.34E−08 | >4.83E−08 | >4.83E−08 |
| 302 | − | CEBI | 3.98E−08 | 2.37E−08 | 4.05E−08 | 4.22E−08 | 4.39E−08 | 4.56E−08 | 4.73E−08 | 4.90E−08 | >4.83E−08 | >4.83E−08 | >4.83E−08 |

### Example 18

### ¹²⁵I Labeling of Various Sugar Chain-Bound Liposome by Chloramine T Method

A chloramine T (Wako Pure Chemical Co., Japan) solution and a sodium disulfite solution were each prepared at 3 mg/ml and 5 mg/ml when they were used. The sugar chain-bound liposomes and tris(hydroxymethyl)aminomethane-bound liposomes prepared in Example 16 were separately added (50 μl each) to Eppen tubes. Subsequently, 15 μl of ¹²⁵I-NaI (NEN Life Science Product, Inc. U.S.A.) and 10 μl of a chloramine T solution were added to perform reaction. 10 μl of the chloramine T solution was added every 5 minutes. At 15 minutes after repeating twice this procedure, 100 μl of sodium disulfite was added as a reducing agent so as to stop the reaction. Next, the resultants were placed on a Sephadex G-50 (Pharmacia Biotech. Sweden) column for chromatography and then subjected to elution with PBS, so that labeled products were purified. Finally, an unlabeled-liposome complex was added and then specific activity (4×10⁶ Bq/mg protein) was adjusted. Thus, ¹²⁵I-labeled liposome solutions were obtained.

### Example 19

### Measurement of the Amounts of Various Sugar Chain-Bound Liposome Complexes that Transferred from the Intestinal Tract into Blood in Mice

0.2 ml of each ¹²⁵I-labeled sugar chain-bound and tris(hydroxymethyl)aminomethane-bound liposome complex prepared in Example 17 was forcedly administered intraintestinally using oral sonde for mice to male ddY mice (7-week-old) that had been fasted overnight (excluding water), so that the amount of protein administered herein was 3 μg/mouse. 10 minutes later, 1 ml of blood was collected from inferior vena cava of each mouse under Nembutal anesthesia. ¹²⁵I radioactivity in blood was then measured using a gamma counter (Alola ARC300). Furthermore, to examine the in vivo stability of various liposome complexes, the serum of each blood was re-chromatographed using Sephadex G-50. In all cases, most of radioactivity was observed in high-molecular-weight void fractions. Various liposome complexes also had stability in vivo. In addition, each amount of radioactivity that had been transferred from the intestinal tract into blood is represented by the proportion of radioactivity per ml of blood (% dose/ml blood) with respect to the total radioactivity administered. The results are shown in Table 22.

TABLE 22

| | Oral |
|---|---|
| No. | administration |
| 3 | NA |
| 27 | + |
| 29 | + |
| 37 | − |
| 38 | NA |
| 40 | ++ |
| 41 | NA |
| 45 | + |
| 50 | + |
| 53 | + |
| 56 | ++ |
| 60 | − |
| 67 | ++ |
| 68 | + |
| 69 | ++ |
| 70 | ++ |
| 71 | + |
| 76 | NA |
| 80 | − |
| 87 | + |
| 91 | NA |
| 93 | − |
| 96 | NA |
| 105 | ++ |
| 106 | NA |
| 111 | − |
| 116 | − |
| 117 | + |
| 120 | + |
| 125 | ++ |
| 127 | NA |
| 129 | NA |
| 130 | NA |
| 137 | NA |
| 139 | ++ |
| 141 | − |
| 142 | ++ |
| 146 | NA |
| 150 | + |
| 151 | NA |
| 152 | ++ |
| 153 | ++ |
| 154 | + |
| 155 | − |
| 175 | ++ |
| 178 | NA |
| 183 | − |
| 184 | ++ |
| 186 | ++ |
| 191 | − |
| 195 | NA |
| 197 | ++ |
| 199 | NA |
| 204 | ++ |
| 207 | − |
| 209 | NA |
| 213 | NA |
| 218 | NA |
| 220 | NA |
| 224 | ++ |
| 225 | ++ |
| 229 | − |
| 230 | ++ |
| 233 | − |
| 234 | − |
| 235 | NA |
| 236 | + |
| 237 | ++ |
| 239 | NA |
| 240 | ++ |
| 249 | − |
| 254 | − |
| 263 | + |
| 273 | ++ |
| 285 | + |
| 288 | ++ |
| 290 | ++ |
| 292 | − |
| 295 | NA |
| 301 | − |

### Example 20

### Study of In Vivo Kinetics of Sugar Chain-Modified Liposome

Of the sugar chain-bound liposomes prepared in the above Examples, liposomes administered herein are: sugar chain-bound liposomes (hereinafter, sugar chain+liposome) and liposomes to which no sugar chain had been bound (hereinafter, sugar chain−liposome). The sugar chain+liposome and the sugar chain-liposome were separately administered to normal mice via oral administration for the purpose of evaluating the accumulation of such liposome in each organ of the mice.

Liposome solutions that had been prepared in advance were administered to mice via oral administration and then all the organs were each excised. Each organ was prepared as a tissue homogenate using a 1% Triton X solution and a HG30 homogenizer (Hitachi Koki Co., Ltd.). Liposomes contained in the tissue homogenates were extracted using 100% methanol and chloroform. Regarding the amount of a liposome, the fluorescence intensity of FITC bound to the liposome was measured using a fluorescent microplate reader Biolumin960 (Molecular Dynamics) at 490 nm of excitation and 520 nm of emission. Regarding data obtained via oral administration, the results obtained via oral administration alone are shown. Regarding the other organs (not obtained via oral administration), the results obtained via intravenous injection are shown. In the case of oral administration, vehicles that had transferred into blood as a result of oral administration showed similar tendency to that in the case of intravenous injection.

TABLE 23

| | Abbreviated | Oral | | | | | | | Inflammation | | Large | | Bone | Blood | | | Thymus | |
|---|---|---|---|---|---|---|---|---|---|---|---|---|---|---|---|---|---|---|
| No. | name | administration | Liver | Heart | Lung | Pancreas | Brain | Tumor | site | Small intestine | intestine | Lymph node | marrow | (in blood) | Kidney | Spleen | gland | Muscle |
| 3 | EEGD1A-BH | NA | ++ | − | − | − | − | − | − | − | − | − | ++ | − | − | ++ | − | − |
| 27 | LDF-5 | + | + | − | + | ++ | − | ++ | ++ | ++ | ++ | + | ++ | + | + | − | + | ++ |
| 29 | 3SL-5 | + | + | ++ | ++ | ++ | ++ | ++ | − | ++ | + | ++ | ++ | ++ | ++ | − | − | − |
| 37 | FEEGD1b | − | ++ | − | − | − | − | − | − | − | ++ | − | − | − | − | − | − | − |
| 38 | G4GN-4 | NA | ++ | ++ | ++ | ++ | + | ++ | ++ | ++ | ++ | ++ | ++ | − | ++ | + | ++ | ++ |
| 40 | BAT-1 | ++ | − | ++ | + | ++ | ++ | + | + | ++ | + | ++ | + | ++ | + | − | ++ | + |
| 41 | SOLX-4 | NA | + | − | − | − | − | + | + | − | − | − | − | − | − | − | − | − |
| 45 | BHD-1 | + | − | + | − | − | ++ | + | − | − | ++ | + | + | ++ | + | − | − | + |
| 50 | Man8 | + | − | ++ | + | − | ++ | − | + | ++ | − | − | ++ | ++ | + | − | − | + |
| 53 | L2F-1 | + | + | + | ++ | ++ | + | ++ | ++ | ++ | ++ | + | ++ | + | ++ | + | + | ++ |
| 56 | Man9 | ++ | − | + | − | + | + | − | + | + | − | − | − | ++ | + | − | − | + |
| 60 | 6SLN-1 | − | − | ++ | ++ | + | ++ | ++ | ++ | ++ | ++ | ++ | ++ | ++ | ++ | − | ++ | ++ |
| 67 | FEEaMDa | ++ | + | ++ | + | − | + | − | − | − | − | − | ++ | ++ | ++ | − | − | ++ |
| 68 | Man7 | + | − | ++ | ++ | ++ | ++ | + | + | ++ | ++ | + | + | ++ | ++ | − | − | + |
| 69 | L4F-1 | ++ | − | − | ++ | + | + | ++ | ++ | ++ | + | + | + | + | + | + | + | ++ |
| 70 | LNT-5 | ++ | + | ++ | ++ | + | ++ | − | ++ | + | + | + | ++ | + | ++ | − | − | ++ |
| 71 | BBT-1 | + | − | + | + | ++ | ++ | + | + | + | + | + | + | ++ | + | − | + | + |
| 76 | BHD-4 | NA | − | + | − | + | + | − | + | − | − | − | − | ++ | + | − | + | + |
| 80 | 6SLN-5 | − | − | + | − | − | + | − | − | + | ++ | ++ | + | + | + | − | + | + |
| 87 | TA-1 | + | − | − | − | + | + | ++ | ++ | ++ | + | ++ | ++ | − | − | − | − | ++ |
| 91 | G6GN-4 | NA | + | + | ++ | ++ | − | + | ++ | ++ | ++ | + | + | − | − | + | ++ | ++ |
| 93 | A36 | − | − | ++ | ++ | ++ | ++ | ++ | ++ | ++ | ++ | ++ | ++ | ++ | ++ | − | ++ | ++ |
| 96 | TA-4 | NA | + | ++ | ++ | ++ | − | ++ | ++ | ++ | ++ | + | ++ | − | ++ | − | + | ++ |
| 105 | Man6 | ++ | − | ++ | + | ++ | ++ | + | ++ | ++ | ++ | − | ++ | ++ | ++ | − | ++ | ++ |
| 106 | LDF-4 | NA | + | + | + | ++ | + | + | ++ | ++ | ++ | ++ | ++ | + | ++ | + | + | ++ |
| 111 | LNB-5 | − | + | + | + | + | − | + | + | − | + | − | − | − | − | − | − | + |
| 116 | LDF-1 | − | − | NA | + | ++ | + | ++ | ++ | ++ | ++ | ++ | ++ | + | + | + | + | ++ |
| 117 | TA-5 | + | + | − | ++ | + | − | + | + | + | ++ | + | − | − | − | + | − | + |
| 120 | A2 | + | − | + | ++ | + | ++ | + | + | ++ | + | + | + | ++ | ++ | − | − | + |
| 125 | LX-1 | ++ | − | − | ++ | ++ | − | ++ | ++ | + | + | + | − | − | − | + | ++ | ++ |
| 127 | G3G-4 | NA | − | + | − | + | + | − | − | − | − | − | − | ++ | + | + | − | − |
| 129 | BBT-4 | NA | − | − | − | − | + | − | − | − | − | − | − | + | − | − | − | − |
| 130 | 2FL-4 | NA | + | − | − | + | − | − | − | + | + | + | − | − | − | − | − | + |
| 137 | Man4B | NA | − | ++ | ++ | ++ | ++ | − | + | ++ | + | + | ++ | ++ | ++ | − | + | ++ |
| 139 | 3FL-5 | ++ | + | + | + | ++ | − | + | ++ | + | ++ | + | − | − | + | + | + | ++ |
| 141 | BBT-5 | − | − | − | − | − | + | − | − | − | − | + | − | + | − | − | − | − |
| 142 | BHD-5 | ++ | − | + | − | + | + | − | − | − | − | ++ | − | ++ | + | − | − | − |
| 146 | 3FL-4 | NA | ++ | + | + | + | + | + | ++ | ++ | + | ++ | ++ | + | ++ | + | ++ | + |
| 150 | TNA-5 | + | + | + | ++ | + | + | ++ | ++ | + | − | + | + | + | + | ++ | + | ++ |
| 151 | SLX-4 | NA | ++ | ++ | ++ | ++ | ++ | + | + | ++ | ++ | ++ | + | ++ | ++ | ++ | ++ | + |
| 152 | LY-1 | ++ | + | + | ++ | + | + | ++ | ++ | ++ | + | − | ++ | − | − | ++ | ++ | ++ |
| 153 | G6GN-5 | ++ | − | + | + | ++ | − | + | ++ | + | ++ | + | + | − | + | + | + | ++ |
| 154 | Lac-5 | + | ++ | ++ | ++ | ++ | + | ++ | ++ | ++ | ++ | ++ | ++ | + | ++ | ++ | ++ | ++ |
| 155 | A6 | − | − | ++ | + | + | ++ | ++ | ++ | ++ | ++ | + | ++ | ++ | ++ | − | ++ | ++ |
| 175 | G3G-5 | ++ | − | ++ | + | + | + | − | − | + | − | + | + | ++ | ++ | − | − | − |
| 178 | LNT-4 | NA | + | + | ++ | − | + | − | − | − | − | − | + | − | + | + | + | − |
| 183 | G4GN-5 | − | ++ | + | ++ | − | − | − | + | − | − | − | − | − | + | ++ | − | + |
| 184 | STNA-1 | ++ | + | ++ | ++ | ++ | + | ++ | ++ | ++ | ++ | ++ | + | + | + | ++ | ++ | ++ |
| 186 | Man3 | ++ | − | ++ | + | − | ++ | + | + | ++ | + | ++ | ++ | ++ | ++ | − | − | ++ |
| 191 | LB-1 | − | − | − | + | + | − | + | ++ | + | + | − | + | − | − | + | − | + |
| 195 | TNA-4 | NA | ++ | ++ | ++ | ++ | − | ++ | ++ | ++ | ++ | + | + | + | ++ | ++ | ++ | ++ |
| 197 | A4 | ++ | − | ++ | + | + | ++ | + | + | + | + | − | ++ | ++ | ++ | − | − | + |
| 199 | STNA-4 | NA | + | ++ | ++ | ++ | − | ++ | ++ | ++ | ++ | ++ | ++ | − | ++ | ++ | ++ | ++ |
| 204 | Lac-1 | ++ | + | + | ++ | + | − | ++ | ++ | + | + | + | + | + | + | + | − | + |
| 207 | 6SL-5 | − | − | ++ | ++ | + | ++ | − | − | + | + | ++ | + | ++ | + | − | + | − |
| 209 | LDFH-4 | NA | ++ | ++ | ++ | ++ | ++ | + | + | ++ | ++ | ++ | ++ | + | ++ | + | − | ++ |
| 213 | LX-4 | NA | − | + | + | ++ | + | ++ | ++ | ++ | ++ | + | + | − | − | + | ++ | ++ |
| 218 | EEGM1-BH | NA | ++ | − | − | − | − | − | − | − | − | − | NA | − | − | − | − | − |
| 220 | LNB-4 | NA | − | + | + | ++ | − | + | ++ | + | ++ | + | ++ | − | + | − | ++ | ++ |
| 224 | Man5 | ++ | − | + | + | ++ | ++ | ++ | ++ | ++ | ++ | − | ++ | ++ | ++ | − | − | ++ |
| 225 | 3FL-1 | ++ | ++ | − | − | + | − | + | − | + | + | + | − | + | − | ++ | − | + |
| 229 | G6GN-1 | − | + | ++ | ++ | ++ | + | ++ | ++ | ++ | ++ | + | ++ | − | + | ++ | + | ++ |
| 230 | G4GN-1 | ++ | + | + | − | ++ | + | ++ | ++ | ++ | ++ | + | ++ | − | + | ++ | + | ++ |
| 233 | 2FL-1 | − | − | − | ++ | ++ | ++ | ++ | ++ | ++ | + | + | ++ | + | + | − | ++ | ++ |
| 234 | TNA-1 | − | ++ | + | ++ | + | − | ++ | ++ | + | + | + | + | − | − | ++ | + | ++ |
| 235 | Lac-4 | NA | − | + | + | ++ | + | ++ | ++ | ++ | + | + | + | + | + | − | ++ | ++ |
| 236 | 2FL-5 | + | + | − | − | − | − | + | − | + | + | + | − | − | − | − | − | + |
| 237 | A3 | ++ | − | ++ | ++ | ++ | ++ | ++ | ++ | ++ | ++ | ++ | ++ | ++ | ++ | − | + | ++ |
| 239 | SLA-4 | NA | ++ | − | + | − | − | − | − | − | − | − | − | − | − | ++ | − | − |
| 240 | SOLA-5 | ++ | ++ | − | − | − | − | + | + | − | − | + | − | − | − | + | − | − |
| 249 | 3SL-1 | − | − | ++ | + | ++ | ++ | − | − | ++ | ++ | ++ | ++ | ++ | ++ | − | − | + |
| 254 | G3G-1 | − | − | + | − | − | − | − | − | − | − | − | − | + | − | − | − | − |
| 263 | 3SLN-1 | + | − | ++ | ++ | ++ | ++ | ++ | ++ | ++ | ++ | ++ | ++ | ++ | ++ | + | − | ++ |
| 273 | BAT-5 | ++ | − | ++ | − | + | ++ | − | − | − | − | − | − | ++ | + | − | ++ | + |
| 285 | SLX-6 | + | ++ | − | − | − | − | ++ | − | − | − | − | − | − | − | − | − | − |
| 288 | LA-1 | ++ | − | − | + | − | − | ++ | + | − | − | ++ | + | − | − | + | − | + |
| 290 | SLA-5 | ++ | ++ | − | − | − | + | + | ++ | − | − | − | + | − | − | + | ++ | − |
| 292 | STNA-5 | − | + | ++ | ++ | ++ | + | ++ | ++ | ++ | ++ | ++ | ++ | + | ++ | ++ | ++ | ++ |
| 295 | 6SLN-4 | NA | − | ++ | + | ++ | ++ | + | + | ++ | ++ | ++ | ++ | ++ | + | − | ++ | ++ |
| 301 | TRIS | − | − | − | − | − | − | − | − | − | − | − | − | ++ | − | − | − | − |

### Example 21

### Preparation of Anticancer Agent Doxorubicin-Encapsulated Liposome

A liposome was prepared using cholic acid dialysis. Specifically, dipalmitoylphosphatidylcholine, cholesterol, dicetylphosphate, ganglioside, and dipalmitoylphosphatidylethanol amine were mixed at a molar ratio of 35:40:5:15:5 so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, thereby obtaining a lipid membrane. The thus obtained lipid membrane was suspended in 10 ml of TAPS buffered saline solution (pH8.4) and then the resultant was ultrasonicated, so that 10 ml of a transparent micelle suspension was obtained. An anticancer agent doxorubicin that had been completely dissolved in a TAPS buffer solution (pH8.4) to a concentration of 3 mg/1 ml was slowly added dropwise to the micelle suspension while agitating the suspension. After homogeneously mixing the suspension, the doxorubicin-containing micelle suspension was subjected to ultrafiltration using a PM10 membrane (Amicon Co., U.S.A.) and a TAPS buffered saline solution (pH8.4) so that 10 ml of a homogeneous anticancer agent doxorubicin-encapsulated liposome particle suspension was prepared. The particle diameter and zeta potential of the anticancer agent doxorubicin-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV.

### Example 22A

### Measurement of Anticancer Effect of Various Sugar Chain-Bound Liposome Complexes in Cancer-Bearing Mice Via Oral Administration

(1) Anticancer Effect in Cancer-Bearing Mice after Injection Via Tail Vein

Cancer-bearing mice were produced as follows. The hair on the back of each ddY 7-week-old mouse (male and body weight: 35 g to 40 g) was shaved using an electric shaver and then Ehrlich Ascites Tumor (approximately 5×10⁶ cells/mouse) was subcutaneously transplanted. The mice were grown and observed for 10 days. The mice in which cancer cells had successfully survived and grown were selected and then used for the experiment. Drug administration and measurement of the volume of cancer were performed as follows. Two types of group were prepared: a group to which doxorubicin-encapsulated liposome No. 155 had been administered, in which the concentration of doxorubicin (encapsulated as a drug to be administered) had been adjusted at 0.0625 mg/kg; and a group to which a physiological saline solution had been administered as a control. The liposome was administered through injection via tail vein to cancer-bearing mice 4 times a week for 2 weeks. The major diameter of cancer and the minor diameter of cancer were measured using a micrometer caliper. The measurement was initiated on day 10 after transplantation of cancer cells and performed twice a week for 4 weeks. The volume of cancer that had grown was calculated by the following formula.

Volume of cancer (mm³)=(major diameter+minor diameter²)/2

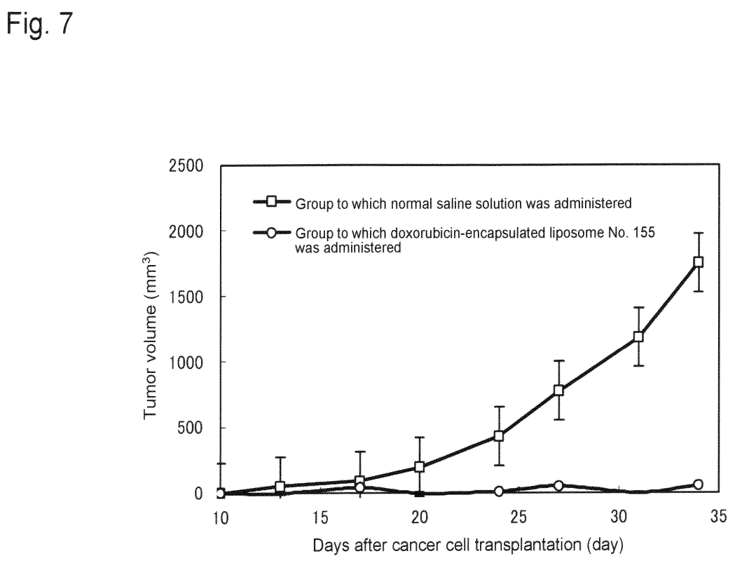

FIG. 1 shows the results. Changes in the volume of tumor that had grown were compared in each of the group to which doxorubicin-encapsulated liposome No. 155 had been administered and the group to which the physiological saline solution had been administered as a control. Although the extremely small dose of the liposome, the effect of suppressing cancer growth was exerted significantly in the group to which doxorubicin-encapsulated liposome No. 155 had been administered, such that cancer growth was suppressed from the initiation of administration. The two groups were compared in terms of cancerous tissue size (volume) on day 34 after transplantation; that is the final measurement day. A more significant antiproliferative effect was observed in the group to which doxorubicin-encapsulated liposome No. 155 had been administered compared with that in the group to which the physiological saline solution had been administered. These results demonstrate that the antineoplastic agent-encapsulated and sugar chain-modified liposome has a strong anticancer effect even when an extremely small dose of the agent is administered (FIG. 7).

FIG. 7 shows the anticancer effect exerted in cancer-bearing mice when the doxorubicin-encapsulated liposome No. 155 was administered through injection via tail vein.

(2) Fluorescent Microscopic Observation of the Transfer of Doxorubicin to Cancerous Tissue when Doxorubicin was Administered Through Injection Via Tail Vein

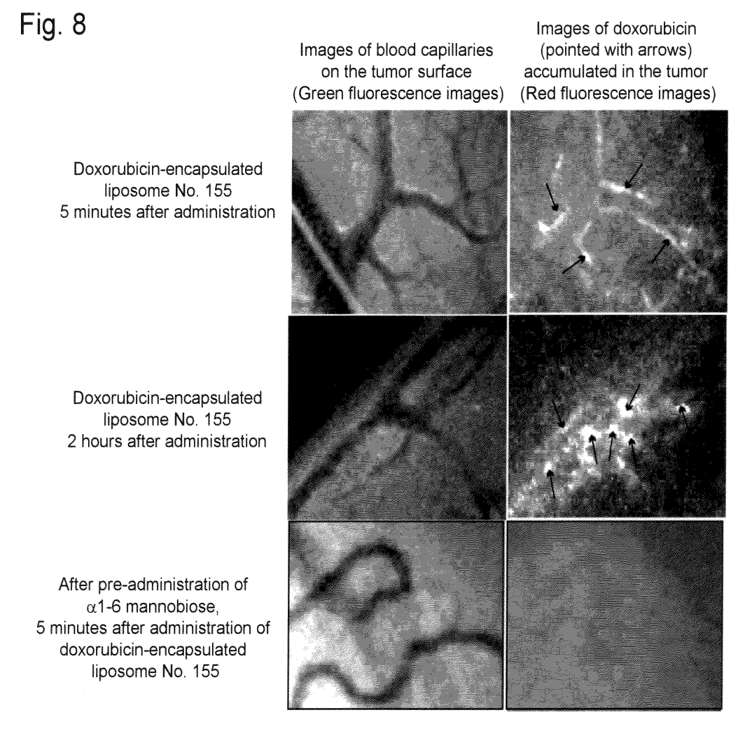

Fluorescent microscopic observation was performed as follows. The skin of a cancer site of each cancer-bearing mouse was excised to expose the cancerous tissue. The cancer site was fixed on a slide glass. Each mouse was placed on the stage of a fluorescence microscope. Blood vessels around the cancerous tissue were searched, so that a position at which the blood vessel image could be clearly observed was determined. 0.2 ml of doxorubicin-encapsulated liposome No. 155 (lipid concentration: 2 mg/mL and doxorubicin concentration: 0.025 mg/mL) was administered through injection via tail vein. Immediately after administration, observation of the accumulation of doxorubicin into the cancerous tissue was initiated under a fluorescence microscope. An inhibition experiment involving pre-administration of a modified sugar was conducted as follows. 0.2 mL of a modified sugar chain (α1-6mannobiose) solution (60 mM) was administered at 5 minutes before administration of doxorubicin-encapsulated liposome No. 155. Observation was performed by the method similar to the above. Photograph 1 shows the result. Immediately after administration of doxorubicin-encapsulated liposome No. 155, doxorubicin fluorescence was observed in the blood vessels in the vicinity of the cancerous tissue. At 5 minutes after administration, red doxorubicin fluorescence was observed in the blood vessel wall part. Thereafter, the transfer of doxorubicin to the relevant tissues with the course of time was observed. Two hours later, doxorubicin fluorescence was observed within the tumor tissues in the periphery of cancerous blood vessels. Pre-administration of the modified sugar chain resulted in complete block of the accumulation of doxorubicin-encapsulated liposome No. 155 into the tumor tissues. Hence, fluorescence was not observed in the blood vessel walls from the time point immediately after administration of doxorubicin-encapsulated liposome No. 155. These results demonstrate that the sugar chain-modified liposomes can achieve highly efficient accumulation and delivery of drugs, fluorescent substances, radiolabeled substances, or the like via active targeting to parts affected by diseases or various organs with the use of the functions of a sugar chain as a ligand (FIG. 8).

FIG. 8 shows fluorescent microscopic photographs showing the effect of accumulating doxorubicin from tumor blood vessels to tumor tissues and cells in cancer-bearing mice when doxorubicin-encapsulated liposome No. 155 was administered through injection via tail vein. Green fluorescent microscopic photographs on the left are of the same tissue or cells. Red fluorescent microscopic photographs on the right are of the same tumor tissue or cells. Green (images on the left) shows natural fluorescence of the blood vessels, tissue, and cells. Red (images on the right) shows doxorubicin fluorescence (doxorubicin is a fluorescent substance) in the tumor tissue and the cancer cells. These results demonstrate that the sugar chain-modified liposomes can achieve highly efficient accumulation and delivery of drugs, fluorescent substances, radiolabeled substances, or the like via active targeting to parts affected by diseases or various organs with the use of the functions of a sugar chain as a ligand. Therefore, the sugar chain-modified liposome of the present invention can visualize the accumulation in target tissues such as tumors. Thus, according to the present invention, in addition to a delivery vehicle for delivering a drug for treatment, a delivery vehicle to be used as a reagent for research or a diagnostic agent is provided.

### Example 22B

### Measurement of Anticancer Effect of Various Sugar Chain-Bound Liposome Complexes in Cancer-Bearing Mice when the Complexes were Administered Via Oral Administration

(1) Anticancer Effect in Cancer-Bearing Mice after Oral Administration

Cancer-bearing mice were produced as follows. The hair on the back of each ddY 7-week-old mouse (male and body weight: 35 g to 40 g) was shaved using an electric shaver and then Ehrlich Ascites Tumor (approximately 5×10⁶ cells/mouse) was subcutaneously transplanted. The mice were grown and observed for about 10 days. The mice in which cancer cells had successfully survived and grown were selected and then used for the experiment. Drug administration and measurement of the volume of cancer were performed as follows. Two types of group were prepared: a group to which doxorubicin-encapsulated liposome No. 237 had been administered, in which the concentration of doxorubicin (encapsulated as a drug to be administered) had been adjusted at 0.375 mg/kg; and a group to which a physiological saline solution had been administered as a control. The liposome was administered via oral administration to cancer-bearing mice 4 times a week for 2 weeks. The major diameter of cancer and the minor diameter of cancer were measured using a micrometer caliper. The measurement was initiated on day 10 after transplantation of cancer cells and performed twice a week for 4 weeks. The volume of cancer that had grown was calculated by the following formula:

Volume of cancer (mm³)=(major diameter+minor diameter²)/2.

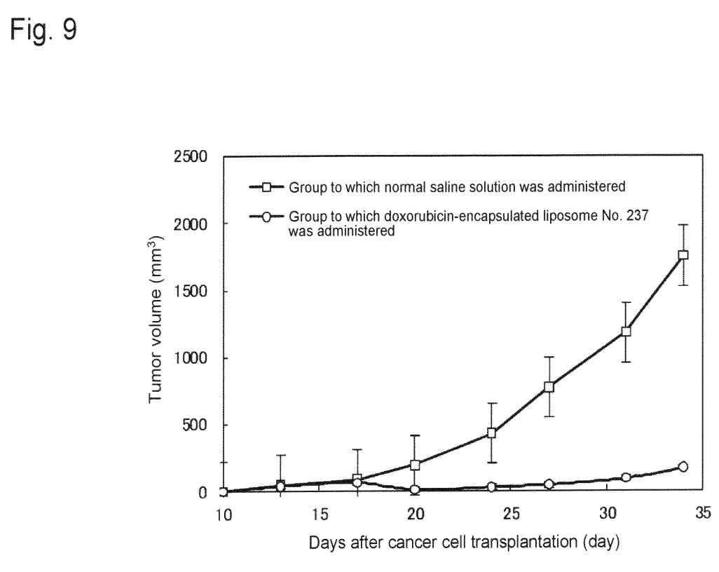

FIG. 1 shows the results. Changes in the volume of tumor that had grown were compared in each of the group to which doxorubicin-encapsulated liposome No. 237 had been administered and the group to which the physiological saline solution had been administered as a control. In spite of the extremely small dose, the effect of suppressing cancer growth was exerted significantly in the group to which doxorubicin-encapsulated liposome No. 237 had been administered, such that cancer growth was suppressed from the initiation of administration. The two groups were compared in terms of cancerous tissue size (volume) on day 34 after transplantation; that is the final measurement day. A more significant antiproliferative effect was observed in the group to which doxorubicin-encapsulated liposome No. 237 had been administered compared with that in the group to which the physiological saline solution had been administered. These results demonstrate that the antineoplastic agent-encapsulated and sugar chain-modified liposome has a strong anticancer effect even when an extremely small dose is administered (FIG. 9).

FIG. 9 shows the anticancer effect of doxorubicin-encapsulated liposome No. 237 in cancer-bearing mice when the liposome was administered via oral administration.

(2) Fluorescent Microscopic Observation of the Transfer of Doxorubicin to Cancerous Tissue when the Liposome was Administered Via Oral Administration

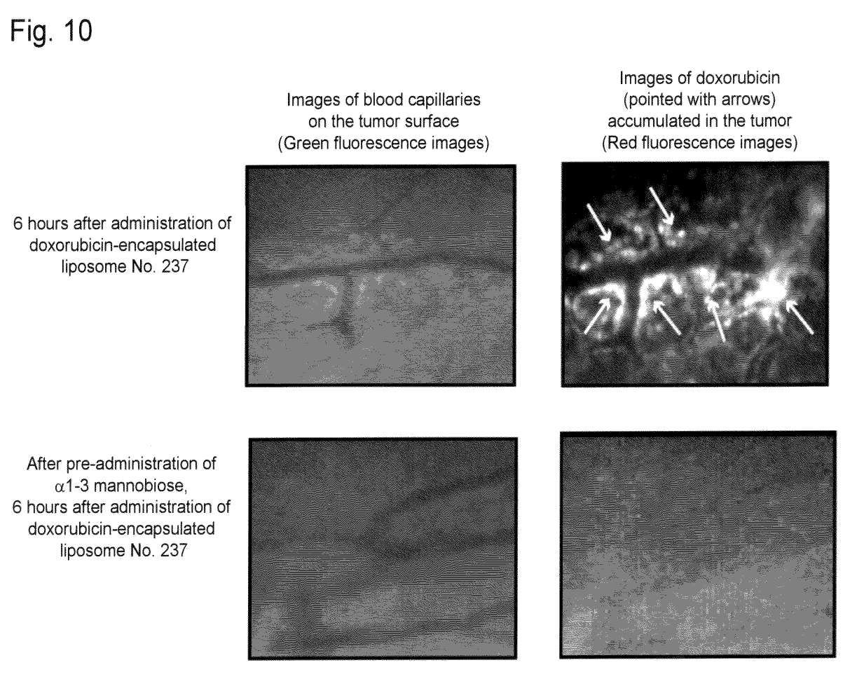

Fluorescent microscopic observation was performed as follows. The skin of a cancer site of each cancer-bearing mouse was excised to expose the cancerous tissue. The cancer site was fixed on a slide glass. The mouse was placed on the stage of a fluorescence microscope. Blood vessels in the vicinity of the cancerous tissue were searched, so that a position at which a blood vessel image could be clearly observed was determined. 0.3 ml of doxorubicin-encapsulated liposome No. 237 (lipid concentration: 4 mg/mL and doxorubicin concentration: 0.050 mg/mL) was administered via oral administration. Immediately after administration, observation of the accumulation of doxorubicin into the cancerous tissues was initiated under a fluorescence microscope. An inhibition experiment involving pre-administration of a modified sugar was conducted as follows. 0.3 mL of a modified sugar chain (α1-3mannobiose) solution (60 mM) was administered at 5 minutes before administration of doxorubicin-encapsulated liposome No. 237. Observation was performed by the method similar to the above. Photograph 1 shows the result. After administration of doxorubicin-encapsulated liposome No. 237, the transfer of doxorubicin to the tissues was observed with the course of time. 6 hours later, doxorubicin fluorescence was observed within the tumor tissues in the vicinity of cancerous blood vessels. Pre-administration of the modified sugar chain resulted in the complete block of the accumulation of doxorubicin-encapsulated liposome No. 237 into the tumor tissues. Hence, fluorescence was not observed in the blood vessel walls from the time point immediately after administration of doxorubicin-encapsulated liposome No. 237. These results demonstrate that the sugar chain-modified liposomes can achieve highly efficient accumulation and delivery of drugs, fluorescent substances, radiolabeled substances, or the like via active targeting to parts affected by diseases or various organs with the use of the functions of a sugar chain as a ligand (FIG. 10).

FIG. 10 shows fluorescent microscopic photographs showing the effect of accumulating doxorubicin from tumor blood vessels to tumor tissues and cells in cancer-bearing mice when doxorubicin-encapsulated liposome No. 237 was administered through injection via tail vein. Green fluorescent microscopic photographs on the left are of the same tumor tissue and cells. Red fluorescent microscopic photographs on the right are of the same tumor tissue and cells. Green (images on the left) shows natural fluorescence of the blood vessel, tissue, and cells. Red (images on the right) shows doxorubicin fluorescence (doxorubicin is a fluorescent substance) in the tumor tissue and the cancer cells. These results demonstrate that the sugar chain-modified liposomes can achieve highly efficient accumulation and delivery of drugs, fluorescent substances, radiolabeled substances, or the like via active targeting to parts affected by diseases or various organs with the use of the functions of a sugar chain as a ligand. Therefore, the sugar chain-modified liposomes of the present invention can visualize the accumulation in target tissues such as tumors.

Thus, according to the present invention, in addition to a delivery vehicle for delivering a drug for treatment, a delivery vehicle to be used as a reagent for research or a diagnostic agent is provided.

### Example 23

### Preparation of Sugar Chain-Modified Liposome Appropriate for Administration Based on Rolling Model

Measurement results (obtained with the use of E-selectin) determined based on Example 7 was simplified and analyzed as follows using the rolling model of the present invention.

Graphs used for calculation of typical IC10, IC20, IC30, IC40, IC50, and IC60 are shown (typically, see FIGS. 2 to 6).

Next, the thus obtained IC10 or the like was compared with in vivo affinity. The results are shown in the following Table. In the Table, typical results obtained for tumors and inflammation sites are listed.

As a result, for example, typically in the case of E-selectin, it was revealed that a threshold value of strong binding and weak binding is present between approximately IC30 and IC31. The presence of similar threshold values is expected in the case of other lectins.

Moreover, it was also revealed that concerning in vitro affinity for a lectin associated with a desired site, an inhibitory concentration at a strong binding IC that is approximately IC30 or less is 10⁻⁹M or less (preferably, 5×10⁻¹⁰ or less) and an inhibitory concentration at a weak binding IC that is approximately IC31 or more is 10⁻⁹M or more (preferably, 5×10⁻⁸M or more, 10⁻⁸M or more).

E-selectin is significantly expressed in, when it is administered via oral administration, the liver, small intestine, large intestine, lymph node, liver, heart, pancreas, inflammation sites, and cancer sites. The expression of E-selectin is particularly significant and characteristic in inflammation sites and cancer sites. It was thus revealed that E-selectin can be used in in vitro convenient rolling model assay performed for these organs. Based on these results, delivery vehicles were prepared as follows.

### (1 Preparation of Liposome)

A liposome was prepared by the techniques of the previous report (Yamazaki, N., Kodama, M. and Gabius, H.-J. (1994) Methods Enzymol. 242, 56-65) using improved cholic acid dialysis. Specifically, dipalmitoylphosphatidylcholine, cholesterol, dicetylphosphate, ganglioside, and dipalmitoylphosphatidylethanol amine were mixed at a molar ratio of 35:40:5:15:5 so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, thereby obtaining a lipid membrane. The thus obtained lipid membrane was suspended in 3 ml of a TAPS buffer solution (pH8.4) and then the resultant was ultrasonicated, so that a transparent micelle suspension was obtained. Furthermore, the micelle suspension was subjected to ultrafiltration using a PM10 membrane (Amicon Co., U.S.A.) and a PBS buffer solution (pH7.2). Thus 10 ml of homogeneous liposome (average particle diameter: 100 nm) was prepared.

### (2. Hydrophilization of Liposome Lipid Membrane Surface)

10 ml of the liposome solution prepared in 1 above was subjected to ultrafiltration using an XM300 membrane (Amicon Co., U.S.A.) and a CBS buffer solution (pH8.5) and the pH of the solution was adjusted to pH8.5. Next, 10 ml of a cross-linking reagent bis(sulfosuccinimidyl)suberate (BS3; Pierce Co., U.S.A.) was added, followed by 2 hours of agitation at 25° C. Subsequently, the solution was further agitated overnight at 7° C. so as to complete the chemical binding reaction between lipid dipalmitoylphosphatidylethanol amine on the liposome membrane and BS3. The liposome solution was then subjected to ultrafiltration using an XM300 membrane and a CBS buffer solution (pH8.5). Next, 40 mg of tris(hydroxymethyl)aminomethane dissolved in 1 ml of a CBS buffer solution (pH8.5) was added to 10 ml of the liposome solution, followed by 2 hours of agitation at 25° C. The solution was then agitated overnight at 7° C., so as to complete the chemical binding reaction between BS3 bound to the lipids on the liposome membrane and tris(hydroxymethyl)aminomethane. Thus, the hydroxy group of tris(hydroxymethyl)aminomethane was coordinated on the lipid dipalmitoylphosphatidylethanol amine of the liposome membrane, so that the liposome membrane surface was hydrated and hydrophilized.

(3. Binding of Human Serum Albumin (HSA) onto Liposome Membrane Surface)

Human serum albumin (HSA) was bound onto a liposome membrane surface according to the technique of the previous report (Yamazaki, N., Kodama, M. and Gabius, H.-J. (1994) Methods Enzymol. 242, 56-65) using a coupling reaction method. Specifically, the reaction was performed as a two-step chemical reaction. First, ganglioside existing on the liposome membrane surface of 10 ml of the liposome obtained in 2 above was added to 43 mg of sodium metaperiodate dissolved in 1 ml of a TAPS buffer solution (pH8.4), followed by 2 hours of agitation at room temperature to perform periodate oxidation. The resultant was subjected to ultrafiltration using an XM300 membrane and a PBS buffer solution (pH8.0) so that 10 ml of the thus oxidized liposome was obtained. 20 mg of human serum albumin (HSA) was added to the liposome solution, followed by 2 hours of agitation at 25° C. Next, 100 μl of 2M NaBH₃CN was added to PBS (pH8.0) and then the solution was agitated overnight at 10° C. Thus, HSA was bound by the coupling reaction between ganglioside on the liposome and HSA. The resultant was then subjected to ultrafiltration using an XM300 membrane and a CBS buffer solution (pH8.5), so that 10 ml of an HSA-bound liposome solution was obtained.

### (4. Preparation of Sugar Chain)

Sugar chains were prepared by the procedures same as those in Example 4.

(5.1. Binding of Sugar Chain onto Liposome Membrane-Surface-Bound Human Serum Albumin (HSA))

50 μg of each sugar chain prepared in 4 above was added to 0.5 ml of an aqueous solution in which 0.25 g of NH₄HCO₃ had been dissolved, followed by 3 days of agitation at 37° C. The resultant was filtered with a 0.45-μm filter to complete the amination reaction of the reducing termini of the sugar chains. Thus, 50 μg of a glycosylamine compound of each sugar chain was obtained. Next, 1 mg of a cross-linking reagent 3,3′-dithiobis(sulfo succinimidyl propionate (DTSSP; Pierce Co., U.S.A.) was added to 1 ml of the liposome solution (a portion of the liposome solution) obtained in Example 3. The solution was agitated for 2 hours at 25° C. and then agitated overnight at 7° C. The resultant was subjected to ultrafiltration using an XM300 membrane and a CBS buffer solution (pH8.5), so that 1 ml of liposome was obtained on which DTSSP was bound to HSA on the liposome. Next 50 μg of the above glycosylamine compound was added to the liposome solution. The solution was agitated for 2 hours at 25° C. and then agitated overnight at 7° C. The resultant was subjected to ultrafiltration using an XM300 membrane and a PBS buffer solution (pH7.2) so as to bind the glycosylated amine compound to DTSSP on the liposome membrane-surface-bound human serum albumin. As a result, as listed in Table 2, liposomes (2 ml each) (total amount of lipid: 2 mg, total amount of protein: 200 μg, and average particle diameter: 100 nm) were obtained, each of which was prepared by binding of a sugar chain, human serum albumin, and the liposome. Unless otherwise clearly specified, binding of these sugar chains onto liposome membrane-surface-bound human serum albumin were performed by the method and the conditions similar to those in Example 5.

(5.2. Binding of Tris(Hydroxymethyl)Aminomethane onto Liposome Membrane-Surface-Bound Human Serum Albumin (HSA))

To prepare a liposome as a sample for comparison, 1 mg of a cross-linking reagent 3,3′-dithiobis(sulfosuccinimidyl propionate (DTSSP; Pierce Co., U.S.A.) was added to 1 ml of the liposome solution (a portion of the liposome solution) obtained in Example 3. The solution was agitated for 2 hours at 25° C. and then agitated overnight at 7° C. The resultant was subjected to ultrafiltration using an XM300 membrane and a CBS buffer solution (pH8.5), so that 1 ml of liposome in which DTSSP was bound to HSA on the liposome was obtained. Next, 13 mg of tris(hydroxymethyl)aminomethane (Wako Co., Japan) was added to the liposome solution. The solution was agitated for 2 hours at 25° C. and then agitated overnight at 7° C. The resultant was subjected to ultrafiltration using an XM300 membrane and a PBS buffer solution (pH7.2) so as to bind the glycosylated amine compound to DTSSP on the liposome membrane-surface-bound human serum albumin. Because of the presence of 13 mg of tris(hydroxymethyl)aminomethane, which was already an extremely excessive amount in this step, hydrophilization on the liposome membrane-surface-bound human serum albumin (HSA) was also completed simultaneously. As a result, the final product, 2 ml of liposome (abbreviated name: TRIS) (total amount of lipid; 2 mg, total amount of protein: 200 μg, and average particle diameter: 100 nm) as a sample for comparison was obtained via binding of hydrophilized tris(hydroxymethyl)aminomethane, human serum albumin, and the liposome.

### (6. Hydrophilization of Liposome Membrane-Surface-Bound Human Serum Albumin (HSA))

Sugar chain-bound liposomes prepared by the means of 5.1 above were separately subjected to hydrophilization of the HSA protein surfaces on the liposomes as described in the following procedures. 13 mg of tris(hydroxymethyl)aminomethane was added to 2 ml of each sugar chain-bound liposome. The solution was agitated for 2 hours at 25° C. and then agitated overnight at 7° C. The resultant was subjected to ultrafiltration using an XM300 membrane and a PBS buffer solution (pH7.2) and then unreacted substances were removed. As a result, the final product, 2 ml of each hydrophilized sugar chain-bound liposome complex (total amount of lipid; 2 mg, total amount of protein: 200 μg, and average particle diameter: 100 nm) was obtained.

(7.)

As described above, compositions appropriate for delivery to organs corresponding to E-selectin could be prepared. When these compositions are actually tested as in the above Examples, it can be confirmed that they are successfully delivered in vivo to desired organs.

### Example 25

### Preparation of Anticancer Agent Doxorubicin-Encapsulated Liposome Appropriate for Oral Administration

Since E-selectin is an indicator of an anticancer agent for oral administration, anticancer agent doxorubicin-encapsulated liposomes were prepared as follows using optimum compounds based on the rolling model and then whether or not the compounds actually had antitumor action was confirmed.

### (1. Preparation of Anticancer Agent Doxorubicin-Encapsulated Liposome

A liposome was prepared by the techniques of the previous report (Yamazaki, N., Kodama, M. and Gabius, H.-J. (1994) Methods Enzymol. 242, 56-65) using improved cholic acid dialysis. Specifically, dipalmitoylphosphatidylcholine, cholesterol, dicetylphosphate, ganglioside, and dipalmitoylphosphatidylethanol amine were mixed at a molar ratio of 35:40:5:15:5 so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, thereby obtaining a lipid membrane. The thus obtained lipid membrane was suspended in 10 ml of a TAPS buffered saline solution (pH8.4) and then the resultant was ultrasonicated, so that 10 ml of a transparent micelle suspension was obtained. An anticancer agent doxorubicin that had been completely dissolved in a TAPS buffer solution (pH8.4) to a concentration of 3 mg/l ml was slowly added dropwise to the micelle suspension while agitating the suspension. After homogeneously mixing the suspension, the doxorubicin-containing micelle suspension was subjected to ultrafiltration using a PM10 membrane (Amicon Co., U.S.A.) and a TAPS buffered saline solution (pH8.4) so that 10 ml of a homogeneous anticancer agent doxorubicin-encapsulated liposome particle suspension was prepared.

The particle diameter and zeta potential of the anticancer agent doxorubicin-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV.

### (2. Hydrophilization of Anticancer Agent Doxorubicin-Encapsulated Liposome Lipid Membrane Surface)

10 ml of the anticancer agent doxorubicin-encapsulated liposome solution prepared in 1 above was subjected to ultrafiltration using an XM300 membrane (Amicon Co., U.S.A.) and a CBS buffer solution (pH8.5). The pH of the solution was adjusted to 8.5. Next, 10 ml of a cross-linking reagent bis(sulfo succinimidyl)suberate (BS3; Pierce Co., U.S.A.) was added. The solution was agitated for 2 hours at 25° C. and then agitated overnight at 7° C. Thus, chemical binding reaction between lipid dipalmitoylphosphatidylethanol amine on the liposome membrane and BS3 was completed. Subsequently, the liposome solution was subjected to ultrafiltration using an XM300 membrane and a CBS buffer solution (pH8.5). Next, 40 mg of tris(hydroxymethyl)aminomethane dissolved in 1 ml of a CBS buffer solution (pH8.5) was added to 10 ml of the liposome solution. The solution was agitated for 2 hours at 25° C. and then further agitated overnight at 7° C. Thus, chemical binding reaction between BS3 bound to the lipids on the liposome membrane and tris(hydroxymethyl)aminomethane was completed. Therefore, the hydroxy group of tris(hydroxymethyl)aminomethane was coordinated on the lipid dipalmitoylphosphatidylethanol amine of anticancer agent doxorubicin-encapsulated liposome membrane, so that the liposome membrane surface was hydrated and hydrophilized.

(3. Binding of Human Serum Albumin (HSA) onto Anticancer Agent Doxorubicin-Encapsulated Liposome Membrane Surface

Human serum albumin (HSA) was bound onto a liposome membrane surface according to the technique of the previous report (Yamazaki, N., Kodama, M. and Gabius, H.-J. (1994) Methods Enzymol. 242, 56-65) using a coupling reaction method. Specifically, the reaction was performed as a two-step chemical reaction. First, ganglioside existing on the membrane surface of 10 ml of the liposomes obtained in 2 above was added to 43 mg of sodium metaperiodate dissolved in 1 ml of a TAPS buffer solution (pH 8.4), followed by 2 hours of agitation at room temperature to perform periodate oxidation. The resultant was subjected to ultrafiltration using an XM300 membrane and a PBS buffer solution (pH 8.0) so that 10 ml of the thus oxidized liposome was obtained. 20 mg of human serum albumin (HSA) was added to the liposome solution, followed by 2 hours of agitation at 25° C. Next, 100 μl of 2M NaBH₃CN was added to PBS (pH 8.0) and then the solution was agitated overnight at 10° C. Thus, HSA was bound by the coupling reaction between ganglioside on the liposome and HSA. The resultant was then subjected to ultrafiltration using an XM300 membrane and a CBS buffer solution (pH 8.5), so that 10 ml of an HSA-bound anticancer agent doxorubicin-encapsulated liposome solution was obtained.

### (4. Preparation of Sugar Chain)

Sugar chains were prepared by procedures similar to those in Example 4.

### (5.1. Binding of Sugar Chain to Anticancer Agent Doxorubicin-Encapsulated Liposome Membrane-Surface-Bound Human Serum Albumin (HSA))

50 μg of each sugar chain prepared in 4 above was added to 0.5 ml of an aqueous solution in which 0.25 g of NH₄HCO₃ had been dissolved, followed by 3 days of agitation at 37° C. The resultant was filtered with a 0.45-μm filter to complete the amination reaction of the reducing termini of the sugar chains. Thus, 50 μg of a glycosylamine compound of each sugar chain was obtained. Next, 1 mg of a cross-linking reagent 3,3′-dithiobis(sulfo succinimidyl propionate (DTSSP; Pierce Co., U.S.A.) was added to 1 ml of the anticancer agent doxorubicin-encapsulated liposome solution (a portion of the liposome solution) obtained in Example 13. The solution was agitated for 2 hours at 25° C. and then agitated overnight at 7° C. The resultant was subjected to ultrafiltration using an XM300 membrane and a CBS buffer solution (pH8.5), so that 1 ml of liposome was obtained on which DTSSP was bound to HSA on the liposome. Next, 50 μg of the glycosylamine compound of the above sugar chain was added to the liposome solution. The solution was agitated for 2 hours at 25° C. and then agitated overnight at 7° C. The resultant was subjected to ultrafiltration using an XM300 membrane and a PBS buffer solution (pH7.2) so as to bind each sugar chain to DTSSP on the liposome membrane-surface-bound human serum albumin. Next, 13 mg of tris(hydroxymethyl)aminomethane (Wako Co., Japan) was added to the liposome solution. The solution was agitated for 2 hours at 25° C. and then agitated overnight at 7° C. The resultant was subjected to ultrafiltration using an XM300 membrane and a PBS buffer solution (pH7.2), so as to bind the glycosylated amine compound to DTSSP on the liposome membrane-surface-bound human serum albumin. As a result, a liposome was obtained, in which tris(hydroxymethyl)aminomethane, human serum albumin, and the liposome were bound and the linker protein (HSA) was hydrophilized. As a result, 2 ml of an anticancer agent doxorubicin-encapsulated liposome (total amount of lipid: 2 mg and total amount of protein: 200 μg) was obtained, in which each sugar chain, human serum albumin, and the liposome were bound and the linker protein (HSA) was hydrophilized.

The particle diameter and zeta potential of the anticancer agent doxorubicin-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV.

(5.2. Binding of Tris(Hydroxymethyl)Aminomethane onto Anticancer Agent Doxorubicin-Encapsulated Liposome Membrane-Surface-Bound Human Serum Albumin (HSA)

To prepare a anticancer agent doxorubicin-encapsulated liposome as a sample for comparison, 1 mg of a cross-linking reagent 3,3′-dithiobis(sulfosuccinimidyl propionate (DTSSP; Pierce Co., U.S.A.) was added to 1 ml of the anticancer agent doxorubicin-encapsulated liposome solution (a portion of the liposome solution) obtained in Example 13. The solution was agitated for 2 hours at 25° C. and then agitated overnight at 7° C. The resultant was subjected to ultrafiltration using an XM300 membrane and a CBS buffer solution (pH8.5), so that 1 ml of liposome in which DTSSP was bound to HSA on the liposome and the linker protein (HSA) was hydrophilized was obtained. Next, 13 mg of tris(hydroxymethyl)aminomethane (Wako Co., Japan) was added to the liposome solution. The solution was agitated for 2 hours at 25° C. and then agitated overnight at 7° C. The resultant was subjected to ultrafiltration using an XM300 membrane and a PBS buffer solution (pH7.2) so as to bind the glycosylated amine compound to DTSSP on the liposome membrane-surface-bound human serum albumin.

As a result, 2 ml of an anticancer agent doxorubicin-encapsulated liposome (abbreviated name: DX-TRIS) (total amount of lipid; 2 mg and total amount of protein: 200 μg) as a sample for comparison was obtained, in which tris(hydroxymethyl)aminomethane, human serum albumin, and the liposome were bound and the linker protein (HSA) was hydrophilized.

The particle diameter and zeta potential of the anticancer agent doxorubicin-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV.

(6. Binding of Sugar Chain onto Anticancer Agent Doxorubicin-Encapsulated Liposome Membrane-Surface-Bound Human Serum Albumin (HSA) and Hydrophilization of Linker Protein (HSA))

HSA protein surfaces on the sugar chain-bound liposomes prepared by the means in 5.1 above were hydrophilized by the following procedures. 13 mg of tris(hydroxymethyl)aminomethane was added to 2 ml of each sugar chain-bound liposome. The resultant was agitated for 2 hours at 25° C. and then agitated overnight at 7° C. Ultrafiltration was performed using an XM300 membrane and a PBS buffer solution (pH7.2), so as to remove unreacted products. As a result, the final product, 2 ml of a hydrophilized sugar chain-bound liposome complex (total amount of lipid; 2 mg, total amount of protein: 200 μg, and average particle diameter: 100 nm) was obtained.

### (7. Determination of Inhibitory Effect of Various Sugar Chain-Bound Liposome Complexes on Lectin Binding Activity

The in vitro lectin binding activity of each sugar chain-bound liposome complex (prepared by the means of 5.1 and 5.2 above) was determined in an inhibition experiment using a lectin-immobilized microplate according to a standard method (Yamazaki, N. (1999) Drug Delivery System, 14, 498-505). Specifically, a lectin (e.g., E-selectin; R&D Systems Co., U.S.A.) was immobilized on a 96-well microplate. 0.1 μg of biotinylated fucosylated fetuin (ligand for comparison) and each of various sugar chain-bound liposome complexes (amounts of protein: 0.01 μg, 0.04 μg, 0.11 μg, 0.33 μg, and 1 μg) varying in concentration were added to the lectin-immobilized plate, followed by 2 hours of incubation at 4° C. After 3 times of washing with PBS (pH7.2), horse radish peroxidase (HRPO)-conjugated streptavidin was added to the resultants. Incubation was further performed at 4° C. for 1 hour and then the resultants were washed 3 times with PBS (pH7.2). Peroxidase substrate was added, the resultants were allowed to stand at room temperature, and then absorbance was measured at 405 nm using a microplate reader (Molecular Devices Corp., U.S.A.). Fucosylated fetuin was biotinylated as follows. Treatment was performed with a sulfo-NHS-biotin reagent (Pierce Co., U.S.A.) and then purification was performed with Centricon-30 (Amicon Co., U.S.A.). HRPO-conjugated streptavidin was prepared by oxidation of HRPO and conjugation of streptavidin via reductive amination using NaBH₃CN.

### (8. ¹²⁵I Labeling of Various Sugar Chain-Bound Liposomes by Chloramine T Method

A chloramine T (Wako Pure Chemical Co., Japan) solution and a sodium disulfite solution were each prepared at 3 mg/ml and 5 mg/ml when they were used. The sugar chain-bound liposomes and tris(hydroxymethyl)aminomethane-bound liposomes prepared in 6 were separately added (50 μl each) to Eppen tubes. Subsequently, 15 μl of ¹²⁵I-NaI (NEN Life Science Product, Inc. U.S.A.) and 10 μl of a chloramine T solution were added to perform reaction. 10 μl of a chloramine T solution was added every 5 minutes. At 15 minutes after repeating twice this procedure, 100 μl of sodium disulfite was added as a reducing agent so as to stop the reaction. Next, the resultants were placed on a Sephadex G-50 (Pharmacia Biotech. Sweden) column for chromatography and then subjected to elution with PBS, so that labeled products were purified. Finally, an unlabeled-liposome complex was added and then specific activity (4×10⁶ Bq/mg protein) was adjusted. Thus, ¹²⁵I-labeled liposome solutions were obtained.

(9. Measurement of the Amounts of Various Sugar Chain-Bound Liposome Complexes Transferred from the Intestinal Tract into Blood in Mice

0.2 ml each of ¹²⁵I-labeled sugar chain-bound and tris(hydroxymethyl)aminomethane-bound liposome complexes prepared in Example 17 was forcedly administered intraintestinally using oral sonde for mice to male ddY mice (7-week-old) that had been fasted overnight (excluding water), so that the protein amount was 3 μg/mouse. 10 minutes later, 1 ml of blood was collected from inferior vena cava of each mouse under Nembutal anesthesia. 1251 radioactivity in blood was then measured using a gamma counter (Alola ARC300). Furthermore, to examine the in vivo stability of various liposome complexes, the serum of each blood was re-chromatographed using Sephadex G-50. In all cases, most of radioactivity was observed in high-molecular-weight void fractions. Various liposome complexes had stability also in vivo. In addition, each amount of radioactivity that had been transferred from the intestinal tract into blood is represented by the proportion of radioactivity per ml of blood (% dose/ml blood) with respect to the total radioactivity administered.

As a result, the types and binding densities of sugar chains, which were optimum for oral administration, were determined.

### (Summary of Examples 22 and 23)

In vivo assay results of the targeting properties (tropism) of the delivery vehicles prepared based on the rolling model were faithfully reproduced in vitro. Furthermore, a more appropriate delivery vehicle could be selected by taking the binding density of a sugar chain into consideration. Such targeting properties remained unchanged regardless if the relevant delivery vehicle contained a drug or did not contain a drug.

### Example 24

### Preparation of Vitamin A-Encapsulated Liposome, and Determination and Storage Stability of Encapsulated Drug

Next, nutritional elements were delivered using delivery vehicles that had been revealed by the rolling model to be appropriate. A liposome was prepared using cholic acid dialysis. Specifically, dipalmitoylphosphatidylcholine, cholesterol, dicetylphosphate, ganglioside, and dipalmitoylphosphatidylethanol amine were mixed at a molar ratio of 35:40:5:15:5 so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, thereby obtaining a lipid membrane. The thus obtained lipid membrane was suspended in 3 ml of a TAPS buffered saline solution (pH8.4) and then the resultant was ultrasonicated, so that 10 ml of a transparent micelle suspension was obtained. Vitamin A that had been completely dissolved in a TAPS buffer solution (pH8.4) to a concentration of 3 mg/1 ml was slowly added dropwise to the micelle suspension while agitating the suspension. After homogeneously mixing the suspension, the vitamin A-containing micelle suspension was subjected to ultrafiltration using a PM10 membrane (Amicon Co., U.S.A.) and a TAPS buffered saline solution (pH8.4) so that 10 ml of a homogeneous vitamin A-encapsulated liposome particle suspension was prepared. The particle diameter and zeta potential of the vitamin A-encapsulated liposome particles in the thus obtained physiological saline suspension (37° C.) were measured using a zeta potential-particle diameter-molecular weight measurement apparatus (Model Nano ZS, Malvern Instruments Ltd., UK). As a result, the particle diameter ranged from 50 nm to 350 nm and the zeta potential ranged from −30 mV to −10 mV. The amount of the drug encapsulated in the liposome was found by measuring the absorbance at 260 nm. It was revealed that vitamin A was encapsulated at a concentration of approximately 280 μg/ml. The vitamin A-encapsulated liposome remained stable without undergoing precipitation and aggregation even after 1 year of storage in a refrigerator.

Liposomes capable of achieving delivery to desired sites could be prepared using such liposome.

### Example 25

### Rolling Model Using Other Cell Surface Molecules

It was confirmed if similar rolling models could be prepared using P-selectin, L-selectin, galectin 1, galectin 2, galectin 3, galectin 4, galectin 5, galectin 6, galectin 7, galectin 8, galectin 9, galectin 10, galectin 11, galectin 12, galectin 13, galectin 14, mannose-6-phosphate receptor, calnexin, calreticulin, ERGIC-53, VIP53, interleukins, interferons, and growth factors as a cell surface molecule(s) other than E-selectin.

Commercially available products of P-selectin, L-selectin, galectin 1, galectin 2, galectin 3, galectin 4, galectin 5, galectin 6, galectin 7, galectin 8, galectin 9, galectin 10, galectin 11, galectin 12, galectin 13, galectin 14, mannose-6-phosphate receptor, calnexin, calreticulin, ERGIC-53, VIP53, interleukins, interferons, and growth factors can be used. Alternatively, they can be produced by gene engineering techniques.

Galectin 1 is expressed in skeletal muscle, neurons, the kidney, the placenta, and the thymus gland. Galectin 2 is expressed in liver tumors. Galectin 3 is expressed in activated macrophages, eosinophils, neutrophils, mast cells, the small intestine, and epithelial and sensory neurons of respiratory organs. Galectin 4 is expressed in the epithelia of the intestine and oral cavity. Galectin 5 is expressed in erythrocytes and reticulum cells. Galectin 6 is expressed in intestinal epithelia. Galectin 7 is expressed in keratinocytes. Galectin 8 is expressed in the lungs, the liver, the kidney, the heart, and the brain. Galectin 9 is expressed in the liver, the small intestine, the kidney, lymphoid tissues, the lungs, cardiac muscles, and skeletal muscles. They can be useful as indicators for delivery vehicle for use in delivery to these organs.

A mannose-6-phosphate receptor is distributed in the trans-Golgi network of each cell, so that it is useful as an indicator for a delivery vehicle for use in delivery to these cell sites.

Calnexin is distributed in the endoplasmic reticulum, so that it is useful as an indicator for a delivery vehicle for use in delivery to the cell site.

Calreticulin is distributed in the endoplasmic reticulum so that it is useful as an indicator for a delivery vehicle for use in delivery to these cell sites.

ERGIC-53 is distributed in the endoplasmic reticulum to cis-Golgi regions so that it is useful as an indicator for a delivery vehicle for use in delivery to these cell sites.

VIP36 is distributed in the endoplasmic reticulum to cell membrane regions so that it is useful as an indicator for a delivery vehicle for use in delivery to these cell sites.

Siglec1 (sialoadhesin) is distributed in macrophages so that it is useful as an indicator for a delivery vehicle to be used for delivery to these sites.

Siglec2 (CD22) is distributed in lymphocytes (B cells) so that it is useful as an indicator for a delivery vehicle to be used for delivery to these sites.

Siglec3 (CD33) is distributed in myeloid cells so that it is useful as an indicator for a delivery vehicle to be used for delivery to these sites.

Siglec4a (MAG) is present in the peripheral nerve so that it is useful as an indicator for a delivery vehicle to be used for delivery to these sites.

Siglec5 (myelin protein) is present in monocytes so that it is useful as an indicator for a delivery vehicle to be used for delivery to these sites.

N-CAM is distributed in the peripheral nerve, so that it is useful as an indicator for a delivery vehicle to be used for delivery to these sites.

Po (intercellular adhesion factor that is present on mammalian peripheral myelin and mature Schwann cells) is distributed in the peripheral nerve so that it is useful as an indicator for a delivery vehicle to be used for delivery to these sites.

L-selectin is distributed in leukocytes so that it is useful as an indicator for a delivery vehicle to be used for delivery to these sites.

P-selectin is present in vascular endothelial cells so that it is useful as an indicator for a delivery vehicle to be used for delivery to these sites.

A mannose binding protein is present in lymphocytes (natural killer cells) so that it is useful as an indicator for a delivery vehicle to be used for delivery to these sites.

An asialo glycoprotein receptor is distributed in the liver so that it is useful as an indicator for a delivery vehicle to be used for delivery to these sites.

A macrophage mannose receptor is distributed in macrophages so that it is useful as an indicator for a delivery vehicle to be used for delivery to these sites.

Antithrombin (blood coagulation factor) is present in blood so that it is useful as an indicator for a delivery vehicle to be used for delivery to these sites.

FGF is distributed in blood so that it is useful as an indicator for a delivery vehicle to be used for delivery to these sites.

Interleukin2 (IL-2) is distributed in blood so that it is useful as an indicator for a delivery vehicle to be used for delivery to these sites.

Interleukin1α (IL-1α) is distributed in blood so that it is useful as an indicator for a delivery vehicle to be used for delivery to these sites.

Interleukin1β (IL-1β) is distributed in blood so that it is useful as an indicator for a delivery vehicle to be used for delivery to these sites.

Interleukin3 (IL-3) is distributed in blood so that it is useful as an indicator for a delivery vehicle to be used for delivery to these sites.

Interleukin6 (IL-6) is distributed in blood so that it is useful as an indicator for a delivery vehicle to be used for delivery to these sites.

Interleukin7 (IL-7) is distributed in blood so that it is useful as an indicator for delivery vehicle to be used for delivery to these sites.

Tumor necrosis factor α (TNF-α) is distributed in blood so that it is useful as an indicator for a delivery vehicle to be used for delivery to these sites.

If these cell surface molecules are subjected to an experiment according to Example 7 and the result is used as a rolling model, delivery vehicle preferable in vivo in corresponding organs and sites can be searched for.

The relationships between cell surface molecules and organs referred in the Examples can be described as follows. Studies concerning various lectins (sugar chain-recognizing proteins including C-type lectin such as selectin, DC-SIGN, DC-SGNR, collectin, or a mannose binding protein, 1-type lectin such as siglec, P-type lectin such as a mannose-6-phosphate receptor, R-type lectin, L-type lectin, M-type lectin, and galectin) as receptors existing on cell membrane surfaces and the like of various tissues in vivo have been advanced. Sugar chains having various molecular structures are attracting attention as new DDS ligands.

Regarding the relationships between cell surface molecules and organs, for example, cell surface molecules the expression of which in human tissues has been revealed are as listed below:

(1) an asialoglycoprotein receptor, CD11b, CD18, CD22, CD23, CD31, CD69, galectin-5, galectin-10, interleukin-2, a macrophage mannose receptor, N-CAM(CD56), NKR-P1, and sialoadhesin that are expressed in hemocytes and bone marrow cells;
(2) C-reactive protein, P35, mannan-binding lectin, and serum amyloid P that are expressed in plasma and serum;
(3) aggrecan that is expressed in bone and cartilage;
(4) asialoglycoprotein receptor (liver), C-reactive protein (liver), galectin-2 (intestine), galectin-4 and galectin-6 (intestine), galectin-7, HIP and PAP (intestine and pancreas), P35 (liver), serum amyloid P component (liver), surfactant protein A (lungs), and surfactant protein D (lungs) that are expressed in epithelial cells of various tissues;
(5) monkey collectin that is expressed in muscles;
(6) brevican, cerebellar soluble lectin, myelin associated glycoprotein, and N-CAM that are expressed in nerve tissues;
(7) a placenta Gp120 receptor that is expressed in the placenta; and calreticulin, CD44, CD54, ERGIC-53, galectin-1, galectin-3, galectin-8, galectin-9, interleukin 1, phosphomannosyl receptor I, phosphomannosyl receptor II, tetranectin, thrombospondin, tumor necrosis factor, and versican that are expressed not specifically to tissues.

Regarding the relationships between cell surface molecules and disease tissues, expression of E-selectin, P-selectin, and the like in general inflammatory diseases (e.g., encephalitis, chorioretinitis, pneumonia, hepatitis, and arthritis) and diseases that cause inflammation successively (e.g., malignant tumor, rheumatism, cerebral infarction, diabetes, and Alzheimer disease) is being elucidated. Furthermore, expression of E-selectin in cancerous tissues has been reported. Most about the relationships between cell surface molecules and organs remains unknown and the future elucidation thereof is expected.

When animal lectins are classified based on their primary structures, they are classified into the following 14 types of family, for example:

(1) C-type; (2) S-type (galectin); (2) 1-type (Siglec and others); (4) P-type (phosphomannosyl receptor); (5) pentraxin; (6) egg lectin; (7) calreticulin and calnexin; (8) ERGIC-53 and VIP-36; (9) discoidin; (10) fucolectin; (11) annexin lectin; (12) ficolin; (13) tachylectin 5A and 5B; and (14) slug lectin. Furthermore, the C-type family is classified into the following subfamilies: (1) hyalectin; (2) asialoglycoprotein receptor; (3) collectin; (4) selectin; (5) NK group transmembrane receptor; (6) macrophage mannose receptor; and (7) single domain lectin.
Furthermore, although the biological significance has not yet been elucidated, the following members of an orphan lectin group having sugar chain-binding activity are known: (1) amphotericin; (2) CD11b and CD18; (3) CEL-111; (4) complement factor H; (5) Entamoeba adhesion lectin; (6) frog sialic acid-binding lectin; (7) tachylectin-1 and tachylectin-P; (8) tachylectin-2, (9) tachylectin-3; (10) thrombospondin; (11) interleukin-1; (12) interleukin-2; (13) interleukin-3; (14) interleukin-4; (15) interleukin-5; (16) interleukin-6; (17) interleukin-7; (18) interleukin-8; (19) interleukin-12; and (20) tumor necrosis factor.

As the above relationships between a wide variety of animal lectins and organs or diseases have been revealed, it is predicted that the delivery vehicle (e.g., sugar chain-modified liposome) of the present invention will be more useful for treatment and diagnosis of diseases and will be able to be applied to wider application fields. Moreover, also for the purpose of elucidation of the biological significance of various animal lectins, the delivery vehicle of the present invention is useful as a reagent for research or the like.

### Example 26

### Preparation of Ganglioside-Free Sugar Chain-Modified Liposome

### (1. Preparation of Liposome)

A liposome was prepared by the techniques of the previous report (Yamazaki, N., Kodama, M. and Gabius, H.-J. (1994) Methods Enzymol. 242, 56-65) using improved cholic acid dialysis. Specifically, dipalmitoylphosphatidylcholine, cholesterol, dicetylphosphate, and dipalmitoylphosphatidylethanol amine were mixed so that the total amount of lipid was 45.6 mg. 46.9 mg of sodium cholate was added to the mixture and then the resultant was dissolved in 3 ml of a chloroform/methanol solution. The solution was evaporated and then the precipitate was dried in vacuum, thereby obtaining a lipid membrane. The thus obtained lipid membrane was suspended in 3 ml of a TAPS buffer solution (pH8.4) and then the resultant was ultrasonicated, so that a transparent micelle suspension was obtained. Furthermore, the micelle suspension was subjected to ultrafiltration using a PM10 membrane (Amicon Co., U.S.A.) and a PBS buffer solution (pH7.2). Thus 10 ml of a homogeneous liposome (average particle diameter: 100 nm) was prepared.

### (2. Hydrophilization of Liposome Lipid Membrane Surface

10 ml of the liposome solution prepared in 1 above was subjected to ultrafiltration using an XM300 membrane (Amicon Co., U.S.A.) and a CBS buffer solution (pH8.5) and the pH of the solution was adjusted to pH8.5. Next, 10 ml of a cross-linking reagent bis(sulfosuccinimidyl)suberate (BS3; Pierce Co., U.S.A.) was added, followed by 2 hours of agitation at 25° C. Subsequently, the solution was further agitated overnight at 7° C. so as to complete the chemical binding reaction between lipid dipalmitoylphosphatidylethanol amine on the liposome membrane and BS3. The liposome solution was then subjected to ultrafiltration using an XM300 membrane and a CBS buffer solution (pH8.5). Next, 40 mg of tris(hydroxymethyl)aminomethane dissolved in 1 ml of a CBS buffer solution (pH8.5) was added to 10 ml of the liposome solution, followed by 2 hours of agitation at 25° C. The solution was then agitated overnight at 7° C., so as to complete the chemical binding reaction between BS3 bound to the lipids on the liposome membrane and tris(hydroxymethyl)aminomethane. Thus, the hydroxy group of tris(hydroxymethyl)aminomethane was coordinated on the lipid dipalmitoylphosphatidylethanol amine of the liposome membrane, so that the liposome membrane surface was hydrated and hydrophilized.

### (3. Binding of Human Serum Albumin (HSA) to Liposome Membrane Surface)

Human serum albumin (HSA) was bound to the liposome membrane surface by techniques similar to those in Example 3. As a result, human serum albumin (HSA) was not bound to the liposome membrane surface. Through evaluation of the thus obtained delivery vehicle using the rolling model, it could be determined whether or not the delivery vehicle was appropriate vehicle.

### Example 27

### Delivery Vehicle Other than Sugar Chain-Modified Liposome

Next, it was verified whether or not the rolling model can be applied to a delivery vehicle other than the sugar chain-modified liposome.

As substances other than lipids (e.g., liposome), polyester, cyclodextrin, polyamino acid, and silicon were selected and used.

It could be determined whether or not the thus obtained delivery vehicles were appropriate via evaluation based on the rolling model.

As described above, the present invention is exemplified using the preferred embodiments of the present invention. The present invention should not be interpreted as being limited to the embodiments. It is understood that the scope of the present invention should be interpreted based only on the claims. It is understood that persons skilled in the art can implement the present invention within the scope equivalent to the specific preferred embodiments of the present invention based on descriptions of the present invention and technical commonsense. It is also understood that patents, patent applications, and publications cited herein are incorporated herein by reference in their entirety.

### INDUSTRIAL APPLICABILITY

The present invention has usefulness such that a delivery vehicle capable of delivering a substance to a target delivery site can be freely designed. Therefore, the present invention provides a delivery vehicle (e.g., sugar chain-modified liposome) in which a substance (e.g., a drug or a gene) is encapsulated and usefulness (e.g., treatment, diagnosis, prevention, and research) relating thereto.

1.-111. (canceled)

112. A delivery vehicle for achieving delivery to a desired site in which a strong binding IC involved in in vitro affinity for a cell surface molecule associated with the desired site is low and a weak binding IC involved in in vitro affinity for the same is high, and the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.

113. The delivery vehicle according to claim 112, in which the delivery vehicle is a liposome, and the boundary between the strong binding IC and the weak binding IC is between 30 and 31 for “n” of ICn.

114. The delivery vehicle according to claim 113, in which the delivery vehicle is a sugar-chain-modified liposome.

115. The delivery vehicle according to claim 112, in which the desired site is selected from the group consisting of an inflammation site and a cancer site.

116. The delivery vehicle according to claim 112, in which an inhibitory concentration at a strong binding IC which is approximately IC30 or less is 10⁻⁹M or less and an inhibitory concentration at a weak binding IC which is approximately IC31 or more is 10⁻⁹M or more as to in vitro affinity for a lectin associated with a desired state.

117. The delivery vehicle according to claim 112, which satisfies at least one condition selected from the group consisting of a condition that the inhibitory concentration at IC 10 is 10⁻⁹M or less, a condition that an inhibitory concentration at IC20 is 10⁻⁹M or less and a condition that an inhibitory concentration at IC30 is 10⁻⁹M or less for the strong binding IC as to in vitro affinity for a lectin associated with a desired site, and satisfies at least one condition selected from the group consisting of a condition that the inhibitory concentration at IC40 is 10⁻⁹M or more, a condition that the inhibitory condition of IC50 is 10⁻⁹M or more and a condition that the inhibitory concentration at IC60 is 10⁻⁹M or more for the weak binding IC as to in vitro affinity for a lectin associated with a desired site.

118. The delivery vehicle according to claim 112, in which the IC is measured based on affinity for E-selectin.

119. The delivery vehicle according to claim 116, which contains a liposome selected from the group consisting of liposome No. 16, liposome No. 27, liposome No. 29, liposome No. 41, liposome No. 45, liposome No. 53, liposome No. 69, liposome No. 71, liposome No. 76, liposome No. 80, liposome No. 87, liposome No. 91, liposome No. 93, liposome No. 96, liposome No. 105, liposome No. 106, liposome No. 117, liposome No. 125, liposome No. 127, liposome No. 137, liposome No. 139, liposome No. 142, liposome No. 146, liposome No. 150, liposome No. 151, liposome No. 152, liposome No. 153, liposome No. 154, liposome No. 184, liposome No. 186, liposome No. 191, liposome No. 195, liposome No. 199, liposome No. 204, liposome No. 207, liposome No. 213, liposome No. 224, liposome No. 225, liposome No. 229, liposome No. 230, liposome No. 234, liposome No. 235, liposome No. 239, liposome No. 240, liposome No. 263, liposome No. 273, liposome No. 285, and liposome No. 295 shown in Tables 3, 14 and 15.

120. The delivery vehicle according to claim 117, which contains a liposome selected from the group consisting of liposome No. 3, liposome No. 16, liposome No. 27, liposome No. 29, liposome No. 38, liposome No. 40, liposome No. 41, liposome No. 45, liposome No. 50, liposome No. 53, liposome No. 56, liposome No. 60, liposome No. 68, liposome No. 69, liposome No. 70, liposome No. 71, liposome No. 76, liposome No. 80, liposome No. 87, liposome No. 91, liposome No. 93, liposome No. 96, liposome No. 105, liposome No. 106, liposome No. 111, liposome No. 116, liposome No. 117, liposome No. 120, liposome No. 125, liposome No. 127, liposome No. 129, liposome No. 130, liposome No. 137, liposome No. 139, liposome No. 141, liposome No. 142, liposome No. 146, liposome No. 150, liposome No. 151, liposome No. 152, liposome No. 153, liposome No. 154, liposome No. 155, liposome No. 175, liposome No. 178, liposome No. 183, liposome No. 184, liposome No. 186, liposome No. 191, liposome No. 195, liposome No. 197, liposome No. 199, liposome No. 204, liposome No. 207, liposome No. 209, liposome No. 213, liposome No. 218, liposome No. 220, liposome No. 224, liposome No. 225, liposome No. 229, liposome No. 230, liposome No. 233, liposome No. 234, liposome No. 235, liposome No. 236, liposome No. 237, liposome No. 239, liposome No. 240, liposome No. 254, liposome No. 263, liposome No. 273, liposome No. 285, liposome No. 290, liposome No. 292, and liposome No. 295 shown in Tables 3, 14 and 15.

121. A method for producing a delivery vehicle for achieving the delivery of a desired substance to a desired site, which comprises the steps of:
A) measuring in vitro affinity of candidate delivery vehicles for a cell surface molecule associated with the desired site; and
B) selecting a delivery vehicle having in vitro affinity corresponding to delivery to the desired site,
in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.

122. The method according to claim 121, which further comprises the step of causing the selected delivery vehicle to contain a substance to be delivered.

123. The method according to claim 121, in which the delivery vehicle contains a sugar-chain-modified liposome.

124. The method according to claim 121, in which the cell surface molecule comprises a lectin selected from the group consisting of (1)C-type lectins such as Hyalectans, Asialoglycoprotein receptors, Collectins (Mannose-binding protein C, Mannose-binding protein A, Pulmonary surfactant protein A, Pulmonary surfactant protein D, Collectin-K1, and Collectin-L1), DC-SIGN, DC-SIGNR, Selectins (E-Selectin, P-Selectin, or L-Selectin), NK group transmembrane receptors, Macrophage mannose receptor, and Single domain lectins; (2) S-type lectins such as galectins (galectin-1, galectin-2, galectin-3, galectin-4, galectin-5, galectin-6, galectin-7, galectin-8, galectin-9, galectin-10, galectin-11, galectin-12, galectin-13, galectin-14, galectin-15, galectin-16, chicken galectin, congerin-1, and congerin-2); (3) I-type lectins such as siglecs (Siglec-1, Siglec-2, Siglec-3, Siglec-4, Siglec-5, Siglec-6, Siglec-7, Siglec-8, Siglec-9, Siglec-10, Siglec-11, Siglec-12, Siglec-12a, Siglec-12b, Siglec-13, Siglec-E, Siglec-F, Siglec-G, or Siglec-H); (4)P-type lectins such as phosphomannosyl receptors; (5) Pentraxins; (6) Calreticulin or calnexin; (7) ERGIC-53 or VIP-36; (8) Discoidins; (9) Fucolectins; (10) Annexin lectins; (11) Ficolins; (12) Tachylectins 5A or 5B; (13) NCAM or Myelin-associated glycoprotein; (14) Amphoterin; (15) CD11b or CD18; (16) CEL-III; (17) Complement factor H; (18) Oxidized LDL receptor; (19) Endosialin; (20) Tachylectin-1 or tachylectin P; (21) Tachylectin-2; (22) Tachylectin-3; (23) Thrombospondin; (24) Interleukin-1; (25) Interleukin-2; (26) Interleukin-3; (27) Interleukin-4; (28) Interleukin-5; (29) Interleukin-6; (30) Interleukin-7; (31) Interleukin-8; (32) Interleukin-12; and (33) Tumor necrosis factor.

125. The method according to claim 121, in which the cell surface molecule contains E-selectin and the site is selected from the group consisting of a site of the liver, a site of the small intestine, a site of the large intestine, a site of the lymph node, a site of the heart, a site of the pancreas, a site of the lungs, a site of the brain, a site of the bone marrow, a site in blood, a site of the kidney, a site of the spleen, a site of the thymus gland, a site of muscle, an inflammation site, and a cancer site.

126. The method according to claim 121, in which the affinity is represented by n % inhibitory concentration (ICn), wherein “n” ranges from 1 to 99.

127. The method according to claim 121, in which the measurement of affinity comprises measurement at a strong binding IC that is approximately IC30 or less and measurement at a weak binding IC that is approximately IC31 or more.

128. The method according to claim 121, in which the measurement of affinity comprises measurement at a strong binding IC that is at least one point between IC30 and IC10 and measurement at a weak binding IC that is at least one point between IC40 and IC60.

129. The method according to claim 121, in which the measurement of affinity comprises measurement at a strong binding IC that is approximately IC30 or less and a candidate having a low inhibitory concentration at the strong binding IC is selected.

130. The method according to claim 121, in which the measurement of affinity comprises measurement at a strong binding IC that is approximately IC30 or less and a candidate having an inhibitory concentration of 10⁻⁹M or less at the strong binding IC is selected.

131. The method according to claim 121, in which the measurement of affinity comprises measurement at a strong binding IC that is approximately IC30 or less and a candidate having a low inhibitory concentration at the strong binding IC is selected, wherein the selection is performed when at least one condition selected from the group consisting of a condition that the inhibitory concentration at IC 10 is 10⁻⁹M or less, a condition that an inhibitory concentration at IC20 is 10⁻⁹M or less and a condition that an inhibitory concentration at IC30 is 10⁻⁹M or less is satisfied.

132. The method according to claim 121, in which the measurement of affinity comprises measurement at a weak binding IC that is approximately IC31 or more and a candidate having a high inhibitory concentration at the strong binding IC is selected.

133. The method according to claim 121, in which the measurement of affinity comprises measurement at a weak binding IC that is approximately IC31 or more and a candidate having an inhibitory concentration of 10⁻⁹M or more at the weak binding IC is selected.

134. The method according to claim 121, in which the measurement of affinity comprises measurement at a weak binding IC that is approximately IC31 or more and a candidate having a high inhibitory concentration at the weak binding IC is selected, wherein the selection is performed when at least one condition selected from the group consisting of a condition that the inhibitory concentration at IC40 is 10⁻⁹M or more, a condition that the inhibitory concentration at IC50 is 10⁻⁹M or more and a condition that the inhibitory concentration at IC60 is 10⁻⁹M or more is satisfied.

135. The method according to claim 121, in which the measurement of affinity comprises measurement at a strong binding IC that is approximately IC30 or less and a weak binding IC that is approximately IC31 or more and a candidate having a low inhibitory concentration at the strong binding IC and a high inhibitory concentration at the weak binding IC is selected.

136. The method according to claim 121, in which the measurement of affinity comprises measurement at a strong binding IC that is approximately IC30 or less and a weak binding IC that is approximately IC31 or more and a candidate having an inhibitory concentration of 10⁻⁹M or less at the strong binding IC and an inhibitory concentration of 10⁻⁹M or more at the weak binding IC is selected.

137. The method according to claim 121, in which the measurement of affinity comprises measurement at a strong binding IC that is approximately IC30 or less and a weak binding IC that is approximately IC31 or more and a candidate having a low inhibitory concentration at the strong binding IC and a high inhibitory concentration at the weak binding IC is selected,
wherein the selection is performed when at least one condition selected from the group consisting of a condition that the inhibitory concentration at IC10 is 10⁻⁹M or less, a condition that an inhibitory concentration at IC20 is 10⁻⁹M or less and a condition that an inhibitory concentration at IC30 is 10⁻⁹M or less is satisfied; and in terms of the weak binding IC, at least one condition selected from the group consisting of a condition that the inhibitory concentration at IC40 is 10⁻⁹M or more, a condition that the inhibitory concentration at IC50 is 10⁻⁹M or more and a condition that the inhibitory concentration at IC60 is 10⁻⁹M or more is satisfied.

138. The method according to claim 121, in which the measurement of affinity is performed by a method selected from the group consisting of a competitive inhibition assay, a noncompetitive inhibition assay, and a binding assay.

139. The method according to claim 121, in which the delivery vehicle contains a sugar-chain-modified liposome and the analysis of the composition comprises analysis of the sugar chain type and density of the sugar-chain-modified liposome.

140. The method according to claim 121, in which a linker is used for the modification of liposome.

141. The method according to claim 140, in which the linker is a protein.

142. The method according to claim 140, in which the linker is an albumin.

143. The method according to claim 121, which further comprises a step of hydrophilizing the liposome.

144. A pharmaceutical composition, which contains a drug to be used for prevention or treatment, and the delivery vehicle according to claim 112.

145. A pharmaceutical composition, which contains a drug to be used for prevention or treatment, and a delivery vehicle that is produced by the method according to claim 121.

146. A method for preventing or treating a subject who requires delivery of a drug to a desired site, which comprises the steps of:
A) measuring in vitro affinity of candidate delivery vehicles, which are intended for achieving delivery to a desired site, for a cell surface molecule associated with the desired site;
B) selecting a delivery vehicle having in vitro affinity corresponding to delivery to the desired site; and
C) administering a drug required for prevention or treatment to the subject with the use of the selected delivery vehicle,
in which the cell surface molecule is selected from the group consisting of a lectin, an adhesion molecule, an integrin, an immunoglobulin, a sialomucin, a cadherin, a protein, a lipid, a receptor, an antigen, an enzyme, a metalloprotease, a tyrosine phosphatase, a glycolipid, a glycoprotein, a proteoglycan, a costimuratory molecule, a membrane protein, and an extracellular matrix.
